(19) United States
(12) Patent Application Publication
(10) Pub. No.: US 2025/0134651 A1
(43) Pub. Date: May 1, 2025

(54) METHODS AND DEVICES FOR HEART VALVE REPAIR

(71) Applicant: Elixir Medical Corporation, Milpitas, CA (US)

(72) Inventors: Motasim Sirhan, Los Altos, CA (US); John Yan, Los Gatos, CA (US); Vinayak Bhat, Cupertino, CA (US); Joseph Paraschac, Campbell, CA (US); Benjamyn Serna, Gilroy, CA (US)

(73) Assignee: Elixir Medical Corporation, Milpitas, CA (US)

(21) Appl. No.: 17/835,833

(22) PCT Filed: Nov. 18, 2020
(86) PCT No.: PCT/US2020/061109

§ 371 Date: Jun. 8, 2022

Related U.S. Application Data
(60) Provisional application No. 62/937,417, filed on Nov. 19, 2019.
(60) Provisional application No. 63/060,987, filed on Aug. 4, 2020.
(60) Provisional application No. 63/055,020, filed on Jul. 22, 2020.

(51) Int. Cl. A61F 2/24

(52) U.S. Cl. CPC A61F 2/24; A61F 2220/0016

(57) ABSTRACT

An implant and system for reshaping a valve annulus includes an elongate template having one or more anchors which couple directly to a body of the implant or may be indirectly couple through a separate anchor array support. When implanted, the template foreshortens and/or reshapes a peripheral length of the valve annulus can be to improve coaption of native valve leaflets and/or to eliminate or decrease regurgitation of a valve. The template may have one or more skirts augmenting or replacing a defective native valve leaflet function. The template in another example may be coupled to a chordae which is attached to a ventricular wall to improve the heart hemodynamic function. The template may be configured to prevent displacement of an anchor.

### CROSS-REFERENCE TO RELATED APPLICATIONS

This application claims the benefit of U.S. Provisional No. 62/937,417 Attorney Docket No. 32016-717.103), filed Nov. 19, 2019, U.S. Provisional No. 63/055,020 (Attorney Docket No. 32016-722.101), filed Jul. 22, 2020, and U.S. Provisional No. 63/060,987 (Attorney Docket No. 32016-722.102), filed Aug. 4, 2020, the entire contents of which are incorporated herein by reference.

### BACKGROUND OF THE INVENTION

Field of the Invention. The invention generally pertains to mammalian body, specifically pertains to body lumens, vessels, openings, annuli, cavities, or organs. In particular this invention relates to the field of cardiology. More particularly, the invention pertains to heart valves treatment, repair, or replacement. More particularly, the invention pertains to devices and methods for repair of heart valves.

2. Description of the Background Art. Heart valves have important biological function, with a wide range of anatomical configuration including shapes, designs, and dimensions, and are subject to an array of different conditions such as disease conditions that can cause impairment or malfunction. The mitral valve, for example, consists of an annulus containing anterior and posterior leaflets located at the junction between the left atrium and the left ventricle. The valve leaflets are attached to the left ventricle heart papillary muscles via chordae tendineae. Valvular impairment or dysfunction can be caused or exacerbated by changes to the valve configuration including shape, size, and dimension of the valve (or annulus), the length or functionality of the chordae, leaflet function and morphology, or any combination thereof.

An array of open-heart surgical procedures have been utilized, including for example, surgical annuloplasty, implantation of artificial chordae or repair of chordae, and resection leaflet surgical valve repair. These procedures are performed typically via open heart typically using bypass surgery, including opening the patient's chest and heart, a risky and invasive procedure with long recovery times and associated complications. In other cases, the protocols may be on a beating heart, presenting other risks and challenges.

Current less invasive surgical devices and less invasive percutaneous devices are undergoing development to replace or repair the mitral valve. Less invasive surgical and percutaneous options for valve repair typically attempt to replicate more invasive surgical techniques. These devices have the disadvantage of one or more of being large, complex to use, and have limited efficacy or applicability to the various anatomical configurations of valves. Imaging of the devices during deployment has remained a challenge. Results have been typically inferior to open surgical valve repair procedures.

What is needed is a device that can be incorporated into less invasive surgical and percutaneous techniques, address valve regurgitation, minimize or eliminate device migration, and that is applicable to broader patient population having various valve configurations while managing the limits of current imaging technology. The invention meets at least some of these needs.

### SUMMARY OF THE INVENTION

The present invention comprises devices and methods for less invasive surgical and/or percutaneous treatment or repair of a body organ, lumen, cavity, or annulus. In a preferred example, the present invention comprises devices and methods for open surgical, less invasive surgical, and percutaneous treatment or repair of heart valves comprising valve annulus and valve leaflets. An example of heart valves comprises aortic, mitral, pulmonary, and tricuspid valves. Although certain examples show a specific valve, the inventions described and claimed herein are applicable to all valves in the body and additionally other body annulus, lumen, cavity, and organs.

In general, the present invention provides implants and methods of using such implants for reshaping a valve annulus, typically a cardiac valve annulus, and more typically a mitral valve annulus, to improve coaption of the valve leaflets and treat valve regurgitation. The implants usually comprise a pre-shaped metallic template having a length in an axial direction and a width in a transverse direction, often comprising or consisting of metal ribbons formed to have one or more of a variety of features as described in detail herein. The metallic templates will usually be elastic and be pre-shaped by conventional metal-shaping to have specific geometries as described to reshape the valves annuluses is particular ways, as described in detail herein. The geometries will typically comprise at least one concavity at a center of the metallic template flanked on each side by a convexity. The convexities are typically formed as right and left arms extending in right and left directions from the center concavity, where the concavity usually has a concave surface configured to be positioned against a peripheral wall of the valve annulus and the convexities have convex surfaces configured to be positioned against regions of the peripheral wall adjacent to the convexity. The metallic template will be secured to a wall of the valve annulus, typically a peripheral wall, at one or more locations distributed along the length of the metallic template. The locations usually include at least a center of the metallic template, typically at the concavity, often also including one or more locations spaced-apart along the right and left arms, including but not limited to the right and left ends or extremities of the arms.

The right and left arms forming convexities on each side of the center will follow a generally curved or everting path which may be continuous (free from disruptions and discontinuities) or may have secondary features and discontinuities, such as local concavities, sinusoidal regions, serpentine regions, and the like. In many embodiments, the implant may comprise two or more concavities separated by intermediate convexities and optionally terminating on each end with right and left arms or other convexities.

In other embodiments, the pre-shaped metallic template may consist of a single concavity flanked by one right arm or other convexity and one left arm or other convexity with optional tethers, extensions, and/or other features coupled to the ends or extremities of either or both of the left and right arms. The distance between the ends of the convexities will often be selected to span at least most of and often all of the peripheral wall of the valve annulus, particularly a mitral valve annulus. For example, a metallic template consisting of a single concavity flanked by left and right convexities may span the peripheral wall of the mitral valve annulus from a right fibrous trigonal region to a left fibrous trigonal region, either with or without extensions, tethers, and other features as described below.

In a first aspect, the present invention provides an implant comprising an implant body, an anchor array support configured to be attached to the implant body, and one or more tissue anchors operatively coupled to the anchor array support. The implant body is typically configured for engagement against a tissue surface, such as a cardiac or other valve, for example to deform the valve annulus to enhance coaption of the valve leaflet to treat valve regurgitation or other conditions.

In particular embodiments, the implant may have a central portion and a peripheral portion. In such instances, the anchor array support is typically configured to be attached to the central portion of the implant body and typically comprises one or more individual anchors attachable to the peripheral portion of the implant.

In particular embodiments, the implant may comprise a metallic template configured to engage and deform the tissue surface where the metallic template is usually pre-shaped to draw at least one segment of a peripheral wall of a valve annulus into a concavity to thereby reduce a diameter of said annulus in a radially inward direction where the metallic template is deployable to its pre-shaped configuration from a crimped configuration.

In particular embodiments, the anchor array support comprises an array support base where at least some of the anchors typically comprise elongate attachment members having one end configured to penetrate tissue and at least one of the elongate attachment members has another end configured to be attached to a driver. For example, each of the elongate attachment members may have another end configured to be attached to a driver and wherein each of the elongate attachment members may be rotated by a respective driver. Alternatively, only a single one of the elongate attachment members may have another end configured to be attached to a driver, wherein the remaining elongate attachment members are mechanically coupled to the single elongate attachment member to rotate therewith, and wherein all the elongate attachment members may be rotated by a single driver attached to the single one of the elongate attachment members. Usually, the elongate attachment members comprise helical connectors.

The implants as just described may be incorporated into a variety of implant deliver systems. In some instances, the implant delivery system comprises a plurality of driver releasably attachable to a plurality of the elongate attachment members. In other instances, the implant delivery system comprises a single one of the elongate attachment members.

In a second aspect, the present invention provides method for securing an implant body to a target site on a tissue surface an anchor array support comprising a plurality of tissue anchors is advanced to the target site. The plurality of tissue anchors is penetrated into tissue at the target site to secure the anchor array support to said target site such that the implant body is secured to the target site by means of the anchor array support.

In particular embodiments, the implant body may be attached to the anchor array support prior to advancing the anchor array support to the target site. In alternative embodiments, the implant body is attached to the anchor array support after the anchor array support has been secured to the target site. In still other instances, the anchor array support is advanced over at least one driver attached to a tissue anchor.

In particular embodiments, at least one driver is actuated to penetrate the plurality of tissue anchors into tissue at the target site, for example, where the at least one driver is mechanically coupled to actuate each of the plurality of tissue anchors simultaneously.

In particular embodiments, the anchor array support may be advanced by a plurality of drivers attached to the plurality of tissue anchors, for example, where each of the plurality of drivers is actuated to penetrate the plurality of tissue anchors into tissue at the target site.

In particular embodiments, the implant body may comprise an implantable template having a tissue-engaging surface. For example, the implant body may be pre-shaped with at least one concavity and may be expanded with an open end of the at least one concavity oriented against a peripheral surface of a valve annulus, and at least one segment of the peripheral surface of the valve annulus may be drawn into the concavity to reduce a diameter of said valve annulus.

In a third aspect, the present invention provides an implant for reshaping a valve annulus comprising a pre-shaped metallic template having a length in an axial direction and at least one concavity in a lateral direction along said length. The concavity may have a concave surface configured to be positioned adjacent to a peripheral wall of the valve annulus, and at least two anchors may be coupled to the pre-shaped metallic template near a center thereof and extend along a lateral axis between a pair of opposed legs. The at least two anchors may be configured to penetrate into said at least one segment of said peripheral wall of the valve annulus and to draw said at least one segment of said peripheral wall into said concavity to thereby reduce a diameter of said annulus in a radially inward direction, where said template is typically deployable to said pre-shaped configuration from a crimped configuration.

In particular embodiments, the implant may further comprise at least three anchors coupled to the pre-shaped metallic template near a center thereof as well as near an anchor location at a tissue-engaging end of each leg. The at least two anchors typically comprise helical anchors rotatably secured in a segment of the pre-shaped metallic template and/or to a body of the pre-shaped metallic template.

In particular embodiments, the pre-shaped metallic template has a single concavity joined by a single curved junction region, where at least two anchors may be configured to rotate about a lateral axis but not translate along the lateral axis relative to the pre-shaped metallic template. For example, at least one anchor may be configured to both rotate about a lateral axis and translate along the lateral axis relative to the pre-shaped metallic template.

In a fourth aspect, the present invention provides systems comprising any one of the implant described above in combination with a driver configured to detachably attach to and rotate at least one of the helical anchors to drive the distal tip of the helical anchor into the annulus and draw at least a segment of an inner surface of the annulus into the concavity.

In particular embodiments of these system, the template may be translatably coupled to the detachable driver and configured to be moved distally relative to the detachable driver to couple with the anchor. Alternatively, the template may be pivotally coupled to the detachable driver and configured to be pivoted relative to the detachable driver to facilitate implantation and/or the template may be rotatably coupled to the anchor.

In a fifth aspect, the present invention provides an implant for reshaping a valve annulus. The implant comprises a pre-shaped metallic template having a length in an axial direction and at least one concavity in a lateral direction along said length. The concavity typically has a concave surface may be configured to be positioned adjacent to a peripheral wall of the valve annulus. First and second anchors may be coupled to the pre-shaped metallic template near a center thereof, where the first anchor is pre-positioned to extend along a lateral axis between a pair of opposed legs and the second anchor is configured to be advanced from a retracted position to an extended position along the lateral axis between the pair of opposed legs. The first and second anchors may be configured to sequentially penetrate into said at least one segment of said peripheral wall of the valve annulus and to draw and stabilize said at least one segment of said peripheral wall into said concavity to thereby reduce a diameter of said annulus in a radially inward direction. Usually, the template is deployable to said pre-shaped configuration from a crimped configuration.

In particular embodiments of the implants, an anchor located at a tissue-engaging end of at least one of said legs, typically at each leg. The first and second anchors may each comprise a helical anchor rotatably secured to the center of the pre-shaped metallic template, where the first anchor is typically axially fixed relative to the pre-shaped metallic template and the second anchor is configured to translate along the lateral axis between the pair of opposed legs. In many instances, the pre-shaped metallic template has a single concavity joined by a single curved junction region.

In a sixth aspect, the present invention provides systems comprising an implant as just described and a driver configured to detachably attach to and individually rotate each of the first and second helical anchors to drive the distal tip of the first helical anchor into the annulus and draw at least a segment of an inner surface of the annulus into the concavity and to thereafter advance and drive the tip of the second anchor into said annulus.

In a seventh aspect, the present invention provides an implant and delivery system for reshaping a valve annulus. The implant delivery system comprises a locating tissue anchor detachably secured to a distal end of a locating control wire and a plurality accessory tissue anchors on a frame detachably coupled to a plurality of accessory control wires. A pre-shaped metallic template has a length in an axial direction and at least one concavity in a lateral direction along said length where the concavity has a concave surface configured to be positioned adjacent to a peripheral wall of the valve annulus. The frame and accessory tissue anchors are typically configured to be advanced over the locating control wire and embedded in tissue after the locating tissue anchor has been embedded at a tissue target site and wherein the pre-shaped metallic template is configured to be advanced over at least one of the locating control wire and the accessory control wires and coupled to the frame after the frame an and accessory tissue anchors are embedded in tissue.

In an eighth aspect, the present invention provides an implant comprising a pre-shaped metallic template having a length in an axial direction and at least one concavity in a lateral direction along the length configured for engagement against a tissue surface. A helical anchor array is rotatably coupled to the pre-shaped metallic template, and the helical anchor is rotatably positioned in a helical track in a wall of the pre-shaped metallic template so that a tissue-penetrating distal tip of the helical anchor can be advanced into a region between a pair of legs of the pre-shaped metallic template by rotation.

Such implants may further comprise a dock element on a proximal end of the helical anchor, wherein the dock element is configured to detachably engage a rotatable driver. The helical anchor may have a proximal straight section that decouples from the helical track to allow the helical anchor to be further rotated to cinch implantation after the helical portion of the anchor has passed distally beyond the wall.

In a ninth aspect, the present invention provides an implant for repairing a valve annulus. The implant comprises a pre-shaped metallic template having at least one concavity configured to be positioned adjacent to a peripheral wall of the valve annulus. At least one anchor is configured to be coupled to the pre-shaped metallic template and to draw at least one segment of said peripheral wall of the valve annulus into said concavity to thereby reduce a diameter of said annulus in a radially inward direction. A “skirt” is attached to the pre-shaped metallic template on a side opposite to that of the concavity. The skirt may take a variety of forms but in many or most of the forms will have a surface configured to engage one or more apposed native valve leaflets during systole when the pre-shaped metallic template is positioned adjacent to the peripheral wall of the valve annulus. The apposition between the native valve leaflet and the skirt will enhance sealing of the valve annulus, thus inhibiting regurgitation.

In particular embodiments, the skirt may have a length in a direction away from the side opposite to that of the concavity, where the length is sufficient to coapt with one or more native valve leaflets in apposition to the skirt. The skirt may have a pliability and length in a direction away from the side opposite to that of the concavity to fold down act as a prosthetic valve leaflet during systole and diastole. Alternatively, the skirt constructed of a semi-rigid or flexible material that is biocompatible and hemo-compatible.

In some instances, the pre-shaped metallic template may have a surface along a length in an axial direction, wherein the concavity is formed in said surface and oriented in a lateral direction relative to said length. The pre-shaped metallic template may be deployable to said pre-shaped configuration from a crimped configuration.

In some instances, the implant may further comprise a tether coupled at one end thereof to at least one of the pre-shaped metallic templates, the anchor, and the skirt, and have another end configured to be implanted in a tissue surface. In particular instances, the tether may be coupled to a free distal end of the skirt. In other instances, the tether may be coupled to a non-sealing surface of the skirt, the tether may be coupled to the at least one anchor, and/or the tether may be coupled to the pre-shaped metallic template. In particular instances, the other end of the tether may be configured to be implanted in an apex of a heart chamber; the other end of the tether may be configured to be implanted in a wall of a heart chamber; the other end of the tether may be configured to be implanted in the peripheral wall of the valve annulus at a location diametrically opposite to a location of the pre-shaped metallic template, and/or the other end of the tether may be configured to be implanted in one of the wall of the ventricle, an annulus, a papillary muscle, a fibrous trigone, a septum, and the wall of the aorta

The tether may have a variety of forms, typically being one or a combination of a metal wire, a metal filament, a polymeric filament, a ePTFE filament, a Dacron filament, a nylon filament, a polypropylene filament, a silk filament, or the like, where filament is understood to encompass both monofilament and multifilament braided constructions, and composites of these materials.

In particular embodiments, the pre-shaped metallic template may have a single concavity with a pair of opposed legs disposed about a lateral axis and joined by a curved junction region. Each of the opposed legs each may have a convex surface which is axially and laterally spaced-apart from the concavity, and the at least one anchor on the template may be further configured to draw adjacent segments of said peripheral wall of the valve annulus against the convex surfaces. One or more additional anchors may located along each of the convex surfaces of the opposed legs.

In particular embodiments, the pre-shaped metallic templates may have at least two concavities separated by a convexity. Each such concavity may have at least one anchor configured to draw at least one segment of said peripheral wall of the valve annulus into said concavity, for example, all or a portion of a posterior mitral valve annulus.

In particular embodiments, the pre-shaped metallic template may comprise an elongate structure having a length in a range from 10 mm to 30 mm and/or a width of the concavity in a range from 1 to 5 times a depth of the concavity.

In particular embodiments, the at least one anchor may comprise a helical anchor having a distal end and a proximal end. The distal end may have a sharpened tip, and said proximal end may be rotatably secured in the concavity of the template. The anchor may be configured to be coupled to the tissue while the template is coupled to the anchor, or the anchor may be configured to be coupled to the tissue before the anchor is coupled to the template.

In a tenth aspect, the present invention provides a system comprising the tethered implants as just described in combination with a driver configured to detachably attach to and drive the distal tip of the at least one anchor into the annulus and draw the at least one segment of the peripheral wall of the annulus into the concavity. The template may be slidably coupled to the detachable driver and can be moved distally relative to the detachable driver to couple with the anchor and/or the template may be rotatably coupled to a helical anchor.

In an eleventh aspect, the present invention provides a method for repairing a valve annulus. The method comprises delivering in a crimped configuration a metallic implantable template having a tissue-engaging surface pre-shaped with at least one concavity. The template with an open end of the at least one concavity oriented against a peripheral surface of the valve annulus. At least one segment of the peripheral surface of the valve annulus is drawn into the concavity to reduce a diameter of said valve annulus, and a skirt is employed from a side of the pre-shaped metallic template opposite to that of the concavity. The skirt has a surface configured to seal against one or more apposed valve leaflets during systole.

In particular embodiments of these methods, the skirt may have a length in a direction away from the side opposite to that of the concavity which is sufficient to coapt with one or more native valve leaflets in apposition to the skirt. The skirt may have a pliability and length in a direction away from the side opposite to that of the concavity to fold down act as a prosthetic valve leaflet during systole and diastole. The skirt may be constructed of a semi-rigid or flexible material that is biocompatible and hemo-compatible.

In particular embodiments of these methods, the pre-shaped metallic template may have a surface along a length in an axial direction, where the concavity is formed in said surface and oriented in a lateral direction relative to said length. The pre-shaped metallic template may be deployable to said pre-shaped configuration from a crimped configuration.

In particular embodiments of these methods, an anchor end of a tether may be coupled at another end to at least one of the pre-shaped metallic templates, the at least one anchor, and the skirt in a tissue surface. The tether may be coupled to a free distal end of the skirt. The tether may be coupled to a non-sealing surface of the skirt. The tether may be coupled to the at least one anchor. The tether may be coupled to the pre-shaped metallic template.

In particular embodiments of these methods, the other end of the tether may be configured to be implanted in an apex of a heart chamber. The other end of the tether may be configured to be implanted in a wall of a heart chamber. The other end of the tether may be configured to be implanted in the peripheral wall of the valve annulus at a location diametrically opposite to a location of the pre-shaped metallic template.

In particular embodiments, these methods may further comprise drawing at least one segment of the peripheral surface of the valve annulus into the concavity aligns the template with the valve annulus. Drawing at least one segment of the peripheral surface of the valve annulus into the concavity comprises may comprise engaging an anchor against the annulus segment to apply tension or compression to draw said annulus segment into said concavity.

In particular embodiments of these methods, the anchor may comprise a helical coil and drawing comprises rotating the helical coil to penetrate the peripheral surface of the valve annulus. The helical coil may be detachably attached to a driver and rotating the helical coil comprises rotating the driver. The metallic implantable template may be slidably coupled to said driver and said method further comprises applying tension to said driver and said helical coil to draw said annulus segment into said concavity.

In particular embodiments, these methods may further comprise locking the template to the helical coil after the annulus segment has been drawn into said concavity the driver may be advanced and rotated to implant the helical coil in the valve annulus, the template then advanced over the driver and coupled to the helical coil after the coil has been implanted in the valve annulus, and the driver then detached from the coil after the template has been advanced over the shaft and coupled to the coil.

In particular embodiments of these methods, the anchor may comprise a helical coil rotatably attached to the template and drawing comprises rotating the helical coil so that the tissue is drawn into the concavity while the anchor remains attached to the template. The template may be constrained in the crimped configuration and expanding comprises releasing the template from constraint. The peripheral surface may include at least a portion of a mitral valve annulus, tricuspid valve annulus, an aortic valve annulus, or a pulmonary valve annulus. Drawing may consist of drawing a single segment of the peripheral surface of the annulus into a single concavity on a single template. Alternatively, drawing may comprise drawing at least two segments of the peripheral surface of the annulus into at least two concavities on a single template and engaging the template against the peripheral surface of the valve annulus may comprise intravascularly advancing the template.

In one example, at least one tissue anchor placed in the annular region of a valve is coupled to a valve replacement implant. In a further example, the valve replacement implant comprises valve leaflets, a support cage, and a sealing skirt. In a further example, the anchor is connected to the valve cage by sutures. In another example, the anchor is connected to the valve skirt by sutures. In another example, the anchor is connected to a magnet arranged to be attracted to a corresponding magnet connected to the valve cage. In another example, the anchor is connected to a magnet arranged to be attracted to a corresponding magnet connected to the valve skirt. In another example, the corresponding magnet can be tracked by magnetic tracking technology. In a further example, one of the magnets is replaced by a magnetic material which is attracted to the remaining magnet. In a further example, the magnet can be removed, leaving the anchor behind. In yet another example, the anchor is connected to an endoscope tip that allows the visualization of the target anatomy. In another example, the endoscope has a lens tip that can be pressed on the tissue to identify the annulus. In another example, the endoscope has a balloon tip that can be pressed on the tissue. In another example, the anchor is positioned on the tip such that it can be placed in the annulus region once it has been identified. In another example, the endoscopic tip has a port that extrudes saline or the like to displace blood and make the adjacent anatomy clear. In another example, an ultrasonic probe can be place near the anchor to identify the annulus.

In still further aspects, the present invention provides a method for securing an implant body to a target region on a valve annulus. The method comprises attaching at least one primary tissue anchor to a target site in the tissue region and advancing the implant to target region. The implant typically has a center, a right arm, and a left arm, and the center of implant can be docked to said at least one center tissue anchor. The center of the implant body will be further docked or otherwise attached to at least one accessory tissue anchor to in the tissue region, and the right and left arms are anchored to locations on the target region on each side of the target site, typically one location on the right side and one location on the left side.

In particular embodiments, anchoring the right and left arms to said locations on the target region on each side of the target site comprises anchoring each arm at at least two sites on the bright and left sides of the center. For example, anchoring the right and left arms to said two locations on the target region on each side of the target site may comprise first anchoring a laterally outward position on each arm, typically at an end or extremity of the arm, and thereafter anchoring a laterally inward position on each arm, typically often but not necessarily midway between the center and the extremity, wherein anchoring the laterally inward positions causes the valve annulus to deform.

In particular embodiments, advancing the implant to the target region may comprise advancing the center of the implant over a primary driver attached to said at least one primary tissue anchor. In such instances, the primary driver may be actuated to attach the primary anchor before the implant is advanced over the primary driver, where the center of the implant fixes to the primary anchor after the implant has been fully advanced.

In particular embodiments, advancing the implant to the target region may further comprise advancing the center over an accessory driver coupled to the accessory anchor. The accessory driver may then be actuated to attach the accessory anchor after the implant is attached to the primary anchor.

In particular embodiments, the implant body may comprise an implantable template having a tissue-engaging surface, where the implant body may be pre-shaped with at least one concavity at the center of the implant. The primary and accessory anchors are typically fixed to the at least one concavity, and distal portions of the right and left arms are typically anchored to trigonal regions of the valve annulus.

In still further aspects, the present invention provides a method for securing an implant body to a target region on a valve annulus, where the method comprises attaching at least one tissue anchor to a target site in the tissue region. The implant is advanced to the target region, where said implant has a center, a right arm, and a left arm. The center of implant is docked to said at least one tissue anchor, and at least a second tissue anchor coupled to said implant body is attached to said target site in the tissue region. The right and left arms are then anchored to said locations on the target region on each side of the target site.

In still further aspects, the present invention provides a method for securing an implant body to a target region on a valve annulus, where the method comprises attaching at least one tissue anchor to a target site in the tissue region. The implant is advanced to target region, where the implant has a center, a right arm, and a left arm and where the right and left arms are constrained as the implant is advanced. The center of implant is docked to said at least one tissue anchor, and at least a second tissue anchor coupled to said implant body is attached to said target site in the tissue region. The right and left arms from are released from constraint after the center has docked to both tissue anchors, enabling the right and left arms deploy toward or against locations on the target region on each side of the target site. The right and left arms may then be anchored to said locations on the target region on each side of the target site.

In still further aspects, the present invention provides, a method for securing an implant body to a target region on a valve annulus comprises attaching at least two tissue anchors to a target site in the tissue region. The implant is attached to a site in the target region, where said implant has a center, a right arm, and a left arm and where the right and left arms are constrained as the implant is advanced. The center of implant is docked to said at least two tissue anchors, and the right and left arms are released from constraint after the center has docked to the at least two tissue anchors. The right and left arms can then deploy toward or against locations on the target region on each side of the target site, and the right and left arms can be anchored to said locations on the target region on each side of the target site.

In still further aspects, the present invention provides a method for securing an implant body to a target region on a valve annulus. The method comprises attaching at least three tissue anchors to at least three spaced-apart target sites spanning the target region from a first end to a second end. The implant is advanced to the target region, where the implant has a center, a right arm, and a left arm. The center of implant is docked to a center one of the at least three anchors, the right arm is docked to a right-most one of the at least three anchors, and the left arm is docked to a left-most one of the at least three anchors.

In particular embodiments, attaching the at least three tissue anchors comprises positioning the at least three tissue anchors with a positioning template or a positioning guide. Alternatively, attaching the at least three tissue anchors may comprise using a probe to place marks at target locations for at least the three anchors. In all cases, attaching the at least three tissue anchors typically comprises implanting the anchors with at least three elongated drivers detachably coupled to the at least three tissue anchors.

In particular embodiments, advancing the implant to the target region comprises sliding the center, the right arm, and the left arm of the implant over the elongate drivers while the elongate drivers remain coupled to the at least three tissue anchors.

In particular embodiments, docking the implant with the anchors comprises coupling a feature on at least one of the center, right arm, and left arm of the implant with a mating feature on at least one of the three anchors. Alternatively, docking the center, right arm, and left arm of the implant to the anchors may comprise securing a fastener between at least one of the anchors and at least one of the center, right arm, and left arm of the implant.

In particular embodiments, the right and left arms are anchored at a laterally outward ends on each arm remote from the center anchor. Anchoring may further comprise anchoring each of the right arm and the left arm at one or more locations intermediate the center and the laterally outward positions to cause the valve annulus to deform. The implant body may comprise an implantable template having a tissue-engaging surface, where the implant body may be pre-shaped with at least one concavity at the center of the implant and the laterally outward ends the right and left arms may be anchored to trigonal regions of the valve annulus.

In particular embodiments, the right and left anchors may be initially positioned at a first distance anterior to the center anchor, where coupling the right and left arms to the right and left anchors may displace the right and left anchors to a second distance anterior to the center anchor, said second distance being shorter than said first distance.

In particular embodiments, the right and left anchors may be initially positioned anterior to the center anchor and wherein coupling the right and left arms to the right and left anchors positions the right and left anchors posterior to the center anchor.

In particular embodiments, the right and left anchors may be initially positioned anterior to the center anchor and wherein coupling the right and left arms to the right and left anchors aligns the right and left anchors with the center anchor in an anterior-posterior direction.

In particular embodiments, the right and left anchors may be initially positioned posterior to the center anchor and wherein coupling the right and left arms to the right and left anchors positions the right and left anchors anterior to the center anchor.

In still further aspects, the present invention provides an implant for reshaping a valve annulus. The implant typically comprises a pre-shaped metallic template having a length in an axial direction, at least one concavity, and right and left arcuate convexities on each side of each concavity. The at least one concavity and the right and left arcuate convexities may be disposed along said length. The concavity may have a concave surface, and the arcuate convexities may each have one or more convex surfaces configured to be positioned adjacent to a peripheral wall of the valve annulus. At least one anchor may be coupled to the pre-shaped metallic template near the concavity and may be configured to secure the concave surface to tissue. One or more support extensions along the convexities extending in a transverse direction, and at least two anchors are typically configured to be coupled to each of the right and left convexities and configured to attach tissue to the convex surfaces.

In particular embodiments, the support extensions may comprise protrusions formed in a body of the pre-shaped metallic template and extending is an atrial direction when the implant is implanted in the valve annulus, and at least two inner anchors may be configured to be coupled proximate to the protrusions formed in the body of the pre-shaped metallic template and at least two trigonal anchors may be configured to be coupled proximate an outer end of each convexity.

In still further aspects, the present invention provides an implant for reshaping a valve annulus. The implant comprises a pre-shaped metallic template having a length in an axial direction, at least one concavity, and right and left convexities on each side of each concavity. The at least one concavity and the right and left convexities are typically disposed along said length, said concavity has a concave surface, and said convexities each have one or more convex surfaces configured to be positioned adjacent to a peripheral wall of the valve annulus. At least one center anchor is typically coupled to the pre-shaped metallic template near the concavity and configured to secure the concave surface to tissue, and right and left anchors are typically coupled to the implant to the right and left of the center anchor and configured to couple tissue to the implant.

In particular embodiments, one or more of the convexities may be defined by curved legs extending from an adjacent concavity, and at least some of the curved legs may have one or more concave regions within a convex surface. In such instances, the concavity may comprise a curved junction region between a pair of adjacent curved legs or alternatively may comprise an angular junction region between a pair of adjacent curved legs. In such instances, at least some of the right and left convexities may have at least one terminal anchor at a free end thereof and one anchor between the terminal anchor and an adjacent concavity.

In particular embodiments, at least two anchors may be coupled to the pre-shaped metallic template near the concavity and configured to secure the concave surface to tissue.

In particular embodiments, at least some of the anchors may comprise a helical anchor having a distal end and a proximal end, said distal end having a sharpened tip and said proximal end configured to couple to the template. Such helical anchors may be configured to be coupled to the tissue while the template is coupled to the anchor or may be configured to be coupled to the tissue before the anchor is coupled to the template.

In particular embodiments, the implant may further comprise at least one trigonal anchor coupled to one end of the pre-shaped metallic template. The at least one trigonal anchor may be coupled to the one end of the pre-shaped metallic template by a control wire configured to adjust a tension between the at least one trigonal anchor and the pre-shaped metallic template, and at least one trigonal anchor may be coupled to the one end of the pre-shaped metallic template by an extension configured to slidably position the at least one trigonal anchor relative to the pre-shaped metallic template.

Any one or more of these implants may be incorporated in systems further comprising a driver configured to detachably attach to and rotate the helical anchor to drive the distal tip of the helical anchor into the annulus and draw at least a segment of an inner surface of the annulus into the concavity. The template may slidably coupled to the detachable driver so that it can be moved distally relative to the detachable driver to couple with the anchor. Alternatively or additionally, the template may rotatably coupled to the anchor.

In still further aspects, the present invention provides an implant for reshaping a valve annulus. The implant comprises a pre-shaped metallic template having a length in an axial direction, at least one concavity, and right and left convexities on each side of each concavity. The at least one concavity and the right and left convexities are disposed along said length, the concavity has a concave surface, and the convexities each have one or more convex surfaces configured to be positioned adjacent to a peripheral wall of the valve annulus. At least one center anchor may be coupled to the pre-shaped metallic template near the concavity and may be configured to secure the concave surface to tissue. At least one lateral anchor may be coupled to each of the right and left convexities and may be configured to couple tissue to the convex surfaces wherein the at least one anchor is disposed in a concave region within the convex surface.

In still further aspects, the present invention provides a method for securing an implant body to a tissue region on a mitral valve annulus. The method comprises placing at least three tissue anchors in the tissue region, wherein a right lateral anchor is placed proximate the P1 leaflet of the mitral valve, a left lateral anchor is placed proximate the P3 leaflet of the mitral valve, and a center anchor is placed on a posterior of the mitral valve annulus proximate the P2 leaflet. The implant may be advanced to the tissue region, wherein said implant has a center, a right arm, and a left arm. The center of the implant is attached the center tissue anchor. The right arm of the implant is attached to the right lateral tissue anchor, and the left arm of the implant is attached to the left lateral tissue anchor. In this way, a shape of the implant deforms the annulus to reduce a diameter of said mitral valve annulus in a radially inward direction.

In particular embodiments, the template has a height from its center to coupling points of the left and right lateral anchors in a range from 5% to 50% of a minor axis of the valve. The right and left arms are typically each attached to valve annulus at locations between the center anchor and the right and left lateral tissue anchors, respectively. A position of at least one the right and the left lateral tissue anchors may be controlled by tensioning at least one control wire extending from at least one of the right and left arms to at least one the right and the left lateral tissue anchors. Alternatively, the position of at least one the right and the left lateral tissue anchors may be controlled by sliding at least one extension extending from at least one of the right and left arms to at least one the right and the left lateral tissue anchors.

In particular embodiments, at least some of the tissue anchors may be placed prior to attachment of the implant. In other embodiments, at least some of the tissue anchors may be attached to the implant prior to placement on the valve annulus.

In particular embodiments, advancing the implant to the target region may comprise advancing the implant over at least one driver attached to at least one of the tissue anchors. In some instances, advancing the implant to the target region may comprise advancing the implant over at least three drivers attached to the at least three tissue anchors. In some instances, at least one driver may be actuated to attach the anchor before the implant is advanced over the at least one driver, wherein the implant fixes to the anchor after the implant has been fully advanced.

In particular embodiments, the implant body may comprise an implantable template having a tissue-engaging surface. In some instances, the implant body may be pre-shaped with at least one concavity at the center of the implant.

In particular embodiments, the lateral anchors may be positioned by comparing the length between lateral anchors coupling points along the implant to the length between lateral anchors along the valve annulus. For example, the length between lateral anchors along the valve annulus may be selected to be between 100% and 120% of the length between lateral anchors coupling points along the implant. Alternatively, the length between lateral anchors along the valve annulus is between 90% and 130% of the length between lateral anchors coupling points along the implant.

In still further aspects, the present invention provides a method for securing an implant body to a tissue region on a mitral valve annulus. The method comprises placing at least three tissue anchors in the tissue region, wherein a right trigonal anchor is placed proximate a right fibrous trigone of the mitral valve annulus, a left trigonal anchor is placed proximate a left fibrous trigone of the mitral valve annulus, and a center anchor is placed on a posterior of the mitral valve annulus between the fibrous trigones. The implant is advanced to the target region, where the implant has a center, a right arm, and a left arm. The center of the implant is attached the center tissue anchor. The right arm of the implant is attached to the right trigonal tissue anchor, and the left arm of the implant is attached. A shape of the implant deforms the annulus to reduce a diameter of said mitral valve annulus in a radially inward direction.

In particular embodiments, the template may have a height from its center to coupling points of the left and right trigonal anchors in a range from 25% of a minor axis of the valve to 75% of the minor axis of the valve. In some instances, the right and left arms are each further attached to valve annulus at locations between the center anchor and the right and left trigonal tissue anchors, respectively.

In particular embodiments, the methods further comprise controlling a position of at least one the right and the left trigonal tissue anchors by tensioning at least one control wire extending from at least one of the right and left arms to at least one the right and the left trigonal tissue anchors. Alternatively, the methods may further comprise controlling a position of at least one of the right and the left trigonal tissue anchors by sliding at least extension extending from at least one of the right and left arms to at least one the right and the left trigonal tissue anchors.

In particular embodiments, at least some of the tissue anchors may be placed prior to attachment of the implant. Alternatively, at least some of the tissue anchors are attached to the implant prior to placement on the valve annulus. In some instances, the implant is advanced to the target region over at least one driver attached to at least one of the tissue anchors. In other instances, the implant is advanced to the target region over at least three drivers attached to the at least three tissue anchors.

In other instances, the at least one driver is actuated to attach the anchor before the implant is advanced over the at least one driver, where the implant fixes to the anchor after the implant has been fully advanced. In other instances, the implant body comprises an implantable template having a tissue-engaging surface. In other instances, the implant body is pre-shaped with at least one concavity at the center of the implant.

In still further aspects, the present invention provides an implant for repairing a valve annulus, said implant comprising a pre-shaped metallic template having at least one concavity configured to be positioned adjacent to a peripheral wall of the valve annulus. At least one anchor is configured to be coupled to the pre-shaped metallic template and to draw at least one segment of said peripheral wall of the valve annulus into said concavity to thereby reduce a diameter of said annulus in a radially inward direction. A skirt is attached to the pre-shaped metallic template and configured to engage tissue of the valve annulus when the pre-shaped metallic template is implanted in the valve annulus.

In particular embodiments, the skirt comprises a porous, knitted, woven, or electrospun fabric configured to promote tissue ingrowth with the peripheral wall of the valve annulus. The skirt may be attached to the pre-shaped metallic and may be configured to spans the concavity. In some instances, the pre-shaped metallic template has a surface along a length in an axial direction, where the concavity is formed in said surface and oriented in a lateral direction relative to said length. In some instances, the pre-shaped metallic template is deployable to said pre-shaped configuration from a crimped configuration.

In one example, one or more tissue anchors are placed in the annular region of a valve and coupled to a partial annular ring. In a further example, the partial annular ring is smaller than the pre-procedural dimensions of the valve annulus. In a further example, coupling the partial annular ring to the tissue anchors reduces at least one linear dimension of the valve annulus. In a further example, coupling the partial annular ring to the tissue anchors reduces the area of the valve annulus. In a further example, coupling the partial annular ring to the tissue anchors decreases the circumference of the annulus. In a further example, at least one of the couplings between the partial ring and at least one of the anchors comprises a slot, to allow circumferential motion of the anchor relative to the partial ring as the anchor is coupled to the ring, as the annular dimension is reduced. In a further example, coupling of partial annular ring to the tissue raises or lifts a portion of the valve annulus. In a further example, the partial annular ring couples to a valve replacement implant. In a further example, two or more anchors are implanted and connected to the ring. In a further example, the two or more anchors are arranged at different positions around at least a region of the valve annulus. In another example, a first anchor is fastened adjacent a valve leaflet, a second anchor is fastened 10 to 45 degrees clockwise from the first anchor, and a third anchor is fastened 10 to 45 degrees counter-clockwise from the first anchor. In another example, at least one anchor is fastened in a position relative to an anatomic marker, and two or more additional anchors are placed at angular intervals from 10 degrees to 60 degrees relative to the first anchor.

In one example, one or more tissue anchors are placed in the annular region of a valve and coupled to a complete annular ring. In a further example, the complete annular ring is smaller than the pre-procedural dimensions of the valve annulus. In a further example, coupling the complete annular ring to the tissue anchors reduces at least one linear dimension of the valve annulus. In a further example, coupling the complete annular ring to the tissue anchors reduces the area of the valve annulus. In a further example, coupling the complete annular ring to the tissue anchors decreases the circumference of the annulus. In a further example, at least one of the couplings between the complete ring and at least one of the anchors comprises a slot, to allow circumferential motion of the anchor relative to the complete ring as the anchor is coupled to the ring, as the annular dimension is reduced. In a further example, the complete annular ring couples to a valve replacement implant. In a further example, two or more anchors are implanted and connected to the ring. In a further example, the two or more anchors are arranged at different positions around at least a region of the valve annulus. In another example, a first anchor is fastened adjacent a valve leaflet, a second anchor is fastened 10 to 45 degrees clockwise from the first anchor, and a third anchor is fastened 10 to 45 degrees counter-clockwise from the first anchor. In another example, at least one anchor is fastened in a position relative to an anatomic marker, and two or more additional anchors are placed at angular intervals from 10 degrees to 60 degrees relative to the first anchor.

In a preferred example, at least one tissue anchor is placed in the annular region of a valve, and a tissue shaping template is coupled to the anchor. In a further example, the template is moved distally along an elongate member releasably attached to the tissue anchor. In a further example, as the template moves distally along an elongate member, the template compresses at least one extensible tab on the anchor to a smaller dimension than the tabs have in the unconstrained state. In a further example, the template is moved distally past the at least one tab, allowing the at least one tab to return to the unconstrained state. In a further example, the at least one tab in its unconstrained state inhibits proximal movement of the template relative to the anchor. In a further example, the at least one tab remains coupled to the anchor after the releasable elongate member is removed. In a further example, at least one tab has a constant cross section, and a flat end. In another example, at least one tab has a variable cross section. In a further example, the cross section of the tab decreases along at least a portion of the distance from the root of the tab to the tip of the tab. In another example, at least one tab has an end that is not flat. In a further example, at least one tab has a concave shape at the end. In another example, at least one tab has a convex shape at the end. In another example, at least one tab has an angular shape at the end. In another example, at least one tab has both a variable cross section, and a non-flat end. In another example, the anchor has at least one tab formed by cutting a pattern in a metallic tube. In another example, the anchor has at least one tab formed from a resilient material.

In another example, at least one tissue anchor is placed in the annular region of a valve, and a tissue shaping template is coupled to the anchor, which is in turn releasably coupled to a tubular elongate member. In a further example, the template is moved distally along the tubular elongate member. In a further example, the tubular elongate member compresses at least one extensible tab on the anchor to a smaller dimension than the tabs have in the unconstrained state. In a further example, releasing the tubular elongate member from the anchor allows at least one tab to return to the unconstrained state, inhibiting proximal movement of the template relative to the anchor.

In another example, at least one tissue anchor is placed in the annular region of a valve, and a tissue shaping template is coupled to the anchor by a helical thread. In a further example, at least one tooth on the template engages a helical thread on the anchor. In a further example, the helical thread has at least one segment with a varying pitch. In a further example, the helical thread has a section with a steep pitch and a section with a shallow pitch. In a further example, the shallow pitch section is at an angle which gives the helix and tooth non-reciprocal relationship, where axial forces will not cause rotation of the anchor relative to the template. In a further example, the helical thread has at least one section with a helix angle of less than 15 degrees. In an additional example, the tissue anchor comprises a helical coil of wire with the opposite handedness of the helical thread. In yet another example, the tissue anchor comprises a helical coil laser cut from a tube.

In another example, at least one tissue anchor which is releasably coupled to an elongate control member is placed in the annular region of a valve, and a tissue shaping template is coupled to the anchor by moving it distally past a detent attached to the anchor. In a further example, the detent comprises a spring and at least one plunger arranged to extend partially outside the dimension of the anchor. In a further example, the spring is compressed as the template is pushed distally over plunger, and the plunger returns to its extended state as the template is pushed distally past the plunger. In a further example, the plunger is rounded where it contacts the template as the template is pushed distally into position. In a further example, once the template is pushed distal to the detent, the detent inhibits proximal motion of the template relative to the anchor.

In a preferred example, a tissue anchor is releasably coupled to an elongate control member by a removable key wire. In a further example, the elongate member comprises at least in part a tubular structure having at least one lumen. In a further example, the key wire resides at least partially inside the lumen of the tubular structure. In a further example, the key wire passes through a hole in the elongate control member and at least into a corresponding hole in the anchor. In a further example, the key wire passes through a first hole in the elongate control member, into an elongate hole in the anchor, and through a second hole in the elongate control member to at least partially re-enter the lumen of the elongate control member. In another example, the key wire passes through a hole in the elongate control member and through a corresponding hole in the anchor. In another example, the key wire passes out of a slot in the elongate member, around a mating feature of the anchor, and returns through the same slot in the elongate member to at least partially re-enter the lumen of the elongate member. In another example, the key wire passes out of a first hole in the elongate member, around a mating feature of the anchor, and returns through a second hole in the elongate member to at least partially re-enter the lumen of the elongate member. In a further example, the mating feature of the anchor comprises one or more coils of wire. In a preferred example, at least a portion of the elongate member resides in a lumen in the anchor. In another example, at least a portion of the anchor resides in a lumen in the elongate member. In a further example, the elongate member is a sleeve that holds the tabs in a constrained position for passage through a mating feature in a tissue shaping template. In a further example, a second elongate member resides at least partially within the lumen of the first elongate member, and at least partially within a lumen in the anchor.

In another example, a tissue anchor is releasably coupled to an elongate control member by at least one extensible tab attached to the elongate control member. In a further example, the extensible tab passes at least partially through a mating feature in the tissue anchor. In a further example, the extensible tab is held in position extending at least partially through the mating feature in the tissue anchor by a wire having a first small diameter and a second larger diameter, the larger diameter in contact with the extensible tabs. In a further example, the larger diameter has a spherical, flared, T-shape or other similar form. In a further example, the key wire resides at least partially inside the lumen of the tubular structure. In a further example, the larger diameter segment of the key wire can be pushed distally to allow the extensible tab to exit the mating feature in the tissue anchor, decoupling the anchor from the elongate member. In another example, the larger diameter segment of the key wire can be pulled proximally to allow the extensible tab to exit the mating feature in the tissue anchor, decoupling the anchor from the elongate member.

In another example, a tissue anchor is releasably coupled to an elongate control member by a detent. In a further example, the detent resides in the anchor, and comprises a spring and at least one plunger arranged to extend partially outside the dimension of the anchor. In a further example, the spring is compressed as the elongate member is pushed distally over the plunger, and the plunger returns at least partially to its extended state as at least a portion of the elongate member is pushed distally past the plunger. In a further example, features in the elongate member engage the plunger. In a further example, the plunger engaging features in the elongate member comprise through-holes. In another example, the plunger engaging features in the elongate member comprise partial depth holes.

In another example, a tissue anchor is releasably coupled to an elongate control member by a detent which resides in the elongate member and comprises a spring and at least one plunger arranged to extend partially outside the dimension of the elongate member. In a further example, the spring is compressed as the anchor moves proximally over the plunger, and the plunger returns at least partially to its extended state as at least a portion of the anchor is pushed distally past the plunger. In a further example, features in the anchor engage the plunger. In a further example, the plunger engaging features in the anchor comprise through-holes. In another example, the plunger engaging features in the anchor comprise partial depth holes.

In one example, a first tissue shaping template and tissue anchor are placed in tissue to have a desired tissue effect, which is measured and evaluated. In a further example, the effect is adjusted to a desired level by adding a second template adjacent to the first tissue shaping template. In a further example, the second template is coupled to the same tissue anchor as the first template. In a further example, the second template adjusts the shape of the first template without coupling with additional area of the annulus. In another example, the second template extends past the first template to couple with additional areas of the annulus. In another example, the second template is independent of the first template. In a further example, the second template treats an area of the annulus distinct from the first template.

In one example, a tissue shaping template and tissue anchor are placed in tissue to have a desired tissue effect, which is measured and evaluated, and a modifying implant placed to change the shape of the tissue shaping template. In a further example, the modifying implant is a ring that goes at least partially around a portion of the tissue shaping template. In a further example, the modifying implant is held in place with one or more tissue anchors. In another example, the modifying implant is a clip, staple, or coil placed at least partially around the tissue shaping template.

In one example, a tissue shaping implant includes a bioresorbable component. In a further example, the bioresorbable component degrades after a period of time sufficient for healing and tissue remodeling to take place. In a further example, the dissolution of the bioresorbable component reduces the stiffness of the tissue shaping implant. In another example, the tissue shaping implant is held in a constrained configuration by the bioresorbable component, and the dissolution of the bioresorbable component allows the tissue shaping implant to return at least partially to its unconstrained state. In another example, the tissue shaping implant comprises at least two separate permanent components connected by a bioresorbable component. In another example, the tissue shaping implant comprises at least two separate permanent components connected by both a bioresorbable component and a permanent connecting component. In another example, the permanent connecting component is held in a constrained state by the bioresorbable component, and the dissolution of the bioresorbable component allows the permanent connecting component to return at least partially to its unconstrained state. In a further example, the permanent connecting component comprises a coil spring, stent, or closed cell elastic structure. In another example, at least a portion of at least one of the permanent components in the tissue shaping template comprises a coil spring, stent, or closed cell elastic structure. In yet another example, the tissue shaping template is made from entirely biodegradable or corrodible material or materials. Examples of corrodible metals or metal alloys (degradable) include nickel, cobalt, tungsten; tungsten alloys of rhenium, cobalt, iron, zirconium, zinc, titanium; magnesium, magnesium alloys, magnesium alloy AZ31, magnesium alloy with less than 20% zinc or aluminum by weight, without or with one or more impurities of less than 3% iron, silicone, manganese, cobalt, nickel, yttrium, scandium or other rare earth metal, AZ31B or MG11B5A11Zn0.034Sc (LAZ1151); zinc or its alloy such as zine alloys such as Zn5al, Zn10Al, Zn18Al, Zn30Al; bismuth or its alloy; indium or its alloy, tin or its alloy such as tin-lead, Sn3.9Ag0.6Cu, Sn-3.8Ag-0.7Cu, SnPb, or SnPbAt; silver or its alloy such as silver-tin alloy; cobalt-iron alloy; iron or its alloys such as 80-55-06 grade cast ductile iron, other cast ductile irons, AISI 1010 steel, AISI 1015 steel, AISI 1430 steel, AISI 8620 steel, AISI 5140 steel, Fe29.7Mn8.7Al1C, 30HGSA alloy steel, 4140, C45 steel, Fe36Ni, low carbon steel or other steels; melt fusible alloys (such as 40% bismuth-60% tin, 58% bismuth-42% tin, bismuth-tin-indium alloys; alloys comprising one or more of bismuth, indium, cobalt, tungsten, bismuth, silver, copper, iron, zinc, magnesium, zirconium, molybdenum, indium, tin; or other material; or the like. Examples of degradable material such as degradable polymeric material comprise one or more of: lactides, caprolactones, trimethylene carbonate, glycolides, poly(L-lactide), poly-DL-Lactide, polylactide-co-glycolide (e.g., poly(L-lactide-co-glycolide), copolymer of poly(L-lactide-co-epsilon-caprolactone (e.g., weight ratio of from around 50 to around 95% L-lactide to about 50 to about 5% caprolactone; poly(L-lactide-co-trimethylene carbonate), polytrimethylene carbonate, poly-caprolactone, poly(glycolide-trimethylene carbonate), poly(lactide-glycolide-trimethylene carbonate) or the like; polyhydroxybutyrate such as poly(3-hydroxybutyrate) and poly(4-hydroxybutyrate); polyhydroxyvalerate; polyhydroxybutyrate/polyhydroxyvalerate copolymers (PHV/PHB); polyhydroxyalkanoate; poly orthoesters; poly anhydride; polyiminocarbonate; tyrosine-derived polycarbonate; tyrosine-derived polyacrylate; iodinated and/or brominated tyrosine-derived polycarbonate; iodinated and/or brominated tyrosine-derived polyacrylates polyesteramide; polycarbonate copolymer, lactone based polymers such as poly(propylene fumarate-co-ethylene glycol) copolymer (aka fumarate anhydride); polyanhydride esters; polyorthesters; polyphosphazene; aliphatic polyurethane; polymers, blends, and/or co-polymers, combination thereof, or the like.

In one example, a tissue shaping template comprises at least two segments connected by a captured pawl with teeth. In a further example, the teeth of the pawl are configured to allow expansion of the tissue shaping template in response to tension, but to resist shrinking of the template in response to compression. In a further example, each segment of the tissue shaping implant is coupled to tissue by a tissue anchor. In a further example, a first segment of the tissue shaping implant comprises a tension member that can be captured by an adjusting catheter. In a further example, tension applied between the adjusting catheter coupled to the first segment and the tissue anchor coupled to the second segment expand the tissue shaping template. In another example, the second segment comprises a second tension member that can be captured by an adjusting catheter. In a further example, an adjusting catheter can apply a tension directly between the two segments to expand the tissue shaping template. In a further example, the tension member comprises a hoop, t-shaped handle, socket, or the like.

In one example, two tissue anchors which are each releasably coupled to an elongate control body are placed adjacent a valve annulus at a first separation distance, and a tissue shaping implant is coupled to each of the tissue anchors. In a further example, the tissue shaping implant holds the tissue anchors at a second separation distance that is smaller than the first separation distance. In a further example, the coupling between the tissue anchors and the tissue shaping implant restricts angular rotation of the tissue anchors such that the axes of the tissue anchors remain substantially parallel. In a further example, the thickness of the tissue shaping implant is sufficient to maintain the substantially parallel alignment of the tissue anchors. In another example, a boss on the tissue anchor is of sufficient width to maintain the substantially parallel alignment of the tissue anchors. In another example, a compression member between the two anchors in combination with the tissue shaping template provides sufficient torque to maintain the substantially parallel alignment of the tissue anchors. In another example, an array of three or more tissue anchors are placed adjacent a valve annulus and connected in pairs to a chain of two or more tissue shaping templates, where adjacent templates share one tissue anchor. In a further example, four tissue anchors are arranged adjacent a valve annulus and separated by between 5 and 60 degrees each, and connected by three tissue shaping implants. In a further example, an array of “n” anchors is connected by “n−1” tissue shaping templates.

In one example, two tissue anchors which are each releasably coupled to an elongate control body are placed adjacent a valve annulus at a first separation distance, and a tissue shaping implant is coupled to each of the tissue anchors. In a further example, the tissue shaping implant holds the tissue anchors at a second separation distance that is larger than the first separation distance.

In one example, two or more tissue anchors are coupled to tissue adjacent a valve annulus and connected by one or more tissue shaping templates. In a further example, three anchors are placed in a triangular configuration. In a further example, a straight line between two of the implants passes over the valve commissure. In another example, a straight line between two of the anchors follows the periphery of the valve annulus. In a further example, one or more of the tissue shaping implants support tension between the tissue anchors. In a further example, the tissue shaping implant is a single, adjustable, flexible member. In a further example, tension in the tissue shaping implant can be adjusted by at least one tension adjustment mechanism attached to one or more of the anchors. In another example, the tension in the tissue shaping implant can be adjusted by at least one tension adjustment mechanism on the tissue shaping implant between two anchors. In another example, the tissue shaping implant is a suture, wire, filament, cable, or the like. In another example, the tissue shaping implant is a rigid body implant. In yet another example, the tissue shaping implant is made partially or fully from one or more biodegradable material.

In one example, an annular tissue anchor is coupled to tissue adjacent a valve annulus, a base tissue anchor is coupled to tissue outside the valve region, and the annular anchor and base anchor are connected by a tissue shaping implant. In a further example, the base anchor is coupled to the apex of the heart. In a further example, the base anchor is coupled to the apex of the heart via a catheter that traverses at least part of the corresponding ventricle of the heart. In another example, the base anchor is coupled to the apex of the heart from an access point outside the heart. In another example, the annular anchors are placed via a catheter that traverses at least part of the corresponding ventricle. In another example, the annular anchors are placed via a catheter that traverses at least part of the corresponding atrium. In another example, the tissue shaping implant is a flexible member. In a further example, the tension in the tissue shaping implant can be adjusted via an adjusting mechanism coupled to the base anchor. In another example, the tension in the tissue shaping implant can be adjusted via an adjusting mechanism coupled to the annular anchor. In another example, the tension in the tissue shaping implant can be adjusted via an adjusting mechanism along the tissue shaping implant between the two anchors. In one example, the annular anchor is coupled to the mitral valve. In another example, the annular anchor is coupled to the tricuspid valve. In another example, two or more tissue shaping implants are coupled to the same base anchor. In a further example, the two or more tissue shaping implants are coupled to two or more annular anchors in the same valve annulus. In another example, the two or more tissue shaping implants are coupled to two or more annular anchors in two or more valves. In a further example, the two or more valves include the mitral valve and the tricuspid valve. In another example, the base anchor is coupled to tissue that moves relative to the annular anchor with every heartbeat, and the tissue shaping implants are configured to bring the anchors towards and away from each other in response to the motion of each heartbeat.

In one example, a tissue shaping template is coupled adjacent a valve annulus by one or more tissue anchors. In a further example, the tissue shaping template is configured to shrink at least one diameter of the annulus. In a further example, the tissue shaping template is configured to shrink the minor diameter. In another example, the tissue shaping template is configured to reduce the area of the valve. In another example, the tissue shaping template is configured to reduce the circumference of the annulus. In another example, the tissue shaping template is configured to fold or plicate a section of the annulus. In another example, the tissue shaping template is configured to fold or plicate a section of the valve leaflet adjacent the annulus. In another example, the tissue shaping template is configured to fold or plicate a section of the valve annulus and a section of the valve leaflet. In another example, the tissue shaping template is configured to pull a section of the valve annulus inward. In a further example, the tissue shaping template pulls a section of the valve annulus inward while pressing in the opposite direction on another section of the valve annulus, on the wall of a chamber of the heart, on the fibrous skeleton of the heart, or another structure adjacent the valve annulus. In another example, the tissue shaping template is configured to displace a section of the valve annulus out of the initial plane of the valve annulus. In a further example, the angle of the tissue anchor in the target tissue affects the out of plane displacement by maintaining a desired angle between the tissue shaping template and the plane of the valve annulus.

In one example, a tissue shaping template is coupled to one or more tissue anchors which are coupled to tissue adjacent a valve leaflet. In a further example, the tissue shaping template and tissue anchor plicate the valve annulus. In a further example, the tissue shaping template and anchor plicate the valve annulus and associated valve leaflet. In a further example, the plicated leaflet remains in close contact with the tissue shaping template.

In another example, a tissue shaping template is covered at least partially with a flexible planar covering and is coupled to one or more tissue anchors which are coupled to tissue adjacent a valve leaflet. In a further example, the planar covering is a woven fabric. In a further example, the woven fabric is of an appropriate thread count to encourage tissue ingrowth. In a further example, the woven fabric is Dacron, or the like. In another example, the planar covering is a non-woven fabric. In a further example, the non-woven fabric is Tyvek, ePTFE, or the like. In a further example, the non-woven fabric is constructed by electrospinning the one or more desired polymers such as PTFE, PVDF-HF, Chronoflex, Tecoflex, Biomer, polyurethanes, or the like.

In another example, a tissue shaping template is covered at least partially with a flexible planar covering and is coupled to one or more tissue anchors which are coupled to tissue adjacent a valve leaflet on the ventricular side of the valve. In a further example, the tissue shaping template is placed on the ventricular side of the posterior leaflets of a mitral valve. In a further example, the tissue shaping template is sized to cover more than one of the posterior leaflets of the mitral valve. In a further example, the flexible planar covering substantially seals against the valve annulus and prevents flow of blood through the planar covering.

In another example, a tissue shaping template is covered at least partially with a flexible planar covering, and is coupled to one or more tissue anchors which are coupled to tissue adjacent a valve leaflet on the ventricular side of the valve, the tissue shaping template being configured to urge the coaptation surface of the posterior leaflets closer to the coaptation surface of the anterior leaflets. In a further example, the tissue shaping template has at least a partially tubular shape. In a further example, the tissue shaping template has a tubular shape. In a further example, the tissue shaping template is an expandable stent. In a further example, the tissue shaping template is a self-expanding stent. In another example, the tissue shaping template is a balloon expandable stent. In another example, the tissue shaping template is placed adjacent the ventricular side of the posterior mitral valve leaflets, and the planar covering on the tissue shaping template contacts the coaptation surface of the anterior leaflets when the valve closes.

In one example, a tissue shaping template having one or more tissue anchors is coupled to a valve annulus from the ventricular side of the valve annulus. In another example, a tissue shaping template having one or more tissue anchors is coupled to an area of the muscular wall of the ventricle. In a further example, the area of the muscular wall of the ventricle has previously enlarged relative to its normative state. In a further example, the enlargement was caused by an ischemic episode.

In one example, a tissue shaping template having one or more tissue anchors and a fabric covering is coupled to tissue adjacent a valve annulus. In a further example, the fabric covering is a woven fabric such as Dacron or a non-woven fabric such as Tyvek or ePTFE. In a further example, the fabric covering covers at least a majority of the structure of the template. In a further example, the fabric covers at least a majority of the area between arms of the template. In another example, the fabric covers at least a majority of the area from the center of the template outward to the valve annulus. In a further example, the fabric cover creates a smooth zone between the template and the valve annulus that a later implant could form a hemostatic seal against. In another example, the fabric is formed by electrospinning one or more polymers such as PTFE, poly(vinylidene fluoride, poly(vinylidene fluoride-co-hexafluoropropylene), polystyrene-b-polyisobutylene-b-polystyrene (30% styrene 70% isobutylene), Chronoflex, Chronoprene, Tecoflex, Biomer, other polyurethanes and its copolymers, poly(n-butyl-methyacrylate), poly(ethylene-co-vinyl acetate), BioLinx, phosphorylcholine, combination thereof, or the like. In another example, the fabric is bonded to the anchor. In a further example, the fabric is bonded to the anchor using an adhesive. In a further example, the adhesive is a cyanoacrylate adhesive. In yet another example, the fabric is held on the anchor with suture or wire. In a further example, the fabric is coated, infused or covalently bonded to heparin.

In one example, a tissue shaping template having one or more fabric covered tissue anchors is coupled to tissue adjacent a valve annulus. In a further example, the fabric covering is a woven fabric such as Dacron or a non-woven fabric such as Tyvek or ePTFE. In a further example, the fabric covering covers at least a majority of the structure of the anchor. In a further example, the fabric covers at least a majority of a helical coil that is twisted to advance into the tissue. In a further example, advancing the anchor into the tissue causes the fabric cover to compress and form a pledget or seal between the template and annulus. In another example, the fabric covers at least a majority of the docking structure of the anchor. In another example, the fabric is formed by electrospinning a polymer. In another example, the fabric is bonded to the anchor. In a further example, the fabric is bonded to the anchor using an adhesive. In a further example, the adhesive is a cyanoacrylate adhesive. In yet another example, the fabric or pledget seals the hole made by the anchor in tissue such as the annulus, muscle, leaflet, or the like underlying the anchor.

In one example, a tissue template is releasably coupled to the distal end of a delivery catheter. In a further example, a tissue anchor is releasably coupled to an elongate control wire, and is anchored to tissue adjacent a valve annulus. In a further example, the proximal end of the elongate control wire is inserted through a mating feature on the tissue template, through a portion of the delivery catheter, and out of a side port in the delivery catheter. In a further example, the tissue template and delivery catheter are advanced distally along the elongate control wire toward the tissue anchor. In a further example, the side port is more than 0.25″ from the distal tip of the delivery catheter, and less than 5″ from the distal tip of the delivery catheter.

In one example, a guidewire, tissue anchor with elongate control wire, or other wire is placed into a vascular system, through a sheath and dilator, the dilator having a port in its distal tip through which the wire passes, and a taper from a diameter slightly larger than the port, to a diameter approximately the inner diameter of the sheath, and a side exit slot through which the wire can pass when both the wire and dilator are inside the sheath. In a further example, there is a transition from the distal port to the side exit slot that guides the proximal end of the wire to the side slot. In a further example, the side slot is long enough to extend out of the proximal end of the sheath when the dilator is fully inserted into the sheath. In a further example, the side slot ends proximal to a dilator handle. In a further example, there is no distal port, and the side slot extends to the distal end of the dilator.

In one example, a tissue anchor is anchored to tissue adjacent to a valve annulus, and releasably coupled to an elongate control wire, which passes through a sheath, the system composed so that tension is applied to the control wire, deforming the annulus in the region of the anchor, the deformation being observed by imaging techniques such as ultrasonography, fluoroscopy, CT Scan, or the like. In a further example, the sheath enters a chamber of the heart through a passage, and an abutment on the sheath, proximal to the passage, and larger in diameter than the passage prevents the sheath from being drawn farther into the heart chamber in response to tension in the elongate control wire. In a further example, the abutment is an expandable basket. In another example, the abutment is an inflatable balloon.

In one example, a tissue anchor is anchored to tissue adjacent to a valve annulus, and releasably coupled to an elongate control wire, which passes through a steerable sheath, the system composed so that tension is applied to the control wire, deforming the annulus in the region of the anchor, the deformation being observed by imaging techniques such as ultrasonography, fluoroscopy, CT scan, or the like. In a further example, the steerable sheath incorporates two curved sections arranged so that the second curve prevents the sheath from being drawn farther into the heart chamber in response to tension in the elongate control wire.

In another example, a tissue anchor is anchored to tissue adjacent to a valve annulus, and releasably coupled to an elongate control wire, which passes through a steerable sheath alongside a stiffening member, the system composed so that tension is applied to the control wire, deforming the annulus in the region of the anchor, the deformation being observed by imaging techniques such as ultrasonography, fluoroscopy, or the like. In a further example, the stiffening member limits deflection of the sheath to prevent the sheath from being drawn farther into the heart chamber in response to tension in the elongate control wire. In a further example, the stiffening member is advanced until it abuts a structure of the body, bracing the system in position to prevent the sheath from being drawn farther into the heart chamber in response to tension in the elongate control wire.

In another example, a tissue anchor is anchored to tissue adjacent to a valve annulus, and releasably coupled to an elongate control wire, which passes through a bracing catheter, the bracing catheter and control wire in turn passing through a steerable sheath, the system composed so that tension is applied to the control wire, deforming the annulus in the region of the anchor, the deformation being observed by imaging techniques such as ultrasonography, fluoroscopy, CT scan, or the like. In a further example, the bracing catheter comprises one or more extended legs with contact points for bracing the system against structures in the heart. In a further example, the bracing catheter comprises two legs with contact points intended to simulate a tissue shaping template configured so that tensioning the control wire relative to the bracing catheter simulates the effect of placing a tissue shaping template in the valve annulus.

In a twelfth aspect, the present invention provides an implantable system comprising multiple components which may be assembled in situ after the components have been introduced to a target tissue site, such as a heart valve or a heart valve annulus. Such implantable systems may comprise at least an implantable component, a tissue anchor, and an elongate driver. The implantable component will typically incorporate a component coupling element, while the tissue anchor will typically incorporate an anchor-coupling element. The elongate driver is typically removably attached to the anchor-coupling element, and the anchor-coupling element is usually configured to be coupled to the component coupling element after the tissue anchor has been embedded in tissue by the elongate driver. The implantable component will usually be configured to be advanced over the elongate driver after the tissue anchor has been imbedded in tissue, and the component coupling element will typically be configured to be attached to the anchor-coupling element after the tissue anchor has been embedded in tissue.

In some examples, the component coupling element and the anchor-coupling element will be configured to form an interlocking connection when brought together after the tissue anchor has been embedded in tissue. For example, the component coupling element and the anchor-coupling element may be configured to provide a “snap-on” mechanism which allows the component coupling element to be engaged against the anchor-coupling element to create a mechanically stable connection which will remain intact unless and until the connection is mechanically reversed, for example by a removal tool.

In other examples, the component coupling element comprises a female connector and the anchor-coupling element comprises a male connector, where the female connector of the implantable component may be advanced over the male connector of the tissue anchor in order to form a desired interlocking mechanical or other link. The link will remain intact unless and until the coupling elements are decoupled or otherwise detached from each other, typically by a removal tool.

In still other examples of the implantable systems of the present invention, the tissue anchor may be configured to be rotationally advanced into tissue by the elongate driver. Examples of such rotational tissue anchors include helices, screws, coils, spirals, and the like. Alternatively, the tissue anchor may be configured to be non-rotationally advanced into tissue by the elongate driver. Examples of non-rotational tissue anchors include ratcheting tethers, hooks, barbs, fasteners, clips, locks, staples, and the like.

The implantable components of the implantable systems of the present invention may have a wide variety of purposes and designs, and in some cases an implantable component may comprise multiple sub-components which themselves may be attached, linked, or otherwise reconfigured in situ to form the implantable component. In certain exemplary embodiments, the implantable component(s) will comprise an implant for reshaping a valve annulus. For example, such valve-reshaping implants may comprise a pre-shaped template having a length in an axial direction and at least one concavity or convexity in a lateral direction along the length of the implant. The concavity or convexity will typically have or form a surface configured to be positioned adjacent to a peripheral wall of the valve annulus. When said templates have a concavity, the tissue anchors will typically be configured to draw annulus tissue into the concavity (invaginate the tissue) in order to decrease an effective circumference of the valve annulus. When the templates have a convexity, the convexity will typically engage or push out the tissue of the annulus to form a radially outward protrusion which will have a similar effect in decreasing an effective circumference of the valve annulus.

In other specific examples and instances of the implantable systems of the present invention, the implantable component may comprise a prosthetic heart valve or heart valve assembly. In some cases, the prosthetic heart valve may comprise an integrated structure where a portion or location on the integrated structure will include one or more component coupling elements, typically having a plurality of component coupling elements distributed about a circumference of the heart valve structure. The component coupling elements of the integrated prosthetic valve structure may then be directly or indirectly connected to the anchor-coupling elements of one or more tissue anchors which have been previously implanted in a heart valve annulus. Alternatively, the component coupling elements of the integrated prosthetic valve structure may be coupled to a template, coupled to the tissue anchors, and/or attached directly to the native valve apparatus, for example, the valve leaflets.

In other instances, the heart valve structure may comprise an assembly including a heart valve component and a base component which partially or fully circumscribes the annulus. In such instances, the base component will include the one or more component coupling elements which configured to directly attach to the anchor-coupling element(s) on the tissue anchors which have been previously embedded in the valve annulus. The heart valve component may then be introduced, typically over the elongate drivers, to engage and be coupled to the previously implanted base structure. Typically, both the valve structure and the base structure will themselves have coupling elements which allow the valve structure to be automatically coupled to the implanted circumscribing structure in a manner similar to the component coupling elements and the anchor-coupling elements.

In still other examples, the implantable component may comprise an annuloplasty ring. For example, the annuloplasty ring may be configured to elastically expand when implanted on a valve annulus such that, when released from constraint, a radially inward force will be applied to the valve annulus by elastic annuloplasty ring. The annuloplasty ring may be have a serpentine, zig zag, or other structure which permits expansion and elastic contraction in order to apply the desired forces on the valve annulus.

In still further examples of the implantable systems, at least a second tissue anchor and at least a second elongate driver may be included in order to provide additional coupling locations between the implantable component and additional tissue anchors. Anywhere from two to 30 tissue anchors, usually two to 15 tissue anchors, and often two to eight tissue anchors, may be provided together with a corresponding number of elongate drivers.

In a thirteenth aspect of the present invention, a method for attaching an implantable component to a target location on a patient's tissue surface, such as a heart valve annulus, comprises providing an elongate driver having a distal end removably attached to a tissue anchor. The elongate driver is advanced to the target location on the patient's tissue and actuated to implant the tissue anchor at the tissue location. The implantable component may then be advanced over the elongate driver to the target location, and a coupling element on the implantable component may be attached to a coupling element on the implanted tissue anchor. A distal end of the elongate driver is then detached from the implantable component, and the elongate driver removed from the target location.

In specific examples of these methods for attaching an implantable component, the elongate driver may be actuated by locating the driver to screw in the tissue anchor. The implantable component may then be advanced over the elongate driver to the target location by pushing the implantable component with a pusher device or feature. The coupling element on the implantable component is attached to the coupling element on the implanted tissue anchor by interlocking the two coupling elements together, typically in a “snap-on” fashion as described previously.

Exemplary tissue locations include a heart valve annulus, such as a mitral valve annulus. In such instances, the implantable component may comprise an implant for reshaping the valve annulus. For example, the implant may comprise a pre-shaped template as described herein above. Alternatively, the implantable component may comprise a heart valve prosthesis, an annuloplasty or other ring for partially or wholly attaching to the valve annulus, or combinations thereof.

In a fourteenth aspect of the present invention, an implantable system comprises an implantable component incorporating a component coupling element and a tissue anchor component incorporating an anchor-coupling element. The tissue anchor-coupling element is configured to self-couple to the component coupling element after the tissue anchor has been embedded in tissue. The implantable component comprises a pre-shaped template having a length and an axial direction and at least one concavity or convexity in a lateral direction along said length period. The concavity or convexity will have a surface configured to be positioned adjacent a peripheral wall of the valve annulus period.

In specific examples, such implantable systems may further comprise an elongate driver removably attached to the anchor-coupling element, where the pre-shaped template of the implantable component is configured to be advanced over the elongate driver after the tissue anchor has been embedded in tissue.

As with previously described implantable systems, the component coupling element may comprise a female connector and the anchor-coupling element may comprise a male connector. The component coupling element may be further configured to be detached from the anchor-coupling element after the tissue anchor has been embedded in tissue, and other aspects of the previously described implantable systems of the present invention may be incorporated with these further embodiments.

In a fifteenth aspect of the present invention, a method for attaching an implant for reshaping a valve annulus at a target location on the valve annulus comprises implanting one or more tissue anchors at one or more target locations on the annulus. The implant typically comprising a pre-shaped template having a length in an axial direction and at least one concavity or convexity in a lateral direction which is advanced to the target location on the annulus. The concavity or convexity has a surface configured to be positioned adjacent to a peripheral wall of the valve annulus, and a coupling element on the pre-shaped template is attached to a coupling element on the implanted tissue anchor in order to effect reshaping of the annulus.

In particular examples, such methods for reshaping a valve annulus may comprise attaching the coupling element on the pre-shaped template to the coupling element on the implanted tissue anchor by bringing the coupling elements together to form an interlocking connection, typically a self-locking connection. Advancing the implant to the target location typically may comprise providing an elongate driver having a distal end removably attached to the tissue anchor. The elongate driver may be advanced to the target location and actuated to implant the tissue anchor at the target location. A distal end of the elongate driver is then detached from the pre-shaped template and the elongate driver removed from the tissue anchor.

In a sixteenth aspect of the present invention, a ring implant is attached to a native valve annulus by implanting a plurality of tissue anchors at target locations on the valve annulus. The ring implant is then advanced to the valve annulus, and a plurality of coupling elements on the ring implant are attached to a like number of coupling elements on the implanted valve annulus. Implanting the plurality of tissue anchors and coupling elements on the valve annulus is typically performed using a plurality of elongate drivers, each having a distal end that is removably attached to one of the plurality of tissues anchors. Elongate drivers are advanced to the tissue locations and actuated to implant the tissue anchors at each of the target locations. The distal ends of each of the elongate drivers are then detached from the tissue anchors after the ring implant has been advanced to the implant site and attached to the tissue anchors.

In preferred aspects, the elongate drivers may be left in place after attachment of the ring implant to the implanted tissue anchors. The elongate drivers may then be used to advance a second implant component, such as a valve prosthesis, over the same elongate drivers that were used to introduce the tissue anchors and thereafter the ring implant. In such instances, coupling elements on both the ring implant and the tissue prosthesis will typically be configured to attach the valve prosthesis to the ring implant, to the tissue anchors, or directly to anatomical components of the valve, valve annulus, atrium, ventricle or other tissue adjacent the valve. Such coupling elements may comprise any type of coupling element as described previously with regard to attachment of the implant component to the tissue anchors.

In one example, the devices comprise a template having a first end, a second end, and a body extending between said first and second ends, wherein the device is configured to reshape a section of annulus from a substantially smooth shape to a convoluted shape which can foreshorten or otherwise tighten the valve or other annulus. Typically, the template is coupled to the annulus in one or more locations between said first and second ends. For example, the first and second ends of said template may be coupled to the annulus at two or more annulus points or regions, and/or said body may include one or more coupled locations which pull said annulus radially inwardly while said first and second ends exert a radially outward force on the annulus, substantially opposing the inward pull of said one or more coupled locations.

In a preferred example, the template is coupled to the annulus at two or more location, and such locations are separated by a segment of the template body that exerts a radially outward force on the annulus substantially opposing the inward pull of said two or more coupled regions. In a preferred example, the body is coupled in one or more location to the annulus between the proximal and distal ends by means comprising one or more of: screws, clips, sutures, barbs, or other means. In another preferred example, the body is coupled to the annulus while the device is in a deflected configuration. In yet another preferred example, the body is coupled to the annulus at or near a mid-point between first and second ends. In yet another example, the body is coupled to the annulus at two locations, said locations lie between first and second ends. In yet another example, the body is coupled to the annulus in three locations wherein the locations lie between first and second ends of the template. In a preferred example, the device is formed from a material wherein the material comprises one or more of: rigid, self-expanding, elastic, super-elastic, plastically deformable, and has a form comprising one or more of: coil, screw, spiral, spring, barb, suture, hook staple, etc. In a preferred example, the means coupling one or more locations means on the body to the annulus comprises one or more of the following actions: penetrating the annulus, and holding together said annulus and body at the coupling location; penetrating the annulus, holding together said annulus and body at the coupling location, and pulling in said annulus to desired shape (configuration) upon deployment of the device; penetrating the annulus, holding together said annulus and body at the coupling location, and pulling in said annulus to desired shape (configuration) upon deployment of the device and coupling said proximal and distal ends to one or more annulus points or regions conforming to said body coupled region to said annulus, reshaping coupled annulus, reshaping coupled annulus to shape of body in the coupled region.

In a further example, the template is coupled to the annulus in one or more locations via a primary anchor and one or more accessory anchors. In a further example, the primary anchor is placed into the target tissue, and then coupled to an implantable frame which is in turn coupled to one or more accessory anchors. In a preferred example, the frame is an integral portion of the template. In another example, the frame is a separate implantable component coupled to the template. In a further example, the frame is formed from one or more of the following materials: elastic, superelastic, shape memory, hard tempered, heat treated. Examples of said materials include one or more of the following: Nitinol, stainless-steel, maraging steel, cobalt chromium, or the like.

In another example, the accessory anchors are helical coils. In a further example, the helical coil anchors are coupled to the frame in a way that restricts axial and radial motion while allowing rotational motion, so that the helical coil anchors can be coupled to the tissue by rotating relative to the frame. In another example, the helical coils anchors are coupled to the frame via a threaded insert, so that rotation of the helical coil anchors cause corresponding motion in the axial direction relative to the frame. In a preferred example, the helical coil anchor has an additional axial segment which disengages from the threaded insert, allowing the helical coil anchor to rotate about its axis relative to the frame without causing corresponding motion in the axial direction relative to the frame.

In another example, a frame is coupled to two or more helical coil tissue anchors, each anchor comprising a gear, the anchors arranged in the frame so that the gears mesh, causing all anchors to rotate simultaneously. In a further example, one or more anchors is releasably coupled to a torque member through which the coupled anchor can turn, causing the other anchors with meshed gears to turn as well. In a further example, the handedness of each helical coil is arranged so that all anchors in the train tend to turn into tissue or out of tissue simultaneously, even as their rotational direction alternates as the gears turn each other in alternating directions.

In a preferred example, the template is coupled to a delivery device between two jaws for delivery to the target tissue over the control wire of a primary tissue anchor. In a further example, the two jaws move relative to each other from a gripping position where the template is coupled to the delivery device to a release position where the delivery device can be withdrawn proximally relative to the template. In a further example, one or more of the jaws comprises teeth which extend partially over the distal facing surface of the template. In a further example, one or more of the jaws comprise a tube or shape cut from a tube. In a further example, at least one jaw is free to pivot, so that the angle between the two jaws changes from the gripping position to the release position.

In another example, the two jaws translate relative to each other to release a template held at an angle to the direction of the translational movement, the angle being measured from the axis of the primary tissue anchor to the vector of the translational movement. In a further example, the angle between the template and the direction of translational movement is between 30 degrees and 60 degrees. In a further example, the angle between the template and the direction of translational movement is between 0 degrees and 70 degrees.

In another example, a delivery device includes lumens, holes, or other clearances for passage of one or more control wires releasably coupled to one or more tissue anchors. In a further example, a control wire releasably coupled to a primary tissue anchor passes through a hole in a jaw of the delivery device. In another example, a control wire releasably coupled to ha primary tissue anchor passes through a lumen of the delivery device. In a further example, the control wires of one or more accessory anchors pass through a lumen in the delivery device. In another example, the control wires of one or more accessory anchors pass outside the jaws of the delivery device.

In a further example, a delivery device includes lumens for passage of one or more control wires releasably coupled to one or more side anchors which are in turn coupled to a tissue shaping template. In a further example, tension applied to the side anchor control wires retracts the lateral arms of the template during placement.

In a further example, a frame coupled to one or more accessory anchors includes clearance areas for teeth coupled to the delivery device, the teeth partially covering the distal facing side of the tissue template, and the clearance areas being sized and located so that as the template coupled to the delivery device approximates the frame, the distance between the frame and template is less than the length that the teeth extend distal to the distal aspect of the template.

In a preferred example, the template and side anchors are constructed of superelastic Nitinol material, the anchors, pods, and frames are constructed of stainless-steel. In a further example, the pod components are laser cut from stainless-steel tubing of an appropriate size and welded together, the center and accessory anchor dock components are laser cut from stainless-steel tubing, the center and accessory anchor washer components are cut from flat sheets of stainless-steel, the center and accessory anchor wire forms are bent to shape from drawn stainless-steel wire, and the side anchors are laser cut from superelastic Nitinol tubing. In a further example, the template is cut from superelastic Nitinol tubing and heat set to the desired shape by clamping in one or more fixtures constructed of high temperature materials (aluminum, stainless-steel, or the like), and heated to 400°-550° C. in a fluidized bed of sand or a pot of molten salt for 1 to 8 minutes, then quenched in a water or oil bath at approximately room temperature. In another example, some or all of the stainless-steel implanted components are replaced with equivalent parts made from cobalt chromium, for example MP35N. In a further example, the tubular delivery device components are laser cut from stainless-steel tubing and the teeth are laser cut from stainless-steel sheet stock. In a further example, stainless-steel components are laser welded together as needed to form a cohesive assembly.

In a preferred example, the template has an undulating shape.

In another example, the template has an undulating shape with an even number of undulations pressing into the annulus, and an odd number of undulations which are coupled to the annulus by coupling mechanism(s) in tension.

In another example, the template has an undulating shape with an odd number of undulations pressing into the annulus, and an even number of undulations which are coupled to the annulus by coupling mechanism(s) in tension.

In another example, the template has an undulating shape with an even number of undulations pressing into the annulus, and an even number of undulations which are coupled to the annulus by coupling mechanism(s) in tension.

In a preferred example, the template surface is compatible with tissue in contact with the template. This compatibility can be achieved through a number of methods known to the arts including template material choice, template surface finish, coatings, and flocking. The compatibility can also be achieved through a variety of covering materials including ePTFE, Dacron knits, other knit fabrics, and the like.

In a preferred example, the valve annulus comprises annulus and tissue adjacent to annulus.

In another example, the device is an implant or a temporary implant.

In one example, the device is formed from one or more of the following categories of material: strong, stiff, resilient, shape memory, elastic, plastically deformable, capable of withstanding cyclic load of at least 10 million cycles.

In another example, multiple devices are implanted along the annulus.

In another example, multiple devices are implanted along the annulus and connected with rigid or semi-rigid connectors.

In one example, the device is formed from a degradable or non-degradable material.

Device comprises a template wherein the device comprises an expandable body, a ring, a body with one end, a body with two ends, a body with three ends or more.

In certain further examples, the implant of the present invention may comprise a tissue coupling mechanism configured to anchor, secure, or stabilize the template and position the undulating body of the template adjacent to the inner surface of the heart valve annulus. The tissue coupling mechanism may comprise a tissue penetrating element. The tissue coupling mechanism may have a first tissue penetrating element at a first connected end of the inner and outer arcuate members and a second tissue penetrating element at second connected end of the inner and outer arcuate members, and the tissue penetrating element(s) may comprise of projection(s) such as barb(s).

In one example, the device comprising a body wherein the body comprises a proximal end, a distal end, and a shaft extending between said proximal and distal ends, wherein the device is configured to being expandable from crimped configuration to an expanded configuration, and wherein the shaft is coupled to the annulus in one or more locations between said proximal and distal ends, and wherein the proximal and distal ends of said body are coupled to two or more annulus points or regions, and wherein said shaft one or more coupled locations pull said inward while proximal and distal ends stretch annulus coupled to said proximal and distal ends. In a preferred example, the shaft is coupled in one or more location to the annulus between the proximal and distal ends by means comprising one or more of: screws, clips, sutures, barbs, or other means. In another preferred example, the shaft is couple to the annulus while the device is in a crimped configuration. In yet another preferred example, the shaft is coupled to the annulus at a mid-point between proximal and distal ends. In yet another example the shaft is coupled to the annulus at two locations, said locations lie between proximal and distal ends. In yet another example, the shaft is coupled to the annulus in three locations wherein the locations lie between proximal and distal ends of the body. In a preferred example, the device is formed from a material wherein the material comprises one or more of: self-expanding, elastic, plastically deformable, coil, spring, etc. In a preferred example, the means coupling one or more locations means on the shaft to the annulus comprises one or more of the following actions: penetrating the annulus, penetrating the annulus and holding together said annulus and shaft at the coupling location, penetrating the annulus, holding together said annulus and shaft at the coupling location, and pulling in said annulus to desired shape (configuration) upon deployment of the device, penetrating the annulus, holding together said annulus and shaft at the coupling location, and pulling in said annulus to desired shape (configuration) upon deployment of the device and coupling said proximal and distal ends each to one or more annulus points or regions, conforming to said shaft coupled region to said annulus, reshaping coupled annulus, reshaping coupled annulus to shape of shaft in the coupled region.

In a preferred example, the valve annulus comprises annulus and tissue adjacent to annulus.

In another example, the device is an implant or a temporary implant.

In another example the device comprises a body and two ends. In another example, the device comprising a body and two ends, wherein each end comprising at least one prong, wherein the two ends push two or more tissue points or regions outwardly. In one example, the two ends bifurcate into two or trifurcate into three prongs. In a preferred example, the two ends are connected by a shaft. In another example, the two ends are connected by one or more shafts. In yet another example the two ends are connected by two or more shafts. In yet another example, the two ends are connected by a shaft wherein the shaft branches into multiple shafts along the path of said shaft. In a preferred example the shaft comprises a solid body, yet it may also in other examples comprise hollow (tubular) body, or other. In a preferred example the shaft has a round shape. In other examples, the shaft shape comprises oblong, rectangle, semi-circle, triangle, elliptical, dog bone, square, or other shapes. The two ends may have the same shape and geometry or may have different shapes and geometries. In yet another example, the two ends have the same shape and geometry as the shaft. In yet another example, the device ends comprise one or more prongs wherein the prongs have a shape or geometry comprising one or more of spear, barb, pad, flat, disc, rough surface, round, square, rectangle, bulbous, arc, or other. In yet another example, the one or more prongs maybe coupled to adjacent tissue wherein the prong coupling to the tissue comprises one or more of suturing, screw, geometry of the prong such as a barb configuration penetrating the tissue, coupling, placating, pressing, surface adhesion, surface friction, or other. In yet another example, each of the two ends comprises one or more prongs, wherein each end prongs have the same or different shape or geometry. In yet another example, each of the two ends comprises two or more prongs, wherein each of the prongs have the same or different shape or geometry. In yet another example, the device comprises at least one end wherein said end comprises at least two or more prongs bifurcate about the same location on the shaft. In yet another example, the two or more prongs bifurcate at different locations along the shaft length. In yet another example, the device comprises at least one end and wherein said at least one end has two or more prongs wherein the function of the two or more prongs may be the same or different. In yet another example, the device comprises at least one end and wherein said at least one end has two or more prongs and wherein at least one prong pushes adjacent tissue outwardly. In yet another example, the device comprises at least one end and wherein said at least one end has two or more prongs and wherein at least one prong pushes adjacent tissue outwardly and at least one prong pulls in adjacent tissue inwardly. In yet another example, the device comprises at least one end and wherein said at least one end has two or more prongs and wherein at least one prong pushes adjacent tissue outwardly and at least one prong holds adjacent tissue in place. In yet another example, the device comprises at least one end and wherein said at least one end comprises at least one prong and wherein the function of said prong comprises one or more of securing an end of the device to adjacent tissue, pushing adjacent tissue outwardly, holding in place adjacent tissue, pulling inward adjacent tissue, aligning tissue regions, configuring tissue regions to be out of plane (misaligned), controlling or limiting penetration depth of the device into the tissue, or other. In yet another example, the device comprising a body and two ends wherein each end comprising at least one prong, wherein the two ends push two or more tissue points or regions outwardly, and wherein other two or more tissue points or regions are pulled inwardly (or pulled in together). In yet another example, the device comprising a body and two ends wherein at least one end comprises at least two prongs, wherein at least one prong pushes adjacent tissue outwardly, and wherein at least one prong is pulling adjacent tissue inwardly, and wherein two or more tissue points or regions between the two ends are pulled inwardly. In a preferred example, the two or more tissue points or regions are adjacent to said body ends. In one example, the device is coupled to one or more tissue points or regions to push said tissue points or regions outwardly, and wherein the device is configured to exert outward force to said one or more tissue points or regions, and wherein the location of device coupling comprises one or more locations comprises the body of the device, device one or more ends, device one or more prongs, to affect an annulus shape wherein two or more points regions on the annulus are pushed outwardly while two or more points or regions on the annulus are pulled inwardly.

In one example, the device comprising a body, wherein said body is connected to at least two ends wherein each end comprising at least one prong, wherein the at least two ends push two or more tissue points or regions outwardly. In yet another example, the device comprising a body and at least two ends, each end comprising at least one prong, wherein the at least two ends push two or more tissue points or regions outwardly, and wherein two or more other tissue points or regions are pulled inwardly. In a preferred example, the two or more tissue points or regions pushed outwardly are adjacent to said body ends and the other two or more tissue points or regions pulled inwardly are located between said body ends. In a preferred example, the device is positioned in a valve annulus wherein two or more tissue points or regions pushed outwardly are adjacent to said body ends at the annulus and the other two or more tissue points or regions pulled inwardly are located between said body ends at said the annulus. In one example, the device is attached or affixed to adjacent tissue in one or more locations to affect outward and/or inward movement of tissue and/or annulus shape.

In another example, the device comprises a body having at least two ends, wherein each end comprises at least one prong and wherein at least one end or at least one prong is pushing an adjacent tissue outwardly. In another example, the device comprising a body, wherein said body has at least two ends wherein each end comprising at least one prong, and wherein the device is coupled, in one or more locations, to one or more tissue points or regions and configured to pushing said tissue points or regions outwardly and wherein other tissue points or regions between said coupled locations are pull inwardly and wherein the device ends affect the tissue (including annulus) wherein an effect may comprise one or more of two ends pushing two or more tissue (including annulus) points or regions comprising one or more of apart, outwardly, in opposite direction, in plane, out of plane. In another example, the device comprising a body, wherein said body is connected to three ends wherein each end comprising at least one prong.

The device may be a rod with two ends, a device having body like a disc or a stent, or other comparable structures.

In one example, the device is formed from a resilient material shaped into a rod having two ends and a shaft connecting said two ends.

In one example, the device is formed from one or more of the following categories of material: strong, stiff, resilient, shape memory, elastic, plastically deformable, capable of withstanding cyclic load of at least 10 million cycles.

In one example, the device is formed from a degradable or non-degradable material.

Device comprises a body wherein the device comprises an expandable body, a ring, a shaft with one end, a shaft with two ends, a shaft with three ends or more.

In a further aspect of the present invention, an implant is configured to reshape a heart valve having a valve annulus and valve leaflets. The implant comprises an inner arcuate member configured to conform to an inner surface of a heart valve annulus and an outer arcuate member configured to conform to an inner surface of a heart wall adjacent to the heart valve annulus. The inner and outer arcuate members are coupled together and are further configured to be attached to tissue in, on, or near the heart valve so that the inner arcuate member applies an inwardly acting radial force on at least a portion of the inner surface of the valve annulus and the outer arcuate member applies an outwardly acting radial force on the inner surface on the heart wall. Such forces will stabilize the annulus to promote enhanced leaflet coaptation with minimum stretching of the valve leaflets.

In certain examples of the implant, the inner and outer arcuate members are connected at their ends and have an annular space between an outer edge of the inner arcuate member and an inner edge of the outer arcuate member. A mechanism may be disposed in the annular space and be configured to adjust the relative positions of the inner and outer arcuate members in order to, for example, allow adjustment of the reshaping and stabilization of the annulus. The mechanism may comprise a threaded member or other suitable linear translation element. Alternatively, the mechanism may comprise a spring or other self-adjusting coupling structure. In still further examples, the implant may comprise a plurality of mechanisms disposed in the annular space and configured to adjust the relative positions of the inner and outer arcuate members.

In certain further examples, the implant of the present invention may comprise a tissue coupling mechanism configured to anchor the implant and position the inner arcuate member adjacent to the inner surface of the heart valve annulus and position the outer arcuate members adjacent to the inner surface of the heart valve wall. The tissue coupling mechanism may comprise a tissue penetrating element. The tissue coupling mechanism may have a first tissue penetrating element at a first connected end of the inner and outer arcuate members and a second tissue penetrating element at second connected end of the inner and outer arcuate members, and in all cases, the tissue penetrating element(s) comprise barb(s).

In yet another aspect of the present invention, a method for treating a heart valve having a heart valve annulus, valve leaflets and a heart valve wall surface adjacent to the annulus comprises providing an implant comprising an inner arcuate member configured to conform to an inner surface of the heart valve annulus and an outer arcuate member configured to conform to an inner surface of the heart wall adjacent to the heart valve annulus. The implant is implanted above the heart valve so that the inner arcuate member applies an inwardly acting radial force on at least a portion of the inner surface of the valve annulus and the outer arcuate member applies an outwardly acting radial force on the inner surface on the heart wall.

These methods may further comprise adjusting a width or other dimension, angle, or shape of an annular space between an outer edge of the inner arcuate member and an inner edge of the outer arcuate member to vary at least one of the inwardly acting radial force on at least a portion of the inner surface of the valve annulus and the outwardly acting radial force on the inner surface on the heart wall. Implanting may comprise anchoring the implant into tissue around the heart valve, for example by anchoring at least a first end on the implant and a second end of the implant wherein the inner and outer arcuate members are connected. Anchoring the implant into tissue around the heart valve may comprise penetrating elements into tissue in or adjacent to the annulus. For example, anchoring the implant into tissue around the heart valve may comprise inserting fasteners, such as barbs, helical anchors, screws, and the like which are attached or otherwise coupled to the implant, into tissue where the fasteners may be located at a first end on the implant and a second end of the implant wherein the inner and outer arcuate members are connected. In a further example, anchoring the implant into tissue in or adjacent to the annulus may comprise one or more tissue penetrating anchors intermediate to the first and second ends of the implant. In a further example, the tissue being pulled inward by the template may comprise valve leaflet tissue, which is stretched toward the opposing valve leaflet.

Various control and delivery mechanisms are illustrated herein, including torsion tubes and delivery devices that interact with the body of the template. These control mechanisms may be actuated manually by the operator, or by a remotely powered actuator system.

In various of the above examples, the surface of the template may be partially or fully covered with ePTFE, velour, knitting, weaving, spray coating, electrospun coatings, combinations thereof, or the like.

In various of the above examples, the surface may be partially or fully coated with anticoagulants, anti-thrombotic agents, thrombolytic agents, anti-thrombin agents, anti-fibrin agents, anti-platelet agents, combination thereof, or the like.

In various of the above examples, the surface or a surface covering may have pores at least partially filled with anticoagulants, anti-thrombotic agents, thrombolytic agents, anti-thrombin agents, anti-fibrin agents, anti-platelet agents, combination thereof, or the like.

In various of the above examples, an anchor surface may be partially or fully coated with anticoagulants, anti-thrombotic agents, thrombolytic agents, anti-thrombin agents, anti-fibrin agents, anti-platelet agents, combination thereof, or the like.

In various of the above examples, a proximal end of one or more anchors may collapse into a minimal structure upon removal of the torque tubing.

In various of the above examples, the annulus may also include tissue adjacent to the annulus.

In one exemplary embodiment, an implant constructed in accordance with the principles of the present invention for reshaping a valve annulus comprises a pre-shaped template and at least one anchor. The pre-shaped template has a length in an axial direction and at least one concavity extending in a lateral direction along the length. The concavity defines a concave surface on one side of the template, which concave surface is typically configured to be positioned against and/or adjacent to a peripheral wall of valve annulus. The at least one anchor on the template is configured to draw at least one segment (region) of the peripheral wall of the valve annulus into the concavity so that the segment (region) is brought up against the concave surface to at least partially conform to the shape and contour of the concave surface.

The pre-shaped template may have a variety of geometries. It will typically be a non-linear elongated member having a surface with a shape or contour which will be imparted to the segment of peripheral wall of valve annulus after the template has been anchored to the annulus tissue. In many examples, the pre-shaped template will be curved along its length, typically having a serpentine, undulating, angulated (having one or more abrupt bends or angles along its length), or have another wave-like or zig-zag profile which will cause the periphery of the annulus to fold or plicate, thereby shortening and/or repositioning a peripheral length of the annulus, in a manner mimicking annuloplasty when the template is attached to the annulus.

Although in many examples, the pre-formed template will be free from angled bends along its length, in other examples the concavity may be formed with angled bends (angulated) along the length of the template, for example the concavity may have a rectilinear periphery (four sequential bends of approximately 90° each defining the concavity). In other examples, angulated bends may be combined with curved or arcuate segments to shape the template.

In many examples, the concavities of the pre-shaped template will be symmetric about a lateral axis, usually having opposed legs joined by a curved junction region forming the bottom of the concavity. In some examples, either or both opposed legs may have a convex surface (convexity) formed at an outer termination or transition region thereof. Usually, such convex surfaces will be axially and laterally spaced-apart from the curved junction region of the concavity, and the at least one anchor of the implant will be further configured to draw adjacent segments of the peripheral wall of the valve annulus against the convex surfaces as well as into the concavity.

In still other specific examples, the pre-shaped template may have at least two concavities separated by a convexity therebetween. In such examples, the concavities may be disposed symmetrically about a lateral axis passing through a mid-point or apex of the convexity therebetween. The convexity will typically comprise a curved junction region which joins a pair of oppose legs, with each leg joined its lower end to one of the concavities, with each concavity being laterally spaced-apart from the mid-point of the convexity. In such examples, the at least one anchor on the template is further configured to draw adjacent segments of the peripheral wall of the valve annulus against the concave surfaces as well as against the convex surfaces therebetween.

In all such examples, the implants of the present invention may be used individually or in groups of two, three, four, or more. When used in groups, the implants may be left unattached after they have been implanted, or alternatively may be further joined together in tandem, for example by bonding or attaching terminal regions of one implant to terminal regions of an adjacent implant.

Implants of the present invention may be implanted in any cardiac valve, venous valve, or other vascular valve of a human or other patient. For example, the implants may be implanted into all or a portion of a patient's posterior mitral valve annulus, posterior tricuspid annulus, anterior-posterior tricuspid annulus, aortic annulus, pulmonary valve annulus, or the like.

The templates of the present invention will generally comprise an elongate structure having at least two of terminal ends, a pair of side edges, a tissue-engaging surface, and an inwardly facing surface, but can have other structures with various number of edges, surfaces, and terminal ends. The length of template, when in its non-linear form, will typically be from 10 mm to 185 mm, often being in a range 10 mm to 75 mm, and sometimes being in the range 20 mm to 60 mm. The width of the template will typically be in a range from 1 mm to 15 mm, usually from 2 mm to 8 mm, and often from 2 mm to 6 mm. The thickness of the template will typically be from 0.1 mm to 2 mm, more usually from 0.2 mm to 1.5 mm. In specific examples, the elongate structures of the templates may comprise of a plate, a ribbon, a mesh, a lattice, a beam, a tube, a rod, a cylinder, a coil, a spiral, a spring, or a combination thereof. Exemplary templates will be elongated, shape-memory metal ribbons which have been heat-set or otherwise shape-set to a desired non-linear geometry with one or more concavities.

The elongate structures of the templates may be formed from any material having sufficient strength, resiliency, and biocompatibility to be implanted in a patient's heart and to conform to a region of the patient's peripheral annulus to effect shortening thereof, typically being a metal, such as a nickel-titanium alloy, a stainless-steel, or the like.

Individual implants according to the present invention may have a single concavity, at least two concavities, at least three concavities, at least four concavities, typically having from one to twelve concavities.

While the pre-shaped of the present invention templates will usually be a curved, elongated structure having and having first and second discrete ends, in other examples, they may comprise or be joined together as a continuous ring intended to be implanted about a full periphery of the patient's valve annulus. In some examples, a plurality implants (typically from two to six) having discrete ends may be configured to be joined end-to-end either before implantation or after implantation (in situ). In both examples, the templates will form a continuous structure about the entire periphery of the valve annulus.

In many examples, the implant templates of the present invention will be pre-shaped, i.e. will have an undulating, serpentine, and/or angulated shape imparted during manufacturing. In other examples, it may be possible to provide templates which are configured to be shaped in situ.

In some examples, the templates of the implants of the present invention may be covered in a biocompatible material, such as ePTFE, polyethylene terephthalate (Dacron®), or other materials intended to encourage tissue in-growth. Such biocompatible materials may be formed into suitable structures including open-cell foam structures, closed-cell foam structures, woven fabrics, non-woven fabrics, texture or surface finishes, and the like.

The anchors of the implants of the present invention will typically be tension anchors configured to draw at least a portion of a segment of an inner surface of annulus into the concavity. For example, the anchors may comprise a helix, a ratcheting tether, a screw, a coil, a spiral, a hook, a barb, a clip, a lock, a staple, or any other type of fastener which can both engage the target tissue and draw the target tissue into the concavity. Suitable tissue anchors may have one or more ribs, wings, barbs, expansion elements, wedges, extensions, protrusions, and combinations thereof.

In a specific instance, at least one anchor may comprise a helical anchor having a distal end and proximal end. The distal end may have a sharpened tip, and the proximal end may be rotatably secured in the concavity of the template, typically at a mid-point of a curved junction region. Usually, the helical anchor will be configured to be engaged by a detachable driver to rotate the helical anchor to drive the sharpened tip into the annulus and draw at least a segment of an inner surface of the annulus into the concavity. Such anchors may comprise a helical coil, a screw, a spiral, or the like, typically being a helical coil.

In further specific examples, the concavity in the template will have a depth in the lateral direction. The helical anchor may have a length which is greater than depth of the concavity. In this way, the sharpened tip will be positioned beyond an outer tissue-engaging surface of the template so that the tip can engage tissue without the need to deform the pre-shaped template. In other examples, however, the helical anchor may have a length which is less than a depth of the concavity. In such examples, the sharpened tip can engage tissue by pressing the template against the target tissue and deforming the template to allow the sharpened tip of the helical anchor to engage the target tissue.

In other exemplary examples of the present invention, the anchor may comprise any one or more of a ratcheting tether, a hook, a barb, a fastener, a clip, a loop, or a staple. Such anchors have a distal end and a proximal end, where the distal may comprise a sharpened tip and the proximal end may be secured in concavity of the template and be configured to push and pull with a detachable driver. In this way, the anchor can push and pierce into the annulus to draw at least a segment of the inner surface of the annulus into the concavity and to lock that segment into place.

In still further specific examples, the implants of the present invention may comprise elements or components for stabilizing tissue. For example, a tissue-coupling mechanism may be attached at either or both of the ends of a pre-shaped template to stabilize the template and hold it in place after implantation. Such tissue coupling mechanisms may comprise, for example, helical anchors or other fasteners configured to be rotatably advanced into tissue, where such anchors are similar to the primary anchor intended to draw tissue into the concavity. Other stabilizing tissue coupling mechanisms may include self-penetrating barbs, staples, clips, or the like that can be used to secure the free ends of the template against the valve annulus tissue.

In still further specific examples of the present invention, the stabilizing mechanism for the template may comprise a stabilizing arm which extends laterally from the pre-shaped template, where the stabilizing arm may engage tissue above the annulus after the template has been implanted. The stabilizing arm may have a pad at its distal end or may alternatively comprise a stabilizing anchor or other fastener similar to those described above for the ends of the template.

In still further aspects, the present invention comprises systems for reshaping a valve annulus. Such systems may include any of the implants described above in combination with a delivery catheter. The delivery catheter typically has proximal end and a distal end, where the implant is removably carried on the distal end. In exemplary examples, the delivery catheters may comprise at least one flexible tension member secured to the at least one anchor on the template, typically comprising a plurality of flexible tension members when the template includes a plurality of anchors. The flexible tension members are removably secured to the anchors so that the catheter may be detached from the implant after implantation has been completed. The flexible tension members are typically further configured to rotate the at least one anchor to advance said anchor into tissue. For example, the flexible tension members may comprise a flexible coil or other rotatable drive shaft having a distal coupling member configured to removably engage a drive element on the proximal end of the at least one anchor.

For example, the coupling member may comprise a sleeve or bushing with a hole or passage or other aperture formed in a wall thereof, and the flexible tension member may comprise a separate wire or elongate element for passing through the aperture in the coupling member so that rotation and attachment of the flexible tension member to the coupling member can controlled by advancing and retracting the elongate element into and out of the aperture.

The systems of the present invention may further comprise elongated control elements detachably secured to the ends of the pre-shaped template. For example, the elongated control elements may be configured to collapse the pre-shaped template about the anchor, typically so that the implant may be collapsed during delivery and opened after advancement from the delivery catheter. Alternatively, the elongated control elements may be configured to pull back the pre-shaped template away from the anchor, again to reduce its profile for delivery while allowing release to its original configuration after advancement toward the valve annulus.

In still further examples, the systems of the present invention may comprise a pre-anchor guide translatably or slidably coupled to delivery catheter. The pre-anchor guide may comprise a guide wire-like shaft having a coil or other tissue anchor at its distal tip. In this way, the pre-anchor guide may be advanced into the valve annulus at particular target location prior to advancement of the implant. The delivery catheter can then be advanced over the pre-anchor guide to properly position the implant prior to implantation.

In still further exemplary examples, the present invention provides methods for reshaping a valve annulus. Typically, the methods comprise engaging a template against a peripheral surface of the valve annulus, where the template has both a tissue-engaging surface and at least one concavity formed in the surface in a radially inward direction relative to the valve annulus. At least one segment of peripheral surface of the annulus is drawn into the concavity, resulting in a shortening and/or repositioning of a peripheral length of the valve annulus, which can mimic annuloplasty and reduce valve regurgitation, particularly mitral valve regurgitation in at least most patients.

In specific examples of the methods herein, the template may be configured to be engaged against a peripheral surface of at least a length a posterior segment of a tricuspid valve annulus, an aortic valve annulus or a pulmonary valve annulus. The lengths of engagement will range from 10 mm to 185 mm, with other specific ranges as set forth above with regard to the implant design of the present invention.

The implanted templates will typically comprise an undulating, serpentine or angulated structure having the at least one concavity. Such undulating, serpentine or angulated structures may have two concavities, three concavities, four concavities, five concavities or more as described previously. The templates will typically be pre-shaped but in other examples could be formed in situ. In still other examples, multiple templates may be implanted and joined together prior to implantation or in situ to provide for a longer engagement against the valve annulus, and in some examples engaging an entire periphery of a valve annulus.

In many examples, applying tension to a peripheral wall segment to draw the segment in the concavity typically comprises advancing the anchor into a target region on the peripheral annulus in a manner that draws that tissue into the concavity on the template. Usually, the anchor comprises a helical coil, screw, or spiral having a proximal portion which is rotatably attached to the template, typically at a bottom of the concavity, so that the anchor will remain laterally fixed relative to the template while the anchor acts as “cork screw” in drawing tissue into the concavity.

In other examples, drawing a segment of the annulus into the concavity may comprise applying compression to the segment to compress the segment into the concavity. For example, compression may be applied by looping, tying, suturing, clipping or the like. In other examples, compression may be effected by a compression anchor configured to secure and stabilize the template to the tissue. Such compression anchors include a helix, a ratcheting tether, a screw, a coil, a spiral, a hook, a barb, a fastener, a clip, a hook, a staple or the like.

The methods of the present invention may comprise advancing the template intravascularly, percutaneously (such as via a transapical approach), or via a minimally invasive approach, such as a throacoscopic approach.

In specific examples, the templates may be attached to the target tissue of the annulus by rotating a helical end anchor on the template, where the anchor has a proximal end and sharpened distal end. Typically, rotating such anchors comprises rotating a flexible tension member in the delivery catheter to drive the sharpened distal tip into tissue and draw the tissue segment of the annulus into the concavity.

In still further exemplary examples, the methods for reshaping a valve annulus may comprise placing a semi-rigid template adjacent to a portion of the peripheral wall of the heart valve annulus. The semi-rigid template is fastened to the portion of the peripheral wall in a manner such that the annulus is caused to approximate the shape of the template. For example, the template may exert opposing radial forces on the inner wall of annulus to cause the annulus to partially plicate and foreshorten. Usually, the semi-rigid template will not substantially increase a diametric dimension of the annulus. The template as with previous examples may comprise multiple segments having substantially the same shape. Alternatively, the template may comprise multiple segments having distinct shapes.

In one example, the invention is a system to reshape a valve annulus comprises a template having a preformed shape with at least one concavity and at least one anchor on the at least one concavity, wherein the template is delivered to appose an annulus region, wherein said anchor is configured to reposition said annulus region into said concavity. In a further example, said template has two additional anchors to hold said template in place and prevent flipping or twisting of the template about its axis. In a further example, the template is releasably coupled to a delivery device, and wherein the delivery device is removed after anchoring said template to said annulus region. In yet another example, the said template having a length along a longitudinal axis and at least one concavity in a lateral direction along said length, and said template has two apex segments each segment connected by a leg to one side of said concavity, wherein each of said apex segment has an anchor configured to affix at least one region of said apex segment to adjacent annulus. In another example, the apex segment comprises one or more of convex region, flat region, and concave region(s). In yet another example, the template comprises a plurality of concavities and a plurality of apex segments, wherein some or all of the concavities has an anchor configured to reposition at least one region of an annulus into said concavities, and some, all, or none of the apex segments may have anchors to attach the apex segments to adjacent annulus regions. In yet another example, the template comprises a plurality of concavities and a plurality of apex segments, wherein each concavity has an anchor configured to reposition at least one region of an annulus into said concavities, and wherein said template further comprises at least two apex segments wherein at least one of said apex segments has an anchor configured to attach at least one region of the apex segment to the adjacent annulus. In another example, the said template has the advantage of repositioning selective regions of valve annulus. In another example, the template is configured to reposition one region of a valve annulus, wherein the region comprises a posterior annulus region, an anterior annulus region, a septal annulus region, or an anterior posterior region. In one example, annulus regions outside said template remain substantially unchanged. In another example, said template is configured to perform one or more of the following: reposition at least one region of an annulus into said concavity of the template, reduction of the valve annulus circumference, reducing an annulus configuration, reducing the annular area, reducing one or more dimensions of the annulus, reduction of a said annulus region circumference, configuration, or one or more dimension's. In another example, the template comprises at least one concavity joined by legs, wherein the legs comprise an apex segment and wherein each apex segment contains an anchor configured to attach at least one region of said segment to the adjacent annulus region, and wherein said concavity containing at least one anchor configured to pull in at least one region of an annulus into said concavity. In a preferred example, the apex segments have a substantially equal but opposite force to said anchor pulling in said annulus into said concavity. In another or same example, the apex segments are configured to prevent flipping or rotation of said template about its axis. In one example, the template is pre-formed before delivery into a patient body, in another example, the template is formed in situ. In a preferred example, the template comprising at least one concavity containing at least one anchor configured to pull in said adjacent and apposing annulus region into said concavity, wherein said annulus region conforms and/or contours substantially to the shape of said concavity. In another example, the template comprising at least one concavity may have various shapes of concavity, partial concavity, or a lateral space for an anchor to pull into said lateral space adjacent annulus region.

In another example, a system to reshape a valve annulus comprises a template having a preformed shape, comprising at least one concavity and at least two apex segments wherein each apex segment has a leg connected to said concavity, and at least one anchor disposed in the at least one concavity, the template being constrained in a first crimped, smaller configuration for delivery to the annulus region, and being configured to appose an annulus region and pull in said annulus region into said concavity. In a variation of this example, the template is released from the first crimped configuration prior to apposing the said annulus and constrained to a second crimped configuration wherein said second crimped configuration is larger or different than said first crimped configuration to reduce the force required to pull in said annulus region into said concavity. In this example, the second crimped configuration constraint means is different than the first crimped configuration constraint. In another example, the template is released from a first and/or second constraint after anchor pulls in annulus region into said concavity. In yet another example, the template is released from a first and/or second constraint prior to anchor pulling in said annulus region into said concavity. In a further example, the template comprises at least one opening in at least one apex segment connected to said concavity via a leg, wherein an anchor affixes at least one portion of said apex segment to an annulus region adjacent to the apex segment. In a preferred example, the template is held in the first crimped configuration inside a tubular body or at least partially inside a tubular body. In another example, the template is constrained in the second crimped configuration by at least one control wire configured to control at least one said apex segment rotation and/or affixing of said segment to adjacent annulus region, the template being releasably attached to said control wire, and said wire extending through a or the tubular body proximally outside the patient body to allow control of the template configurations at a distance from the template. In a further example, the template is releasably coupled to a delivery catheter and the delivery catheter is removed after anchoring the template to the annulus region. In a further example, the delivery catheter is inserted into the body or vasculature percutaneously, or surgically, or a hybrid procedure. In one example, the template is pre-formed prior to delivery into the annulus region. In another example, the template is formed in situ. In yet another example, the said template having a length along a longitudinal axis and at least one concavity in a lateral direction along said length, and said template has two apex segments each segment connected by a leg to one side of said concavity, wherein each of said apex segment has an anchor configured to affix at least one region of said apex segment to adjacent annulus. In another example, the apex segment comprises one or more of convex region, flat region, and concave region(s). In yet another example, the template comprises a plurality of concavities and a plurality of apex segments, wherein each concavity has an anchor configured to reposition at least one region of an annulus into said concavities, and wherein said template further comprises at least two apex segments wherein at least one of said apex segments has an anchor configured to attach at least one region of the apex segment to the adjacent annulus. In another example, the said template has the advantage of repositioning selective regions of valve annulus. In one example, the template is configured to reposition one region of a valve annulus, wherein the region comprises a posterior annulus region, an anterior annulus region, a septal annulus region, or an anterior posterior region. In one example, annulus regions outside said template remain substantially unchanged. In another example, said template is configured to perform one or more of the following: reposition at least one region of an annulus into said concavity of the template, reduction of the valve annulus circumference, reducing annulus configuration, reducing one or more dimensions of the annulus, reduction of a said annulus region circumference, configuration, area, or one or more dimensions. In another example, the template comprises at least one concavity joined by legs, wherein the legs comprise an apex segment and wherein each apex segment contains an anchor configured to attach at least one region of said segment to the adjacent annulus region, and wherein said concavity containing at least one anchor configured to pull in at least one region of an annulus into said concavity. In a preferred example, the apex segments have a substantially equal but opposite force to said anchor pulling in said annulus into said concavity. In another example, the apex segments are configured to prevent flipping or rotation of said template about its axis. In one example, the template is pre-formed before delivery into a patient body, in another example, the template is formed in situ. In a preferred example, the template comprising at least one concavity containing at least one anchor configured to pull in said adjacent and apposing annulus region into said concavity, wherein said annulus region conforms and/or contours substantially to the shape of said concavity. In another example, the template comprising at least one concavity may have various shapes of concavity, partial concavity, or a lateral space for an anchor to pull into said lateral space adjacent annulus region. In one example, the template is pre-formed into a substantially Omega shape comprising a concavity connected to two apex segments via legs connected to said concavity, and wherein said concavity having at least one anchor configured to pull in an annulus region into said concavity, and wherein the two apex segments each has an anchor configured to connect to adjacent annulus region to the said apex segment, and wherein the template is crimped into first crimped configuration having substantially U shape, wherein said U shaped template is constrained inside a first constraint comprising a tubular catheter and delivered in proximity to a valve annulus, and wherein the U shaped template is at least partially released from the tubular catheter, and wherein the concavity anchor apposes the desired annulus region and engages said annulus region pulling in said region into said concavity, and wherein the apex segments are positioned apposing to annulus regions and affixed to said annulus regions. The concavity anchor and apex segments anchors are controlled and/or constrained by (second constraint or second crimped configuration constraint) one or more wires, tubes, or the like that extend to outside the patient body and are configured to control anchoring of the template to the annulus, adjust the position of the template or template component, and/or to release the template. In one example, the template is pre-formed into a substantially Omega shape comprising a concavity connected to two apex segments via legs connected to said concavity, and wherein said concavity having at least one anchor configured to pull in an annulus region into said concavity, and wherein the two apex segments each has an anchor configured to connect to adjacent annulus region to the said apex segment, and wherein the template is crimped into first crimped configuration, wherein said template is constrained inside a first constraint comprising a tubular catheter and delivered in proximity to a valve annulus, and wherein the template is at least partially released from the tubular catheter, and wherein the concavity anchor apposes the desired annulus region and engages said annulus region pulling in said region into said concavity, and wherein the apex segments are positioned apposing to annulus regions and affixed to said annulus regions. The concavity anchor and apex segments anchors are controlled and/or constrained by (second constraint or second crimped configuration constraint) one or more wires, tubes, or the like that extend to outside the patient body and are configured to control anchoring of the template to the annulus, adjust the position of the template or template component, and/or to release the template. The template maybe crimped into various shapes inside a constraint comprising U shape, helical shape, pre-formed shape, or other shapes configured to be deliverable into a patient body to an annulus region. In a preferred example, the apex segments are pulled or held in a proximal direction relative to the anchor to facilitate an easier anchoring of the concave anchor to the annulus and then the apex segments are positioned, anchored and released. In this example, the apex anchors enhance or augment the amount (or mass or volume or area) annulus region pulled into said concavity. In another example, the template concavity anchor engages the annulus pulling said annulus region into said concavity, while the apex segments are apposing an annulus region, and then said apex segments apposing said annulus regions are affixed to said annulus region.

In one example, a system to reshape a valve annulus comprising advancing an anchor which is releasably attached to an elongate control wire through a tubular body and attaching that anchor to an annulus region, placing a template having a preformed shape having a concavity (such as a template comprising a concavity and two apex segments connected to said concavity via legs forming a substantially Omega shape template) in a crimped (smaller) configuration into a constraint catheter, sliding the template concavity over the control wire and coupling the template concavity to the anchor. In a further example, said template further has at least one apex segment and has at least one additional anchor-coupling the at least one apex segment to an adjacent annulus region. In a further example, the template is releasably coupled to a delivery catheter and the delivery catheter is removed after anchoring the template to the annulus. In another example, the delivery catheter is inserted into the body or vasculature percutaneously.

In another example, a one or more segments of the template is coupled to an anchor to prevent translation along the axis of the anchor. In a further example, the one or more segments of the template is coupled to an anchor allowing the anchor to rotate about its axis relative to the template. In a further example, the anchor is coupled to the template in the region of a concavity.

In one example, an implant having a preformed template wherein the template comprises at least one concavity and at least one connected apex segment wherein the at least one apex segment has at least one tissue anchor to affix the template to adjacent annulus, and at least one anchor is releasably attached to at least one elongate anchor control device extending from the implant to outside the delivery catheter of the implant. In a further example, the anchor control device is a tube with cut features to control flexibility. In a further example, the anchor control device is a tube with a key wire in the lumen configured to releasably engage the anchor. In a further example pulling the key wire releases the anchor from the anchor control device.

In a preferred example, the template has at least one concave base and at least two apexes. In a further example, the width of the concave base is equal to the depth of the concavity. In a further example, the width of the concave base is greater than the depth of the concavity. In a further example, the width of the concavity is at least 1.5 times the depth of the concavity. In a further example, the width of the concavity is at least 2.5 times the depth of the concavity. In a further example, the width of the concavity ranges from 1× to 5× the depth of the concavity. In another example, the apex of the template has a flat or convex portion to it. In a preferred example, the template has a concave segment and two apexes, where the apexes have flat and/or convex segments. In a further example, the flat and/or convex segments of the apexes range from 2-40 mm long. In a further example, the flat and/or convex segments remain apposed to and/or affixed to the tissue. In another example, the implant has an apex segment comprising a length sufficient to inhibit tilting of the implant relative to the target tissue.

In a preferred example, the template flexes in at least one direction during contraction of target tissue. In a further example, the template flexes to allow a change in distance between ends of the template as tissue flexes under one or more of the following physiologic conditions: heartbeat, annulus contraction, blood pressure changes, atrial expansion, ventricular expansion, blood flow, etc. In another example, the maximum dimension of the template in situ changes in response to tissue motion and physiologic forces.

In one example, the implant template has at least one concave base and at least one apex connected to the concave base, wherein the apex and concave base are configured to be deformable and formed from one or more of the following types of materials: elastic, superelastic, shape memory, hard tempered, heat treated. Examples of said materials include one or more of the following: Nitinol, stainless-steel, maraging steel, cobalt chromium, or the like.

In another example, the implant template has at least one concave segment and at least one apex segment wherein the at least one apex segment is apposed and/or affixed to the annulus. In a further example, the implant has two or more concave segments separated by one or more apex segments, the concave segments being apposed and affixed to the annulus while one or more apex segments are apposed and/or affixed to the annulus. In another example, said implant has at least one apex on each end of the implant, wherein said apexes are apposed and/or affixed to the annulus. In a further example, the implant has at least one concave segment and at least one apex segment wherein the at least one apex segment is apposed and affixed to the tissue to inhibit tilting and/or rotation of the implant relative to the annulus or tissue.

In a preferred example, the implant template is deployable from a crimped smaller configuration, to a larger or deployed configuration. In a further example, the crimped smaller configuration is smaller in at least one dimension than the deployed configuration. In a preferred example, the crimped smaller configuration passes through a smaller diameter tube than the larger or deployed configuration. In one example, in the crimped configuration, the ends of the implant are folded distally from the middle of the implant. In another example, in the crimped configuration, the ends of the implant are folded proximally from the middle of the implant. In a further example, in the crimped configuration, the ends of the implant are compressed toward the middle of the implant. In a further example, in the crimped configuration, the ends of the implant are compressed toward the middle of the implant and folded out of plane to form a substantially tubular shape.

In a preferred example, the implant template having a preformed shape is deployable from a crimped smaller configuration, to a larger or deployed configuration. In another example, the deployed configuration is the unconstrained shape of the implant. In a further example, the crimped configuration is elastically deformed from the preformed shape. In another example, the implant is held in the crimped configuration until delivered adjacent to the annulus and/or tissue. In another configuration the implant is held in the crimped configuration by being at least partially inserted into a tubular body. In another example, the implant is deployed from the crimped shape to the deployed configuration by disengaging it from the tubular body, allowing it to return substantially to its preformed shape.

In one example, the implant template comprises a preformed template configuration wherein the template comprises at least one concavity and at least one apex connected to said concavity, and a tissue anchor from the concavity and extending beyond the apex of the template in the preformed template configuration. In another example, the length of the anchor extends at least half way from the base of the concavity to the apex. In a further embodiment, the length of the anchor is greater than the depth from the base of the concavity to the apex such that as implant is in proximity to the tissue the anchor contacts tissue in advance of the apex of the template.

In another example, an implant template has a crimped configuration and a deployed configuration, where the implant is delivered in the crimped configuration adjacent to the annulus and/or tissue, and then deployed by forming it in situ to the desired template shape. In another example, an implant having a delivery configuration and a deployed configuration, where the implant is in the delivery configuration is delivered adjacent to the annulus and/or tissue, and then deployed by forming it in situ to the desired template shape.

In one example, an implant having a preformed template wherein the template comprises at least one concavity and at least one apex segment, wherein the apex and concavity are connected, defining the implant depth, and wherein the at least one apex segment has tissue engaging element to affix the template to adjacent annulus, and wherein the concavity comprises an opening to slidably engage a tissue engaging anchor element and lock to the tissue engaging anchor element. In another example, the implant system comprises a template having at least one concavity and at least one apex and having a passage through which a tissue engaging anchor is translatably or slidably coupled.

In one example, an implant having a preformed template wherein the template comprises at least one concavity and at least one apex segment, wherein the apex and concavity are connected, and the radius of curvature of the apex segment is greater than the radius of curvature of the concavity, wherein the apex segment in one example comprises a radius of curvature of at least 1.5 times the radius of curvature of the convex segment, wherein the apex segment in one example comprises a radius of curvature of at least 2.5 times the radius of curvature of the convex segment, wherein the apex segment in one example comprises a radius of curvature ranging from 1× to 5× the radius of curvature of the convex segment.

In one example, an implant having a preformed template wherein the template comprises at least one concavity and at least one apex segment, wherein the apex and concavity are connected by legs, and the shape of the concavity and apex segments are configured to contact the tissue along substantially the entire inner surface of the implant when said tissue is pulled into said template.

In one example, an implant having a preformed template wherein the template comprises at least one concavity and at least one apex segment, wherein the apex and concavity are connected, the concavity having a substantially rounded shape to receive and be coupled substantially along the length of the implant to the tissue and/or annulus when the implant is deployed in the tissue and/or annulus.

In another example, an implant having a preformed template wherein the template comprises at least one concavity and at least one apex segment, wherein the apex and concavity are connected, and wherein the apex and concavity having substantially rounded shapes to receive and be coupled substantially along the length of the implant to the tissue and/or annulus when the implant is deployed in the tissue and/or annulus.

In a preferred example, an implant having a template with one or more concavities connected to one or more apex regions by one or more legs, wherein the one or more concavities have annulus pulling anchors configured to pull inward said annulus region into said concavities, and wherein said one or more apex regions have one or more regions positioned against the annulus region to exert a radially outward force on the annulus, substantially opposing the inward pull force of the one or more concavities annulus pulling anchors. In a preferred example, the outward forces exerted by the apex segments do not reposition the annulus, or do not substantially reposition the annulus, outwardly. In another example, the template concavity repositions an annulus region into said concavity, wherein the circumference of the annulus remains substantially the same.

In another example, an implant having a preformed template wherein the template comprises at least one concavity and at least one apex segment, wherein the apex and concavity are connected, and a tissue engaging anchor configured to draw at least a portion of a peripheral wall of a valve annulus at least partially into the concavity so that a peripheral length of the valve annulus can be foreshortened and/or reshaped to improve coapting of the valve leaflets and/or to eliminate or decrease regurgitation of a valve.

In still another aspect of the present invention, a stent prosthesis for valve repair or replacement comprises a scaffold having patterned structural elements, said stent being expandable from a crimped configuration to an expanded configuration and having sufficient strength to support a body annulus in the expanded configurations, wherein the scaffold comprises at least one circumferential ring comprising struts and crowns, wherein at least one strut in said at least one ring comprises at least one separation region and wherein said at least one separation region comprises a male-female junction and a biodegradable polymer and/or adhesive, said separation region being held together in the crimped configuration and is configured to separate after expansion of the stent under physiologic environment, and at least one valve configured to be coupled to said at least one ring, said valve allowing blood to flow in one direction during the cardiac cycle.

In a seventeenth aspect, the present invention provides an implant for repairing a valve annulus, such as a bicuspid or tricuspid cardiac valve, typically but not necessarily a mitral valve. Implant according to the present invention comprise an attachment component that can be delivered to and secured to a region of the valve annulus. Such attachment components often comprise a plicating or other structure that can be anchored to a region of the annulus, typically being a pre-shaped metallic template having at least one concavity configured to be positioned adjacent to a peripheral wall of the valve annulus. At least one anchor will usually be configured to be coupled to the pre-shaped metallic template and to draw at least one segment of said peripheral wall of the valve annulus into said concavity to thereby reduce a diameter of said annulus in a radially inward direction. A skirt, typically configured to replace, mimic or supplant a valve leaflet, may be attached to the pre-shaped metallic template on a side opposite to that of the concavity, wherein said skirt has a surface configured to seal against one or more apposed valve leaflets during systole. Such sealing may be complete but will more often be only partial while still being effective to reduce valve regurgitation to a clinically significant degree.

In particular examples of these implants, the skirt may have a length in a direction away from the side opposite to that of the concavity which is sufficient to coapt with one or more native valve leaflets in apposition to the skirt. The skirt may have a pliability and length in a direction away from the side opposite to that of the concavity to fold down act as a prosthetic valve leaflet during systole and diastole. The skirt may be constructed of a semi-rigid or flexible material that is biocompatible and hemo-compatible.

In particular examples of these implants, the pre-shaped metallic template may have a surface along a length in an axial direction, wherein the concavity may be formed in said surface and oriented in a lateral direction relative to said length. The pre-shaped metallic template may be deployable to said pre-shaped configuration from a crimped configuration.

In particular examples of these implants, a tether may be coupled at one end to at least one of the pre-shaped metallic templates, the at least one anchor, and the skirt, where the tether has another end configured to be implanted in a tissue surface. The one end of such tethers may be coupled to a free distal end of the skirt, may be coupled to a non-sealing surface of the skirt, may be coupled to the at least one anchor, and/or may be coupled to the pre-shaped metallic template. In certain examples, the other end of the tether may be configured to be implanted in an apex of a heart chamber, may be configured to be implanted in a wall of a heart chamber, may be configured to be implanted in the peripheral wall of the valve annulus at a location diametrically opposite to a location of the pre-shaped metallic template, and/or may be configured to be implanted in one of the wall of the ventricle, an annulus, a papillary muscle, a fibrous trigone, a septum, and the wall of the aorta. Such tethers may comprise any one of a metal wire, a metal filament, a polymeric filament, ePTFE filament, a Dacron filament, a nylon filament, a polypropylene filament, a silk filament, or the like, where term “filament” is understood to encompass both monofilament and multifilament braided constructions, and composites of these materials.

In particular examples of these implants, the pre-shaped metallic template may have a single concavity with a pair of opposed legs disposed about a lateral axis and joined by a curved junction region. Each of the opposed legs each may have a convex surface which is axially and laterally spaced-apart from the concavity and wherein the at least one anchor on the template is further configured to draw adjacent segments of said peripheral wall of the valve annulus against the convex surfaces. In some instances, an additional anchor may be located on one or both of the convex surfaces of the opposed legs.

In other examples of these implants, the pre-shaped metallic template may have at least two concavities separated by a convexity. In such instances, each concavity may have at least one anchor configured to draw at least one segment of said peripheral wall of the valve annulus into said concavity.

In particular examples of these implants, the at least one region of the annulus may comprise all or a portion of a posterior mitral valve annulus. The pre-shaped metallic template may comprise an elongate structure having a length in a range from 10 mm to 30 mm and may have a width of the concavity in a range from ½ to 5 times the depth of the concavity. The at least one anchor comprises a helical anchor having a distal end and a proximal end, said distal end having a sharpened tip, and said proximal end being rotatably secured in the concavity of the template, where the anchor may be configured to be coupled to the tissue while the template is coupled to the anchor or may be configured to be coupled to the tissue before the anchor is coupled to the template.

In a eighteenth aspect, the present invention provides systems comprising an implant as described above in combination with a driver for endovascularly delivering the implant to a target location typically a valve annulus, more typically a mitral or other cardiac valve annulus. Such drivers may be configured to detachably attach to and drive the distal tip of the at least one anchor of the implant into the annulus and draw the at least one segment of the peripheral wall of the annulus into the concavity of the template or other implant component. In some instances, the template may be slidably coupled to the detachable driver so that the template can be moved distally relative to the detachable driver to couple with the anchor. In other instances, the template may be rotatably coupled to a helical anchor, typically being in a rotary bearing structure that prevents or inhibits distal/proximal movement.

In particular examples of these systems, the implant may have any one or more of the features described above with respect to the implants of the present invention.

In a nineteenth aspect, the present invention provides a method for repairing a valve annulus, such as a bicuspid or tricuspid cardiac valve, typically but not necessarily a mitral valve. Such methods may comprise delivering in a crimped configuration a metallic implantable template having a tissue-engaging surface pre-shaped with at least one concavity. The template may be expanded with an open end of the at least one concavity oriented against a peripheral surface of the valve annulus. At least one segment of the peripheral surface of the valve annulus is drawn into the concavity to reduce a diameter of said valve annulus (plicate) A skirt may be deployed from a side of the pre-shaped metallic template opposite to that of the concavity, where said skirt has a surface configured to seal against one or more apposed valve leaflets during systole.

In particular examples of these methods, the implant may have any one or more of the features described above with respect to the implants of the present invention.

In particular examples of these methods, implanting may further comprise implanting an anchor end of a tether coupled at another end to at least one of the pre-shaped metallic template, the at least one anchor, and the skirt in a tissue surface. The one end of such tethers may be coupled to a free distal end of the skirt, may be coupled to a non-sealing surface of the skirt, may be coupled to the at least one anchor, and/or may be coupled to the pre-shaped metallic template. In certain examples, the other end of the tether may be implanted in an apex of a heart chamber, may be implanted in a wall of a heart chamber, may be implanted in the peripheral wall of the valve annulus at a location diametrically opposite to a location of the pre-shaped metallic template, and/or may be implanted in one of the wall of the ventricle, an annulus, a papillary muscle, a fibrous trigone, a septum, and the wall of the aorta. Such tethers may comprise any one of a metal wire, a metal filament, a polymeric filament, ePTFE filament, a Dacron filament, a nylon filament, a polypropylene filament, a silk filament, or the like, where term “filament” is understood to encompass both monofilament and multifilament braided constructions, and composites of these materials.

In particular examples of these methods, implanting may further comprise drawing at least one segment of the peripheral surface of the valve annulus into the concavity aligns the template with the valve annulus, for example by engaging an anchor against the annulus segment to apply tension or compression to draw said annulus segment into said concavity. In instances where the anchor comprises a helical coil, drawing may comprise rotating the helical coil to penetrate the peripheral surface of the valve annulus, where the helical coil may be detachably attached to a driver and rotating the helical coil comprises rotating the driver. In other instances, the metallic implantable template is slidably coupled to said driver, and the method further comprises applying tension to said driver and said helical coil to draw said annulus segment into said concavity.

In particular examples of these methods, the template may be locked to the helical coil after the annulus segment has been drawn into said concavity. For example, the driver may be advanced and rotated to implant the helical coil in the valve annulus, the template may be advanced over the driver and coupled to the helical coil after the coil has been implanted in the valve annulus, the driver may be detached from the coil after the template has been advanced over the shaft and coupled to the coil, and helical coil then locked to the template to prevent counter-rotation and accidental loosening and/or detachment of the template to the anchor.

In particular examples of these methods, the anchor comprises a helical coil rotatably attached to the template and drawing comprises rotating the helical coil so that the tissue is drawn into the concavity while the anchor remains attached to the template. The template may be constrained in the crimped configuration and expanding comprises releasing the template from constraint. The peripheral surface may include at least a portion of a mitral valve annulus, tricuspid valve annulus, an aortic valve annulus, or a pulmonary valve annulus.

In particular examples of these methods, drawing may consist of drawing a single segment of the peripheral surface of the annulus into a single concavity on a single template. Alternatively, drawing may comprise drawing at least two segments of the peripheral surface of the annulus into at least two concavities on a single template. In all instances, engaging the template against the peripheral surface of the valve annulus may comprise intravascularly advancing the template.

In a twentieth aspect, the present invention provides an implant for reshaping a valve annulus in a heart chamber, where the implant may comprise a pre-shaped metallic template having a length in an axial direction and at least one concavity in a lateral direction along said length. The concavity may have a concave surface configured to be positioned adjacent to a peripheral wall of the valve annulus. At least one anchor may be configured to be coupled to the pre-shaped metallic template and to draw at least one segment of said peripheral wall of the valve annulus into said concavity to thereby reduce a diameter of said annulus in a radially inward direction. The template is typically deployable to said pre-shaped configuration from a crimped configuration, and one or more tether may be coupled at one end to at least one of the pre-shaped metallic templates and the at least one anchor, and having another end configured to be implanted in a tissue surface in the heart chamber.

In particular examples of these implants, the tether may comprise any one of a metal wire, a metal filament, a polymeric filament, ePTFE filament, dacron filament, nylon filament, polypropylene filament, silk filament, or the like, where filament is understood to encompass both monofilament and multifilament braided constructions and composites of these materials. The other end of the tether may comprise at least one tissue anchoring mechanism selected from a suture, a pledget, a staple, a clip, a helical coil, and a barb, and the tether may have an adjustable length. The adjustment can take place where the tether is coupled to the implant, where the tether is coupled to the tissue anchoring mechanism, or along the length of the tether. The other end of the tether may be configured to be implanted in at least one of a septum, an annulus, a fibrous trigone, the wall of the aorta, and an apex of the heart chamber.

In particular examples of these implants, the implant may comprise a plurality of tethers where at least some of the plurality of tethers may be interconnected into a network, or other two- or three-dimensional structure. In some instances, at least two tethers may be connected to at least one of the pre-shaped metallic templates and the at least one anchor. In other instances, at least two of tethers may be configured to be implanted in the tissue surface in the heart chamber.

In particular examples of these implants, the pre-shaped metallic template may have a single concavity with a pair of opposed legs disposed about a lateral axis and joined by a curved junction region. Each of the opposed legs may have a convex surface which is axially and laterally spaced-apart from the concavity, and the at least one anchor on the template may be further configured to draw adjacent segments of said peripheral wall of the valve annulus against the convex surfaces. Often, an anchor may be disposed on each of the convex surfaces of the opposed legs.

In particular examples of these implants, the pre-shaped metallic template may have at least two concavities separated by a convexity. Each concavity may have at least one anchor configured to draw at least one segment of said peripheral wall of the valve annulus into said concavity, and the annulus may comprise all or a portion of a posterior mitral valve annulus.

In particular examples of these implants, the pre-shaped metallic template may comprise an elongate structure having a length in a range from 10 mm to 30 mm, where the width of the concavity may be in a range from 1 to 5 times the depth of the concavity.

In particular examples of these implants, at least one anchor may comprise a helical anchor having a distal end and a proximal end, where the distal end has a sharpened tip and the proximal end is rotatably secured in the concavity of the template.

In particular examples of these implants, the anchor may be configured to be coupled to the tissue while the template is coupled to the anchor. Alternatively, the anchor may be configured to be coupled to the tissue before the anchor is coupled to the template.

In a twenty-first aspect, the present invention provides a system comprising an implant as described previously in combination with a driver configured to detachably attach to the anchor to implant anchor into the annulus and draw at least a segment of an inner surface of the annulus into the concavity. The template may be slidably coupled to the detachable driver and can be moved distally relative to the detachable driver to couple with the anchor. Alternatively, the template may be rotatably coupled to the anchor.

In a twenty-second aspect, the present invention provides method for reshaping a valve annulus comprising: delivering a metallic implantable template having a tissue-engaging surface pre-shaped with at least one concavity into a heart chamber. At least one concavity may be engaged against a peripheral surface of the valve annulus, and at least one segment of the peripheral surface of the valve annulus may be drawn into the concavity to reduce a diameter of said valve annulus. At least one of the pre-shaped metallic templates and the at least one anchor may be tethered to a tissue surface in the heart chamber.

In particular examples of these methods, tethering may comprise implanting one end of a tether in the tissue surface of the heart chamber, sometimes comprising implanting ends of a plurality of tethers in the tissue surface of the heart chamber. In certain instances, tethering may comprise attaching ends of a plurality tethers to at least one of the pre-shaped metallic templates and the at least one anchor. For example, tethering may comprise attaching a network of tethers between (1) at least one of the pre-shaped metallic templates and the at least one anchor and (2) the tissue surface in the heart chamber. The tether may be adjustable at either end, or along the length of the tether.

In particular examples of these methods, drawing at least one segment of the peripheral surface of the valve annulus into the concavity aligns the template with the valve annulus. In certain instances, drawing at least one segment of the peripheral surface of the valve annulus into the concavity may comprise engaging an anchor against the annulus segment to apply tension or compression to draw said annulus segment into said concavity. For example, a helical coil may be rotated to penetrate the peripheral surface of the valve annulus into the concavity. The helical coil may be detachably attached to a driver and rotating the helical coil comprises rotating the driver. The metallic implantable template may be slidably coupled to said driver where applying tension to said driver and said helical coil draws the annulus segment into the concavity.

In particular examples of these methods, the template may be locked to the helical coil after the annulus segment has been drawn into said concavity. For example, the driver may be advanced and rotated to implant the helical coil in the valve annulus, and the template may be advanced over the driver and coupled to the helical coil after the coil has been implanted in the valve annulus. The driver may then be detached from the coil after the template has been advanced over the shaft and coupled to the coil.

In particular examples of these methods, the anchor may comprise a helical coil rotatably attached to the template and the helical coil may be rotated to draw the tissue into the concavity while the anchor remains attached to the template. The template may be constrained in the crimped configuration and released from constraint to expand.

In particular examples of these methods, the peripheral surface may include at least a portion of a mitral valve annulus, tricuspid valve annulus, an aortic valve annulus, or a pulmonary valve annulus. Drawing may consist of drawing a single segment of the peripheral surface of the annulus into a single concavity on a single template. Alternatively, drawing may comprise drawing at least two segments of the peripheral surface of the annulus into at least two concavities on a single template. Drawing the template against the peripheral surface of the valve annulus may comprise intravascularly advancing the template.

In a twenty-third aspect, the present invention provides an implant for repairing a valve annulus comprising both a skirt and a tether as described with respect to previous embodiments. In particular, the implant comprises a pre-shaped metallic template having at least one concavity configured to be positioned adjacent to a peripheral wall of the valve annulus. At least one anchor is configured to be coupled to the pre-shaped metallic template and to draw at least one segment of said peripheral wall of the valve annulus into said concavity to thereby reduce a diameter of said annulus in a radially inward direction. A skirt attached to the pre-shaped metallic template on a side opposite to that of the concavity, wherein said skirt has a surface configured to seal against one or more apposed valve leaflets during systole, and a tether is coupled at one end to at least one of the pre-shaped metallic template, the at least one anchor, and the skirt and having another end configured to be implanted in a tissue surface. The tether may have an adjustable length. The adjustment can take place where the tether is coupled to at least one of the pre-shaped metallic template, the at least one anchor, and the skirt, or at the other end configured to be implanted in a tissue surface, or along the length of the tether.

Particular examples of these implants comprising both a skirt and a tether may further incorporate any of the features of the implants incorporating either the skirt or the tether, as described previously herein.

In a twenty-fourth aspect of the present invention, a system may comprise an implant including both a skirt and a tether in combination with a driver configured to detachably attach to the anchor to implant anchor into the annulus and draw at least a segment of an inner surface of the annulus into the concavity. The template may be slidably coupled to the detachable driver and can be moved distally relative to the detachable driver to couple with the anchor. Alternatively, the template may be rotatably coupled to a helical anchor.

Particular examples of these systems for delivering implants comprising both a skirt and a tether may further incorporate any of the features of the systems for delivering implants incorporating either the skirt or the tether, as described previously herein.

In a twenty-fifth aspect of the present invention, a method for repairing a valve annulus comprises delivering in a crimped configuration a metallic implantable template having a tissue-engaging surface pre-shaped with at least one concavity. The template is expanded with an open end of the at least one concavity oriented against a peripheral surface of the valve annulus. At least one segment of the peripheral surface of the valve annulus is drawn into the concavity to reduce a diameter of said valve annulus. A skirt from a side of the pre-shaped metallic template opposite to that of the concavity, wherein said skirt has a surface configured to seal against one or more apposed valve leaflets during systole, and at least one of the pre-shaped metallic template, the at least one anchor, and the skirt is tethered to a tissue surface in the heart chamber.

Particular examples of these methods for delivering implants comprising both a skirt and a tether may further incorporate any of the features of the methods for delivering implants incorporating either the skirt or the tether, as described previously herein.

In a twenty-sixth aspect of the present invention, a method for positioning a cardiac valve leaflet, typically a native valve leaflet, in a patient comprises implanting a spacer beneath the valve leaflet and against a ventricular wall, wherein the spacer repositions an inner surface of the valve leaflet away from the ventricular wall and toward an opposed valve leaflet, typically improving coaption between the repositioned valve leaflet and the opposes valve leaflet is improved.

In particular examples of these methods for repositioning the valve leaflet, the spacer may be cylindrical and may be positioned to have an axis aligned with the patient's horizontal plane. Alternatively, the spacer may be cylindrical and may positioned to have an axis aligned with the patient's sagittal plane. Implanting may comprise introducing the spacer in a radially constrained configuration and releasing the spacer to self-expand in situ. For example, the spacer is delivered by a catheter over a guidewire.

In a twenty-seventh aspect of the present invention, an implant for repositioning a valve leaflet by placing the implant between the target leaflet and adjacent ventricular wall in a crimped state and expanding the implant in situ to move the target leaflet toward an opposed coapting leaflet.

In particular examples, the implant comprises a scaffold, for example a stent-like structure. The stent scaffold may be expanded via a balloon and plastically deformed from the crimped state to the expanded state. The stent scaffold may be constrained in the crimped state and released to the expanded state. Typically, the implant is maintained in place by interaction with the chordae of the target leaflet and may comprise features to partially surround the chordae of the target leaflet. The implant may have a hemostatic coating which creates an additional coaptation surface for the opposed coapting leaflet. The volume of the implant may be substantially closed, substantially filled, or combinations thereof. The implant may be placed via a catheter traversing the aorta. The catheter may be placed over a guidewire positioned between the target leaflet and the ventricular wall, and an enclosed volume of the spacer may be adjusted to adjust the apposition of the target valve leaflet and the opposed coapting leaflet.

In a twenty-eighth aspect of the present invention, an implant delivery system comprises an implant configured to engage and reshape a cardiac valve annulus and a control wire having a proximal end and a distal end. The implant is configured to be advanced in a distal direction over the control wire while being free the implant is rotatable with respect to the control wire. At least one anchor detachably secured to the distal end of the control wire, and an interlock is disposed on the anchor. The interlock is configured to receive and lock the implant as after the anchor has been penetrated into tissue and implant has been advanced to the distal end of the control wire.

In particular examples of this implant delivery system, the interlock inhibits at least one of relative axial and rotational motion between the anchor and the implant. The interlock typically inhibits both relative axial and rotational motion between the anchor and the implant after the implant has been full advanced distally over the control wire and onto the anchor. The interlock may comprise any one of protrusions, splines, and flat surfaces to prevent rotation. For example, the at least one anchor comprises a helical anchor having a distal end and a proximal end, said distal end having a sharpened tip, and said proximal end being rotatably secured in the concavity of the template, wherein the rotatable anchor is configured to be engaged and rotated by at least one flexible tension member to rotate the helical anchor into the annulus and draw at least a segment of the inner surface of the annulus into the concavity.

The following numbered clauses describe other examples, aspects, and embodiments of the inventions described herein:

Clause 1. An implantable system comprising:
an implantable component having a component coupling element;
a tissue anchor having an anchor-coupling element; and
an elongate driver removably attached to the anchor-coupling element;
wherein the anchor-coupling element is configured to be coupled to the component coupling element after the tissue anchor has been embedded in tissue by the driver;
wherein the implantable component is configured to be advanced over the elongate driver after the tissue anchor has been embedded in tissue; and
wherein the component coupling element is configured to be attached to the anchor coupling element after the tissue anchor has been embedded in tissue.

Clause 2. The implantable system of clause 1, wherein the component coupling element and the anchor coupling element form an interlocking connection when brought together.

Clause 3. The implantable system of clause 1, wherein the component coupling element comprises a female connector and the anchor coupling element comprises a male connector.

Clause 4. The implantable system of clause 1, wherein the component coupling element is further configured to be detached from the anchor coupling element after the tissue anchor has been embedded in tissue.

Clause 5. The implantable system of clause 1, wherein the tissue anchor is configured to be rotationally advanced into tissue by the driver.

Clause 6. The implantable system of clause 5, wherein the tissue anchor comprises a helix, a screw, a coil, or a spiral.

Clause 7. The implantable system of clause 1, wherein the tissue anchor comprises a ratcheting tether, a hook, a barb, a fastener, a clip, a lock, or a staple.

Clause 8. The implantable system of clause 1, wherein the implantable component comprises an implant for reshaping a valve annulus.

Clause 9. The implantable system of clause 8, wherein the implant for reshaping a valve annulus comprises a pre-shaped template having a length in an axial direction and at least one concavity or convexity in a lateral direction along said length, said concavity or convexity having a surface configured to be positioned adjacent to a peripheral wall of the valve annulus.

Clause 10. The implantable system of clause 1, wherein the implant comprises a prosthetic heart valve.

Clause 11. The implantable system of clause 1, wherein the implant comprises an annuloplasty ring.

Clause 12. The implantable system of clause 1, further comprising at least a second tissue anchor and at least a second driver, wherein the implantable component comprises at least a second component coupling element; and wherein the anchor coupling element is configured to be coupled to the component coupling element after the tissue anchor has been embedded in tissue by the driver; wherein the implantable component is configured to be advanced over the elongate driver after the tissue anchor has been embedded in tissue; and wherein the component coupling element is configured to be attached to the anchor coupling element after the tissue anchor has been embedded in tissue.

Clause 13. The implantable system of clause 1, further comprising an elongate tool configured to detach and remove component coupling element from the anchor coupling element after the tissue anchor has been embedded in tissue.

Clause 14. The implantable system of clause 1, further comprising a pusher configured to be advanced over the elongate driver to push the implantable component.

Clause 15. A method for attaching an implantable component at a target location on a patient's tissue surface, said method comprising:
providing an elongate driver having a distal end removably attached to a tissue anchor;
advancing the elongate driver to the target location;
actuating the elongate driver to implant the tissue anchor at the tissue location;
advancing an implantable component over the elongate driver to the target location;
attaching a coupling element on the implantable component to a coupling element on the implanted tissue anchor;
detaching the distal end of the elongate driver from the implantable component; and
removing the elongate driver from the target location.

Clause 16. The method of clause 15, wherein the implantable component is a template, and when coupled to the tissue anchor pulls tissue into an inner surface of the template.

Clause 17. The method of clause 16, wherein the template has one or more arms on either side of said anchor configured to substantially maintain in place the tissue pulled into said template.

Clause 18. The method of clause 15, wherein actuating the elongate driver to implant the tissue anchor at the tissue location comprises rotating the driver to screw in the tissue anchor.

Clause 19. The method of clause 15, wherein advancing the implantable component over the elongate driver to the target location comprises pushing the implantable component with a pusher.

Clause 20. The method of clause 19, wherein the pusher is advanced over the elongate driver.

Clause 21. The method of clause 19, wherein attaching the coupling element on the implantable component to a coupling element on the implanted tissue anchor comprises interlocking the coupling elements together.

Clause 22. The method of clause 15, wherein the tissue location is a heart valve annulus.

Clause 23. The method of clause 22, wherein access to the heart valve annulus is through the wall of the atrium.

Clause 24. The method of clause 22, wherein access to the heart valve annulus is through the atrial septum.

Clause 25. The method of clause 22, wherein access to the heart valve annulus is through a hole formed at the apex of the heart.

Clause 26. The method of clause 15, wherein the tissue location is a mitral valve annulus.

Clause 27. The method of clause 15, wherein the implantable component comprises an implant for reshaping a valve annulus.

Clause 28. The method of clause 27, wherein the implant for reshaping a valve annulus comprises a pre-shaped template having a length in an axial direction and at least one concavity or convexity in a lateral direction along said length, said concavity or convexity having a surface configured to be positioned adjacent to a peripheral wall of the valve annulus.

Clause 29. The method of clause 15, wherein the implantable component comprises a prosthetic heart valve.

Clause 30. The method of clause 15, wherein the implantable component comprises an annuloplasty ring.

Clause 31. The method of clause 15, wherein the implantable component comprises an annuloplasty ring.

Clause 32. The method of clause 15, further comprising:
providing at least a second elongate driver having a distal end removably attached to a second tissue anchor;
advancing the second elongate driver to the target location;
actuating the second elongate driver to implant the second tissue anchor at the tissue location;
advancing the implantable component over the second elongate driver simultaneously with advancement over the first elongate driver;
attaching a coupling element on the implantable component to a coupling element on the second implanted tissue anchor;
detaching the distal end of the second elongate driver from the implantable component; and
removing the second elongate driver from the target location.

Clause 33. An implantable system for reshaping a heart valve annulus comprising: an implantable component having a component coupling element; and
a tissue anchor component having an anchor coupling element;
wherein the tissue anchor coupling element is configured to self-couple to the component coupling element after the tissue anchor has been embedded in tissue; and
wherein the implant for reshaping a valve annulus comprises a pre-shaped template having a length in an axial direction and at least one concavity or convexity in a lateral direction along said length, said concavity or convexity having a surface configured to be positioned adjacent to a peripheral wall of the valve annulus.

Clause 34. The implantable system of clause 33, further comprising:
an elongate driver removably attached to the anchor coupling element;
wherein the implantable component is configured to be advanced over the elongate driver after the tissue anchor has been embedded in tissue.

Clause 35. The implantable system of clause 33, wherein the component coupling element comprises a female connector and the anchor coupling element comprises a male connector.

Clause 36. The implantable system of clause 33, the component coupling element is further configured to be detached from the anchor coupling element after the tissue anchor has been embedded in tissue.

Clause 37. The implantable system of clause 36, further comprising an elongate tool configured to detach and remove component coupling element from the anchor coupling element after the tissue anchor has been embedded in tissue.

Clause 38. The implantable system of clause 33, further comprising a pusher configured to be advanced over the elongate driver to push the implantable component.

Clause 39. The implantable system of clause 34, wherein the anchor coupling element is configured to be rotationally advanced into tissue by the driver.

Clause 40. The implantable system of clause 39, wherein the anchor coupling element comprises a helix, a screw, a coil, or a spiral.

Clause 41. The implantable system of clause 39, wherein the anchor coupling element comprises a ratcheting tether, a hook, a barb, a fastener, a clip, a lock, or a staple.

Clause 42. The implantable system of clause 34, further comprising at least a second tissue anchor and at least a second driver, wherein the implantable component comprises at least a second component coupling element; and wherein the anchor coupling element is configured to be coupled to the component coupling element after the tissue anchor has been embedded in tissue by the driver; wherein the implantable component is configured to be advanced over the elongate driver after the tissue anchor has been embedded in tissue; and wherein the component coupling element is configured to be attached to the anchor coupling element after the tissue anchor has been embedded in tissue.

Clause 43. A method for attaching an implant to a patient's valve annulus, said method comprising:
implanting a tissue anchor at the target tissue location;
advancing the implant to the valve annulus, wherein the implant comprises a pre-shaped template having a length in an axial direction and at least one concavity or convexity in a lateral direction along said length, said concavity or convexity having a surface configured to be positioned adjacent to a peripheral wall of the valve annulus;
attaching a coupling element on the pre-shaped template to a coupling element on the implanted tissue anchor.

Clause 44. The method of clause 43, wherein attaching the coupling element on the pre-shaped template to the coupling element on the implanted tissue anchor comprises bringing the coupling elements together to form an interlocking connection.

Clause 45. The method of clause 43, further comprising:
providing an elongate driver having a distal end removably attached to the tissue anchor;
advancing the elongate driver to the target location;
actuating the elongate driver to implant the tissue anchor at the target tissue location;
detaching the distal end of the elongate driver from the implantable component; and
removing the elongate driver from the target location.

Clause 46. The method of clause 45, wherein actuating the elongate driver to implant the tissue anchor at the tissue location comprises rotating the driver to screw in the tissue anchor.

Clause 47. The method of clause 46, wherein advancing the implantable component over the elongate driver to the target location comprises pushing the implantable component with a pusher.

Clause 48. The method of clause 47, wherein the pusher is advanced over the elongate driver.

Clause 49. The method of clause 47, wherein attaching the coupling element on the implantable component to a coupling element on the implanted tissue anchor comprises interlocking the coupling elements together.

Clause 50. The method of clause 45, wherein the tissue location is a heart valve annulus.

Clause 51. The method of clause 50, wherein the tissue location is a mitral valve annulus.

Clause 52. The method of clause 45, further comprising:
providing at least a second elongate driver having a distal end removably attached to a second tissue anchor;
advancing the second elongate driver to the target location;
actuating the second elongate driver to implant the second tissue anchor at the tissue location;
advancing the implantable component over the second elongate driver simultaneously with advancement over the first elongate driver;
attaching a coupling element on the implantable component to a coupling element on the second implanted tissue anchor;
detaching the distal end of the second elongate driver from the implantable component; and
removing the second elongate driver from the target location.

Clause 53. The method of clause 52, wherein the tissue anchors are implanted at a distance from each other which is greater than a distance between the coupling elements on the implantable component.

Clause 54. A method for attaching a ring implant to a native valve annulus, said method comprising:
implanting a plurality of tissue anchors at target tissue locations on the valve annulus;
advancing the ring implant to the valve annulus,
attaching a plurality of coupling element on the ring implant to the plurality of coupling elements implanted in the valve annulus.

Clause 55. The method of clause 54, wherein the tissue anchors are implanted at a distance from each other which is greater than a distance between the coupling elements on the ring implant.

Clause 56. The method of clause 54, wherein the ring implant forms a base for further implants.

Clause 57. The method of clause 56 wherein the base is formed from one or more partial ring implants.

Clause 58. The method of clause 56 wherein the further implant is a valve prosthesis configured to replace a native valve.

Clause 59 The method of clause 58 wherein the valve prosthesis is coupled directly to one or more of the tissue anchors.

Clause 60. The method of clause 58 wherein the valve prosthesis is coupled directly to a portion of the valve anatomy including one or more of the following; the annulus, the leaflet, the chordae, the muscular wall of the ventricle, the fibrous skeleton of the heart.

Clause 61. The method of clause 54, wherein attaching the coupling elements on the ring implant to the coupling elements on the implanted tissue anchors comprises bringing the coupling elements together to form interlocking connections.

Clause 62. The method of clause 54, further comprising:
providing a plurality of elongate drivers, each having a distal end removably attached to one tissue anchor of the plurality of the tissue anchors;
advancing the elongate drivers to the target tissue locations;
actuating the elongate driver to implant the tissue anchors at each of the target tissue locations in the valve annulus;
detaching the distal ends of each of the elongate drivers from the tissue anchors; and
removing the elongate drivers from the valve annulus.

Clause 63. The method of clause 62, wherein actuating the elongate drivers to implant the tissue anchor at the tissue location comprises rotating the elongate driver to screw in the tissue anchor.

Clause 64. The method of clause 62, wherein advancing the ring implant to the valve annulus comprises advancing the ring implant over the elongate drivers to the target location by pushing the ring implant with a pusher.

Clause 65. The method of clause 62, wherein attaching the coupling element on the implantable component to a coupling element on the implanted tissue anchor comprises interlocking the coupling elements together.

Clause 66. The method of clause 54, wherein the tissue location is a mitral valve annulus.

Clause 67. The method of clause 54, further comprising:
advancing a heart valve prosthesis over the plurality of elongate drivers; and
attaching the heart valve prosthesis to the ring implant.
detaching the distal end of the second elongate driver from the implantable component; and
removing the second elongate driver from the target location.

Clause 68. An elongated template comprising at least two ends and a body disposed therebetween wherein an annulus region is pulled into said template by means disposed on the body of the template.

Clause 69. The template of clause 68 wherein the force of the pulling means is offset by the sum of the forces at the two ends of the template.

Clause 70. The template of clause 68 wherein the force of the pulling means is offset by the sum of the forces and moments at the two ends of the template.

Clause 71. The template of clause 68 wherein the template is deformed from a first configuration to a second configuration after said annulus region is pulled into said template.

Clause 72. The template of clause 71 wherein the deformation is caused by triggered shape memory in-situ.

Clause 73. The template of clause 71 wherein the deformation is caused by triggered biphasic shape in-situ.

Clause 74. The template of clause 71 wherein the deformation is caused by triggered metastable shape in-situ.

Clause 75. The template of clause 68 wherein one of the ends provides the pulling means.

Clause 76. The template of clause 68 which reshapes a segment of a valve annulus such that the segments of annulus in contact with each end of the template are moved closer to each other, while the segment of the annulus coupled to the concavity of the template moves closer to the opposite wall of the annulus.

Clause 77. A partial ring implant having at least two ends and having a delivery configuration and a functional configuration, wherein the delivery configuration a first distance between the two ends, and wherein the functional configuration has a second distance between the two ends.

Clause 78. The partial ring of clause 77 wherein the first distance is greater than the second distance.

Clause 79. The partial ring of clause 77 wherein the first distance is less than the second distance.

Clause 80. An elongated template comprising a body having at least 2 ends wherein the body comprises an undulating configuration, said template is configured to have at least one anchor configured to draw annular tissue against said undulating body.

Clause 81. The template of clause 8 wherein the undulating means comprises at least one concavity.

Clause 82. The template of clause 80 wherein the undulating means comprises at least one convexity.

Clause 83. The template of clause 80 wherein the undulating means comprises at least one concavity and at least one convexity.

Clause 84. The template of clause 80 wherein the undulating means comprises at least one concavity and at least one substantially linear segment.

Clause 85. The template of clause 80 wherein the undulating means comprises one or more of W, M, Z, U, V, S, J, L shaped bodies.

Clause 86. A ring implant comprising an undulating body wherein said implant is configured to have at least one concavity and at least one anchor configured to draw annular tissue against said one concavity.

Clause 87. The ring implant of clause 86 having at least 2 concavities.

Clause 88. The ring implant of clause 87 wherein the at least 2 concavities are separated by at least one convexity.

Clause 89. The ring implant of clause 87 wherein the at least 2 concavities are separated by at least one substantially linear segment.

Clause 90. The ring implant of clause 86 wherein the ring is folded substantially back on itself, and the resulting folded configuration is straightened for delivery through a tubular body of a smaller diameter than the unconstrained elongated ring.

Clause 91. The implant of clause 90 wherein the ring comprises a plurality of nconcavities.

Clause 92. The ring implant of clause 86 wherein the body comprises a first undulating configuration configured to pass through a tubular body and a second undulating configuration having a shape and size configured to reduce the dimensions of a valve annulus, and a second undulating configuration having a shape and size smaller than the first undulating configuration and configured to pass through a tubular body.

Clause 93. The implant of clause 92 wherein the ring comprises a plurality of concavities.

Clause 94. The ring implant of clause 87 wherein the undulating ring is crimped in a first substantially tubular crimped configuration and wherein the undulating body is deployed to a second, larger, unconstrained configuration.

Clause 95. The implant of clause 94 wherein the ring comprises a plurality of concavities.

Clause 96. The ring implant of clause 87 wherein one or more anchor is attached to one or more concavities prior to delivery of the ring implant to the annular site.

Clause 97. The ring implant of clause 87 wherein one or more anchor is placed at the annular site prior to delivery of the ring implant to the annular site.

Clause 98. The ring implant of clause 97 wherein one or more anchor docks to the ring implant after delivery of the ring implant to the annular site.

Clause 99. A tissue shaping template having at least one concavity and at least two convexities, and tissue coupling means to couple a segment of annulus to the at least one concavity.

Clause 100. The template of clause 99 wherein the annulus segment coupled to the concavity is brought closer to the opposite side of the valve annulus.

Clause 101. The template of clause 99 wherein the two convexities are brought into apposition with corresponding segments of the annulus.

Clause 102. The template of clause 101 wherein the segments of the annulus in apposition with the two convexities are separated by a first separation distance before the template is coupled to the annulus, and by a second separation distance after the template is coupled to the annulus, the second separation distance being less than the first separation distance.

Clause 103. The template of clause 102 wherein the second separation distance is at least 10% less than the first separation distance.

Clause 104. The template of clause 102 wherein the second separation distance is at least 30% less than the first separation distance.

Clause 105. The template of clause 102 wherein the second separation distance is at least 50% less than the first separation distance.

Clause 106. The template of clause 102 wherein the second separation distance is at least 1 mm less than the first separation distance.

Clause 107. The template of clause 102 wherein the second separation distance is at least 5 mm less than the first separation distance.

Clause 108. The template of clause 102 wherein the second separation distance is at least 10 mm less than the first separation distance.

Clause 109. A ring-shaped implant having a functional configuration which is substantially planar and sized to appose a valve annulus, and having a delivery configuration that is substantially non-planar and sized to pass through a catheter.

Clause 110. The implant of clause 109 wherein the ring is a partial ring.

Clause 111. The implant of clause 109 wherein the ring is a complete ring.

Clause 112. The implant of clause 109 wherein the ring is folded in the delivery configuration.

Clause 113. The implant of clause 109 wherein the ring is rolled into a semi-circular form in the delivery configuration.

Clause 114. The implant of clause 110 wherein the ends of the rings are farther apart in the delivery configuration than in the functional configuration.

Clause 115. The implant of clause 112 wherein the folds in the ring are farther apart in the delivery configuration than in the functional configuration.

Clause 116. A system comprising multiple anchors in or around a valve annulus disposed adjacent to each other.

Clause 117. The system of clause 116 wherein the anchors are deployed on a substantially vertical axis.

Clause 118. The system of clause 116 wherein the anchors are deployed on a substantially horizontal axis.

Clause 119. The system of clause 116 wherein the anchors are placed in a first relationship to each other and are coupled to an implant which urges the anchors into a second relationship.

Clause 120. The system of clause 119 wherein the second relationship brings one or more of the anchors closer to one or more of the other anchors than it was in the first relationship.

Clause 121. A template coupled to an annulus region between two other regions on an annular circumference wherein said template is configured to pull said annulus region inwardly bringing closer together said two other regions.

Clause 122. The template of clause 121 further comprising at least one anchor coupled to said annulus region.

Clause 123. The template of clause 122 further comprising additional means to couple said template to said other two regions.

Clause 124. Template coupled to an annulus region between two other regions on an annular circumference wherein said template is configured to pull said annulus region inwardly without “substantially” changing the distance between said two other regions.

Clause 125. The template of clause 124 further comprising at least one anchor coupled to said annulus region.

Clause 126. The template of clause 125 further comprising additional means to couple said template to said other two regions.

Clause 127. A template formed from a substantially flat shape to a substantially undulating shape.

Clause 128. The template of clause 127 cut from a flat sheet of material.

Clause 129. The template of clause 127 cut from a tube of material.

Clause 130. The template of clause 127 formed from a drawn wire of material.

Clause 131. The template of clause 130 where the drawn material has a circular cross section.

Clause 132. The template of clause 130 where the drawn material has a rectangular cross section.

Clause 133. The template of clause 130 where the drawn material has a polygonal cross section.

Clause 134. The template of clause 130 where the drawn material is coupled to at least one anchor interface segments.

Clause 135. The template of clause 134 where the coupling is a crimp.

Clause 136. The template of clause 134 where the coupling is a weld.

Clause 137. The template of clause 134 where the coupling is a braze or solder.

Clause 138. The template of clause 134 where the coupling is glued.

Clause 139. An implant comprising:
an implant body configured for engagement against a tissue surface;
an anchor array support attachable to the implant body; and one or more tissue anchors operatively coupled to the anchor array support.

Clause 140. The implant of clause 139, wherein the implant has a central portion and a peripheral portion and wherein the anchor array support is attachable to the central portion of the implant body.

Clause 141. The implant of clause 140, further comprising one or more individual anchors attachable to the peripheral portion of the implant.

Clause 142. The implant of clause 139, wherein the implant comprises a metallic template configured to engage and deform the tissue surface.

Clause 143. The implant of clause 142, wherein the metallic template is pre-shaped to draw at least one segment of a peripheral wall of a valve annulus into a concavity to thereby reduce a diameter of said annulus in a radially inward direction.

Clause 144. The implant of clause 143, wherein the metallic template is deployable to its pre-shaped configuration from a crimped configuration.

Clause 145. The implant of clause 139, wherein the anchor array support comprises a array support base.

Clause 146. The implant of clause 145, wherein at least some of the anchors comprise elongate attachment members having one end configured to penetrate tissue and at least one of the elongate attachment members has another end configured to be attached to a driver.

Clause 147. The implant of clause 146, wherein each of the elongate attachment members has another end configured to be attached to a driver and wherein each of the elongate attachment members may be rotated by a respective driver.

Clause 148. The implant of clause 146, wherein only a single one of the elongate attachment members has another end configured to be attached to a driver, wherein the remaining elongate attachment members are mechanically coupled to the single elongate attachment member to rotate therewith, and wherein all the elongate attachment members may be rotated by a single driver attached to the single one of the elongate attachment members.

Clause 149. The implant of clause 146, wherein the elongate attachment members comprise helical connectors.

Clause 150. An implant delivery system comprising:
the implant of clause 146; and
a plurality of driver releasably attachable to a plurality of the elongate attachment members.

Clause 151. An implant delivery system comprising:
the implant of clause 148; and
a driver releasably attachable to the single one of the elongate attachment members.

Clause 152. A method for securing an implant body to a target site on a tissue surface, said method comprising:
advancing an anchor array support comprising a plurality of tissue anchors to the target site;
penetrating the plurality of tissue anchors into tissue at the target site to secure the anchor array support to said target site;
   wherein the implant body is secured to the target site by means of the anchor array support.

Clause 153. The method of clause 152, wherein the implant body is attached to the anchor array support prior to advancing the anchor array support to the target site.

Clause 154. The method of clause 152, further comprising attaching the implant body to the anchor array support after the anchor array support has been secured to the target site.

Clause 155. The method of clause 152, wherein the anchor array support is advanced over at least one driver attached to a tissue anchor.

Clause 156. The method of clause 155, wherein the at least one driver is actuated to penetrate the plurality of tissue anchors into tissue at the target site.

Clause 157. The method of clause 156, wherein the at least one driver is mechanically coupled to actuate each of the plurality of tissue anchors simultaneously.

Clause 158. The method of clause 152, wherein the anchor array support is advanced by a plurality of drivers attached to the plurality of tissue anchors.

Clause 159. The method of clause 157, wherein each of the plurality of drivers is actuated to penetrate the plurality of tissue anchors into tissue at the target site.

Clause 160. The method of clause 152, wherein the implant body comprises an implantable template having a tissue-engaging surface.

Clause 161. The method of clause 160, wherein the implant body comprises is pre-shaped with at least one concavity.

Clause 162. The method of clause 161, further comprising expanding the implantable template with an open end of the at least one concavity oriented against a peripheral surface of a valve annulus.

Clause 163. The method of clause 162, wherein further comprising drawing at least one segment of the peripheral surface of the valve annulus into the concavity to reduce a diameter of said valve annulus.

Clause 164. An implant for reshaping a valve annulus, said implant comprising:
a pre-shaped metallic template having a length in an axial direction and at least one concavity in a lateral direction along said length, said concavity having a concave surface configured to be positioned adjacent to a peripheral wall of the valve annulus; and
at least two anchors coupled to the pre-shaped metallic template near a center thereof and extending along a lateral axis between a pair of opposed legs, wherein said at least two anchors are configured to penetrate into said at least one segment of said peripheral wall of the valve annulus and to draw said at least one segment of said peripheral wall into said concavity to thereby reduce a diameter of said annulus in a radially inward direction, wherein said template is deployable to said pre-shaped configuration from a crimped configuration.

Clause 165. The implant of clause 164, comprising at least three anchors coupled to the pre-shaped metallic template near a center thereof.

Clause 166. The implant of clause 164, further comprising an anchor location at a tissue-engaging end of each leg.

Clause 167. The implant of clause 164, wherein the at least two anchors comprise helical anchors rotatably secured in a segment of the pre-shaped metallic template.

Clause 168. The implant of clause 164, wherein the at least two anchors comprise helical anchors rotatably secured in an anchor array support attachable to a body of the pre-shaped metallic template.

Clause 169. The implant of clause 164, wherein the pre-shaped metallic template has a single concavity joined by a single curved junction region.

Clause 170. The implant of clause 164, wherein at least one anchor is configured to rotate about a lateral axis but not translate along the lateral axis relative to the pre-shaped metallic template.

Clause 171. The implant of clause 170, wherein at least one anchor is configured to both rotate about a lateral axis and translate along the lateral axis relative to the pre-shaped metallic template.

Clause 172. A system comprising:
the implant of clause 164; and
a driver configured to detachably attach to and rotate at least one if the helical anchors to drive the distal tip of the helical anchor into the annulus and draw at least a segment of an inner surface of the annulus into the concavity.

Clause 173. The system of clause 172 wherein the template is translatably coupled to the detachable driver and can be moved distally relative to the detachable driver to couple with the anchor.

Clause 174. The system of clause 172 wherein the template is pivotally coupled to the detachable driver and can be pivoted relative to the detachable driver to facilitate implantation.

Clause 175. The system of clause 172 wherein the template is rotatably coupled to the anchor.

Clause 176. An implant for reshaping a valve annulus, said implant comprising:
a pre-shaped metallic template having a length in an axial direction and at least one concavity in a lateral direction along said length, said concavity having a concave surface configured to be positioned adjacent to a peripheral wall of the valve annulus; and
first and second anchors coupled to the pre-shaped metallic template near a center thereof, wherein the first anchor is pre-positioned to extend along a lateral axis between a pair of opposed legs and the second anchor is configured to be advanced from a retracted position to an extended position along the lateral axis between the pair of opposed legs, wherein said first and second anchors are configured to sequentially penetrate into said at least one segment of said peripheral wall of the valve annulus and to draw and stabilize said at least one segment of said peripheral wall into said concavity to thereby reduce a diameter of said annulus in a radially inward direction, wherein said template is deployable to said pre-shaped configuration from a crimped configuration.

Clause 177. The implant of clause 176, further comprising an anchor located at a tissue-engaging end of each leg.

Clause 178. The implant of clause 176, wherein said first and second anchors each comprise a helical anchor rotatably secured to the center of the pre-shaped metallic template, wherein the first anchor is axially fixed relative to the pre-shaped metallic template and the second anchor is configured to translate along the lateral axis between the pair of opposed legs.

Clause 179. The implant of any one of clauses 176-178, wherein the pre-shaped metallic template has a single concavity joined by a single curved junction region.

Clause 180. A system comprising:
the implant of clause 178; and
a driver configured to detachably attach to and individually rotate each of the first and second helical anchors to drive the distal tip of the first helical anchor into the annulus and draw at least a segment of an inner surface of the annulus into the concavity and to thereafter advance and drive the tip of the second anchor into said annulus.

Clause 181. An implant and delivery system for reshaping a valve annulus, said implant delivery system comprising:
a locating tissue anchor detachably secured to a distal end of a locating control wire;
a plurality accessory tissue anchors on a frame detachably coupled to a plurality of accessory control wires; and
a pre-shaped metallic template having a length in an axial direction and at least one concavity in a lateral direction along said length, said concavity having a concave surface configured to be positioned adjacent to a peripheral wall of the valve annulus;
wherein the frame and accessory tissue anchors are configured to be advanced over the locating control wire and embedded in tissue after the locating tissue anchor has been embedded at a tissue target site and wherein the pre-shaped metallic template is configured to be advanced over at least one of the locating control wire and the accessory control wires and coupled to the frame after the frame an and accessory tissue anchors are embedded in tissue.

Clause 182. An implant comprising:
a pre-shaped metallic template having a length in an axial direction and at least one concavity in a lateral direction along said length configured for engagement against a tissue surface;
a helical anchor array rotatably coupled to the pre-shaped metallic template; and
wherein the helical anchor is positioned in a helical track in a wall of the pre-shaped metallic template so that a tissue-penetrating distal tip of the helical anchor can be advanced into a region between a pair of legs of the pre-shaped metallic template by rotation.

Clause 183. The implant of clause 182, further comprising a dock element on a proximal end of the helical anchor, wherein the dock element is configured to detachably engage a rotatable driver.

Clause 184. The implant of clause 182 or 183, wherein the helical anchor has a proximal straight section that decouples from the helical track to allow the helical anchor to be further rotated to cinch implantation after the helical portion of the anchor has passed distally beyond the wall.

Clause 185. An implant for reshaping a valve annulus, said implant comprising:
a pre-shaped metallic template having a length in an axial direction and at least one central concave surface and two lateral convex surfaces configured to engage a posterior peripheral wall of the valve annulus;
a central anchor coupled to the pre-shaped metallic template and extending into a concavity formed by the concave surface to capture and draw a segment of the posterior peripheral wall against said concave surface a radially inward direction,
at least two side anchors coupled to the pre-shaped metallic template on each of right and left sides of the central anchor and extending into from the convex surfaces surface to capture and draw segments of the posterior peripheral wall against said convex surfaces surface.

Clause 186. The implant of clause 185, wherein the pre-shaped metallic template is deployable to said pre-shaped configuration from a crimped configuration.

Clause 187. The implant of clause 185, wherein the pre-shaped metallic template has a single concave surface with left and right convex surfaces on right and left sides thereof.

Clause 188. The implant of clause 187, wherein each of the left and right convex surfaces is free from concave surface regions.

Clause 189. The implant of clause 188, wherein one side anchor is located near a mid-point of each convex surface and one side anchor is located near an end of each convex surface remote from the central concave surface.

Clause 190. The implant of clause 187, wherein each of the left and right convex surfaces has a concave surface region therein.

Clause 191. The implant of clause 190, wherein one side anchor is located in each of the concave surface regions and one side anchor is located near an end of each convex surface remote from the central concave surface.

Clause 192. The implant of any one of the preceding clauses, further comprising a trigonal anchor attached to each of the right and left and right convex surfaces, wherein said trigonal anchors are configured to be coupled to fibrous tissue in a trigon region of the valve annulus.

Clause 193. The implant of clause 192, wherein right and left trigonal anchors are attached to right and left ends of the right and left convex surfaces, respectively.

Clause 194. The implant of clause 192 and clause 193, wherein the right and left trigonal anchors are attached to right and left ends of the right and left convex surfaces by control wires.

Clause 195. The implant of clause 192 and clause 193, wherein the right and left trigonal anchors are slidably coupled to the right and left ends of the right and left convex surfaces by extensions.

Clause 196. The implant of clause 192 and clause 193, wherein the right and left trigonal anchors each comprise a plurality of tension members secured between (a) at least two spaced apart locations on each of the lateral convex surfaces and (b) the fibrous tissue in the trigon region of the valve annulus.

Clause 197. The implant of clause 192 and clause 193, wherein the right and left trigonal anchors each comprise a plurality of tension members secured between (a) the at least one central concave surface and at least two spaced apart locations on each of the lateral convex surfaces and (b) the fibrous tissue in the trigon region of the valve annulus.

Clause 198. A method for reshaping a valve annulus, said method comprising:
delivering a metallic implantable template having at least one central concave surface and two lateral convex surfaces configured to engage a posterior peripheral wall of the valve annulus; anchoring the at least one central concave surface to the posterior peripheral wall to draw at least one segment of the posterior peripheral wall against the concave surface to reduce a diameter of said valve annulus; and
anchoring each of the lateral convex surfaces to the posterior peripheral wall at at least two locations spaced apart along of the valve annulus.

Clause 199. The method of clause 198, wherein delivering comprises expanding the pre-shaped metallic template from a crimped configuration to an expanded configuration to open the at least one central concave surface and deploy the two lateral convex surface and engaging said surfaces against the posterior peripheral wall of the valve annulus.

Clause 200. The method of clause 198 or 199, further comprising attaching free ends of the pre-shaped metallic template to fibrous tissue in a trigon region of the valve annulus

Clause 201. The method of clause 200, wherein the free ends of the pre-shaped metallic template are attached to the fibrous tissue in the trigon region by securing control wires between each end and said fibrous tissue.

Clause 202. The method of clause 200, wherein the free ends of the pre-shaped metallic template are attached to the fibrous tissue in the trigon region by securing template extensions between each end and said fibrous tissue and adjusting the attachment length.

Clause 203. The method of clause 200, wherein the free ends of the pre-shaped metallic template are attached to the fibrous tissue in the trigon region by securing a plurality of tension members between (a) at least two spaced apart locations on each of the lateral convex surfaces and (b) the fibrous tissue in the trigon region of the valve annulus.

Clause 204. The method of clause 200, wherein the free ends of the pre-shaped metallic template are attached to the fibrous tissue in the trigon region by securing a plurality of tension members between (a) the at least one central concave surface and at least two spaced apart locations on each of the lateral convex surfaces and (b) the fibrous tissue in the trigon region of the valve annulus.

Clause 205. A method for reshaping a valve annulus, said method comprising:
measuring a major axis of the valve annulus generally between valve commissures to determine a valve width;
selecting a metallic implantable template having at least one central concave surface and two lateral convex surfaces configured to engage a posterior peripheral wall of the valve annulus, wherein said metallic implantable template has a length between a right end and a left end thereof and wherein said length is within ±______% of the measured valve width; and implanting the metallic implantable template in the valve annulus.

Clause 206. The method of clause 205, wherein delivering comprises;
anchoring the at least one central concave surface to the posterior peripheral wall to draw at least one segment of the posterior peripheral wall against the concave surface to reduce a diameter of said valve annulus; and
anchoring each of the lateral convex surfaces to the posterior peripheral wall at at least two locations spaced apart along of the valve annulus.

Clause 207. The method of clause 206, wherein delivering further comprises expanding the metallic template from a crimped configuration to an expanded configuration to open the at least one central concave surface and deploy the two lateral convex surface and engaging said surfaces against the posterior peripheral wall of the valve annulus.

Clause 208. The method of any one of clauses 205-207, further comprising attaching free ends of the metallic template to fibrous tissue in a trigon region of the valve annulus

Clause 209. The method of clause 208, wherein the free ends of the metallic template are attached to the fibrous tissue in the trigon region by securing control wires between each end and said fibrous tissue.

Clause 210. The method of clause 208, wherein the free ends of the metallic template are attached to the fibrous tissue in the trigon region by securing template extensions between each end and said fibrous tissue.

Clause 211. The method of clause 208, wherein the free ends of the pre-shaped metallic template are attached to the fibrous tissue in the trigon region by securing a plurality of tension members between (a) at least two spaced apart locations on each of the lateral convex surfaces and (b) the fibrous tissue in the trigon region of the valve annulus.

Clause 212. The method of clause 208, wherein the free ends of the pre-shaped metallic template are attached to the fibrous tissue in the trigon region by securing a plurality of tension members between (a) the at least one central concave surface and at least two spaced apart locations on each of the lateral convex surfaces and (b) the fibrous tissue in the trigon region of the valve annulus.

Clause 213. An implant comprising:
an implant body configured for engagement against a tissue surface;
an anchor array support attachable to the implant body; and
one or more tissue anchors operatively coupled to the anchor array support.

Clause 214. The implant of clause 213, wherein the implant has a central portion and a peripheral portion and wherein the anchor array support is attachable to the central portion of the implant body.

Clause 215. The implant of clause 214, further comprising one or more individual anchors attachable to the peripheral portion of the implant.

Clause 216. The implant of clause 213, wherein the implant comprises a metallic template configured to engage and deform the tissue surface.

Clause 217. The implant of clause 216, wherein the metallic template is pre-shaped to draw at least one segment of a peripheral wall of a valve annulus into a concavity to thereby reduce a diameter of said annulus in a radially inward direction.

Clause 218. The implant of clause 217, wherein the metallic template is deployable to its pre-shaped configuration from a crimped configuration.

Clause 219. The implant of clause 213, wherein the anchor array support comprises a array support base.

Clause 220. The implant of clause 219, wherein at least some of the anchors comprise elongate attachment members having one end configured to penetrate tissue and at least one of the elongate attachment members has another end configured to be attached to a driver.

Clause 221. The implant of clause 220, wherein each of the elongate attachment members has another end configured to be attached to a driver and wherein each of the elongate attachment members may be rotated by a respective driver.

Clause 222. The implant of clause 220, wherein only a single one of the elongate attachment members has another end configured to be attached to a driver, wherein the remaining elongate attachment members are mechanically coupled to the single elongate attachment member to rotate therewith, and wherein all the elongate attachment members may be rotated by a single driver attached to the single one of the elongate attachment members.

Clause 223. The implant of clause 220, wherein the elongate attachment members comprise helical connectors.

Clause 224. An implant delivery system comprising:
the implant of clause 220; and
a plurality of driver releasably attachable to a plurality of the elongate attachment members.

Clause 225. An implant delivery system comprising:
the implant of clause 222; and
a driver releasably attachable to the single one of the elongate attachment members.

Clause 226. A method for securing an implant body to a target site on a tissue surface, said method comprising:
advancing an anchor array support comprising a plurality of tissue anchors to the target site;
penetrating the plurality of tissue anchors into tissue at the target site to secure the anchor array support to said target site;
wherein the implant body is secured to the target site by means of the anchor array support.

Clause 227. The method of clause 226, wherein the implant body is attached to the anchor array support prior to advancing the anchor array support to the target site.

Clause 228. The method of clause 226, further comprising attaching the implant body to the anchor array support after the anchor array support has been secured to the target site.

Clause 229. The method of clause 226, wherein the anchor array support is advanced over at least one driver attached to a tissue anchor.

Clause 230. The method of clause 229, wherein the at least one driver is actuated to penetrate the plurality of tissue anchors into tissue at the target site.

Clause 231. The method of clause 230, wherein the at least one driver is mechanically coupled to actuate each of the plurality of tissue anchors simultaneously.

Clause 232. The method of clause 226, wherein the anchor array support is advanced by a plurality of drivers attached to the plurality of tissue anchors.

Clause 233. The method of clause 231, wherein each of the plurality of drivers is actuated to penetrate the plurality of tissue anchors into tissue at the target site.

Clause 234. The method of clause 226, wherein the implant body comprises an implantable template having a tissue-engaging surface.

Clause 235. The method of clause 234, wherein the implant body comprises is pre-shaped with at least one concavity.

Clause 236. The method of clause 235, further comprising expanding the implantable template with an open end of the at least one concavity oriented against a peripheral surface of a valve annulus.

Clause 237. The method of clause 236, wherein further comprising drawing at least one segment of the peripheral surface of the valve annulus into the concavity to reduce a diameter of said valve annulus.

Clause 238. An implant for reshaping a valve annulus, said implant comprising:
a pre-shaped metallic template having a length in an axial direction and at least one concavity in a lateral direction along said length, said concavity having a concave surface configured to be positioned adjacent to a peripheral wall of the valve annulus; and
at least two anchors coupled to the pre-shaped metallic template near a center thereof and extending along a lateral axis between a pair of opposed legs, wherein said at least two anchors are configured to penetrate into said at least one segment of said peripheral wall of the valve annulus and to draw said at least one segment of said peripheral wall into said concavity to thereby reduce a diameter of said annulus in a radially inward direction, wherein said template is deployable to said pre-shaped configuration from a crimped configuration.

Clause 239. The implant of clause 238, comprising at least three anchors coupled to the pre-shaped metallic template near a center thereof.

Clause 240. The implant of clause 238, further comprising an anchor location at a tissue-engaging end of each leg.

Clause 241. The implant of clause 238, wherein the at least two anchors comprise helical anchors rotatably secured in a segment of the pre-shaped metallic template.

Clause 242. The implant of clause 238, wherein the at least two anchors comprise helical anchors rotatably secured in an anchor array support attachable to a body of the pre-shaped metallic template.

Clause 243. The implant of clause 238, wherein the pre-shaped metallic template has a single concavity joined by a single curved junction region.

Clause 244. The implant of clause 238, wherein at least one anchor is configured to rotate about a lateral axis but not translate along the lateral axis relative to the pre-shaped metallic template.

Clause 245. The implant of clause 244, wherein at least one anchor is configured to both rotate about a lateral axis and translate along the lateral axis relative to the pre-shaped metallic template.

Clause 246. A system comprising:
the implant of clause 238; and
a driver configured to detachably attach to and rotate at least one if the helical anchors to drive the distal tip of the helical anchor into the annulus and draw at least a segment of an inner surface of the annulus into the concavity.

Clause 247. The system of clause 246, wherein the template is translatably coupled to the detachable driver and can be moved distally relative to the detachable driver to couple with the anchor.

Clause 248. The system of clause 246, wherein the template is pivotally coupled to the detachable driver and can be pivoted relative to the detachable driver to facilitate implantation.

Clause 249. The system of clause 246, wherein the template is rotatably coupled to the anchor.

Clause 250. An implant for reshaping a valve annulus, said implant comprising:
a pre-shaped metallic template having a length in an axial direction and at least one concavity in a lateral direction along said length, said concavity having a concave surface configured to be positioned adjacent to a peripheral wall of the valve annulus; and
first and second anchors coupled to the pre-shaped metallic template near a center thereof, wherein the first anchor is pre-positioned to extend along a lateral axis between a pair of opposed legs and the second anchor is configured to be advanced from a retracted position to an extended position along the lateral axis between the pair of opposed legs, wherein said first and second anchors are configured to sequentially penetrate into said at least one segment of said peripheral wall of the valve annulus and to draw and stabilize said at least one segment of said peripheral wall into said concavity to thereby reduce a diameter of said annulus in a radially inward direction, wherein said template is deployable to said pre-shaped configuration from a crimped configuration.

Clause 251. The implant of clause 250, further comprising an anchor located at a tissue-engaging end of each leg.

Clause 252. The implant of clause 250, wherein said first and second anchors each comprise a helical anchor rotatably secured to the center of the pre-shaped metallic template, wherein the first anchor is axially fixed relative to the pre-shaped metallic template and the second anchor is configured to translate along the lateral axis between the pair of opposed legs.

Clause 253. The implant of any one of clauses 250-252, wherein the pre-shaped metallic template has a single concavity joined by a single curved junction region.

Clause 254. A system comprising:
the implant of clause 252; and
a driver configured to detachably attach to and individually rotate each of the first and second helical anchors to drive the distal tip of the first helical anchor into the annulus and draw at least a segment of an inner surface of the annulus into the concavity and to thereafter advance and drive the tip of the second anchor into said annulus.

Clause 255. An implant and delivery system for reshaping a valve annulus, said implant delivery system comprising:
a locating tissue anchor detachably secured to a distal end of a locating control wire;
a plurality accessory tissue anchors on a frame detachably coupled to a plurality of accessory control wires; and
a pre-shaped metallic template having a length in an axial direction and at least one concavity in a lateral direction along said length, said concavity having a concave surface configured to be positioned adjacent to a peripheral wall of the valve annulus;
wherein the frame and accessory tissue anchors are configured to be advanced over the locating control wire and embedded in tissue after the locating tissue anchor has been embedded at a tissue target site and wherein the pre-shaped metallic template is configured to be advanced over at least one of the locating control wire and the accessory control wires and coupled to the frame after the frame an and accessory tissue anchors are embedded in tissue.

Clause 256. An implant comprising:
a pre-shaped metallic template having a length in an axial direction and at least one concavity in a lateral direction along said length configured for engagement against a tissue surface; a helical anchor array rotatably coupled to the pre-shaped metallic template; and
wherein the helical anchor is positioned in a helical track in a wall of the pre-shaped metallic template so that a tissue-penetrating distal tip of the helical anchor can be advanced into a region between a pair of legs of the pre-shaped metallic template by rotation.

Clause 257. The implant of clause 256, further comprising a dock element on a proximal end of the helical anchor, wherein the dock element is configured to detachably engage a rotatable driver.

Clause 258. The implant of clause 256 or 257, wherein the helical anchor has a proximal straight section that decouples from the helical track to allow the helical anchor to be further rotated to cinch implantation after the helical portion of the anchor has passed distally beyond the wall.

Clause 259. An implant for repairing a valve annulus, said implant comprising:
a pre-shaped metallic template having at least one concavity configured to be positioned adjacent to a peripheral wall of the valve annulus;
at least one anchor configured to be coupled to the pre-shaped metallic template and to draw at least one segment of said peripheral wall of the valve annulus into said concavity to thereby reduce a diameter of said annulus in a radially inward direction; and
a skirt attached to the pre-shaped metallic template on a side opposite to that of the concavity, wherein said skirt has a surface configured to seal against one or more apposed valve leaflets during systole.

Clause 260. The implant of clause 259, wherein the skirt has a length in a direction away from the side opposite to that of the concavity which is sufficient to coapt with one or more native valve leaflets in apposition to the skirt.

Clause 261. The implant of clause 259, wherein the skirt has a pliability and length in a direction away from the side opposite to that of the concavity to fold down act as a prosthetic valve leaflet during systole and diastole

Clause 262. The implant of any one of the preceding clauses, wherein the skirt is constructed of a semi-rigid or flexible material that is biocompatible and hemo-compatible.

Clause 263. The implant of any one of the preceding clauses, wherein the pre-shaped metallic template has a surface along a length in an axial direction, wherein the concavity is formed in said surface and oriented in a lateral direction relative to said length.

Clause 264. The implant of any one of the preceding clauses, wherein the pre-shaped metallic template is deployable to said pre-shaped configuration from a crimped configuration.

Clause 265. The implant of any one of the preceding clauses, further comprising a tether coupled at one end to at least one of the pre-shaped metallic template, the at least one anchor, and the skirt and having another end configured to be implanted in a tissue surface.

Clause 266. The implant of clause 265, wherein the tether is coupled to a free distal end of the skirt.

Clause 267. The implant of clause 265, wherein the tether is coupled to a non-sealing surface of the skirt.

Clause 268. The implant of clause 265, wherein the tether is coupled to the at least one anchor.

Clause 269. The implant of clause 265, wherein the tether is coupled to the pre-shaped metallic template.

Clause 270. The implant of any one of clauses 265-269 wherein the other end of the tether is configured to be implanted in an apex of a heart chamber.

Clause 271. The implant of any one of clauses 265-269 wherein the other end of the tether is configured to be implanted in a wall of a heart chamber.

Clause 272. The implant of any one of clauses 265-269 wherein the other end of the tether is configured to be implanted in the peripheral wall of the valve annulus at a location diametrically opposite to a location of the pre-shaped metallic template.

Clause 273. The implant of any one of clauses 265-269 wherein the other end of the tether is configured to be implanted in one of the wall of the ventricle, an annulus, a papillary muscle, a fibrous trigone, a septum, and the wall of the aorta

Clause 274. The implant of any one of clauses 265-272 wherein the tether comprises any one of a metal wire, a metal filament, a polymeric filament, ePTFE filament, dacron filament, nylon filament, polypropylene filament, silk filament, or the like, where filament is understood to encompass both monofilament and multifilament braided constructions, and composites of these materials.

Clause 275. The implant of any one of the preceding clauses, wherein the pre-shaped metallic template has a single concavity with a pair of opposed legs disposed about a lateral axis and joined by a curved junction region.

Clause 276. The implant of any one of the preceding clauses, wherein each of the opposed legs each have a convex surface which is axially and laterally spaced-apart from the concavity and wherein the at least one anchor on the template is further configured to draw adjacent segments of said peripheral wall of the valve annulus against the convex surfaces.

Clause 277. The implant of clause 276, further comprising an anchor on each of the convex surfaces of the opposed legs.

Clause 278. The implant of clause any one of the preceding clauses, wherein the pre-shaped metallic template has at least two concavities separated by a convexity.

Clause 279. The implant of clause 278, wherein each concavity has at least one anchor configured to draw at least one segment of said peripheral wall of the valve annulus into said concavity.

Clause 280. The implant of clause any one of the preceding clauses, wherein the at least one region comprises all or a portion of a posterior mitral valve annulus.

Clause 281. The implant of clause any one of the preceding clauses, wherein the pre-shaped metallic template comprises an elongate structure having a length in a range from 10 mm to 30 mm.

Clause 282. The implant of clause 281, wherein the width of the concavity is in a range from 1 to 5 times the depth of the concavity.

Clause 283. The implant of clause any one of the preceding clauses, wherein the at least one anchor comprises a helical anchor having a distal end and a proximal end, said distal end having a sharpened tip, and said proximal end being rotatably secured in the concavity of the template.

Clause 284. The implant of clause any one of the preceding clauses, wherein the anchor is configured to be coupled to the tissue while the template is coupled to the anchor.

Clause 285. The implant of clause any one of the preceding clauses, wherein the anchor is configured to be coupled to the tissue before the anchor is coupled to the template.

Clause 286. A system comprising:
the implant of any one of the preceding clauses; and
a driver configured to detachably attach to and drive the distal tip of the at least one anchor into the annulus and draw the at least one segment of the peripheral wall of the annulus into the concavity.

Clause 287. The system of clause 286 wherein the template is slidably coupled to the detachable driver and can be moved distally relative to the detachable driver to couple with the anchor.

Clause 288. The system of clause 286 wherein the template is rotatably coupled to a helical anchor.

Clause 289. A method for repairing a valve annulus, said method comprising:
delivering in a crimped configuration a metallic implantable template having a tissue-engaging surface pre-shaped with at least one concavity;
expanding said template with an open end of the at least one concavity oriented against a peripheral surface of the valve annulus;
drawing at least one segment of the peripheral surface of the valve annulus into the concavity to reduce a diameter of said valve annulus; and
deploying a skirt from a side of the pre-shaped metallic template opposite to that of the concavity, wherein said skirt has a surface configured to seal against one or more apposed valve leaflets during systole.

Clause 290. The method of clause 289, wherein the skirt has a length in a direction away from the side opposite to that of the concavity which is sufficient to coapt with one or more native valve leaflets in apposition to the skirt.

Clause 291. The method of clause 289, wherein the skirt has a pliability and length in a direction away from the side opposite to that of the concavity to fold down act as a prosthetic valve leaflet during systole and diastole

Clause 292. The method of any one of clauses 289-291, wherein the skirt is constructed of a semi-rigid or flexible material that is biocompatible and hemo-compatible.

Clause 293. The method of any one of clauses 289-292, wherein the pre-shaped metallic template has a surface along a length in an axial direction, wherein the concavity is formed in said surface and oriented in a lateral direction relative to said length.

Clause 294. The method of any one of clauses 289-293, wherein the pre-shaped metallic template is deployable to said pre-shaped configuration from a crimped configuration.

Clause 295. The method of any one of clauses 289-294, further comprising implanting an anchor end of a tether coupled at another end to at least one of the pre-shaped metallic template, the at least one anchor, and the skirt in a tissue surface.

Clause 296. The method of clause 295, wherein the tether is coupled to a free distal end of the skirt.

Clause 297. The method of clause 295, wherein the tether is coupled to a non-sealing surface of the skirt.

Clause 298. The method of clause 295, wherein the tether is coupled to the at least one anchor.

Clause 299. The method of clause 295, wherein the tether is coupled to the pre-shaped metallic template.

Clause 300. The method of any one of clauses 295-299 wherein the other end of the tether is configured to be implanted in an apex of a heart chamber.

Clause 301. The method of any one of clauses 295-300 wherein the other end of the tether is configured to be implanted in a wall of a heart chamber.

Clause 302. The method of any one of clauses 295-300 wherein the other end of the tether is configured to be implanted in the peripheral wall of the valve annulus at a location diametrically opposite to a location of the pre-shaped metallic template.

Clause 303. The method of any one of clauses 289-302, wherein drawing at least one segment of the peripheral surface of the valve annulus into the concavity aligns the template with the valve annulus.

Clause 304. The method of any one of clauses 290-303, wherein drawing at least one segment of the peripheral surface of the valve annulus into the concavity comprises engaging an anchor against the annulus segment to apply tension or compression to draw said annulus segment into said concavity.

Clause 305. The method of clause 304, wherein the anchor comprises a helical coil and drawing comprises rotating the helical coil to penetrate the peripheral surface of the valve annulus.

Clause 306. The method of clause 305, wherein the helical coil is detachably attached to a driver and rotating the helical coil comprises rotating the driver.

Clause 307. The method of clause 306, wherein the metallic implantable template is slidably coupled to said driver and said method further comprises applying tension to said driver and said helical coil to draw said annulus segment into said concavity.

Clause 308. The method of clause 307, further comprising locking the template to the helical coil after the annulus segment has been drawn into said concavity.

Clause 309. The method of clause 307, wherein the driver is advanced and rotated to implant the helical coil in the valve annulus, the template is advanced over the driver and coupled to the helical coil after the coil has been implanted in the valve annulus, and the driver is detached from the coil after the template has been advanced over the shaft and coupled to the coil.

Clause 310. The method of any one of clauses 289-309, wherein the anchor comprises a helical coil rotatably attached to the template and drawing comprises rotating the helical coil so that the tissue is drawn into the concavity while the anchor remains attached to the template.

Clause 311. The method of any one of clauses 289-310, wherein template is constrained in the crimped configuration and expanding comprises releasing the template from constraint.

Clause 312. The method of any one of clauses 289-311, wherein the peripheral surface includes at least a portion of a mitral valve annulus, tricuspid valve annulus, an aortic valve annulus, or a pulmonary valve annulus.

Clause 313. The method of any one of clauses 289-312, wherein drawing consists of drawing a single segment of the peripheral surface of the annulus into a single concavity on a single template.

Clause 314. The method of any one of clauses 289-312, wherein drawing comprises drawing at least two segments of the peripheral surface of the annulus into at least two concavities on a single template.

Clause 315. The method of any one of clauses 289-312, wherein engaging the template against the peripheral surface of the valve annulus comprises intravascularly advancing the template.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative examples, in which the principles of the invention are utilized, and the accompanying drawings of which:

FIG. 1 shows a tissue shaping template having a tissue anchor that connects to the template via a deformable tab aligned substantially with the axis of the tissue anchor.

FIG. 2 shows a tissue shaping template having a tissue anchor that connects to the template via a deformable tab aligned at an angle to the axis of the tissue anchor.

FIG. 3 shows a tissue shaping template having a tissue anchor that connects to the template via a deformable tab aligned perpendicular to the axis of the tissue anchor.

FIG. 4A through 4D show deformable connecting tabs with alternate shapes.

FIG. 5 shows a tissue anchor with a reverse thread on it, such that turning the anchor clockwise to engage the tissue will also cause the reverse thread to engage a template (not shown.)

FIG. 6 shows a tissue anchor with a thread on it engaged with a template having one or more teeth that engage the thread. The thread has a flat segment at the distal end to reduce the tendency for the anchor to unscrew from the template.

FIG. 7A and FIG. 7B show a template engaged with an anchor having a deformable wire lock, which prevents the template from disengaging from the anchor once the template is placed against the distal aspect of the anchor. FIG. 7B is a section view of FIG. 7A.

FIGS. 8A and 8B show a system for placating tissue having two or more tissue anchors coupled to the target tissue at a first distance, and an anchor coupling plate coupled to the anchors such that they are separated by a second distance which is shorter than the first distance.

FIG. 9 shows a heart chamber having a valve annulus with multiple tissue anchors arrayed around the annulus. A valve replacement having leaflets and a cage is disposed within the annulus and coupled to the tissue anchors.

FIG. 10 shows a heart chamber having a valve annulus with multiple tissue anchors arrayed around the annulus. A valve replacement having leaflets and a cage is disposed within the annulus and coupled to the tissue anchors via magnets, with one magnet coupled to the anchor, and a mating magnet coupled to the valve replacement cage.

FIG. 11 shows a heart chamber having a valve annulus with multiple tissue anchors arrayed around the annulus. A valve replacement having leaflets and a cage is disposed within the annulus and coupled to the tissue anchors via sutures.

FIG. 12 shows an annular ring disposed around a valve annulus and coupled to the annulus by multiple tissue anchors. One or more of the couplings comprises an elongate slot, which allows for tolerance in the placement of the anchors.

FIG. 13 shows an annular ring disposed around a valve annulus and coupled to the annulus by multiple tissue anchors. A replacement valve is coupled to the annular ring, and the space between the annular ring and the replacement valve is covered by a flexible skirt, providing a hemostatic seal between the annulus and replacement valve.

FIG. 14 shows a valve annulus coupled to multiple tissue anchors. The tissue anchors are in turn coupled to one or more tissue shaping implants which ends at one or more tissue anchors. The end coupling allows for adjustment of the tension in the tissue shaping implants.

FIG. 15 shows a valve annulus coupled to multiple tissue anchors. The tissue anchors are in turn coupled to one or more tissue shaping implants forming a loop, having an adjustable connection mechanism which allows for adjustment in the tension of the tissue shaping implant.

FIG. 16 shows a template delivery catheter configured to be placed into a body cavity over an elongate control member coupled at least temporarily to a tissue anchor. The elongate control member exits the template delivery catheter through a sidewall port in the vicinity of the distal end of the template delivery catheter.

FIG. 17 shows a dilator having a tapered distal end and a handle at the proximal end. A port sized to receive a guidewire or other elongate member extends from the distal tip through the tapered section of the dilator, and then transitions to a slot near the surface of the dilator. This slot ends in the vicinity of the proximal end of the dilator.

FIG. 18 shows an elongate control member for coupling a tissue anchor to tissue having multiple segments connected by a rotatable puzzle interlock.

FIG. 19 shows a tissue anchor coupled to a valve annulus and an elongate control member which passes through a curved sheath. The curved sheath enters the chamber of the heart adjacent the annulus via an opening. Applying tension to the elongate control member at its distal end will cause deformation to the annulus in the region of the tissue anchor which can be observed via ultrasound, fluoroscopy, or other imaging modality.

FIG. 20 shows a tissue anchor coupled to a valve annulus and an elongate control member which passes through a curved sheath. The curved sheath incorporates an expandable basket. The segment of the curved sheath distal to the expandable basket enters the chamber of the heart adjacent the annulus via an opening. Applying tension to the elongate control member at its distal end will cause deformation to the annulus in the region of the tissue anchor which can be observed via ultrasound, fluoroscopy, or other imaging modality. The expandable basket allows for application of tension to the elongate control member without advancing the curved sheath further through the opening and into the chamber of the heart.

FIG. 21 shows a tissue anchor coupled to a valve annulus and an elongate control member, which passes through a curved sheath. The curved sheath incorporates an inflatable balloon. The segment of the curved sheath distal to the inflatable balloon enters the chamber of the heart adjacent the annulus via an opening. Applying tension to the elongate control member at its distal end will cause deformation to the annulus in the region of the tissue anchor which can be observed via ultrasound, fluoroscopy, or other imaging modality. The inflatable balloon allows for application of tension to the elongate control member without advancing the curved sheath further through the opening and into the chamber of the heart.

FIG. 22 shows a tissue anchor coupled to a valve annulus and an elongate control member which passes through a curved sheath. The curved sheath incorporates two curves in different directions, one proximal to the other. The segment of the curved sheath distal to the curved segments enters the chamber of the heart adjacent the annulus via an opening. Applying tension to the elongate control member at its distal end will cause deformation to the annulus in the region of the tissue anchor which can be observed via ultrasound, fluoroscopy, or other imaging modality. The two curves in the curved sheath allow for application of tension to the elongate control member without advancing the curved sheath further through the opening and into the chamber of the heart.

FIG. 23A shows a tissue anchor coupled to a valve annulus and an elongate control member which passes through a curved sheath. The curved sheath enters the chamber of the heart adjacent the annulus via an opening. A flexible support member passes over the elongate control member and contacts the wall of the chamber of the heart. Applying tension to the elongate control member at its distal end will cause deformation to the annulus in the region of the tissue anchor which can be observed via ultrasound, fluoroscopy, or other imaging modality. The flexible support member stiffens the curved sheath and allows for application of tension to the elongate control member without advancing the curved sheath further through the opening and into the chamber of the heart. The flexible support member may also be advanced to contact the wall of the chamber of the heart to offer additional support while applying tension to the elongate control member.

FIG. 23B shows a stiffener having a distal end and a handle at the proximal end. The distal end has a slot or open lumen to receive a guidewire or other elongate member extends from the distal tip to the vicinity of the proximal end of the stiffener.

FIG. 24 shows a tissue anchor coupled to a valve annulus and an elongate control member which passes through a curved sheath. The curved sheath enters the chamber of the heart adjacent the annulus via an opening. A guided support member passes over the elongate control member and contacts the wall of the chamber of the heart at two or more contact pads. Applying tension to the elongate control member at its distal end will cause deformation to the annulus in the region of the tissue anchor which can be observed via ultrasound, fluoroscopy, or other imaging modality. The guided support member with contact pads allows for application of tension to the elongate control member without advancing the curved sheath further through the opening and into the chamber of the heart.

FIGS. 25A and 25B show a tissue anchor releasably connected to an elongate control member via a key wire. The key wire extends through an inner lumen of the elongate control member, out through a hole in the side of the elongate control member and continues through a mating hole in the tissue anchor. FIG. 25B is a section view of FIG. 25A.

FIGS. 26A and 26B show a tissue anchor releasably connected to an elongate control member via a key wire. The key wire extends through an inner lumen of the elongate control member, out through a first hole in the side of the elongate control member, continues through a mating slot in the tissue anchor, then back through the mating slot in the tissue anchor and through a second hole in the side of the elongate control member. As shown, it extends through a hole in the opposite side of the anchor, for ease of assembly. FIG. 26B is a section view of FIG. 26A.

FIGS. 27A and 27B show a one-piece wire tissue anchor releasably connected to an elongate control member via a key wire. The key wire extends through an inner lumen of the elongate control member, out through a slot in the side of the elongate control member, around one or more coils of wire forming the one-piece wire tissue anchor and returning through the slot in the elongate control member. As shown, the wire continues through the lumen of the elongate control member and extends from its distal end, for ease of assembly. FIG. 27B is a section view of FIG. 27A.

FIGS. 28A and 28B show a tissue anchor releasably connected to an elongate control member via one or more tabs formed in the elongate control member and held in an extended position by a key wire with an increased diameter feature adjacent the distal end of the key wire. The key wire extends through an inner lumen of the elongate control member and is pulled proximally until the increased diameter feature is disposed between or among the tabs, forcing them outward. Pushing the key wire distally moves the increased diameter feature distal to the tabs, allowing them to collapse to a smaller diameter, releasing the anchor. FIG. 28A shows this assembly in the coupled condition, and FIG. 28B shows this assembly in the ready to release condition.

FIG. 29 shows dual stacked templates. The distalmost template is applied first. At this point, measurements may be taken, the positioning and effect of the template examined, and if satisfactory, the single template will suffice. If additional effect is required, the second, proximal most template may be placed over the first distal-most template and secured in place on the tissue anchor.

FIG. 30 shows a template and tissue anchor which includes exterior pledgets on the exterior aspect of the template, and interior pledgets on the interior aspect of the template.

FIG. 31 shows a template and tissue anchor which includes pledgets on the helical component of the tissue anchor and on the docking component of the tissue anchor. These pledgets may be constructed of a material such as ePTFE designed to flex out of the way as the anchor is coupled to the tissue, and to encourage tissue ingrowth.

FIG. 32 shows a template and tissue anchor in place on a valve annulus, the template being attached to an external skirt designed to create a smooth profile for tissue ingrowth or interaction with subsequent implants.

FIG. 33 shows a mitral valve with an annulus, and a tissue anchor coupled to that annulus. As shown, the tissue anchor resides in the left ventricle, where it can later be docked to an implant.

FIG. 34A and FIG. 34B show a tissue template designed to elevate the posterior area of the mitral annulus by entering the annulus from the atrial side at an angle to the annular plane.

FIG. 35 shows a template and tissue anchor in place on a valve annulus, creating a plication in the valve annulus.

FIG. 36 shows a template and tissue anchor in place on a valve annulus, creating a plication in the valve leaflet.

FIG. 37 shows an apical tension system, having an anchor near the apex of the left ventricle, one anchor on each side of the mitral valve annulus, and tension members between the annular anchors and apical anchor. The annular anchors may be placed in the vicinity of the fibrous trigones, the anterior-posterior commissures, or other advantageous locations on the annulus. The apical anchor may be placed at the apex of the ventricle, in the area of the papillary muscles, or other advantageous locations within the left ventricle.

FIG. 38 shows an apical tension system, having an anchor near the apex of the heart, at least one anchor on the mitral valve annulus or the tricuspid valve annulus, and tension members between the annular anchors and apical anchor. The annular anchors may be placed in the vicinity of the mitral fibrous trigones, the mitral commissures, the antero-posterior annulus of the tricuspid valve, or other advantageous locations on the annuli. The apical anchor may be placed at the apex of the heart, or other advantageous locations within the ventricle.

FIG. 39 shows a cross section of a mitral valve having a posterior valve leaflet that is tethered by chordae into a position that inhibits its normal motion, for example due to ischemic heart disease displacing the papillary muscles. A spacer is inserted between the posterior leaflet and the ventricular wall, in order to reposition the posterior leaflet and allow improved sealing against the anterior leaflet. The spacer may take the form of a stent, covered stent, basket, balloon, or other expandable device or material.

FIG. 40 shows a tissue shaping template having a tissue anchor that connects to the template via a deformable tab aligned substantially with the axis of the tissue anchor. One or more other anchors (2 shown) can be adjacent to tissue anchor for stabilizing the template to the annulus.

FIG. 41 shows an anchor having a ball with a loaded spring that is strong enough to prevent disengagement of the template from the anchor, but weak enough for you to pull the center anchor through the template.

FIG. 42A-42B show a tissue shaping template having an adjustment ring or the like that slips over the tissue and templet to squeeze the implanted template, increasing upward and downward force and therefore effect. The adjustment ring can be anchored into place with one or more anchors.

FIG. 43 shows a tissue shaping template having a self-collapsing adjustment member that squeezes the implanted tissue shaping template. Examples of self-collapsing adjustment members include staples, clips, coils or the like

FIG. 44 shows a tissue shaping template with self-expanding adjustment. A spring is compressed and fixed at two points adjacent to the end and center. It is at first constraint when implanted but self-expands (or lengthen to its original formed state) slowly after implantation similar to a nickel titanium stent.

FIG. 45 shows a tissue shaping template with biodegradable release. The template has arms in the form of coils, springs (as shown) or closed cells (diamond like cells like a closed cell stent) that are in held in compression by a corrodible or biodegradable wire (as shown), filament, tubing, or the like or embedded in biodegradable polymer. Upon resorption in the body, the coil expands to its original formed stretched length.

FIG. 46 shows a tissue shaping implant having post-implant adjustment features. The arms of the template have features such as ratchets (as depicted side view of arm), telescopic extenders (rod in a tube, expandable closed cells, or the like) that allow lengthening after implantation. A ring or the like is attached to the dock that may be coated with heparin. After implantation and tissue ingrowth, the ring can be pulled to extend the arm with or without pushing the ends of the template.

FIG. 47 shows a prolapse restrictor template that has one or more tissue anchors on or adjacent to valve annulus and prolapse leaflet of valve such as the mitral or tricuspid valve. The template is covered with a porous, knitted, weave fabric or shirt or the like and provides a backstop for prolapsing leaflet before or after tissue ingrowth into fabric or skirt. There may or may not be any reduction in length of the said annulus upon anchoring. Fabric or skirt can be made from ePTFE, weave or knitted PET, electrospun polymer or composite, or other materials that lead to tissue ingrowth with the prolapse leaflet, annulus, and or adjacent tissue.

FIG. 48 shows a sleeve over the center anchor such that it prevents the tab from springing open until the template has reached the center anchor. Once the template is inside the dock, the sleeve can be removed and the tabs spring out. The sleeve may be constructed from extremely thin materials (0.00025″ to 0.001″ wall thickness) such as PET shrink tube, polyimide, or the like. The user may deliver the tissue shaping template system, temporarily engage the anchor with the template by pulling the anchor and/or pushing the template and measuring the effect on the valve or valve annulus. If the results are acceptable, the sleeve can be removed, docking the anchor to the template. If the desired results are not attained, the anchor can be unscrewed and repositioned.

FIG. 49A-49C show a partial annular ring template being delivered to a valve annulus by rolling it to a delivery configuration having a relatively small diameter.

FIG. 50A-50E show a full annular ring being delivered to a valve annulus by folding it to the form of a partial ring and stretching the ends apart so the folded ring fits in a delivery device having a relatively small diameter. The ring is guided into place by one or more anchor control wires (placed previously) and is coupled to the one or more anchors to affix it adjacent the valve annulus. A valve replacement is further guided into position along the one or more anchor control wires, and coupled to the anchors, the ring, the valve apparatus directly, or some combination of these coupling methods.

FIG. 51A-51C show an annulus ring which is crimped to a reduced diameter for delivery through a delivery catheter. It is expanded and rotated to a flat configuration for placement adjacent a valve annulus.

FIG. 52A-52B show a template configured to plicate a local segment of a valve annulus, bringing two points on the valve annulus closer together and effectively reducing the effective valve annulus circumference, and minor diameter of the valve annulus.

FIG. 53A-53B show a template configured to invert a local segment of a valve annulus, reducing the minor diameter of the valve annulus without substantially changing the distance between two points on the annulus outside of the area of influence of the template, and leaving the effective valve annulus circumference substantially unchanged.

FIG. 54A-54B show a template and docking anchor coupled by extensible tabs, with a removal tool which can compress the extensible tabs inward, allowing de-coupling of the anchor from the template.

FIG. 55 shows a template constructed of one or more wireforms coupled to at least one attachment point for coupling one or more anchors to the wireform.

FIGS. 56A-56C show template blanks cut from either flat (56A) or tubular (56B, 56C) material.

FIG. 57 shows a top down sectional view of the heart, illustrating the relative positions of the major valves of the heart.

FIG. 58 shows a top view of the mitral valve in a closed configuration as visible from the left atrium.

FIG. 59 shows a top view of the mitral valve having a gap between mitral valve leaflets preventing it from attaining a closed configuration thus causing Mitral Regurgitation (MR) or Functional Mitral Regurgitation (FMR). The valve typically has an enlarged annulus configuration.

FIG. 60 shows the valve of FIG. 59 stretched (extended) in accordance with the present invention (device not shown), in this example stretched in the Commissure to Commissure (C-C) dimension as shown, causing the gap between the leaflets to close as shown, thus reducing or eliminating MR or FMR. In this example, the annulus configuration changes wherein the annulus dimension becomes larger across the stretched dimension and becomes smaller across a perpendicular or offset dimension to the stretched dimension.

FIG. 61 shows an implant applied to a segment of the valve annulus having a curved shape

FIG. 62 shows an implant applied to a segment of the valve annulus having a shape with multiple curves

FIG. 63 shows an implant applied to an enlarged valve annulus consisting of multiple elastic segments, shown in the extended position.

FIG. 64 shows an implant applied to an enlarged valve annulus consisting of multiple elastic segments, shown in the contracted position.

FIG. 65 shows an implant with a combination of substantially rigid segment and elastic segments, shown in the contracted position.

FIG. 66 shows an anchor for fastening implants to tissue which includes a helical coil, a torque member, and a key wire locking the two together against translational and rotational motion.

FIG. 67 shows an implant which includes a helical coil in position in tissue prior to activation of the helical coil

FIG. 68 shows an implant which includes a helical coil in position in tissue after activation of the helical coil

FIG. 69 shows an implant with helical coils in place against a substantially straight section of tissue which is significantly longer than the implant itself.

FIG. 70 shows the implant of FIG. 69 with the same tissue of FIG. 69 having been drawn into the concavities of the implant, bringing the ends of the tissue into approximation with the ends of the implant.

FIG. 71 shows projected shapes of a model mitral annulus, that annulus treated with a flattening implant, and that annulus treated with an undulating implant.

FIG. 72 illustrates an undulating implant that is assembled in place from sub-sections of the implant.

FIG. 73 shows a subsection of an undulating implant folded to a reduced diameter for ease of delivery through a tube or tubular structure

FIG. 74 shows a subsection of an undulating implant expanded to allow ease of anchor placement

FIG. 75 shows a pair of subsections of an undulating implant arranged one in front of the other for simultaneous delivery through a tube or tubular structure.

FIG. 76 shows an implant template that is placed in a substantially straight configuration, with deforming members in apposition to the implant template.

FIG. 77 shows the implant template of FIG. 76 having been deformed by the deforming members as they are moved distal relative to the anchor.

FIG. 78 shows a plurality of subsections of an undulating implant pinned together via a pin extending through the two subsections with a locking cap to hold the two subsections together.

FIG. 79 shows a plurality of subsections of an undulating implant having extensions that are substantially parallel to the anchor member which are held together with locking devices.

FIG. 80 shows a plurality of subsections of an undulating with ends that are held together with locking devices.

FIG. 81 shows a partial ring template with multiple anchors, the partial ring template being smaller than the mitral annulus, and the multiple anchors being used to draw the annulus towards the template.

FIG. 82 shows a two-anchor segment with a convex profile for shaping the valve annulus

FIG. 83 shows a template constructed from two two-anchor segments with convex profiles

FIG. 84 shows an undulating template with a single undulation composed of straight segments aligned horizontally and vertically.

FIG. 85 shows an undulating template with a single undulation composed of a combination of straight and curved segments aligned perpendicularly to each other.

FIG. 86 shows an undulating template with a single undulation composed of a combination of straight and curved segments aligned at non-perpendicular angles to each other.

FIG. 87 shows an undulating template with a single undulation composed of curved segments with the ends configured so that the tangent to the curved segment at the end is parallel to the tangent at the location where the tissue coupling mechanism is attached.

FIG. 88 shows an undulating template with a single undulation composed of curved segments with the ends extending past the point at which the tangent to the curved segment is parallel to the tangent at the location where the tissue coupling mechanism is attached

FIG. 89 shows an undulating template with a single undulation with a continuous non-circular shape.

FIG. 90 shows an undulating template where the distance from the point where the tissue coupling mechanism is attached to the highest peaks of the body of the template is greater than the length of the tissue coupling mechanism

FIG. 91 shows an undulating template where the distance from the point where the tissue coupling mechanism is attached to the highest peaks of the body of the template is less than the length of the tissue coupling mechanism

FIG. 92 shows an undulating template with tissue held in place by a tissue coupling mechanism, causing the template to exert forces in a tensile manner normal to the original position of the tissue (via the tissue coupling mechanism) and in an inward manner, tangential to the original position of the tissue.

FIG. 93 shows an undulating template with tissue held in place by a tissue coupling mechanism, causing the template to exert forces in a tensile manner normal to the original position of the tissue (via the tissue coupling mechanism) and in a compressive manner normal to the original position of the tissue at the peaks of the undulations.

FIG. 94 shows an undulating template with tissue held in place by a tissue coupling mechanism, causing the template to exert forces in a tensile manner normal to the original position of the tissue (via the tissue coupling mechanism) and in a combined inward compressive manner, directed between normal and tangential directions to the original position of the tissue.

FIG. 95 shows an undulating template with tissue held in place by a tissue coupling mechanism, causing the template to exert forces in a tensile manner normal to the original position of the tissue (via the tissue coupling mechanism) and in a combined inward compressive manner, directed between normal and tangential directions to the original position of the tissue.

FIG. 96 shows an undulating template with stabilizing tissue coupling mechanisms at each end, in addition to the primary tissue coupling mechanism in the middle. Also shown are removable devices for placing and manipulating the tissue coupling mechanisms.

FIG. 97 shows and undulating template with an additional stabilizing arm extending from the body, as well as stabilizing penetrating points.

FIG. 98 shows an undulating template with the ends folded away from the attachment point of the tissue coupling mechanism to a delivery position, where the tissue coupling mechanism attachment allows the template to fold alongside the tissue coupling mechanism.

FIG. 99 shows an undulating template in position adjacent to a mitral annulus in the untreated state.

FIG. 100 shows an undulating template with a mitral annulus, where the tissue coupling mechanism has drawn the annulus tightly against the template. The original position of the annulus from FIG. 99 is also shown.

FIG. 101 illustrates a delivery device for placing an undulating template over a pre-anchor guide. The pre-anchor guide runs through a receiving slot in the delivery device.

FIG. 102 shows percent area change for various templates implanted in-vivo.

FIG. 103 shows percent circumference change for various templates implanted in-vivo.

FIG. 104 shows percent minor axis change for various templates implanted in-vivo.

FIG. 105 shows percent A-P (minor axis) reduction for various multi-wave templates implanted in excised porcine mitral annuli.

FIG. 106 shows percent A-P (minor axis) reduction for various single-wave templates implanted in excised porcine mitral annuli.

FIG. 107 shows a continuous template with one area of undulations.

FIG. 108 shows a continuous template with two areas of undulations.

FIG. 109 shows a continuous template with undulations on the entire circumference.

FIG. 110 shows a template where the compression points form an angle with an anchor point.

FIG. 111 shows a side view of a template where the compression points are offset to a different plane than the anchor point

FIG. 112 shows a top view of a template where the compression points are offset to a different plane than the anchor point

FIG. 113A shows a template in the preformed shape.

FIG. 113B shows a template in a crimped or partially crimped configuration with both ends pressed toward the center.

FIG. 113C shows a template in a crimped or partially crimped configuration with both ends rotated towards each other to a substantially circular shape.

FIG. 114A shows a template with anchor, the template being in the preformed shape

FIG. 114B shows a template with anchor, the template being constrained in a crimped state with the ends or wings of the template pulled proximally relative to the anchor.

FIG. 115 shows a template, illustrating distance between ends, distance between apexes, width of concavity, and depth of concavity.

FIG. 116A shows a template translatably or slidably coupled to an anchor control device, in position to move toward the anchor

FIG. 116B shows the template, anchor, and anchor control device of FIG. 116A, with the template coupled to the anchor by a template coupling mechanism.

FIG. 117 shows a tissue shaping template having two central anchors near the apex of the concavity.

FIG. 118 shows a tissue shaping template with three anchors, one approximating the center of the apex and two on either side of the center anchor.

FIG. 119A shows a tissue shaping template having two anchors, one of which is attached to the template via a threaded pod. The anchor attached to the threaded pod is in the proximal position, prior to coupling to tissue (not shown).

FIG. 119B shows the tissue shaping template of FIG. 119A, with the anchor coupled to the threaded pod in the proximal position, coupled to the tissue (not shown).

FIG. 120 shows a section view of the pod and anchor illustrating the relationship of the anchor shape and the threaded pod.

FIG. 121 shows accessory barbs placed adjacent a helical coil tissue anchor

FIG. 122 shows an array of tissue anchors coupled to a tissue shaping template

FIG. 123 shows an array of tissue anchors coupled to each other by gears to that they turn simultaneously

FIGS. 124A and 124B shows a tissue shaping template with an accessory latch to hold tissue against the concavity of the template

FIG. 125A-125H show placement of a primary anchor, a frame coupled to accessory anchors, and a tissue shaping template with side anchors in a target tissue.

FIGS. 126A and 126B show a tissue anchor releasably connected to an elongate control member via a key wire. The key wire extends through an inner lumen of the elongate control member, out through a hole in the side of the elongate control member and continues through a mating hole in the tissue anchor. FIG. 126B is a section view of FIG. 126A.

FIGS. 127A and 127B show a tissue anchor releasably connected to an elongate control member via a key wire. The key wire extends through an inner lumen of the elongate control member, out through a first hole in the side of the elongate control member, continues through a mating slot in the tissue anchor, then back through the mating slot in the tissue anchor and through a second hole in the side of the elongate control member. As shown, it extends through a hole in the opposite side of the anchor, for ease of assembly. FIG. 127B is a section view of FIG. 127A.

FIGS. 128A and 128B show a template having two anchors gripped in a delivery device having a movable jaw and teeth that wrap partially around the template for delivery to the target tissue site.

FIGS. 129A-129H show a device for delivering a template to a frame, the delivery device comprising telescoping tubes with teeth that wrap partially around the template and fit into clearance features in the template.

FIG. 130A shows a tissue shaping template having an attached skirt to offer a valve sealing surface to one or more apposed valve leaflets.

FIG. 130B shows a tissue shaping template having an attached skirt of sufficient length to act as a prosthetic valve leaflet.

FIG. 131 shows a cutaway view of a heart with a tissue shaping template in place at the valve annulus, the template having an attached skirt to offer a valve sealing surface to one or more apposed valve leaflets, not shown.

FIG. 132 shows a cutaway view of a heart with a tissue shaping template in place at the valve annulus, the template having an attached skirt to offer a valve sealing surface to one or more apposed valve leaflets, not shown, the skirt being attached to a stabilizing chord which is anchored into the tissue of the ventricle.

FIG. 133 shows a cutaway view of a heart with a tissue shaping template in place at the valve annulus, the template having an attached skirt to offer a valve sealing surface to one or more apposed valve leaflets, not shown. The template is coupled to a stabilizing chord which is anchored into the tissue of the ventricle. This stabilizing chord also stabilized the attached skirt in a bent configuration

FIG. 134 shows a tissue shaping template having multiple attached skirts, 3 as shown, to offer a valve sealing surface to one or more apposed valve leaflets.

FIG. 135A shows a cutaway view of a heart having a valve leaflet that is tethered by chordae coupling the leaflet to a papillary muscle in the ventricle. The leaflet tethering as shown holds the leaflet in close apposition to the ventricular wall, limiting its motion.

FIG. 135B shows a side view of the heart shown in FIG. 135A.

FIG. 135C shows the heart of FIG. 135A, having a guidewire placed between the tethered leaflet and the wall of the ventricle.

FIG. 135D shows the heart and guidewire of FIG. 135C, with a balloon stent catheter in place between the tethered leaflet and the wall of the ventricle.

FIG. 135E shows the heart of FIG. 135A with the stent of FIG. 135D fully expanded, moving the tethered leaflet out away from the ventricular wall and closer to one or more other leaflets (not shown).

FIG. 136 shows the stent of FIG. 135E with a hemostatic coating on the outside surface of the stent. This coating can offer an additional coaptation surface in situations where the tethered leaflet surface is inadequate for coaptation with one or more apposed leaflets.

FIG. 137 shows a stent in place between a tethered leaflet and the wall of the ventricle, the stent having a basket shape with substantially closed ends. The closed ends may reduce the possibility of embolized thrombus.

FIG. 138 shows the stent of FIG. 135E with end caps to substantially close the ends. The closed ends may reduce the possibility of embolized thrombus.

FIG. 139 shows the stent of FIG. 135E with a filler material in the stent. This material may be beneficial to control thrombus formation.

FIG. 140A shows a tissue anchor having a washer with a protrusion, the tissue anchor being releasably coupled to a control wire, and a tissue template slidably coupled to the control wire.

FIG. 140B shows the system of FIG. 140A after the tissue template has been moved distally against the washer. The protrusion interacts with the tissue template to prevent rotation of the anchor relative to the tissue template.

FIG. 141 shows a tissue anchor having a washer with a protrusion having a proximal extension, the tissue anchor being releasably coupled to a control wire, and a tissue template engaged with the anchor and in apposition with the washer. The proximally extension on the protrusion interacts with the tissue template to prevent rotation of the anchor relative to the tissue template.

FIG. 142 shows a tissue anchor having a washer with a multiple protrusions, the tissue anchor being releasably coupled to a control wire, and a tissue template engaged with the anchor and in apposition with the washer. One or more of the protrusions interact with the tissue template to prevent rotation of the anchor relative to the tissue template.

FIG. 143 shows a system having two or more cylindrical stents placed between a valve leaflet and the wall of the ventricle. The stents are held in place by their interaction with the chordae of the valve.

FIG. 144 shows a stent having extension bosses which interact with valve chordae to hold the stent in a position between a valve leaflet and the wall of the ventricle.

FIG. 145 shows a single D-shaped stent placed between a valve leaflet and the wall of the ventricle. The stent is held in place by interaction with the chordae of the valve.

FIG. 146 shows a tissue shaping template in place on the posterior aspect of a mitral annulus, with a prosthetic leaflet coupled to the template. The leaflet is supported in position in the annular plane by two tethers attached to anchors in the fibrous trigones.

FIG. 147 shows a tissue shaping template in place on the posterior aspect of a mitral annulus, with a prosthetic leaflet coupled to the template. The leaflet is supported in position in the annular plane by one tether attached to an anchor in the anterior valve annulus.

FIG. 148 shows a tissue shaping template in place on the posterior aspect of a mitral annulus, the template being stabilized in position in the annular plane by one or more tethers coupled to one or more tissue anchors in the anterior annulus, the fibrous trigones, or a combination of the two.

FIG. 149 shows a tissue shaping template in place on the posterior aspect of a mitral annulus, the template being stabilized against motion in the posterior direction by a tether coupled to the atrial septum, by a basket, clip, suture, hook, anchor, clip, staple, or other method known to the art.

FIG. 150 shows a cutaway view of a heart with a tissue shaping template in place at the valve annulus, the template having an attached skirt to offer a valve sealing surface to one or more apposed valve leaflets, not shown. The underside of the skirt is tethered to the tissue of the ventricle.

FIG. 151A-151C show a template having coupling features for a center anchor, two inner side anchors, and two outer side anchors. The three center points of the coupling features for the center anchor and the two inner side anchors define a plane, and the outer ends of the implant extend to one side of said plane.

FIG. 152A-152B show a template having coupling features for a center anchor, two inner side anchors, and two outer side anchors. The three center points of the coupling features for the center anchor and the two inner side anchors define a plane, and the outer ends of the implant lie along said plane.

FIG. 153 shows a template having coupling features for two center anchors, two inner side anchors, and two outer side anchors. The two center anchor coupling features are connected with a line that has a mid-point. The two center points of the coupling features for the inner side anchors and the midpoint of the line connecting the two center anchor coupling features define a plane, and the outer ends of the implant extend to one side of said plane.

FIG. 154A-154C show a left atrial view of a heart having a mitral valve comprising leaflets and an annulus. A probe is placed between the ventricle and the valve leaflets and raised to lift the leaflet, allowing accurate visualization of the attachment point between the leaflet and ventricle. A mark is placed at this attachment point, to guide a surgical procedure.

FIG. 155A-155B show a tissue shaping template having center, inner, and outer anchors along a broadly curved shape in place in a valve annulus.

FIG. 156A-156B shows a tissue shaping template having center, inner, and outer anchors, the template being long enough to couple with the outer anchors in the fibrous trigones of the valve annulus.

FIG. 157A-157B show a tissue shaping template having center, inner, and outer anchors along a broadly curved shape in place in a valve annulus. In addition, one or more anchors are placed in the fibrous trigones of the annulus and coupled to the tissue shaping template by one or more tension members. The tension members are adjusted to move the template closer to the anchors in the fibrous trigones.

FIG. 158A-158B a tissue shaping template having center, inner, and outer anchors along a broadly curved shape in place in a valve annulus. In addition, the system has one or more tension members coupling different areas of the template. The length of the tension members can be adjusted to adjust the shape of the tissue shaping template.

FIG. 159A-159D shows a tissue shaping template coupled to a valve annulus by two side anchors and one center anchor which is releasably coupled to an anchor control wire, the tissue shaping template having a removable stretching member to increase the distance between the side anchors compared to the distance between the side anchors in the unstressed template. After the two side anchors are coupled to the valve annulus, the stretching member is removed, creating a lower tension segment of the valve annulus. The center anchor draws the lower tension segment of valve annulus into the concavity in the template. After the center anchor is coupled to the template, the center anchor is de-coupled from the control wire and the control wire is removed.

FIG. 160A-160B show a tissue shaping template having three convexity anchors and two concavity anchors, the concavity anchors each having releasable anchor control wires. Once the concavity anchors are coupled to the template, the anchors are decoupled from the anchor control wires, and the anchor control wires are removed. This tissue shaping template may also include a stretching member as shown in FIG. 159, not shown in this figure.

FIG. 161 shows a tissue shaping template having one or more central anchors, outer side anchors coupled to a valve annulus at approximately the midpoint along the minor diameter dimension of the annulus. The center anchor draws tissue into a concavity. The template may have additional inner side anchors to further stabilize the template on the annulus.

FIG. 162 shows a tissue shaping template having outer side anchors coupled to a valve annulus at approximately the midpoint along the minor diameter dimension of the annulus and two or more inner side anchors which draw tissue into corresponding concavities in the tissue shaping template. The template may have one or more center anchors to further stabilize the template on the annulus.

FIG. 163A-163B show a tissue shaping template coupled to trigonal anchors in turn coupled to the fibrous trigones of a valve. The trigonal anchors are releasably coupled to control wires which are in turn coupled to the tissue shaping template in a manner that allows the template to move closer to or farther from the trigonal anchors. A stop mechanism is placed along the control wire and coupled to the anchor to limit the template motion in the direction away from the trigonal anchors. Once the stop mechanism is coupled to the anchors, the control wires may be decoupled from the trigonal anchors and removed. A horizontal line defines the boundary between posterior/distal directions and anterior/proximal directions relative to the anatomy and implant.

FIG. 164A-164B show a tissue shaping template having movable extensions coupled to trigonal anchors in turn coupled to the fibrous trigones of a valve. An adjustment mechanism couples the movable extensions to the tissue shaping template. The adjustment mechanism can vary the distance between the template and the trigonal anchors to achieve the desired effect.

FIG. 165A shows a tissue shaping template having a central concavity with a center anchor and an accessory anchor, and two lateral concavities, each comprising a lateral accessory anchor.

FIG. 165B shows the implant of FIG. 165A with the addition of mid-lateral stabilizing anchors.

FIG. 166 shows a tissue shaping implant having stabilizing protrusions extending toward the atrial side of the implant.

FIG. 167 shows a mitral valve having three posterior leaflets which define regions along the posterior valve annulus.

### DETAILED DESCRIPTION OF THE INVENTION

The phrase “valve annulus” as used herein and in the claims means a ring-like tissue structure surrounding the opening at base of a heart valve that supports the valve's leaflets. For example, the annulus of the mitral valve, the tricuspid valve, the aortic valve, the pulmonary valve, venous valves and other annuluses of valves in the body. In the mitral valve, the annulus typically is a saddle-shaped structure that supports the leaflets of the mitral valve.

The phrase “peripheral wall” as used herein and in the claims as applied to a valve annulus means a surface or portion of the tissue of the valve annulus, and/or a portion of the tissue adjacent to the valve annulus.

“Concavity” as used herein and in the claims means a depression or well-formed in a surface of the template. The concavity may comprise flat regions joined at angles, e.g. being rectilinear, but will more typically have a curved bottom portion joining a pair of generally straight and/or curved walls or legs. The curved bottom portion will typically span an arc of at least 45°, often at least 60°, usually at least 90°, typically at least 135°, and sometimes spanning a full 180°, with exemplary ranges from 45° to 180°, from 60° to 180°, from 60° to 135°, and from 90° to 135°. The concavities of the present invention will typically be symmetric having opposed walls or legs on each side of a central axis. In other cases, however, a concavity may be asymmetric with walls or legs on opposite sides having unequal lengths and, in some cases, having only a single wall or leg. Examples of concavities include the inner surface of a circle or sphere or other curved structure.

“Convexity” as used herein and in the claims means a curved surface on the template like an exterior of a circle, parabola, ellipse, or the like. A convexity will typically be formed on a surface of the template on the side opposite to that of a concavity, and vice versa. Examples of convexities include the outer surface of a circle or sphere or other.

As used herein and in the claims, an “implant” means an article or device that is introduced into and left in place in a patient's body by surgical methods, including open surgery, intravascular surgical methods, percutaneous surgical methods, endoscopic methods, and least invasive or other methods. For example, aortic valve replacement implant, coronary stent implant, or other types of implants.

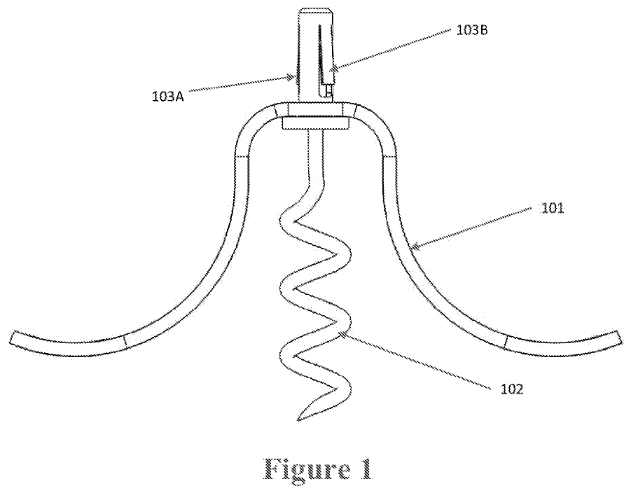

As shown in FIG. 1, a tissue shaping template 101 is held in apposition to tissue by a tissue anchor 102 having extensible tabs 103A and 103B. The extensible tabs 103A and 103B are aligned axially with the body of the tissue anchor 102 and are configured to flex as they pass through a hole in the template 101 in a first direction, then return to an extended position, preventing passage through the hole in the template 101 in the opposite direction.

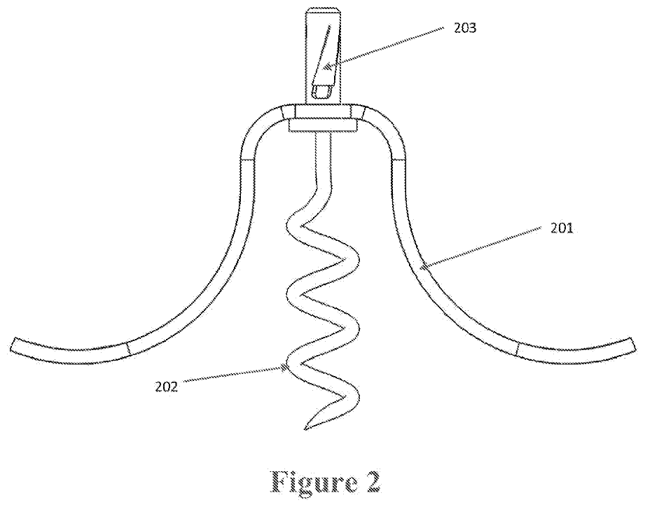

As shown in FIG. 2, a tissue shaping template 201 is held in apposition to tissue by a tissue anchor 202 having at least one extensible tab 203. The extensible tab 203 is aligned at an angle to the axis of the body of the tissue anchor 202 and is configured to flex as it passes through a hole in the template 201 in a first direction, then return to an extended position, preventing passage through the hole in the template 201 in the opposite direction.

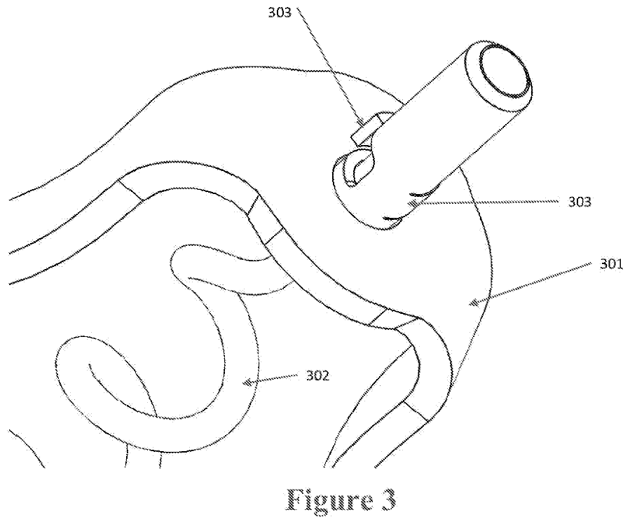

As shown in FIG. 3, a tissue shaping template 301 is held in apposition to tissue by a tissue anchor 302 having at least one extensible tab 303. The extensible tab 303 is aligned approximately perpendicularly to the axis of the body of the tissue anchor 302 and is configured to flex to a smaller diameter to allow passage through a hole in the template 301, then to return to an extended position, preventing passage through the hole in template 301. Flexing of the tab 303 can be actuated via a key wire holding the tab in the flexed position, by rotating the template 303 relative to the main body of the anchor 302, or by other similar means.

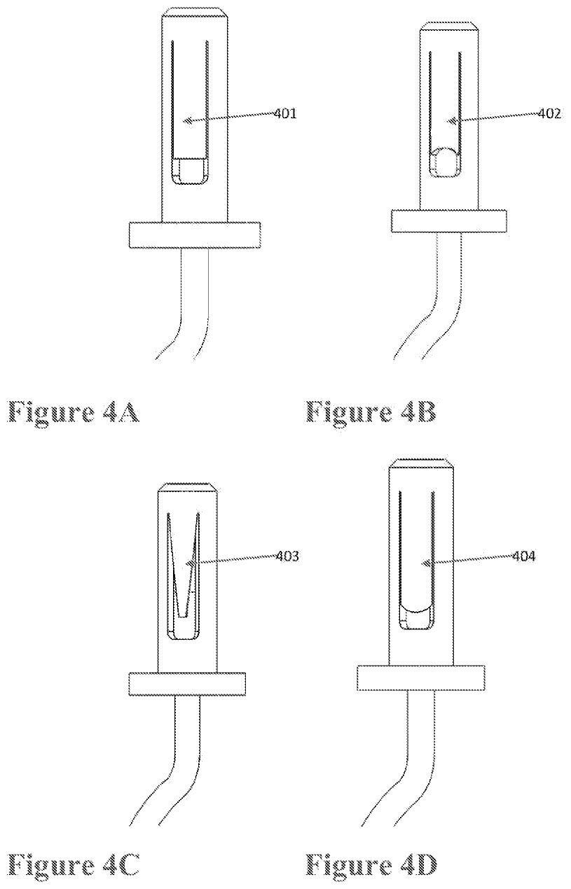

As shown in FIGS. 4A-4D, tabs can have a variety of shapes. FIG. 4A shows a flat ended tab 401 of constant cross section, FIG. 4B shows a concave ended tab 402 of constant cross section, FIG. 4C shows a flat ended tab 403 of variable cross section, and FIG. 4D shows a convex ended tab 405 of constant cross section. While these figures are for illustration, variations on tab end geometry can include symmetrical or asymmetrical shapes, shapes perpendicular to the tab, or shapes at an angle to the tab, wavy shapes, toothed shapes, and other variations on end shape. The end shape interacts with the template (not shown), and the template may include features designed to interact with the tab end shape. The varying tab cross section can be decreasing from the root of the tab (as shown in FIG. 4C), increasing from the root of the tab, or have sections which are increasing and other sections which are decreasing. The varying tab cross section can also be continuous or discontinuous, having sections that change abruptly, in one example from large to small, in another example from small to large.

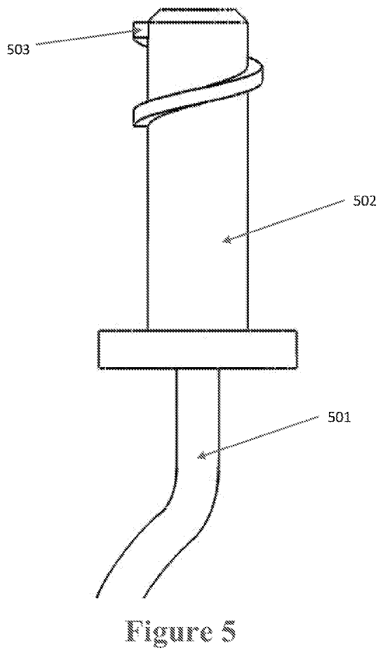

As shown in FIG. 5, a tissue shaping template (not shown) can be coupled to a tissue anchor 501 via helical threads 503 arrayed around the body 502 of the tissue anchor 501. For a tissue anchor 501 that engages tissue by twisting (for example, a helical coil), the helical threads 503 can have an opposite handedness relative to the helical coil of the tissue anchor 501, so that the template engages the threads as the tissue anchor 501 is twisted in the same direction required to engage tissue.

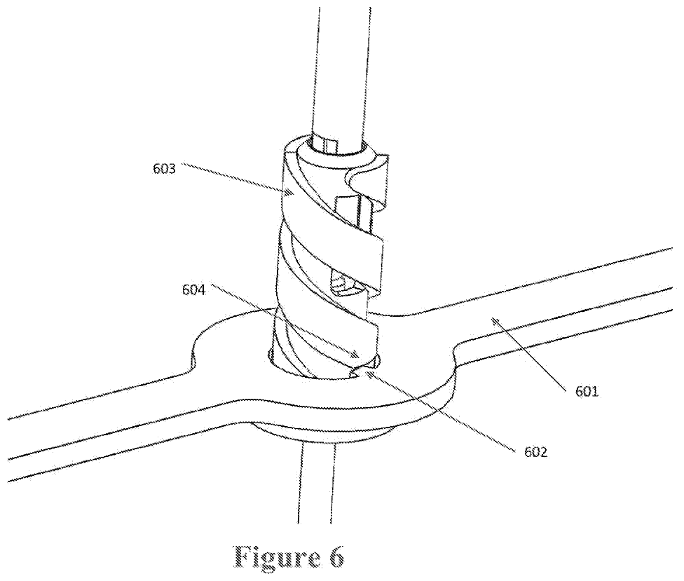

As shown in FIG. 6, a tissue shaping template 601 has a tooth 602 which engages a docking cam 603 on the body of a tissue anchor. The docking cam 603 has a helical segment and a flat segment 604. This configuration allows the helical segment to have a steeper pitch to allow for rapid tightening, while the flat segment 604 has a shallow pitch to allow friction locking with the tooth 602, which prevents the anchor from rotating to disengage with the tissue shaping template 601.

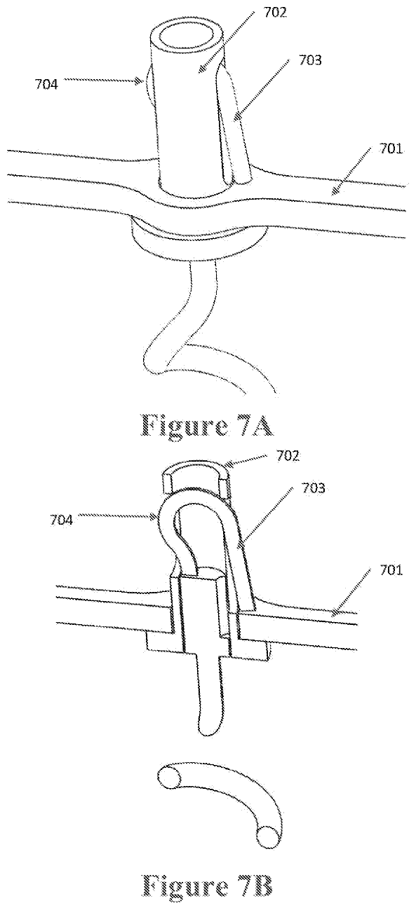

As shown in FIGS. 7A and 7B, a tissue shaping template 701 is coupled to an anchor dock 702 via a straight wire segment 703. The straight wire segment 703 extends through a slot on the anchor dock 702 and is held in place by a curved spring segment 704. The curved spring segment 704 allows the straight wire segment 703 to retract into the slot in the anchor dock 702 as the template 701 is slid distally over the anchor dock 702, then the straight wire segment 703 recovers to its position outside the slot, preventing the template 701 from moving proximally relative to the anchor dock 702.

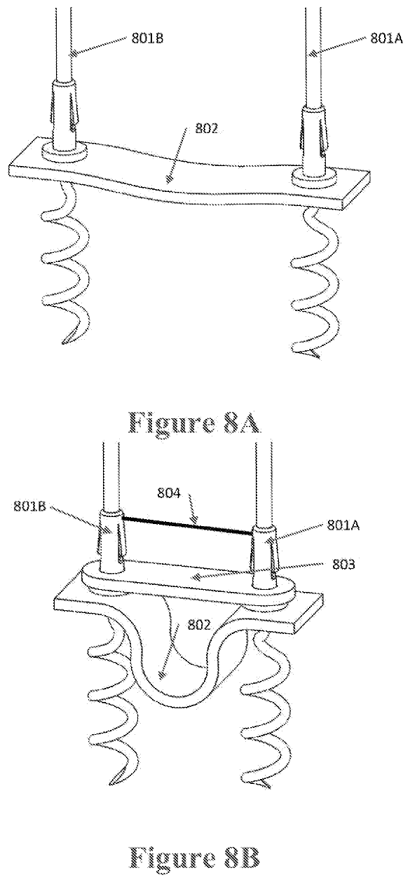

As shown in FIG. 8A, a system of 2 or more anchors 801A and 801B are placed into target tissue 802 at a first separation distance. This first separation distance can be set via visualization methods including ultrasonography, fluoroscopy, CT scan, endoscope, light-based cameras, or direct visualization through an opening adjacent the target tissue. Alternately, this first separation distance can be set via an anchor application device that spaces the second anchor a desired distance from the first.

As shown in FIG. 8B, a shortening member 803 can be coupled to both anchors 801A and 801B, drawing them to a second separation distance which is shorter than the first separation distance, plicating the target tissue 802. A compression member 804 may be added to maintain desired angular alignment of the anchors.

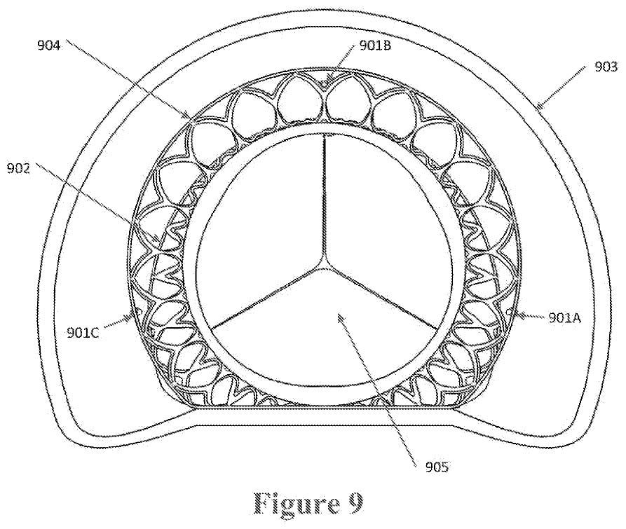

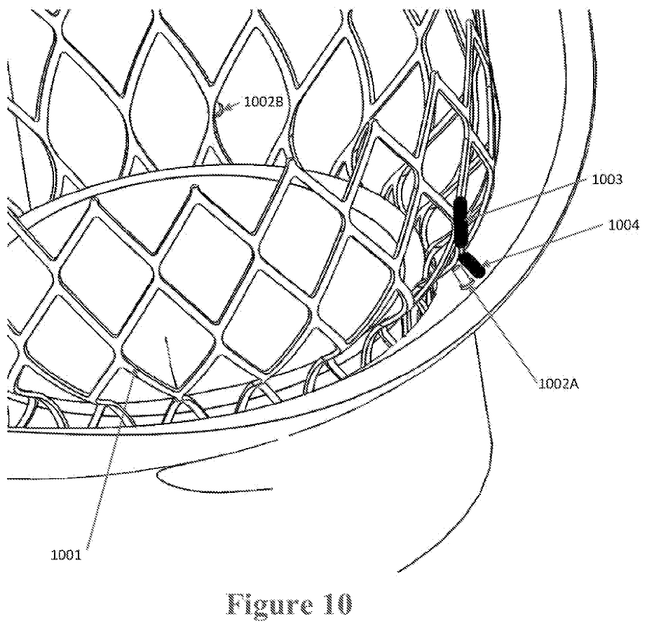

As shown in FIG. 9, one or more anchors (901A, 901B, 901C) are placed into the annular region 902 of a natural heart valve in a heart chamber 903. The natural heart valve has been replaced by an artificial valve 905 held in place by an expanded cage 904. The expanded cage 904 is coupled to the anchors 901A, 901B, and 901C.

As shown in FIG. 10, one or more anchors (1002A, 1002B) coupled to anchor magnets 1004 are placed into the annular region of a natural heart valve in a heart chamber. The natural heart valve has been pushed outwardly by an expanded cage 1001. The expanded cage 1001 is coupled to cage magnets 1003. When the cage is placed in the heart valve, the anchor magnets 1004 are attracted to the cage magnets 1003, coupling the cage 1001 to the anchor 1002A. The magnetic fields of the cage magnet 1003 and anchor magnet 1004 are arranged to encourage this attraction by placing opposing magnetic poles next to each other in the desired configuration. Alternately, one of the magnets can be constructed of a magnetic material that has no permanent magnetic field, so that it is attracted to the other magnet regardless of orientation.

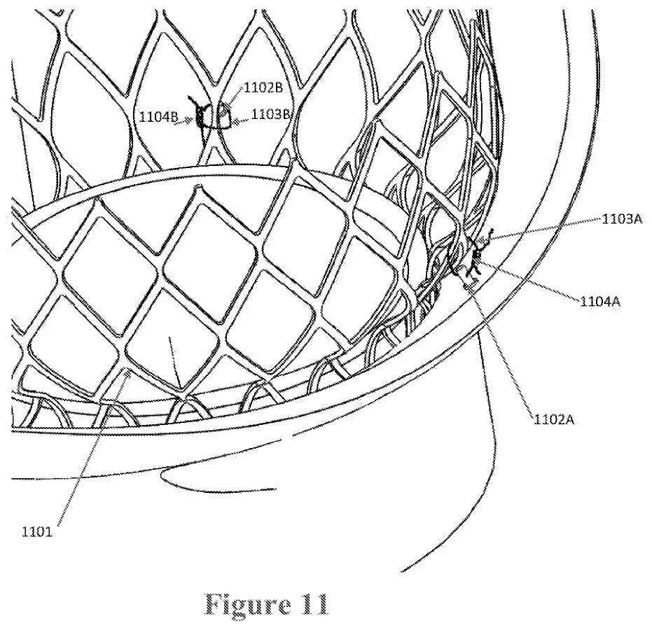

As shown in FIG. 11, one or more anchors (1102A, 1102B) are placed into the annular region of a natural heart valve in a heart chamber. The natural heart valve has been pushed outwardly by an expanded cage 1101. The expanded cage 1101 is coupled to the anchors 1102A and 1102B via sutures 1103A and 1103B. The sutures 1103A and 1103B are terminated via suture knots 1104A and 1104B. These suture knots 1104A and 1104B can be knots, clips, suture locks, or the like, and maintain tension in the sutures 1103A and 1103B to hold the cage 1101 in proximity with the anchors 1102A and 1102B

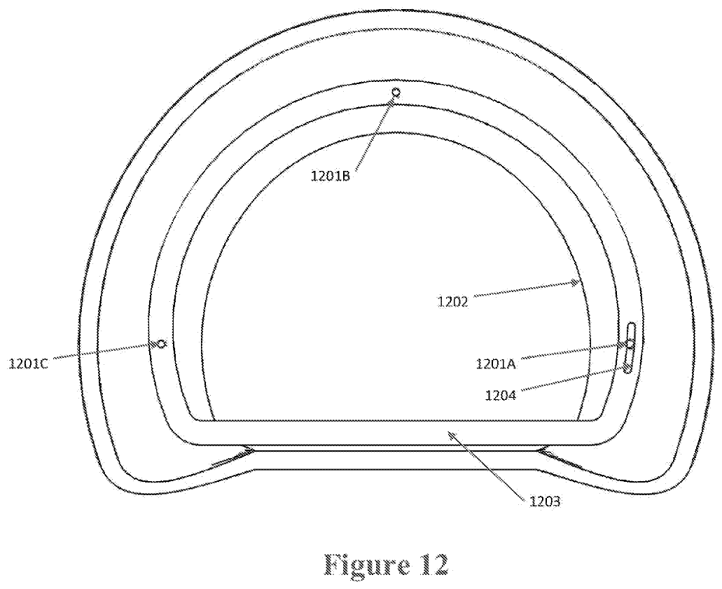

As shown in FIG. 12, one or more anchors (1201A, 1201B, 1201C) are placed into the annular region 1202 of a natural heart valve. A ring 1203 is coupled to one or more of the anchors 1201A, 1201B, and 1201C. One or more of the couplings between the anchors 1201A, 1201B, 1201C and ring 1203 may include a slot 1204 to allow some motion of the anchor (1201A as shown) with respect to the ring 1203. The ring 1203 may be a closed ring as shown or a partial ring. The ring 1203 in combination with the anchors 1201A, 1201B, and 1201C, may be used to re-shape a valve annulus.

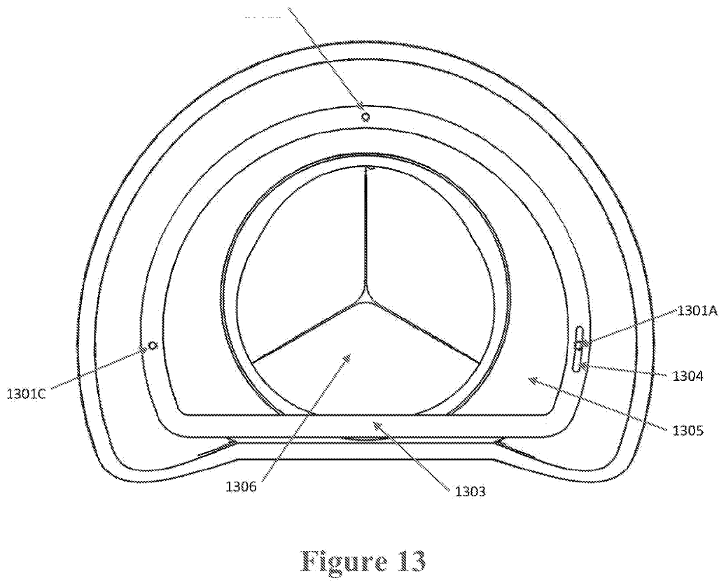

As shown in FIG. 13, one or more anchors (1301A, 1301B, 1301C) are placed into the annular region 1302 of a natural heart valve. A ring 1303 is coupled to one or more of the anchors 1301A, 1301B, and 1301C. One or more of the couplings between the anchors 1301A, 1301B, 1301C and ring 1303 may include a slot 1304 to allow some motion of the anchor (1301A as shown) with respect to the ring 1303. The ring 1303 is in turn coupled to a skirt 1305 which is coupled to a replacement valve 1306. The ring 1303 may be a closed ring as shown or a partial ring. The ring 1303 in combination with the anchors 1301A, 1301B, and 1301C, may be used to re-shape a valve annulus, as well as forming a platform for attachment of a replacement heart valve. The skirt 1305 prevents any leakage of blood from between the replacement valve 1306 and the ring 1303.

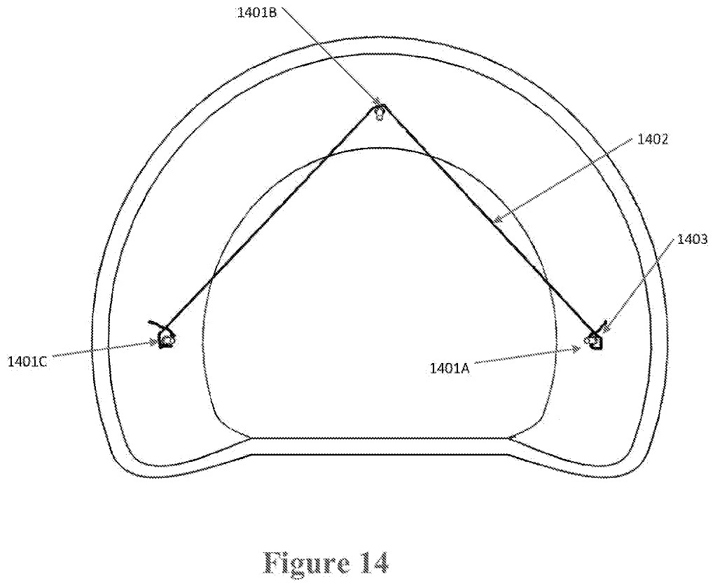

As shown in FIG. 14, two or more anchors (1401A, 1401B, 1401C) are placed into the annular region of a natural heart valve. A tissue shaping implant or array of implants 1402 is coupled to two or more of the anchors 1401A, 1401B, and 1401C. The implant array 1402 has two ends, at least one of which is an adjustable end 1403. The adjustable end 1403 can slide relative to the anchor 1401A as tension is adjusted to achieve the desired effect, then locked to maintain the desired tension.

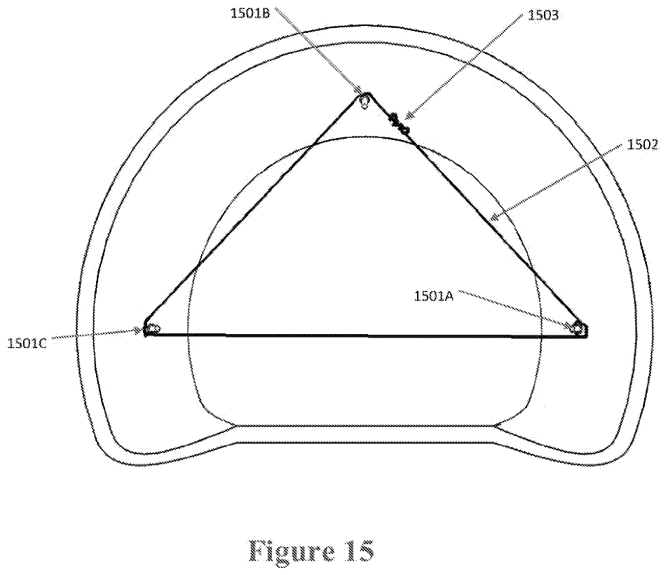

As shown in FIG. 15, three or more anchors (1501A, 1501B, 1501C) are placed into the annular region of a natural heart valve. A tissue shaping implant loop 1502 is coupled to three or more of the anchors 1501A, 1501B, and 1501C. The loop 1502 has a circumference adjuster 1503. The circumference of the loop 1502 is adjusted to achieve the desired effect, and the circumference adjuster 1503 maintains the desired circumference. The circumference adjuster 1503 may be a slipknot, a worm gear, a screw, or other known methods to adjust and maintain the length of a loop 1502 of tissue shaping implants.

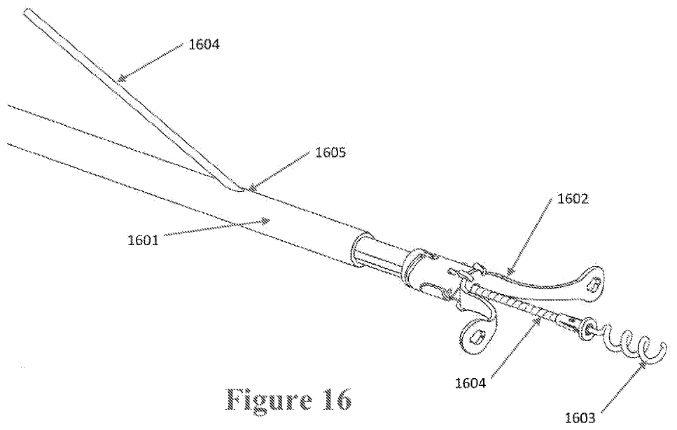

As shown in FIG. 16, a delivery catheter 1601 for a tissue template 1602 slides over a torque tube 1604 used to place a helical tissue anchor 1603. The torque tube 1604 extends through a mating hole in the tissue template 1602, into the main body of the delivery catheter 1601, and out of an exit port 1605 in the side of the delivery catheter 1601. This rapid exchange configuration of the delivery catheter 1601 allows for use with a shorter torque tube 1604 than a more proximal exit would, while maintaining control over the proximal end of the torque tube. For an over the wire configuration which is also possible, the exit port 1605 will be at the end of the delivery catheter 1601.

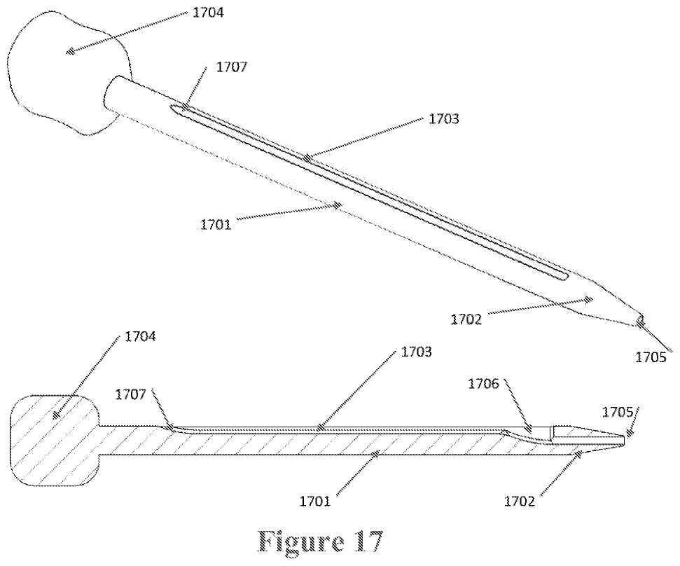

As shown in FIG. 17, a dilator 1701 has a tapered distal tip 1702, a side slot 1703, which runs along the outer diameter of the dilator 1701. A proximal handle 1704, and a distal port 1705 which is approximately centered on the diameter of the dilator 1701. The distal port 1705 is connected to the side slot 1703, so that a wire or tubing passing through the distal port 1705 exits from the side slot 1703. The section view illustrates that the distal port 1705 connects to the sides slot 1703 via a distal transition zone 1706, which tapers gradually toward the side slot 1703 to guide a wire from the (approximately centered) distal port 1705 to the (displaced outwardly) side slot 1703 minimizing friction on the proximal end of the wire. At the proximal end of the side slot 1703 is a proximal transition zone 1707, which forces the wire to exit the side slot 1703 distal to the handle 1704.

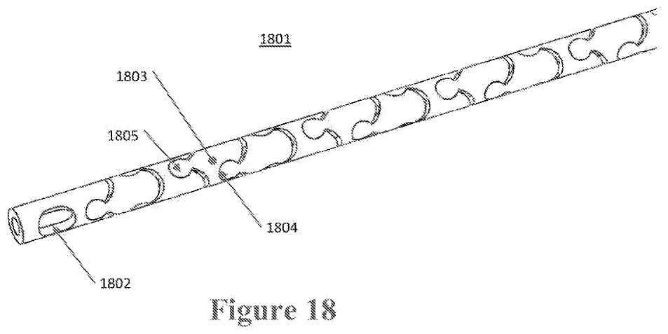

As shown in FIG. 18, a torque tube 1801 is composed of multiple segments. The distal most segment includes a locking feature 1802 to attach the torque tube 1801 to a tissue anchor (not shown). A more proximal segment 1803 has a rotatable boss 1805 and a boss capture port 1804. The rotatable boss 1805 mates with a capture port in a first segment adjacent to segment 1803, while the capture port 1804 mates with a rotatable boss in a second segment adjacent to segment 1803. In this way, segments are linked to each other, forming a tubular chain of segments capable of flexing and delivering a torque.

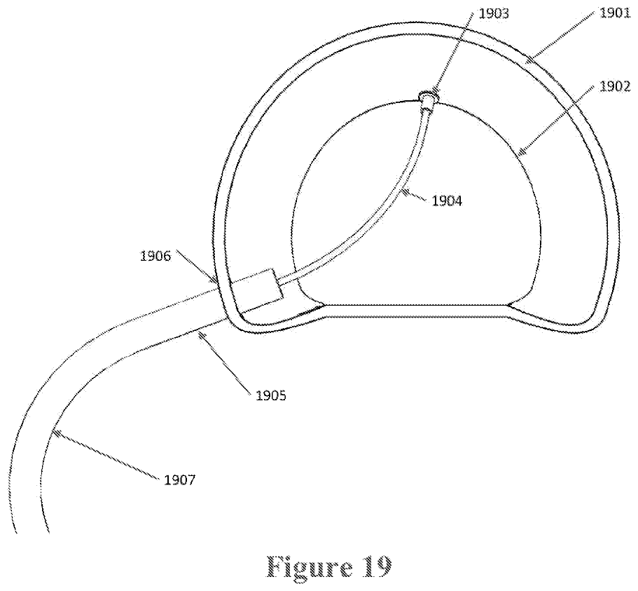

As shown in FIG. 19, a heart chamber 1901 contains a valve annulus 1902 to which a tissue anchor 1903 is attached. The anchor 1903 is releasably coupled to a control wire or tube 1904, which enters the heart chamber 1901 through a sheath 1905. The sheath 1905 enters the heart chamber 1901 through a hole, port, or other opening 1906 in the wall of the heart chamber 1901. A bend 1907 in the sheath 1905 allows the tip of the sheath 1905 to enter the heart chamber 1901 at a desired angular orientation. Applying tension to the control wire or tube 1904 will deflect the annulus 1902 of the valve of the heart chamber 1901, which can be observed via echocardiography, fluoroscopy, CT scan, or other imaging modality.

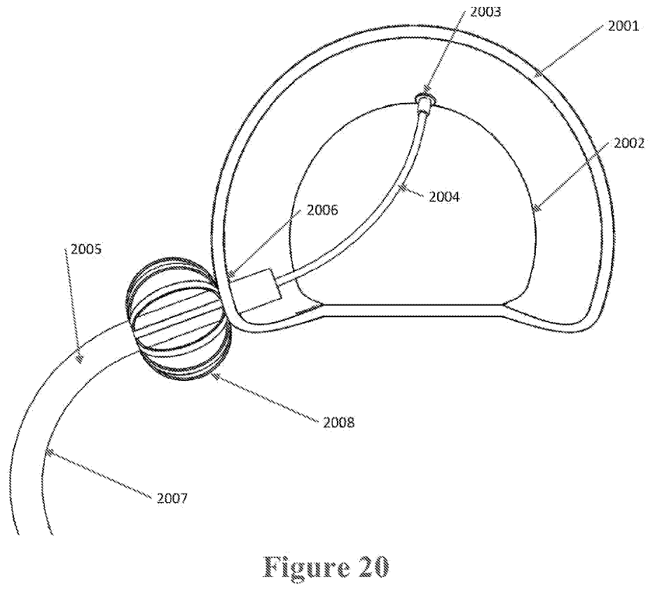

As shown in FIG. 20, a heart chamber 2001 contains a valve annulus 2002 to which a tissue anchor 2003 is attached. The anchor 2003 is releasably coupled to a control wire or tube 2004, which enters the heart chamber 2001 through a sheath 2005. The sheath 2005 enters the heart chamber 2001 through a hole, port, or other opening 2006 in the wall of the heart chamber 2001. A bend 2007 in the sheath 2005 allows the tip of the sheath 2005 to enter the heart chamber 2001 at a desired angular orientation. Applying tension to the control wire or tube 2004 will deflect the annulus 2002 of the valve of the heart chamber 2001, which can be observed via echocardiography, fluoroscopy, CT scan, or other imaging modality. In response to the tension in the control wire or tube 2004, the sheath 2005 tends to move further into the heart chamber 2001. An expandable basket 2008 inhibits said motion of the sheath by being larger than opening 2006.

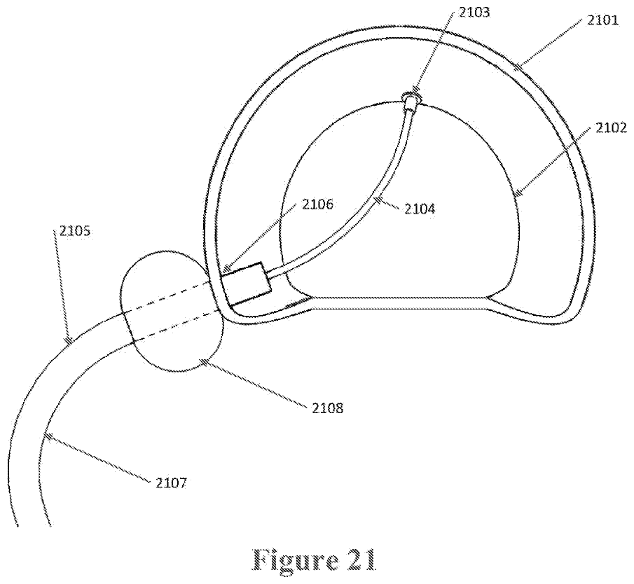

As shown in FIG. 21, a heart chamber 2101 contains a valve annulus 2102 to which a tissue anchor 2103 is attached. The anchor 2103 is releasably coupled to a control wire or tube 2104, which enters the heart chamber 2101 through a sheath 2105. The sheath 2105 enters the heart chamber 2101 through a hole, port, or other opening 2106 in the wall of the heart chamber 2101. A bend 2107 in the sheath 2105 allows the tip of the sheath 2105 to enter the heart chamber 2101 at a desired angular orientation. Applying tension to the control wire or tube 2104 will deflect the annulus 2102 of the valve of the heart chamber 2101, which can be observed via echocardiography, fluoroscopy, CT scan, or other imaging modality. In response to the tension in the control wire or tube 2104, the sheath 2105 tends to move further into the heart chamber 2101. An expandable balloon 2108 inhibits said motion of the sheath by being larger than opening 2106.

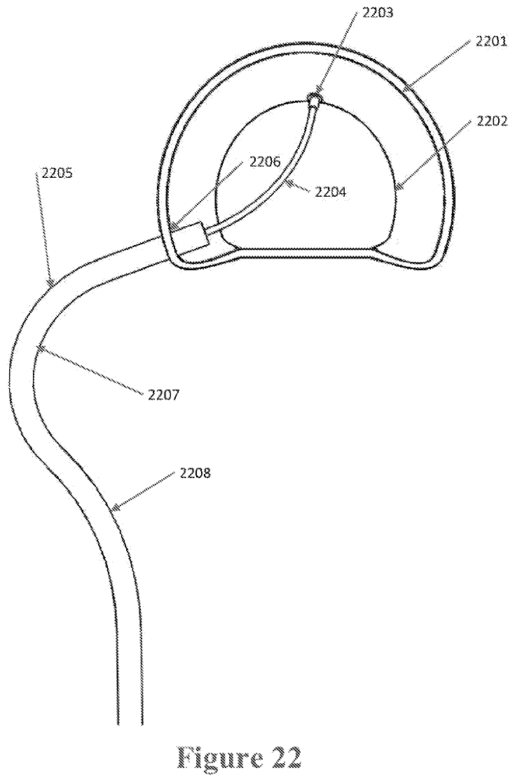

As shown in FIG. 22, a heart chamber 2201 contains a valve annulus 2202 to which a tissue anchor 2203 is attached. The anchor 2203 is releasably coupled to a control wire or tube 2204, which enters the heart chamber 2201 through a sheath 2205. The sheath 2205 enters the heart chamber 2201 through a hole, port, or other opening 2206 in the wall of the heart chamber 2201. A first bend 2207 in the sheath 2205 allows the tip of the sheath 2205 to enter the heart chamber 2201 at a desired angular orientation. Applying tension to the control wire or tube 2204 will deflect the annulus 2202 of the valve of the heart chamber 2201, which can be observed via echocardiography, fluoroscopy, or other imaging modality. In response to the tension in the control wire or tube 2204, the sheath 2205 tends to move further into the heart chamber 2201. As an option, a second bend 2208 on the sheath 2205 can be used to counteract or resist said motion of the sheath 2205.

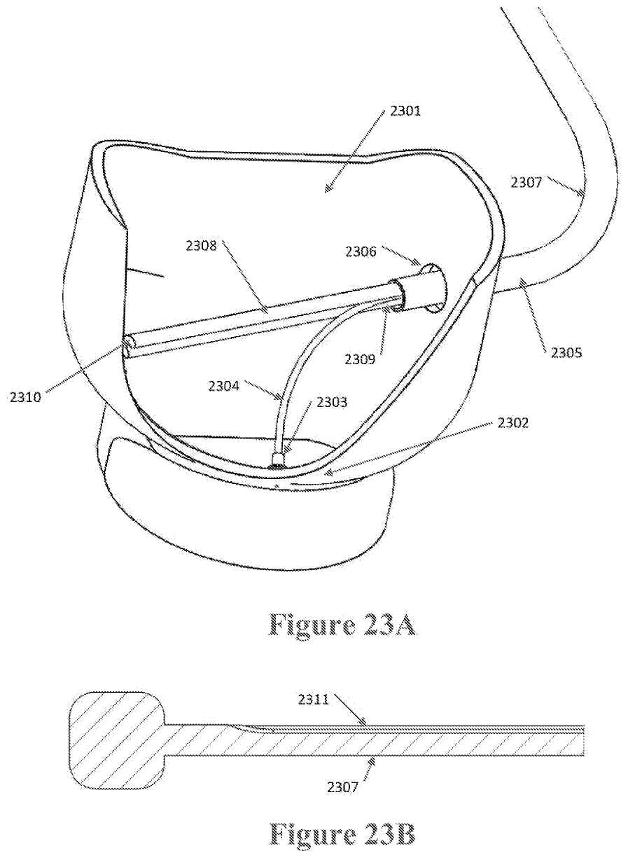

As shown in FIG. 23A, a heart chamber 2301 contains a valve annulus 2302 to which a tissue anchor 2303 is attached. The anchor 2303 is releasably coupled to a control wire or tube 2304, which enters the heart chamber 2301 through a sheath 2305. The sheath 2305 enters the heart chamber 2301 through a hole, port, or other opening 2306 in the wall of the heart chamber 2301. A bend 2307 in the sheath 2305 allows the tip of the sheath 2305 to enter the heart chamber 2301 at a desired angular orientation. Applying tension to the control wire or tube 2304 will deflect the annulus 2302 of the valve of the heart chamber 2301, which can be observed via echocardiography, fluoroscopy, CT scan, or other imaging modality. In response to the tension in the control wire 2304, the sheath 2305 tends to move further into the heart chamber 2301. As an option, a stiffening member 2308 can increase the stiffness of the sheath 2305 and counteract or resist said motion of the sheath 2305. In addition, the stiffening member 2308 can be extended until at least a portion of the distal end 2310 of the stiffening member 2308 contacts the inner wall of the heart chamber 2301, to counteract or resist said motion of the sheath 2305.

As shown in FIG. 23B, the cross section of this stiffening member 2307 includes a slot 2311 through which the control wire or tube can pass.

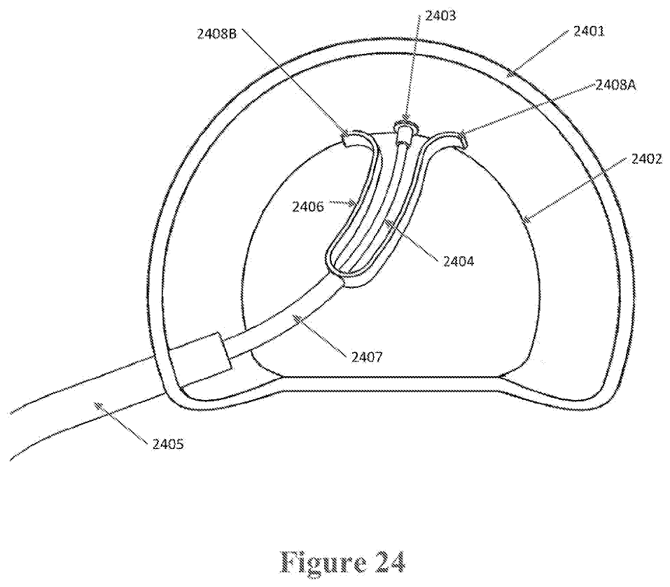

As shown in FIG. 24, a heart chamber 2401 contains a valve annulus 2402 to which a tissue anchor 2403 is attached. The anchor 2403 is releasably coupled to a control wire or tube 2404, which enters the heart chamber 2401 through a sheath 2405. The sheath 2405 enters the heart chamber 2401 through a hole, port, or other opening in the wall of the heart chamber 2401. A bend in the sheath 2405 allows the tip of the sheath 2405 to enter the heart chamber 2401 at a desired angular orientation. Applying tension to the control wire or tube 2404 will deflect the annulus 2402 of the valve of the heart chamber 2401, which can be observed via echocardiography, fluoroscopy, CT scan, or other imaging modality. In response to the tension in the control wire or tube 2404, the sheath 2405 tends to move further into the heart chamber 2401. A footplate 2406 with or without padding attached to a footplate catheter 2407 supports the tissue of the annulus 2402 to either side of the anchor 2403 while tension is applied to the control wire or tube 2404.

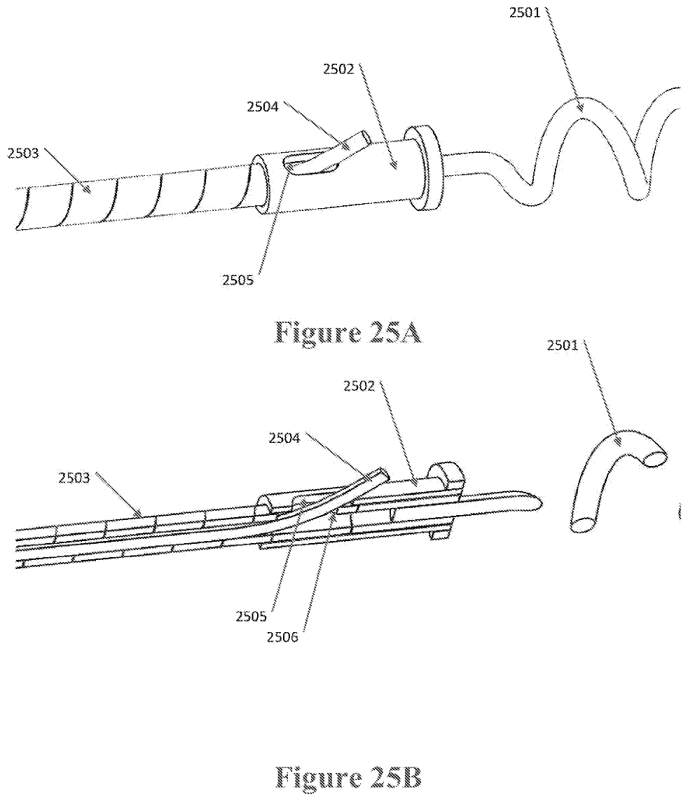

As shown in FIG. 25A and FIG. 25B, a tissue anchor 2501 with an anchor body 2502 is coupled to a torque tube 2503 by a key wire 2504. The key wire 2504 extends through a torque tube hole 2506 in the torque tube 2503 and a lock hole 2505 in the anchor body 2502. The rotational and longitudinal alignment of torque tube hole 2506 and lock hole 2505 allows passage of the key wire 2504, and the key wire 2504 through in place through torque tube hole 2506 and lock hole 2505 prevents or limits their misalignment, effectively coupling the anchor body 2502 to the torque tube 2503. Pulling the key wire 2504 proximally removes it from the torque tube hole 2506 and lock hole 2505, de-coupling the anchor body 2502 from the torque tube 2503.

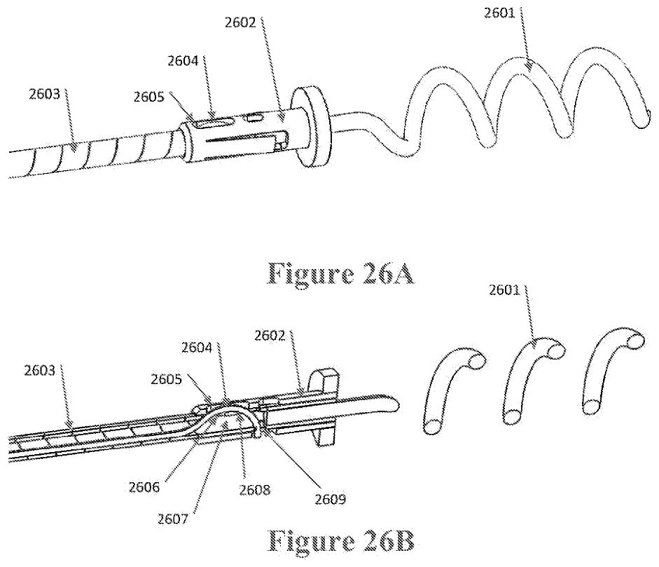

As shown in FIG. 26A and FIG. 26B, a tissue anchor 2601 with an anchor body 2602 is coupled to a torque tube 2603 by a key wire 2604. The key wire 2604 extends through a first torque tube hole 2606, over a bridging segment 2607, and back in through a second torque tube hole 2608. The key wire 2604 as it passes over the bridging segment 2607 extends into a lock slot 2605 in the anchor body 2602. The rotational and longitudinal alignment of first torque tube hole 2606, bridging segment 2607 and second torque tube hole 2608 with the lock slot 2605 allows passage of the key wire 2604, and the key wire 2604 through in place through the first torque tube hole 2606, over the bridging segment 2607 and back through the second torque tube hole 2608 while passing through the lock slot 2605 prevents or limits their misalignment, effectively coupling the anchor body 2602 to the torque tube 2603. Pulling the key wire 2604 proximally removes it from the torque tube holes 2606 and 2608 and lock slot 2605, de-coupling the anchor body 2602 from the torque tube 2603. The anchor body may include a key wire exit hole 2609 through which the key wire 2604 may pass, allowing for tension to be applied to the key wire during assembly. When the diameter of the key wire 2604 is approximately the same as the wall thickness of the anchor body 2602, the profile of the coupled assembly is comparable to the anchor body 2602 alone, allowing unrestricted passage of a tissue shaping template (not shown) over the anchor body 2602.

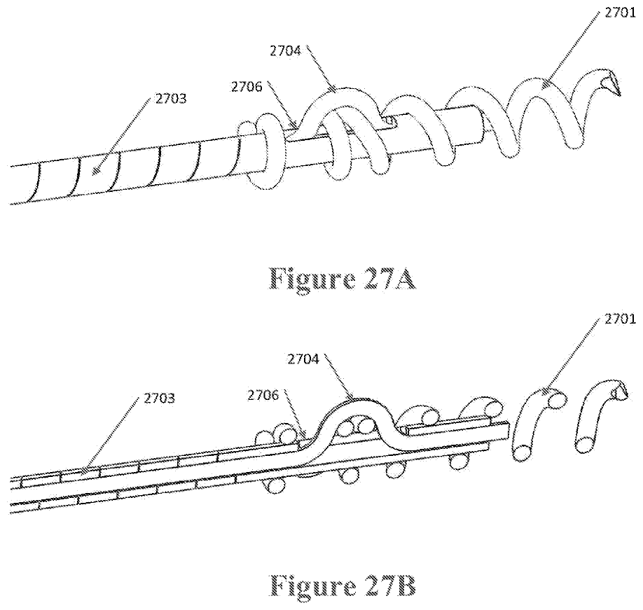

As shown in FIG. 27A and FIG. 27B, a coiled wire tissue anchor 2701 is coupled to a torque tube 2703 by a key wire 2704. The key wire 2704 extends through a torque tube hole 2706 in the torque tube 2703, around at least one coil of the anchor 2701, and back into the torque tube hole 2706. The key wire 2704 maintains the longitudinal alignment of torque tube hole 2706 and captured coil or coils of the anchor 2701. The spacing of the coils of the anchor 2701 allows the key wire 2704 to maintain the rotational alignment of the captured coil or coils of the anchor 2701 and the torque tube 2703 as well. Pulling the key wire 2704 proximally removes it from the torque tube hole 2706, de-coupling the anchor 2701 from the torque tube 2703. As an option, the proximal end of the coiled wire tissue anchor 2701 can be inserted into a short tube welded to the torque tube 2703.

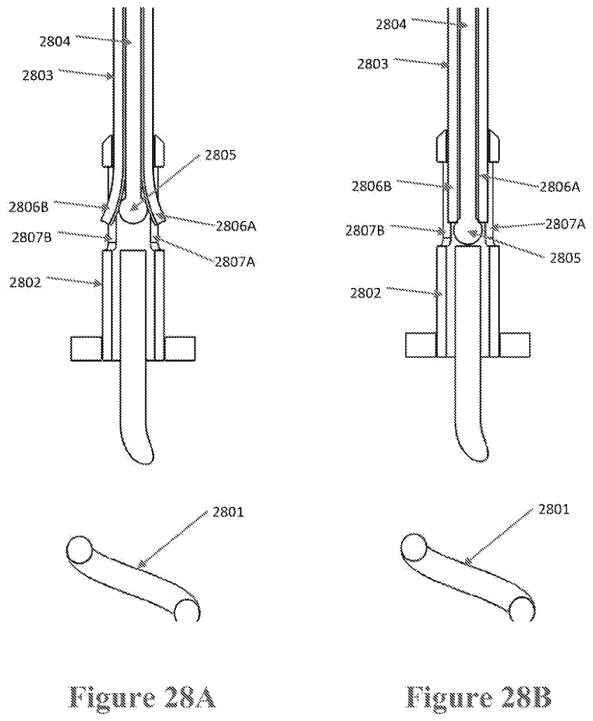

As shown in FIG. 28A and FIG. 28B, a tissue anchor 2801 with an anchor body 2802 is coupled to a torque tube 2803 by tabs 2806A and 2806B forced outward by a key wire 2804 having an enlargement 2805 at its distal end. The tabs 2806A and 2806B extend through tab holes 2807A and 2807B in the anchor body 2802. The rotational and longitudinal alignment of torque tube hole 2806 and lock hole 2805 is maintained by tabs 2806A and 2806B extending through the tab holes 2807A and 2807B, effectively coupling the anchor body 2802 to the torque tube 2803. Pushing the key wire 2804 distally moves the enlargement away from the tabs, allowing them to flex inward, de-coupling the anchor body 2802 from the torque tube 2803.

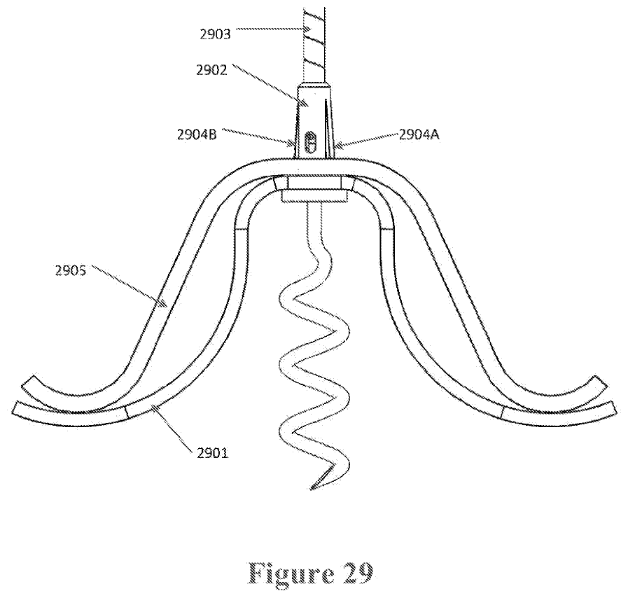

As shown in FIG. 29, a tissue anchor 2902 is coupled to tissue (not shown). The tissue anchor is releasably coupled to a control wire or tube 2903. A tissue template 2901 has a hole through which the control wire 2903 can pass, allowing the template 2901 to slide over the control wire or tube 2903 for placement in contact with the target tissue. After the template 2901 is placed and while the anchor 2902 and control wire or tube 2903 are still coupled, the effect can be assessed. If the desired effect has not been achieved, a reinforcement 2905 can be applied over the control wire to add to the effect. Both the template 2901 and reinforcement 2905 are coupled to the anchor 2902 by extensible tabs 2904A and 2904B. The reinforcement 2905 may act to further shape the template 2901 as shown, or may interact directly with the target tissue, or tissue adjacent to the tissue template 2901.

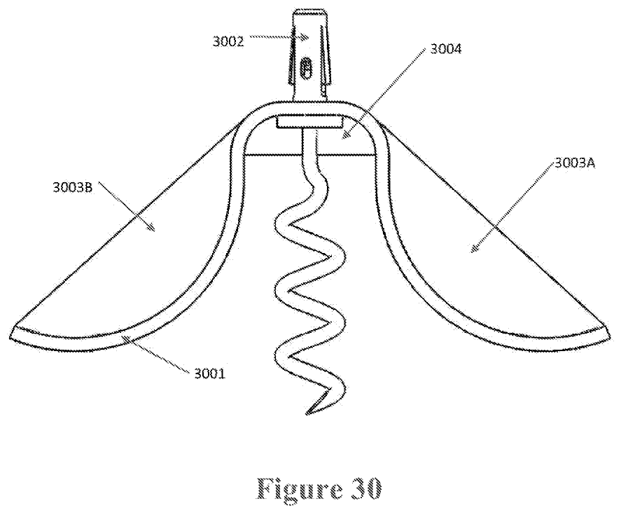

As shown in FIG. 30, a tissue template 3001 is coupled to a tissue anchor 3002 which holds it in apposition to a target tissue (not shown). The template 3001 includes an outer skirt 3003A and 3003B, which smooth the profile of the template 3001 in the tissue. The template 3001 also includes an inner skirt 3004, which fills the space between the template 3001 and the tissue drawn up against the inner surface of the template 3001. The skirts may be constructed of a woven or knitted fabric such as Dacron, or non-woven material, such as ePTFE, Tyvek, electrospun fibers, or the like. The skirts provide material for control of blood flow in the short term following implantation, and for tissue in growth in the long term. As an option, outer skirt 3003 or inner skirt 3004 may be omitted.

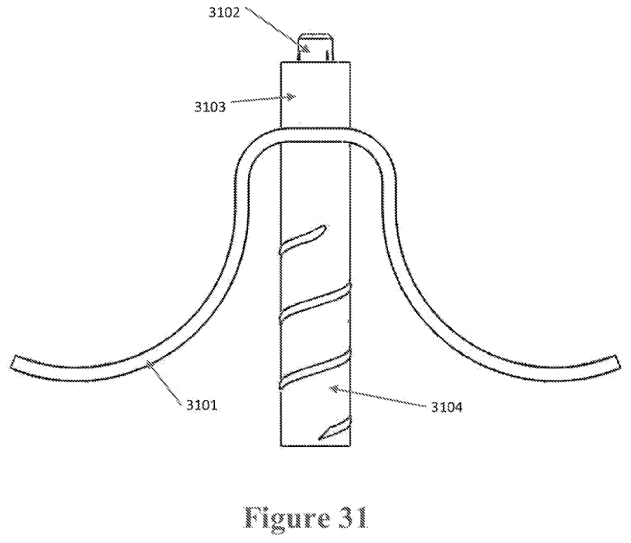

As shown in FIG. 31, a tissue template 3101 is coupled to a tissue anchor 3102 which holds it in apposition to a target tissue (not shown). The anchor 3102 has a proximal covering 3103 and a distal covering 3104. The proximal covering 3103 allows for tissue in growth in the region of the anchor 3102 proximal to the template 3101, while the distal covering 3104 protects the tip of the anchor during delivery to the target tissue, then collapses into a bunch as the tissue anchor 3102 is twisted into the target tissue, helping to seal any space between the target tissue and the template 3101 or hole in the target tissue. The covers may be constructed of a woven or knitted fabric such as Dacron, or non-woven material, such as ePTFE, Tyvek, electrospun fibers, or the like. The covers provide for tissue ingrowth or sealing in the long term.

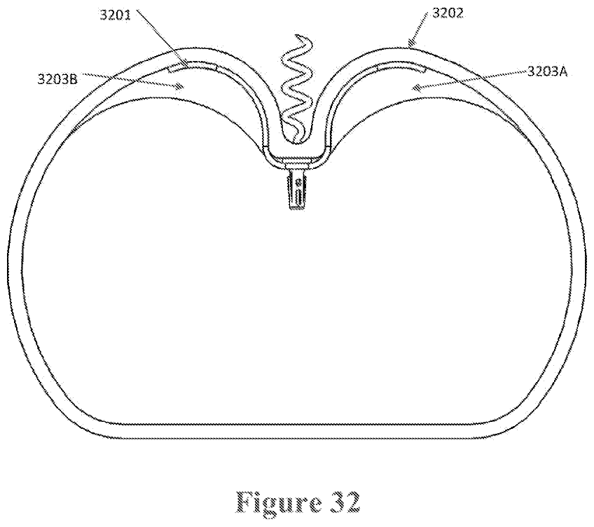

As shown in FIG. 32, a tissue template 3201 is attached to an annulus 3202 via a tissue anchor. The tissue template 3201 includes skirts 3203A and 3203B, which smooth the transition between the tissue shaped by the template 3201 and the rest of the annular tissue. In this way, the skirts 3203A and 3203B create a healed annular area which will be easier to seal with a replacement valve, if such a procedure is needed in the future.

As shown in FIG. 33, a tissue anchor 3301 is placed in the annulus 3302 of a heart valve. The heart valve divides two chambers of the heart, the atrium 3303 and the ventricle 3304. While the anchor 3301, and any tissue shaping templates to be attached to the anchor, can be applied from either side of the annulus, this figure shows the anchor 3301 applied from the ventricular side.

As shown in FIGS. 34A and 34B, when a tissue shaping template is applied to the posterior side of a mitral valve annulus by a tissue anchor 3402 at an appropriate angle to couple with the mitral annulus, the posterior side of the mitral annulus moves from a flat position 3401 to an elevated position 3403.

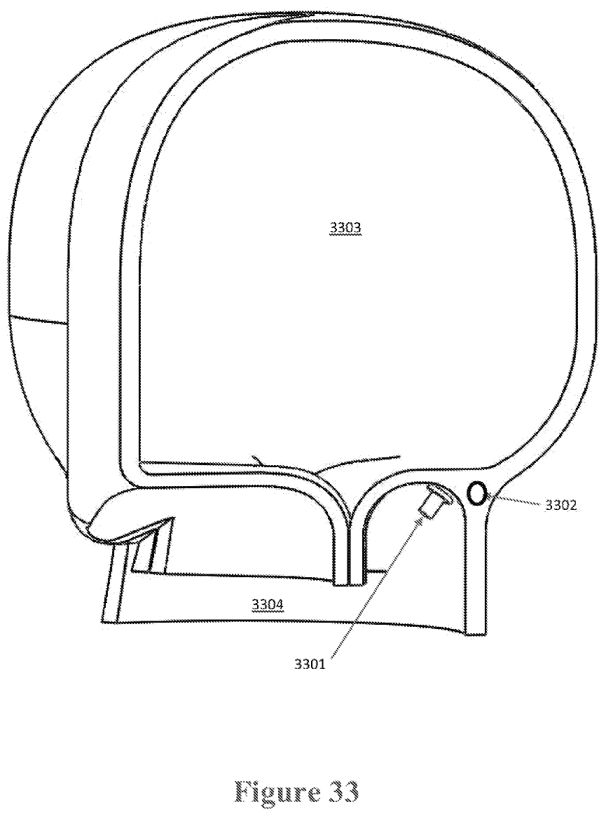

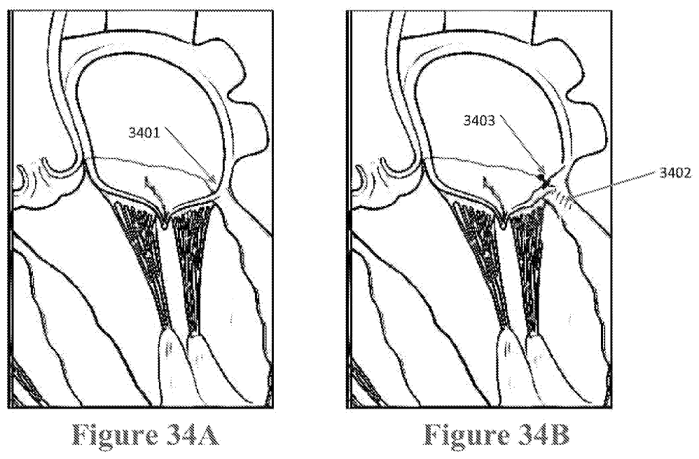

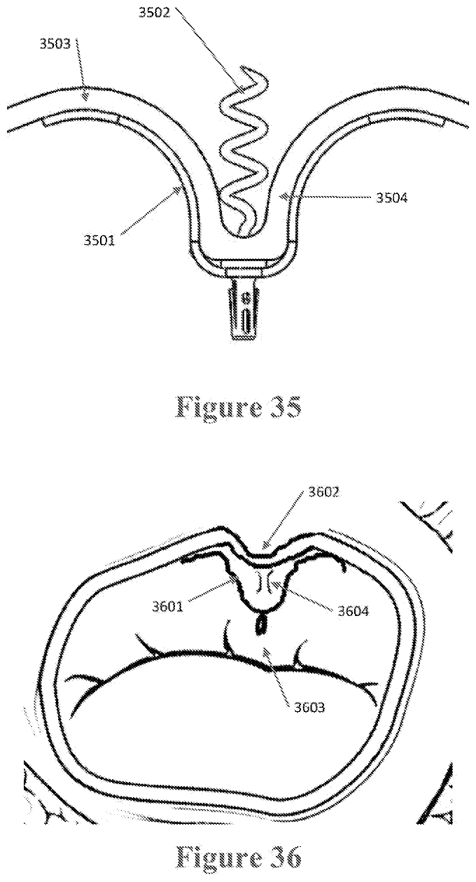

As shown in FIG. 35, a tissue shaping template 3501 is coupled to a target annulus 3503 by a tissue anchor 3502. The annulus is folded or plicated 3504 by this reshaping.

As shown in FIG. 36, a tissue shaping template 3601 is coupled to a target annulus 3602 in the vicinity of a target leaflet 3603. The leaflet is folded or plicated 3604 by the template 3601. This results in shrinking and stiffening of the leaflet to prevent or improve leaflet prolapse.

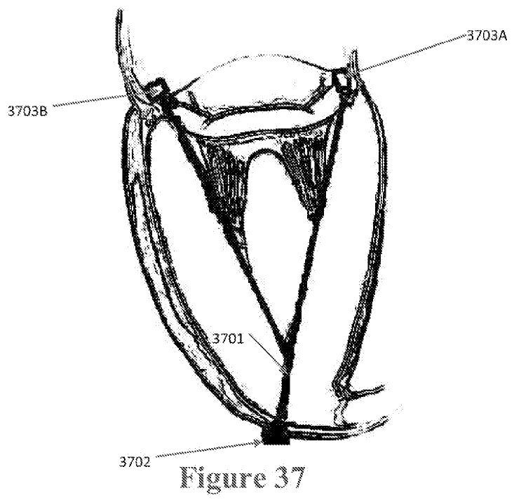

As shown in FIG. 37, a system for applying apical tension to the mitral annulus includes a tension member 3701, an apical anchor 3702, and annular anchors 3703A and 3703B. The apical anchor 3702 may be placed inside the left ventricle by catheter access (trans-catheter approach), or outside the left ventricle by thoracotomy (trans-apical approach). The apical anchor may include means to adjust the tension applied by the tension member 3701. The annular anchors 3703A and 3703B may be placed from the atrial or ventricular side of the annulus and may include means to adjust the tension applied by the tension member 3701. This system could be applied to the tricuspid annulus, or other valve annuli as well as the mitral. Anchor points may be chosen based on valve annulus shape such as saddle points, they may target features on the valve leaflets such as clefts or commissures, or they may target features of the fibrous skeleton of the heart, such as trigones.

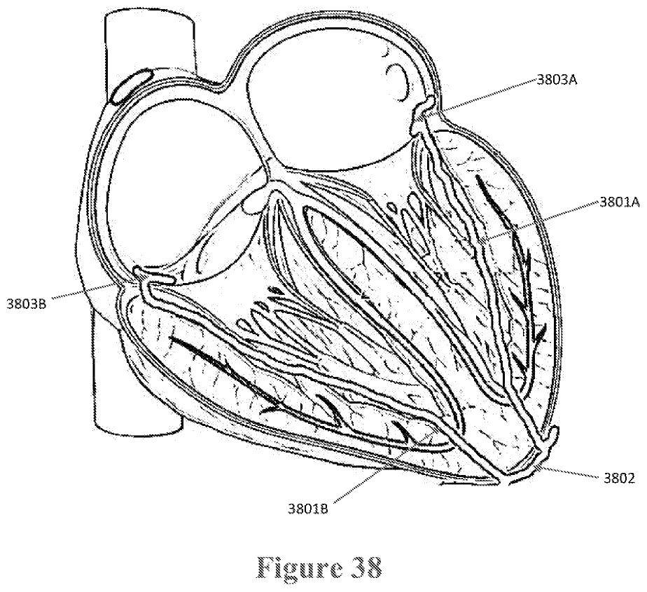

As shown in FIG. 38, a system for simultaneously applying tension to the mitral and tricuspid annuli includes tension members 3801A and 3801B, an apical anchor 3802, a mitral annular anchor 3803A and a tricuspid anchor 3803B. The apical anchor 3802 is coupled to at least two tension members 3801A and 3801B, at least one of which traverses at least part of the right ventricle and is coupled to a tricuspid anchor 3803B, and at least a second of which traverses at least part of the left ventricle and coupled to a mitral anchor 3803A. Tension in the tension members 3801A and 3801B moves the anchor points 3803A and 3803B on the valve annuli closer to the apex of the heart. Anchor points may be chosen based on valve annulus shape such as saddle points, they may target features on the valve leaflets such as clefts or commissures, or they may target features of the fibrous skeleton of the heart, such as trigones.

As shown in FIG. 39, a spacer 3902 is placed between the posterior leaflet 3901 and the wall of the left ventricle in a valve having leaflet tethering caused at least in part by enlargement of the left ventricle, and the spacer 3902 acts to move the sealing surface of the posterior leaflet closer to the anterior leaflet. The spacer may take the form of a basket, balloon, an expanding foam material, or a stent or stent like structure. The stent or stent like structure may or may not be covered with a material that creates a hemostatic seal against the anterior leaflet. The stent or stent like structure may or may not be anchored to the ventricle wall or annulus.

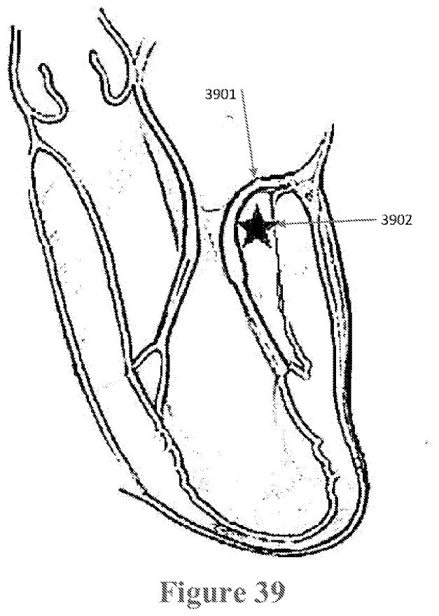

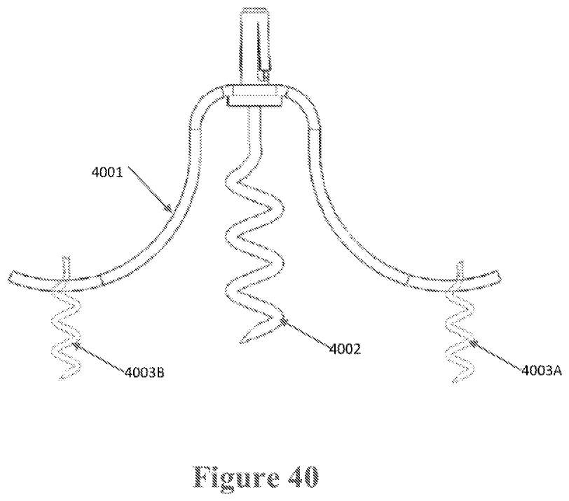

As shown in FIG. 40, a tissue shaping template 4001 is coupled to a tissue anchor 4002, which is anchored to target tissue (not shown.) Stabilizing anchors 4003A and 4003B are coupled to the template 4001 and further anchor to the target tissue, to provide additional stability to the template 4001. For some configurations, more than two stabilizing anchors 4003 may be advantageous. One stabilizing anchor 4003 may be all that is necessary.

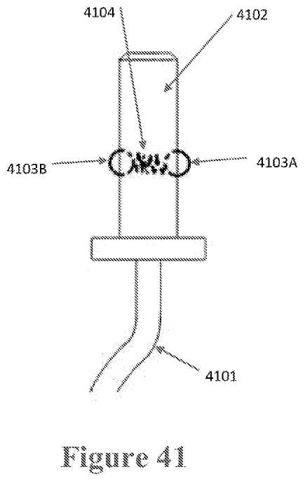

As shown in FIG. 41, a tissue anchor 4101 is shown with an anchor body 4102 having detent balls 4103A and 4103B disposed partially within the body, and held in an outward position by a spring 4104. A torque tube (not shown) can slide over the anchor body 4102, depressing the detent balls 4103A and 4103B during insertion. The detent balls 4103A and 4103B then are urged outward by the spring 4104, effectively coupling the torque tube to the anchor body by fitting into mating holes or depressions in the torque tube.

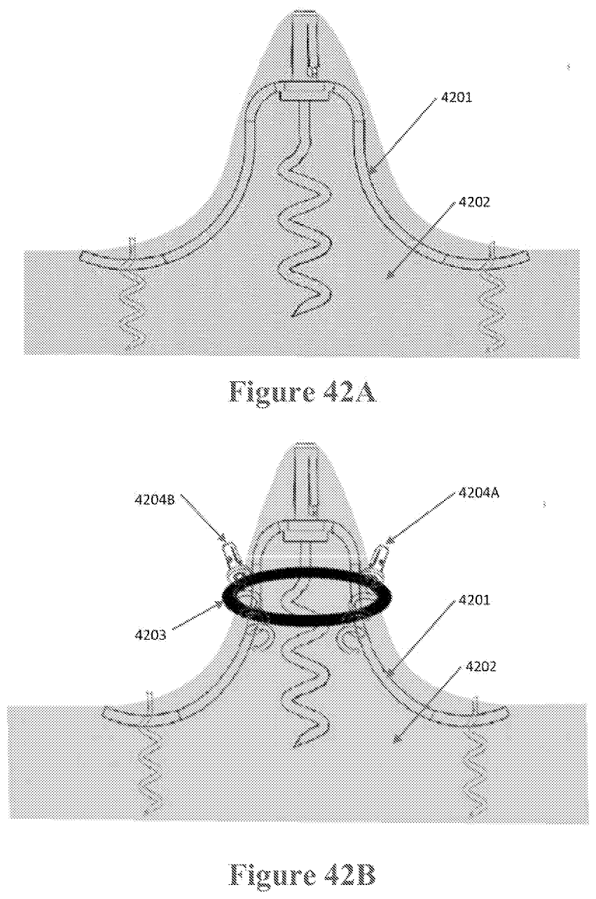

As shown in FIGS. 42A and 42B, a tissue shaping template 4201 is in place and shaping target tissue 4202. In FIG. 42A, the effect on the target tissue is measured, and it is determined that adjustment would be advantageous. In FIG. 42B, a ring 4203 has been applied to the tissue shaping template and target tissue 4202, in order to create additional gathering forces on the target tissue 4202. The ring 4203 may be locked in place with one or more ring anchors 4204A and 4204B

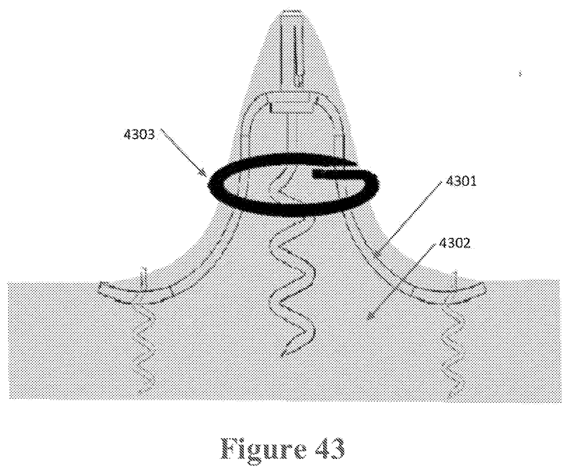

As shown in FIG. 43, a tissue shaping template 4301 is in place and shaping target tissue 4302. A self-closing ring 4303 has been applied to the tissue shaping template and target tissue 4302, in order to create additional gathering forces on the target tissue 4302. The ring 4303 take the form of a clip, staple, coil, or the like.

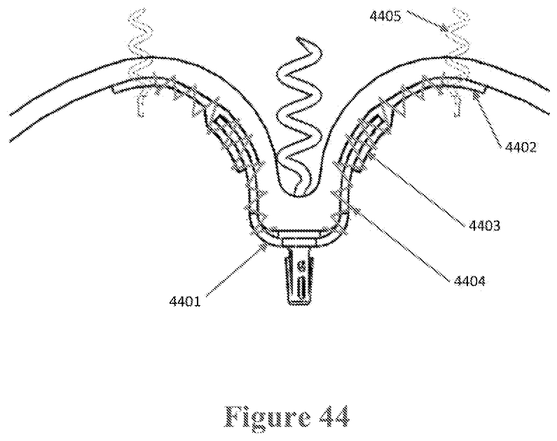

As shown in FIG. 44, a tissue template has a center portion 4401 and one or more end portions 4402, which are connected at a junction zone 4403. A spring 4404 is constrained at one end on the center portion 4401 and at the other end on the end portion 4402. The spring may be configured to open slowly over time, for example by forming it of a shape memory material. Alternately, the junction zone 4403 can be bonded closed with a corrodible or bio-resorbable material, with the spring in a compressed or extended state. Over time, as the corrodible or bio-resorbable material degrades, the spring will activate and alter the shape of the template.

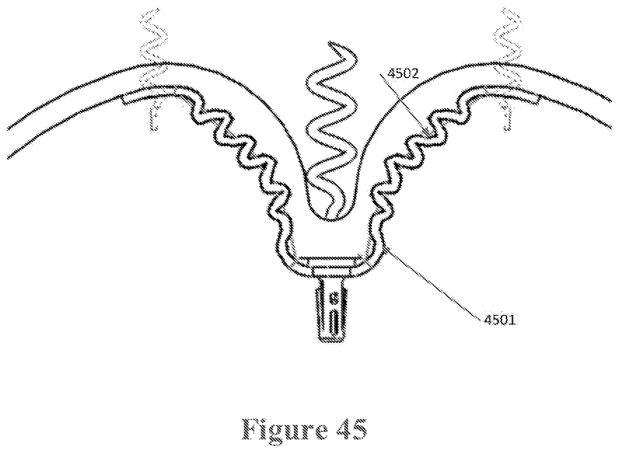

As shown in FIG. 45, a template 4501 in the form of coils, springs, closed cells, or other such construction is held in a compressed or extended state by a temporary member 4502 composed of corrodible or biodegradable material in the form of filament, tubing, wire, coating, or the like. Upon degradation of the temporary member 4502, the template returns to the unloaded state and shape.

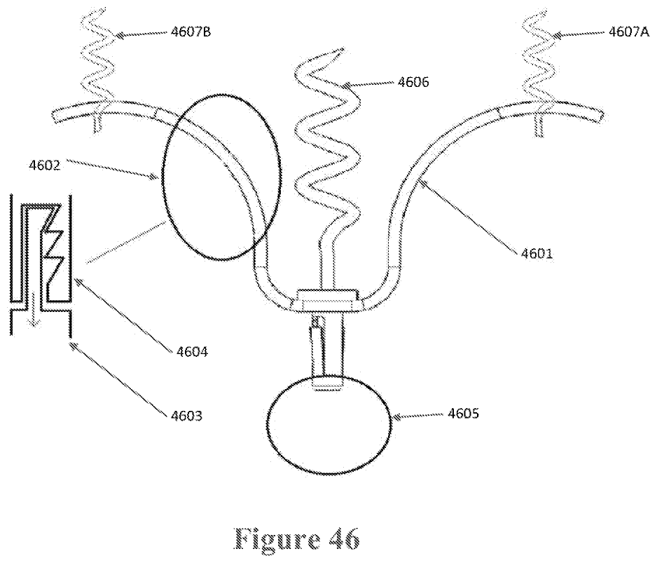

As shown in FIG. 46, a template 4601 has an extensible portion 4602 having a female end 4603 and a male end 4604 which are movable relative to each other. The center of the template is fastened to tissue via a tissue anchor 4606, while the ends are stabilized via stabilizing anchors 4607A and 4607B. A group of interlocking teeth hold the male 4604 and female 4603 ends fixed relative to each other in response to forces in one direction but allow a change of length in response to forces in the opposite direction. A hoop 4605 arranged at an accessible point along the template 4601 can be used to adjust the male 4604 and female 4603 portions by applying a force in the appropriate direction with or without pushing at the ends of the template 4601.

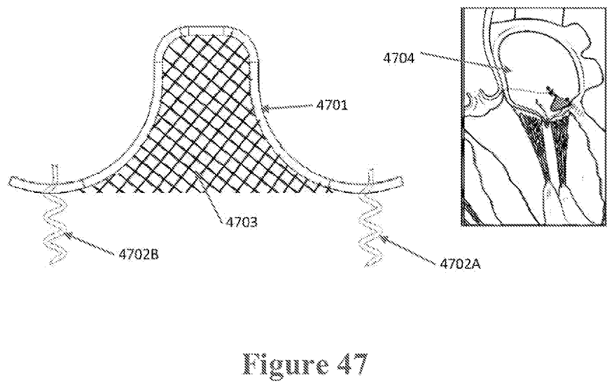

As shown in FIG. 47, a tissue template 4701 is coupled to tissue via one or more anchors 4702A and 4702B and is covered with sleeve 4703 made of a porous, knitted, weave fabric, electrospun fibers, or the like. The sleeve 4703 provides support for prolapsing leaflet and tissue ingrowth media. The tissue template 4701 can be placed above the target leaflet in the atrium 4704, below the target leaflet in the ventricle, or it can bridge several leaflets for additional support.

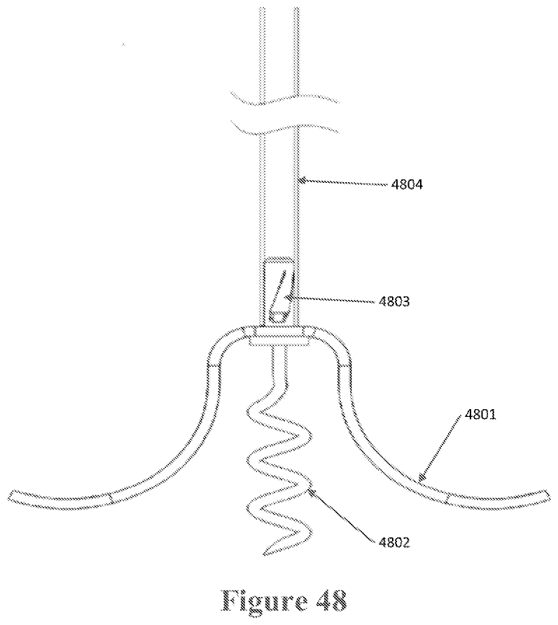

As shown in FIG. 48, a tissue shaping template 4801 is coupled to a tissue anchor 4802 by at least one extensible tab 4803, having a low-profile delivery position and an extended position. The extensible tab 4803 is held in the delivery position by a thin tube 4804, and the template 4801 slides over the thin tube 4804 into position on the anchor 4802. When the thin tube 4804 is removed, the tab 4803 is free to move to the extended position capturing the template 4801. A torque tube (not shown) may be placed within the thin tube 4804, and can be used to steer, torque, and manipulate the anchor 4802 as required.

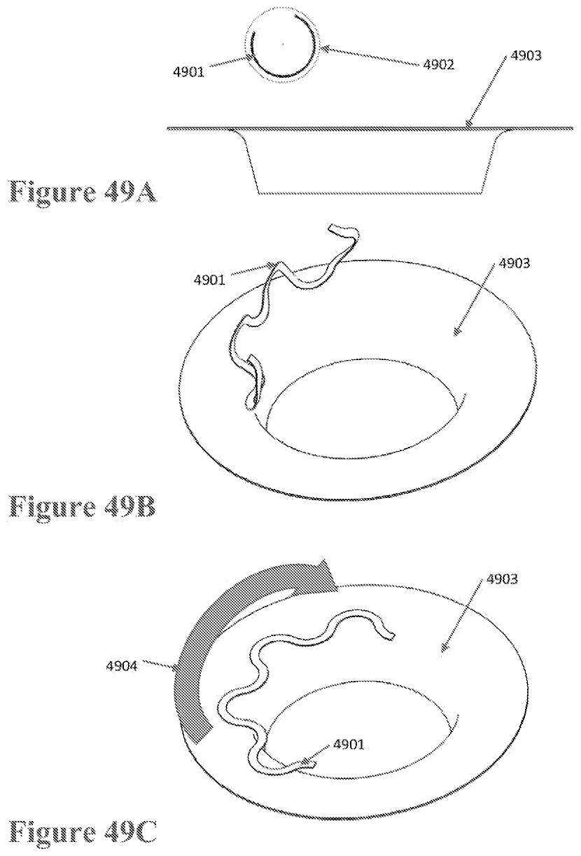

As shown in FIG. 49A, a tissue shaping template 4901 is rolled to fit within a smaller diameter tube 4902 for delivery to the region of a valve annulus 4903. As shown in FIG. 49B, the tissue shaping template 4901, still in the rolled configuration, approaches the valve annulus 4903. As shown in FIG. 49C, the tissue shaping template 4901 is unrolled 4904 to a deployed shape adjacent the valve annulus 4903. FIGS. 49A through 49C show the delivery of a partial ring tissue shaping template 4901 which could be guided into place by anchor control wires, and could dock to previously placed tissue anchors, or could be coupled to the valve annulus through other means known to the art.

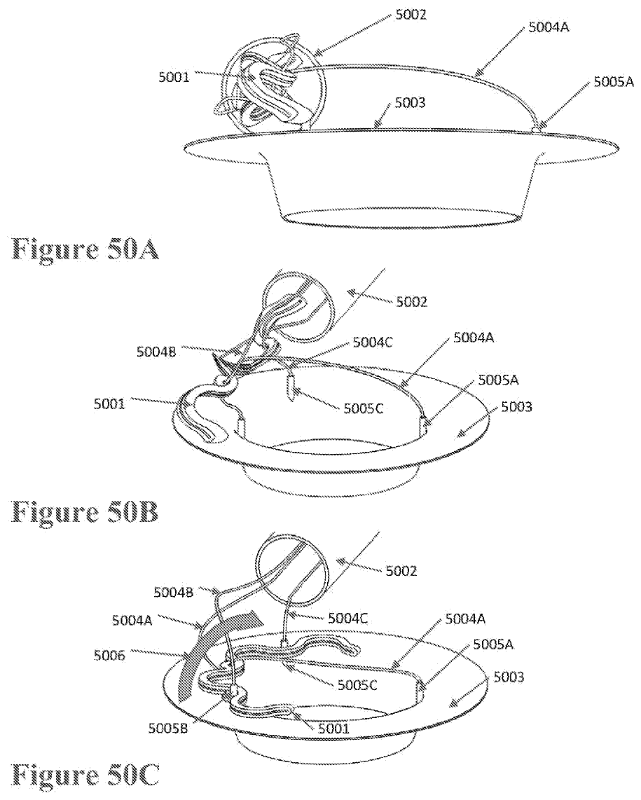

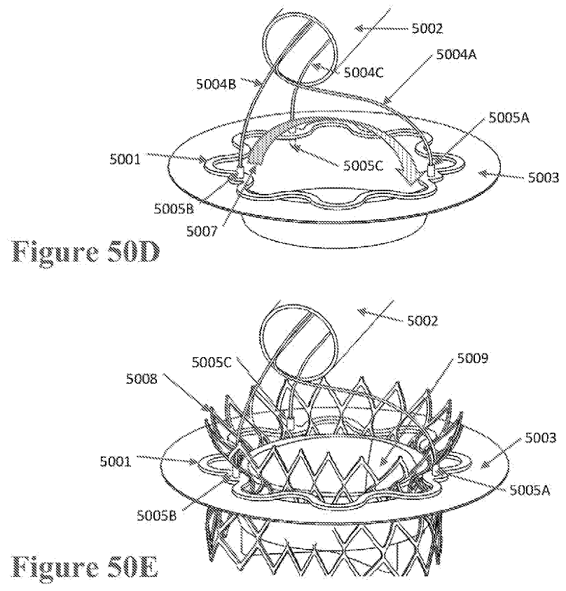

As shown in FIG. 50A, a ring-shaped template 5001 is placed in a delivery configuration and passed through a delivery catheter 5002 while coupled to one or more tissue anchors 5005A via one or more anchor control wires 5004A. The one or more tissue anchors 5005A are coupled adjacent a valve annulus 5003. The ring-shaped template 5001 is reduced in size for passage through the delivery catheter 5002 by folding the ring-shaped template 5001 approximately in half into a crescent shape, and pulling the two folded sections of the ring farther apart from each other, substantially straightening the crescent shape. FIG. 50B shows the valve annulus 5003 and ring-shaped template 5001 of FIG. 50A from a different view, so that two additional anchors, 5005B and 5005C, and their control wires 5001B and 5004C are visible. Also illustrated is the path of the control wires through mating features on the tissue shaping template 5001. FIG. 50C shows the ring-shaped template having returned 5006 to the folded crescent shaped configuration, placed adjacent the valve annulus 5003. As shown, the ring-shaped template 5001 is docked to anchors 5005B and 5005C, although the docking may not be completed until the ring-shaped template 5001 is unfolded. FIG. 50D shows the ring-shaped template 5001 unfolded 5007, and docked to three tissue anchors 5005A, 5005B, and 5005C. The control wires 5004A, 5004B, and 5004C may be released if placement of this template is all that is required, or they may be retained to guide additional implants into place. FIG. 50E shows the ring-shaped implant 5001 with a replacement valve consisting of a cage 5008 and a valve body 5009, having been guided into position along the control wires 5004A, 5004B, and 5004C. The valve body 5009 may be coupled to the valve cage 5008, to one or more of the tissue anchors 5005A, 5005B, 5005C, directly to the valve 5003 anatomy, or by some combination of coupling mechanisms. The valve cage 5008 may be coupled to one or more of the tissue anchors 5005A, 5005B, 5005C, directly to the valve 5003 anatomy, or by some combination of coupling mechanisms.

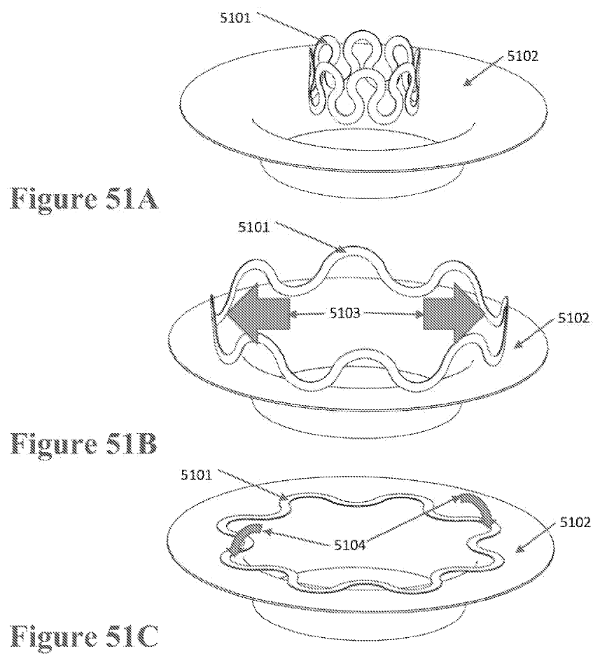

As shown in FIG. 51, a ring-shaped implant 5101 is placed adjacent a valve annulus 5102. Relative to the flat functional configuration of the ring-shaped implant 5101, the delivery configuration is a smaller diameter achieved by first twisting the flat into a cylindrical shape, and then compressing or crimping the cylindrical shape to a deliver configuration. FIG. 51A shows the ring-shaped implant 5101 in the delivery configuration adjacent valve annulus 5102. FIG. 51B shows the ring-shaped implant 5101 in the un-crimped or expanded 5103 configuration adjacent valve annulus 5102. FIG. 51C shows the ring-shaped implant 5101 having twisted 5104 from the expanded configuration to the flat functional configuration adjacent valve annulus 5102. FIGS. 51A through 51C show the delivery of a ring-shaped template 5101 which could be guided into place by anchor control wires, and could dock to previously placed tissue anchors, or could be coupled to the valve annulus through other means known to the art.

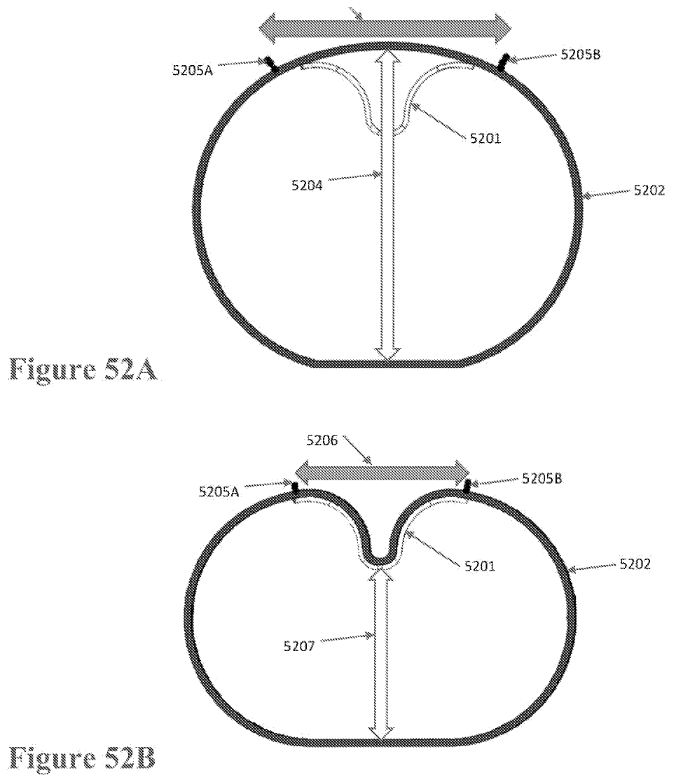

As shown in FIGS. 52A and 52B, a tissue shaping template 5201 is coupled to a valve annulus 5202 having the effect of moving two points 5205A and 5205B on the annulus 5202 closer together, and reducing the effective circumference of the annulus 5202. As shown in FIG. 52A, the valve annulus 5202 has an initial minor diameter 5204 and two points 5205A and 5205B on the annulus 5202 separated by an initial distance 5203. FIG. 52B shows the system of FIG. 52A after coupling the template 5201 to the annulus 5202. This coupling has the effect of changing the initial minor diameter 5204 to a reduced minor diameter 5207, while moving two points 5205A and 5205B on the annulus 5202 from the initial distance 5203 to a shorter distance 5206. In this manner, the effective circumference of the valve annulus 5202 is reduced by the difference between initial distance 5203 and shorter distance 5206.

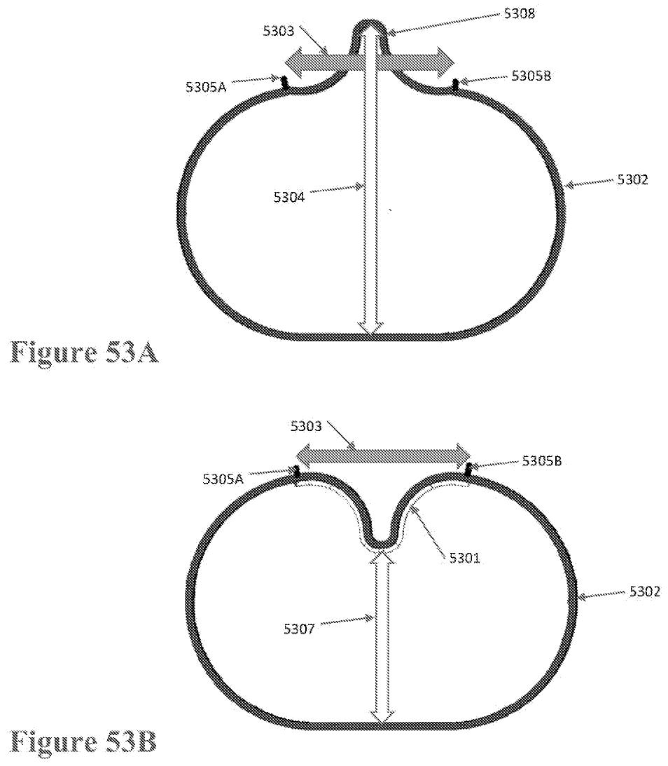

As shown in FIGS. 53A and 53B, a tissue shaping template 5301 is coupled to a valve annulus 5302 with a locally extended segment 5308, having the effect reducing the minor diameter of a valve annulus 5302 while maintaining substantially the same effective circumference of the annulus 5302. As shown in FIG. 53A, the valve annulus 5302 has an initial minor diameter 5304 and two points 5305A and 5305B on the annulus 5302 separated by a distance 5303. FIG. 53B shows the system of FIG. 53A after coupling the template 5301 to the annulus 5302 in the region of the locally extended segment 5308. This coupling has the effect of changing the initial minor diameter 5304 to a reduced minor diameter 5307, while maintaining two points 5305A and 5305B on the annulus 5302 at substantially the same distance 5303. In this manner, the effective circumference of the valve annulus 5302 is unchanged, while a local segment 5308 of the annulus is moved toward the opposite wall, decreasing the annular area.

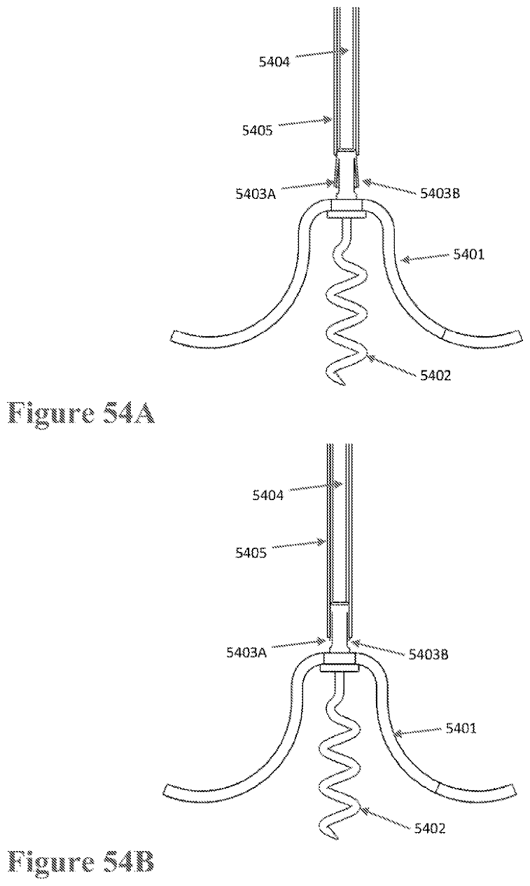

FIGS. 54A and 54B show a template 5401 with a docking anchor 5402 using extensible tabs 5403A and 5403B and releasably coupled to a control wire 5404. The extensible tabs 5403A and 5403B compress to allow the template 5401 to move in one direction over the tabs 5403A and 5403B, but prevent motion in the opposite direction. In FIG. 54A, a release device 5405 arranged at least partially around the control wire 5404 is in the retracted position, and the extensible tabs 5403A and 5403B are extended, locking the template 5401 to the tissue anchor 5204. In FIG. 54B, the release device 5405 arranged at least partially around the control wire 5404 is in the forward position, compressing the extensible tabs 5403A and 5403B and releasing the template 5401 from the tissue anchor 5204.

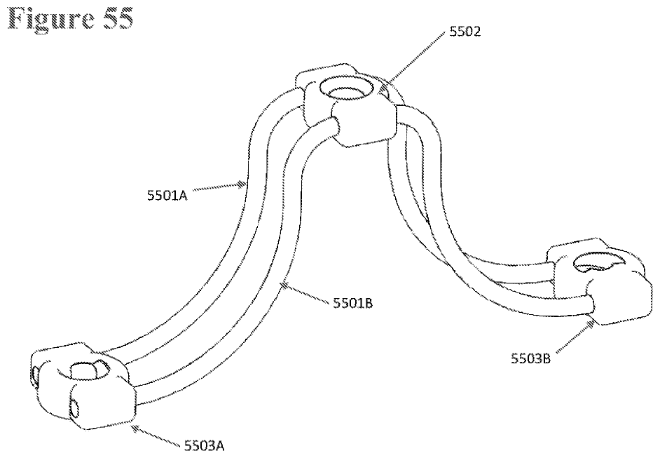

FIG. 55 shows an alternate method of forming a tissue template, which consists of one or more wireforms 5501A and 5501B, coupled to an anchor coupling boss 5502. As shown, the wireforms 5501A and 5501B are also coupled to two end bosses 5503A and 5503B. The wireforms 5501A and 5501B may be circular in cross section, rectangular, hexagonal, or some other substantially constant cross section. The wireforms 5501A and 5501B may be coupled to the anchor coupling boss 5502 and the end bosses 5503A and 5503B with one or more of the following coupling techniques; glue, crimping, welding, brazing, soldering, press fit, or a combination of one or more of these coupling techniques or others known to the art. The anchor coupling boss 5502 may include features which interact with one or more tissue anchors including holes, chamfers, tab docking surfaces, helixes, inner threads, outer threads, or the like. The end bosses 5503A and 5503B may include features which interact with one or more tissue anchors including holes, chamfers, tab docking surfaces, helixes, inner threads, outer threads, or the like. As shown, there are two distinct wireforms 5501A and 5501B, but by looping the wire they could both be formed of a single continuous piece of wire, with two ends of the wire at the same end boss (resulting in a single end-boss design), or with the two ends of the wire at the center anchor boss 5502 (resulting in a design having no end bosses.)

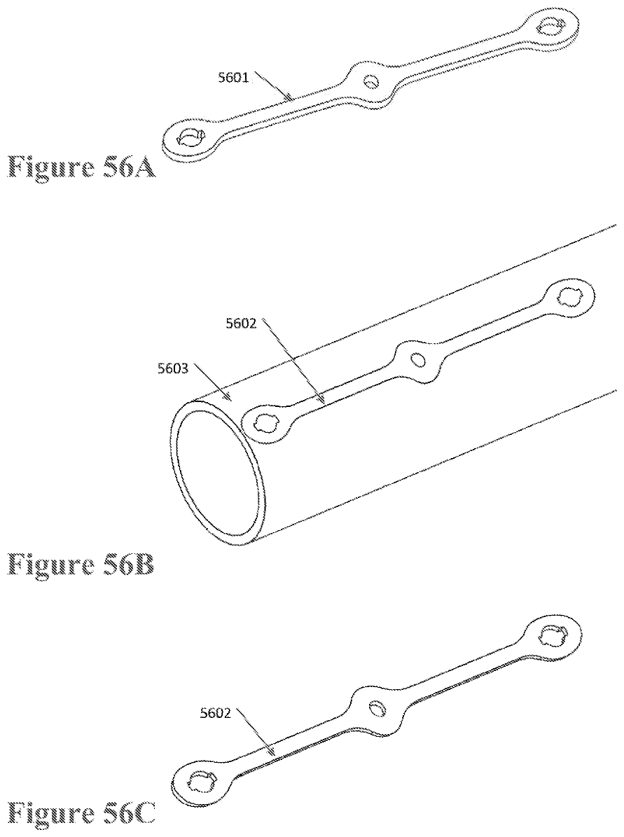

As shown in FIGS. 56A through 56C, an anchor blank 5601 may be formed by cutting from a flat sheet, or a similar anchor blank 5602 may be formed by cutting from a tube 5603. FIG. 56A shows a blank 5601 cut from a flat, FIG. 56B shows a blank 5602 cut from a tube in place on the tube, and FIG. 56C shows the bland 5602 cut from a tube with the tube removed. Advantages of cutting a blank 5602 from a tube 5603 may include tubes having different material properties than a sheet, tubes having improved properties in the direction of the draw compared to a sheet, or other such material property advantages inherent in the process of forming tubes. Additional advantages of forming a blank 5602 from a tube 5603 may include conferring a curved shape to the blank (best seen in FIG. 56C) based on the diameter of the tube 5603 of the base material. Cutting methods may include laser cutting, mechanical milling, waterjet cutting, photochemical etching, or other subtractive manufacturing processes known to the art.

As shown in FIG. 57, the heart 5705, contains four major valves: the mitral or bicuspid valve 5701, the pulmonary valve 5702 with the Right Cusp 5702a, Left Cusp 5702b, and Anterior Cusp 5702c, the aortic valve 5703 with the Non-Coronary Cusp 5703a, the Right Coronary Cusp 5703b, and the Left Coronary Cusp 5730c, and the tricuspid valve 5704 with the Posterior leaflet 5704a, the Anterior Leaflet 5704b, and the Septal Leaflet 5704c. Each valve has three leaflets, except for the mitral valve which has two.

As shown in FIG. 58, the mitral valve 5701 comprises a mitral valve annulus 5801, and has an anterior leaflet 5803 with a first scallop (A1) 5803a, a second scallop (A2) 5803b, and a third scallop (A3) 5803c, and a posterior leaflet 5804 with a first scallop (P1) 5804a, a second scallop (P2) 5804b, and a third scallop (P3) 5804c, which join at commissures 5802a and 5802b. Referring to FIG. 58, the septal aspect of the valve 5806 is at the bottom of the figure, and the lateral aspect of the valve 5805 is at the top.

As shown in FIG. 59, the mitral valve 5701 can enlarge, leaving a gap 5901 between the anterior 5803 and posterior 5804 leaflets. This gap 5901 prevents the valve from closing, allowing blood to return from the left ventricle to the left atrium, a condition referred to as MR or Functional Mitral Regurgitation, or FMR.

One object of this invention is to change the configuration of the valve to minimize or reduce MR. In one example, decreasing one dimension of a heart valve may be accomplished by increasing another dimension using a device. Referring to FIG. 60, a septal-lateral dimension of the mitral valve is decreased as shown by arrows 6002a-6002b, thus reducing any gap between anterior and posterior valve leaflets, by increasing the distance between the commissures moving them in the directions of arrows 6001a and 6001b. A decrease in the gap between the anterior and posterior leaflets may also be achieved by stretching locations adjacent to the annulus but not necessarily adjacent to the commissures, at an offset angle to the septal-lateral direction of the valve and stretching sufficiently to achieve the desired valve configuration and/or gap dimensions in the lateral septal-lateral direction.

FIG. 61 shows a main implant template 6164 designed to create two areas of outward force 6161A and 6161B counterbalanced by an inward force 6162. These forces are applied by an anchor 6163, applying the inward force 6162, and a main implant template 6164, applying the outward forces 6161A-B. The curvature shape of the main implant template 6164 approximates a desired shape for the target segment of the annulus. An array of these implants could be applied to different annular segments to vary the total level of effect.

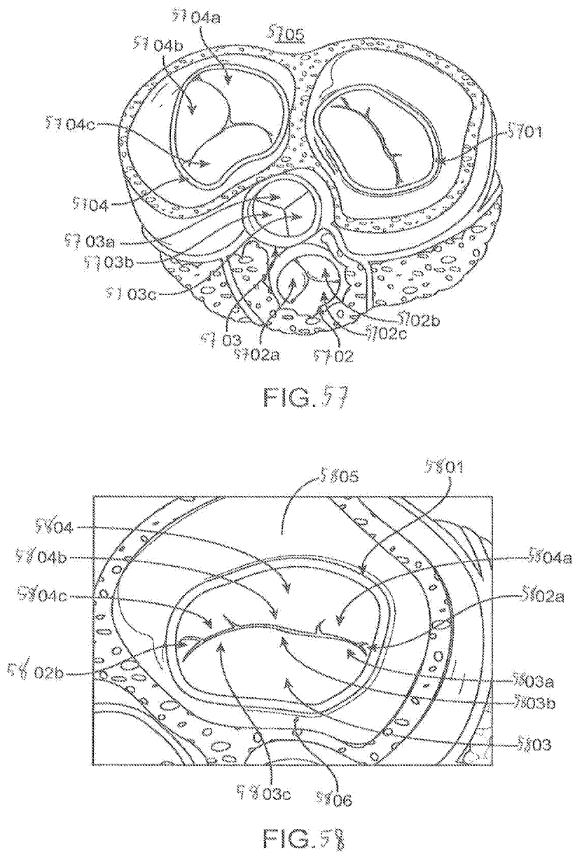

FIG. 62 shows a wavy implant 6174 having a repeating pattern of areas creating outward forces 6171A-D counterbalanced by inward forces 6172A-C. Each area of inward force is attached to the wavy implant 6174 by anchors 6173A-C. As shown, three inward force areas and 4 outward force areas are shown, but these numbers can be varied as needed to offer differing levels of effect.

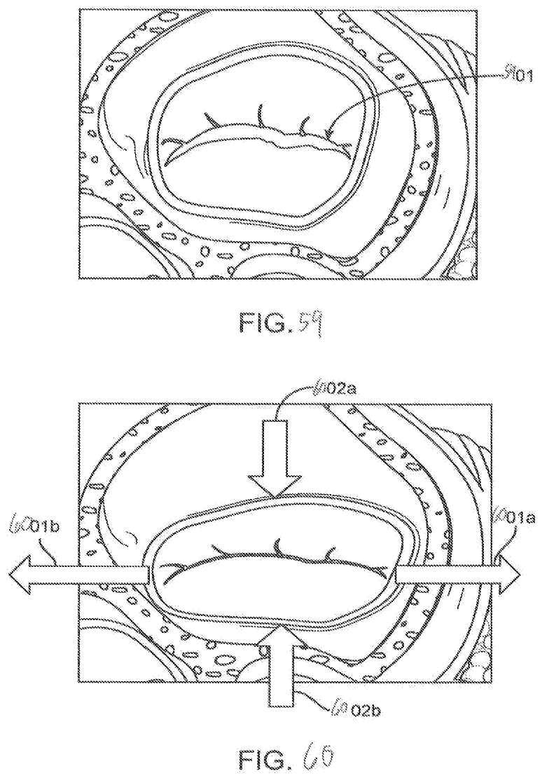

FIG. 63 shows an implant consisting of an array of extensible members in the extended position 6182A-D, anchored to an annulus by a corresponding array of anchors 6181A-E. As shown, the extensible members in the extended position 6182A-D are attached to the enlarged annulus to be treated. Extensible members can be constructed of a resilient material or using a spring design known to the art to allow a sufficient range of elastic deformation. The materials of the extensible members can be superelastic nitinol, muscle fibers, flexinol), rubber, plastics, metals, or alloys with a high yield strength to provide appropriate elastic range for the desired function. Alternately, the extensible members may be constructed in a manner makes them transformable between an elongated configuration (as shown) and a shorter configuration (see FIG. 81.) Various transformable structures that would fit this purpose (including stents, balloons, linkages, or closed cellular structures) are known to the art. The numbers of extensible segments and anchors may be altered as needed to provide varying degrees of effect.

FIG. 64 shows an implant consisting of an array of extensible members in the compressed position 6192A-D, anchored to an annulus by a corresponding array of anchors 6191A-E. As shown, the extensible members in the compressed position 6192A-D have compressed the previously enlarged annulus to effect a reduction in the annular circumference, the annular area, the annular diameter, or some combination thereof.

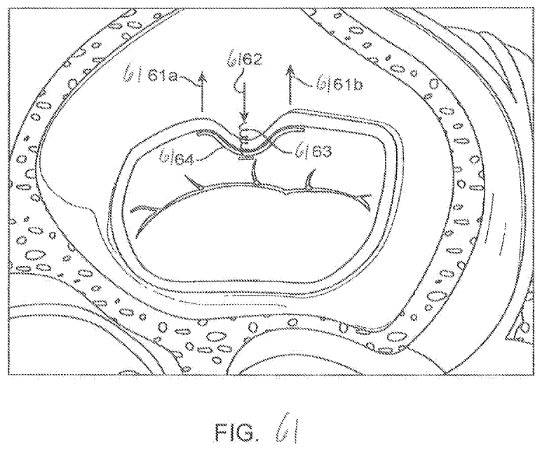

FIG. 65 shows a combination implant, including a semi-rigid shaping segment 6503, which is attached to the annulus by an array of anchors 6501B-6501D. The motion of this semi rigid shaping element 6503 is augmented by extensible elements 6502A and 6502B having both an extended and a contracted configuration, which are attached to the semi rigid shaping element 6503 and/or the anchors 6501B and 6501D and are further anchored to the annulus by anchors 6501A and 6501D in the extended configuration at a distance from the shaping element 6503. The materials of the extensible members can be superelastic nitinol, muscle fibers (flexinol), rubbers, plastics, metal, or alloys with a high yield strength to provide appropriate elastic range for the desired function. Alternately, the extensible members may be constructed in a manner makes them transformable between an extended configuration and a contracted configuration. Various transformable structures that would fit this purpose (including stents, balloons, linkages, or closed cellular structures) are known to the art. When the extensible elements 6502A and 6502B are released/transformed to their contracted configuration, they act to additionally reduce the annular circumference, the annular area, the annular diameter, or some combination thereof.

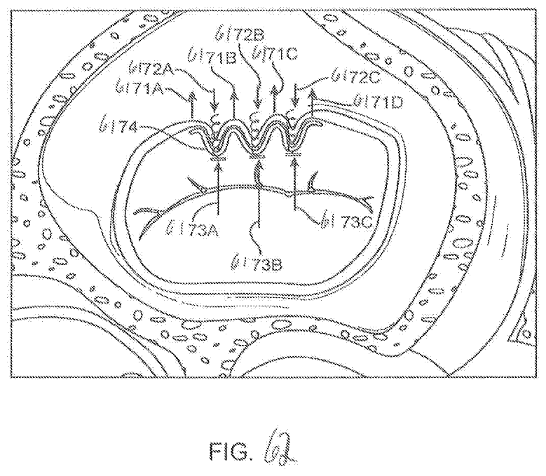

FIG. 66 shows an anchor for fastening an implant to tissue, including an anchor member 6610 having a helical coil section 6611, an implant stop feature 6612, and a locking feature 6615. The anchor system also includes a torque member 6613 and a locking wire 6614. The helical coil section 6611 of the anchor member 6610 can be fastened into the tissue by twisting the torque member 6614, which transfers the torque through the locking wire 6614 to the anchor member 6610 via the locking feature 6615. The locking wire also holds the anchor member 6610 to the torque member 6614 in the longitudinal Withdrawing the locking wire 6614 by pulling it proximally releases the anchor member 6610 from the torque member 6613 allowing removal of the torque member 6613 and locking wire 6614.

FIG. 67 shows an implant 6621 which defines a concave space 6622. The concave space is also referred to herein as a “concavity,” as defined previously. The tissue 6623 is shown in place in contact with both the implant 6621 and a helical coil 6624 having a sharpened tip of the implant, but not entering the concave space 6622. Rotating the helical coil 6624 in the direction of the arrow 6625 will cause the helical coil 6624 to draw the tissue 6623 into the concave space 6622. A single implant could define multiple concave spaces, and include multiple helical coils, or multiple single-concave space implants could be used. Prior to rotating the helical coil, its sharpened tip extends beyond both sides of the implant to facilitate penetration of the tissue.

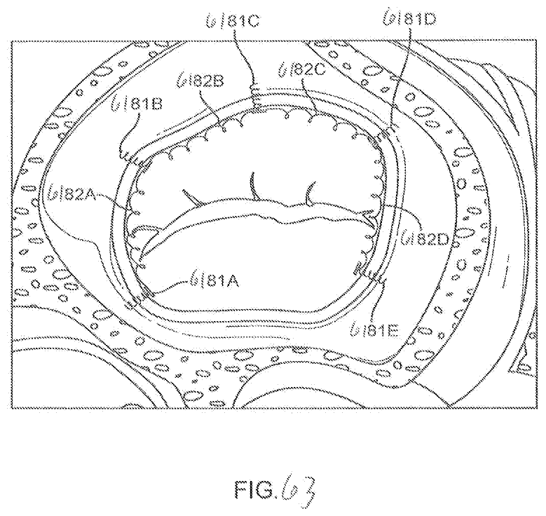

FIG. 68 shows an implant 6631 in place in tissue 6633. In this figure, the helical coil 6634 has been activated to draw the surrounding tissue 6633 into the concave space 6632, substantially filling the space 6632.

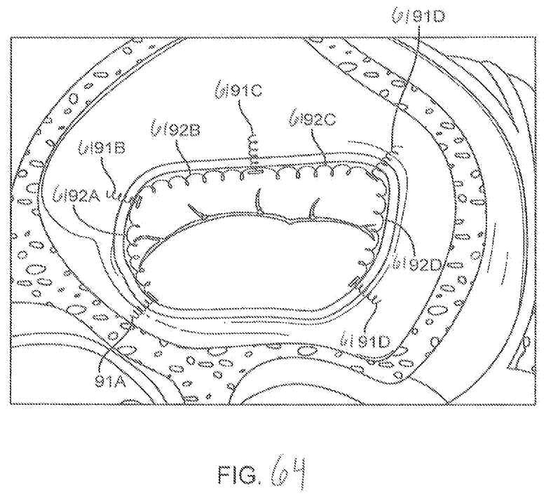

FIG. 69 shows an undulating template 6641 in place against a substantially straight segment of tissue 6642, with three helical anchors 6643A-6643C connecting the undulating implant 6641 to the tissue segment 6642 without substantially deforming the tissue segment 6642. The ends of the tissue segment 6642 are substantially farther apart than the ends of the undulating template 6641, although their lengths are comparable.

FIG. 70 shows the undulating template 6641 of FIG. 69 with the now undulating tissue segment 6652 having been pulled tightly against the undulating template 6641 by the helical anchors 6643A-6643C. The ends of the now undulating tissue segment 6652 are proximate to the ends of the undulating template 6641, although its length is comparable to the substantially straight segment of tissue 6642 from FIG. 69.

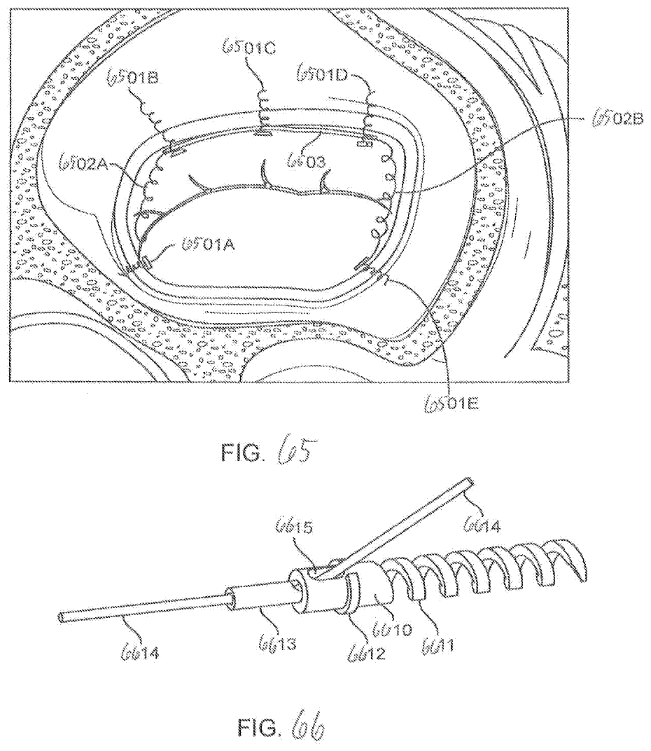

FIG. 71 shows the deformations projected on an untreated mitral valve annulus 6661 by a flattening template 6662 and an undulating template 6663. The undulating template creates similar reduction in vertical dimension as shown, without substantially increasing horizontal dimension as shown.

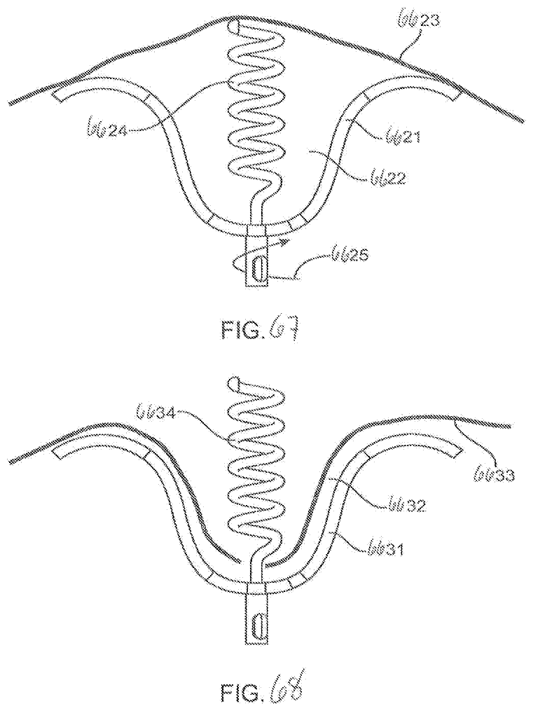

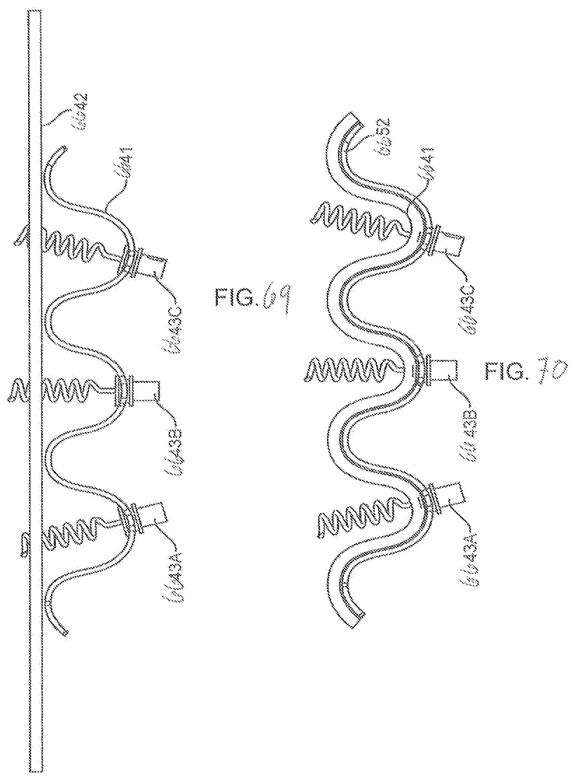

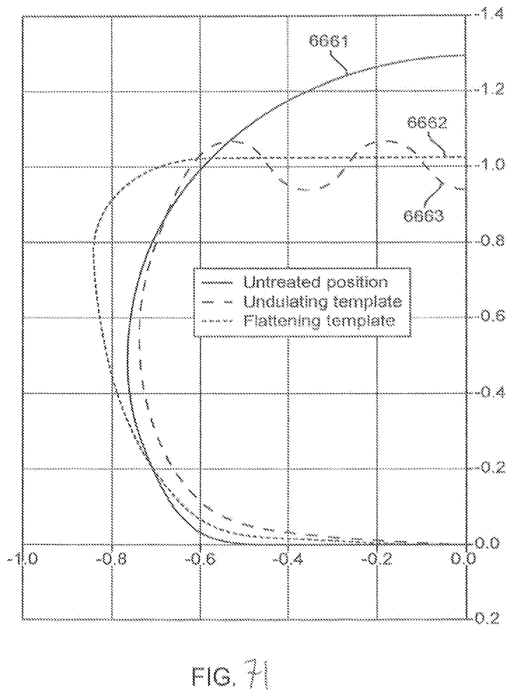

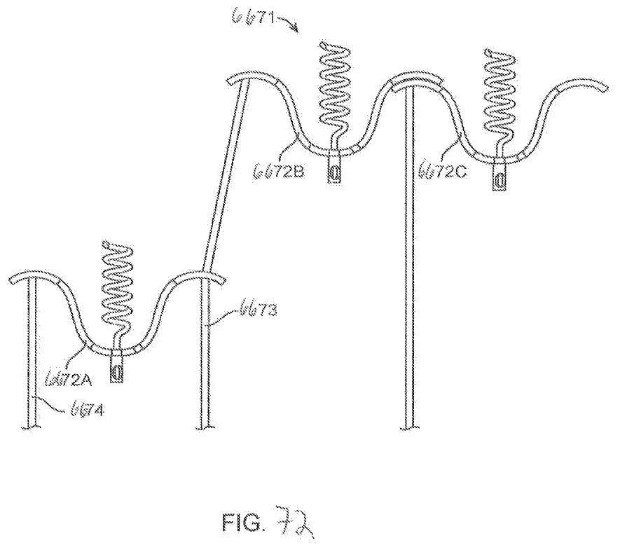

FIG. 72 shows a segmented undulating template 6671, consisting of segments 6672A-6672C. As shown, segments 6672B and 6672C have been delivered into the desired position, and segment 6672A is being delivered to the desired position by sliding it along an elongate locating member 6673 which is attached to the already placed segment 6672B. An elongate locating member 6674 is attached to the segment 6672A which is in the process of being placed, offering guidance for placement of an additional segment (not shown). In this way, it is possible to place an arbitrary number of segments by sliding the next segment up the outermost elongate locating member 6674.

FIG. 73 shows a segment of an undulating template 6681 folded distally for delivery through a tube or tubular structure. The ends of the segment of the undulating template 6681 are held together by a removable shaper 6682 which holds the undulating template 6681 in its folded configuration during delivery. In addition, two elongated control elements such as control wires 6683A and 6683B are shown attached near the ends of the segment of the undulating template 6681.

FIG. 74 shows a segment of an undulating template 6691 which has been expanded by applying tension to the control wires 6693A and 6693B. The anchor 6692 extends away from the segment of the undulating template 6691 to allow easy anchoring in tissue (not shown)

FIG. 75 shows two segments of undulating templates 7501A and 7501B attached to control wires 7502A-R, 7502A-L, 7502B-R, and 7502B-L and torque members 7503A and 7503B, arranged one behind the other for delivery through a tubular structure (not shown). The alignment of the segments 7501A and 7501B is shown slightly offset, but should be adjusted to allow for minimum tube diameter that allows passage of the segments of undulating templates 7501A and 7501B, control wires 7502A-R, 7502A-L, 7502B-R, and 7502B-L and torque members 7503A and 7503B through as small a diameter tubular structure as practical. Additional segments of undulating template (not shown) can be arranged in similar fashion for placement through a tubular structure as needed.

FIG. 76 shows a substantially flat, shapeable template 7611 with an anchor 7612 with torque member 7614 and forming dies 7613A and 7613B. The orientation of the forming dies 7613A and 7613B relative to the anchor 7612 and torque member 7614 is such that the forming dies 7613A and 7613B appose the shapeable template 7611 in its substantially flat configuration. In this substantially flat configuration, the shapeable template 7611 can be firmly attached to the target tissue (not shown) through activation of the anchor 7612.

FIG. 77 shows a shapeable template 7621 in the shaped configuration, created by relative motion between the anchor 7622 and forming dies 7623A and 7623B. When the shapeable template 7621 is firmly attached to tissue via the anchor 7623, the tissue will move along with the template as it is shaped, creating a desired shaping and/or shortening effect.

FIG. 78 shows an assembled undulating template 7631 made up of three undulating template segments 7632A-7632C. The segments are connected with pin connectors 7633A and 7633B, each of which is made up of a pin element 7634 attached to an undulating template segment through an attachment device 7635. Typical attachment devices known to the art which may be applicable to this mechanism include threaded fasteners such as nuts, crimp connectors, and push-on retaining rings.

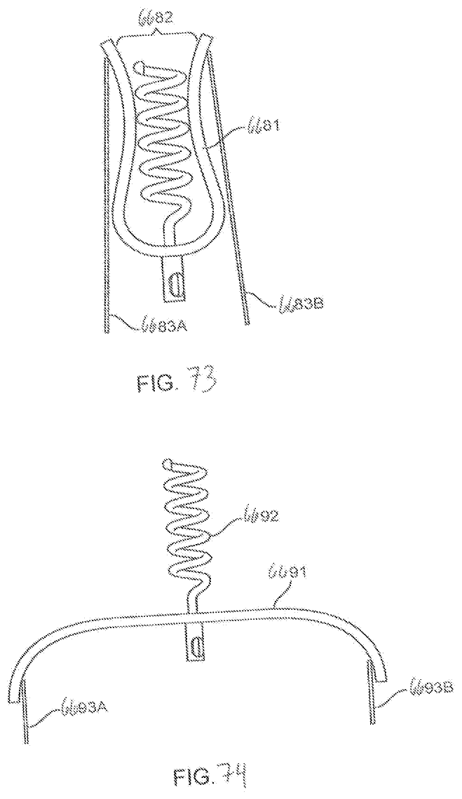

FIG. 79 shows an assembled undulating template 7641 made up of three undulating template segments 7642A-7642C. The segments are connected by an integral post 7643 joined by an attachment device 7644. Typical attachment devices known to the art which may be applicable to this mechanism include threaded fasteners such as nuts, crimp connectors, and push-on retaining rings.

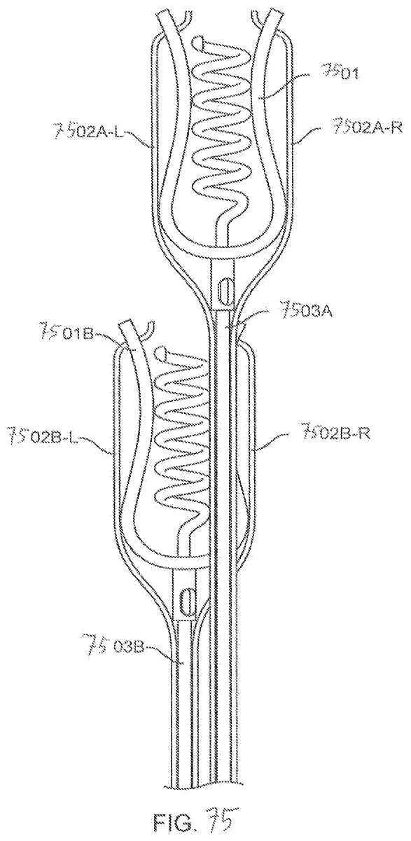

FIG. 80 shows an assembled undulating template 7651 made up of three undulating template segments 7652A-7652C. The segments are connected by mechanical connectors 7654. Typical mechanical connectors applicable to this mechanism include crimp connectors and clips.

FIG. 81 shows a partial annular ring 7661 with multiple anchors 7662 disposed within a valve annulus 7663. The anchors 7662 are of sufficient length to bridge the gap between the partial annular ring and the valve annulus 7663. Activating the anchors 7662 draws the annulus 7663 toward the partial ring 7661, reshaping the annulus 7663 to the desired configuration. It is possible to apply this approach to a closed ring of the desired shape as well as the partial annular ring 7661 as shown. Desired shapes for the partial or close ring may include circular, D-shaped, oval, elliptical, or with a concave section corresponding to one or more anchor 7662 positions.

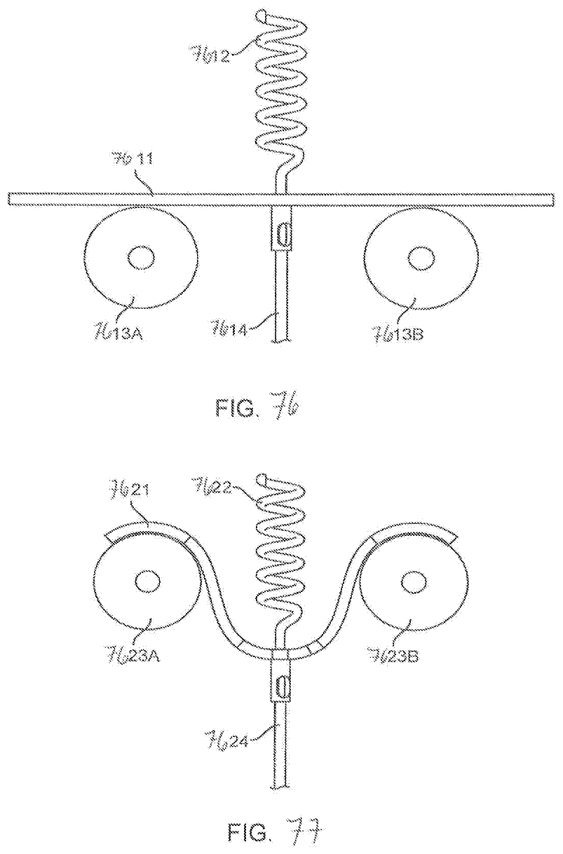

FIG. 82 shows an alternate segment of an undulating template 7671 having two anchors 7672A and 7672B separated by a convex segment 7673.

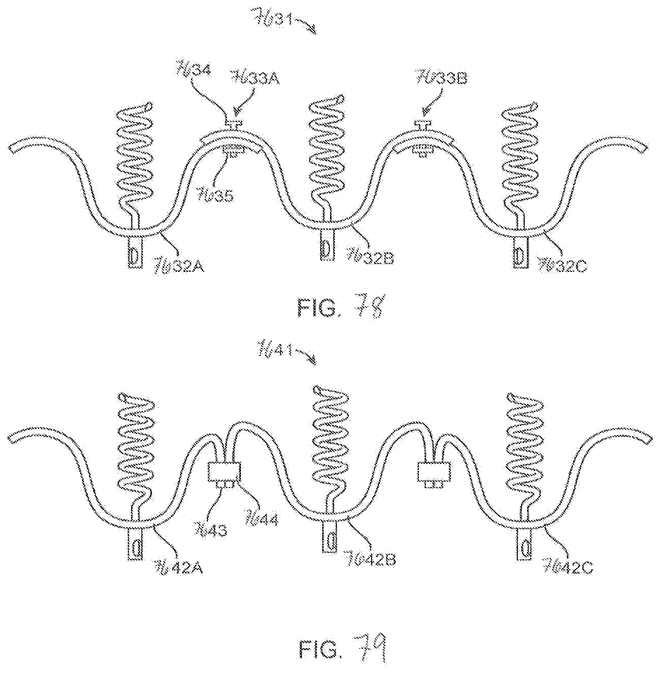

FIG. 83 shows an undulating template 7681 consisting of two alternate segments 7682A and 7682B each having two anchors separated by a convex segment, joined by an attachment mechanism 7683. Typical mechanical connectors applicable to this mechanism include crimp connectors, clips, sutures, or the like.

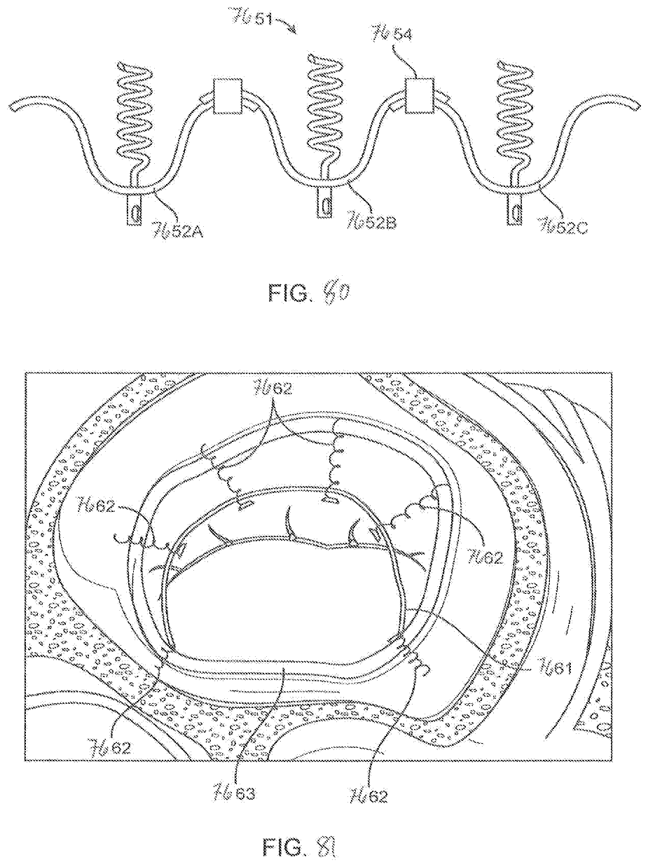

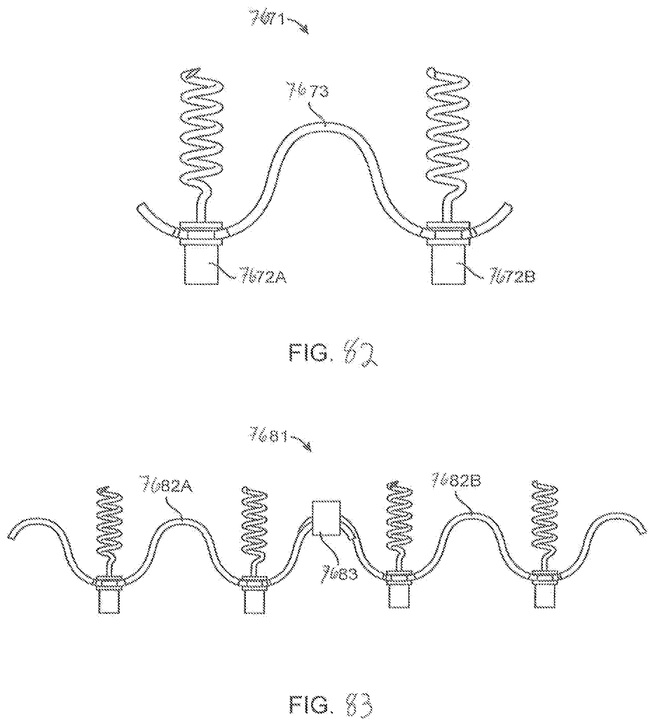

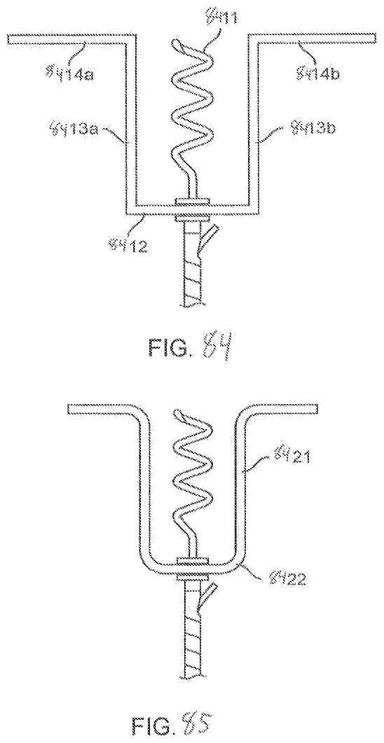

FIG. 84 shows an undulating template 8412 composed of approximately straight segments arranged in a rectilinear pattern with angled bends or corners, with a tissue coupling mechanism 8411 attached approximating the mid-point of the body of the template, two body segments 8413a and 8413b rising from the point of attachment of the tissue coupling mechanism 8411, and two compressive peaks 8414a and 8414b. The area of the undulating template 8412 where the tissue coupling mechanism is attached as well as the area of the compressive peaks 8414a and 8414b are substantially horizontal, while the rising body segments 8413a and 8413b are substantially vertical.

FIG. 85 shows an undulating template 8421 composed of approximately straight segments connected by arcuate segments including the lower right arcuate segment 8422.

FIG. 86 shows an undulating template where the rising body segments 8431a and 8431b form a diverging angle relative to each other and the point of attachment of the tissue coupling mechanism. As tissue is drawn towards the base of the tissue coupling mechanism, the gap between the rising body segments 8431a and 8431b narrows, causing increasing compressive forces on the tissue.

FIG. 87 shows an undulating template 8441 composed of arcuate segments ending so that the segment ends near the compressive peak 8442.

FIG. 88 shows an undulating template 8451 composed of arcuate segments ending so that the segment end 8452) extends past the compressive peak 8453.

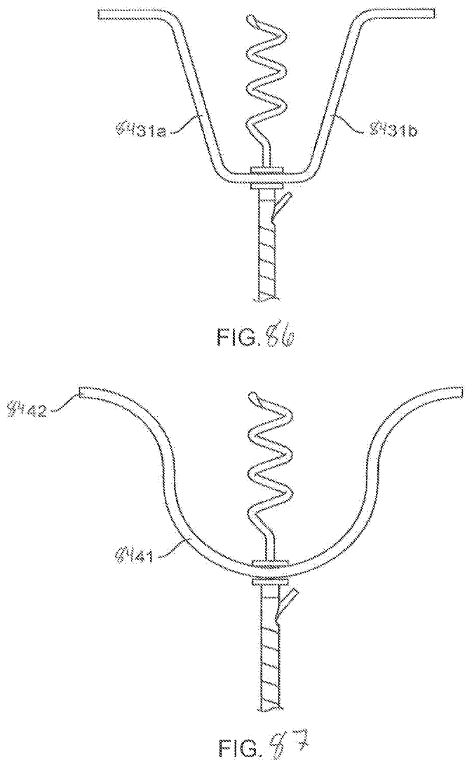

FIG. 89 shows an undulating template 8461 composed of a continuous, non-circular shape. As shown, the shape is a sinusoidal curve.

FIG. 90 shows an undulating template 8471 where the distance from the point where the tissue coupling mechanism is attached to the compression peaks of the body of the template is greater than the length of the tissue coupling mechanism, e.g. a helical anchor. A line 8472 tangent to the compression peaks of the template is not crossed by the distal tip of the helical tissue coupling mechanism. Placement of such a template can be accomplished, for example, by deflecting the compression peaks of the template proximally (e.g. by pressing them against a wall of a valve annulus) so that the tissue coupling mechanism can penetrate the target tissue.

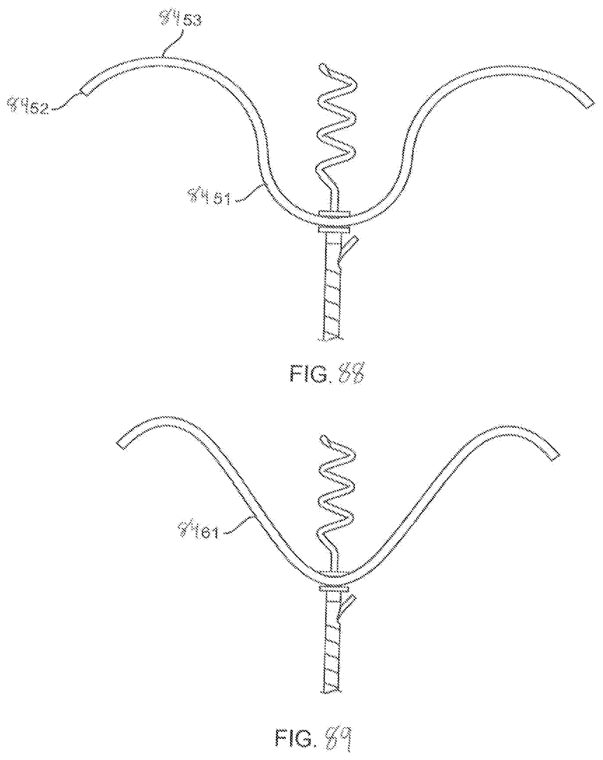

FIG. 91 shows the undulating template 8481 where the distance from the point where the tissue coupling mechanism is attached to the compression peaks of the body of the template is less than the length of the helical or other tissue coupling mechanism. The distal tip of the coupling mechanism crosses a line 8482 tangent to the compression peak. Placement of such a template can be accomplished, for example, with the ends of the template in the relaxed and non-deflected position.

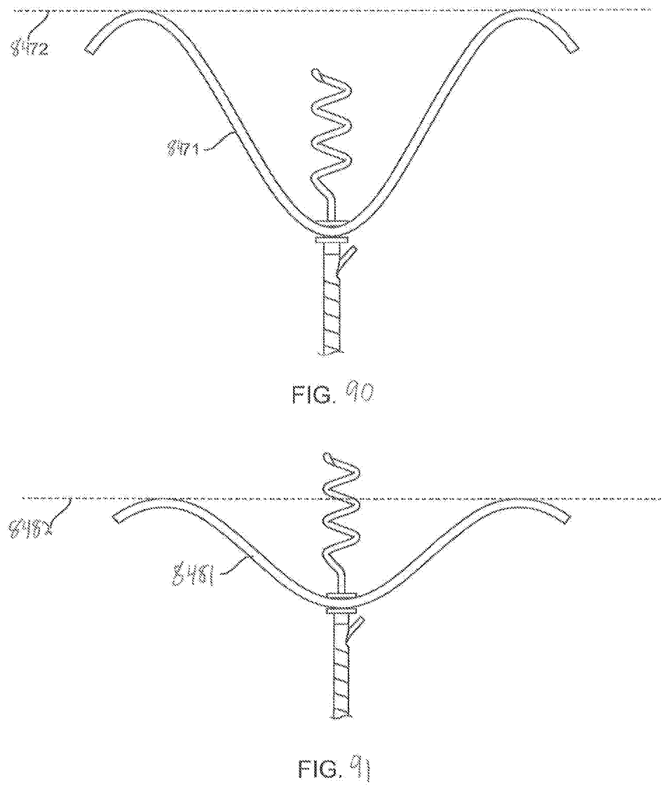

FIG. 92 shows an undulating template with tissue 8493 held in place by a tissue coupling mechanism, causing the template to exert tensile force 8491 normal to the original position of the tissue (via the tissue coupling mechanism) and inward forces 8492a and 8492b, tangential to the original position of the tissue.

FIG. 93 shows an undulating template with tissue 9303 held in place by a tissue coupling mechanism, causing the template to exert tensile force 9301 normal to the original position of the tissue (via the tissue coupling mechanism) and compressive forces 9302a and 9302b, normal to the original position of the tissue in substantially the opposite direction as the tensile force 9301.

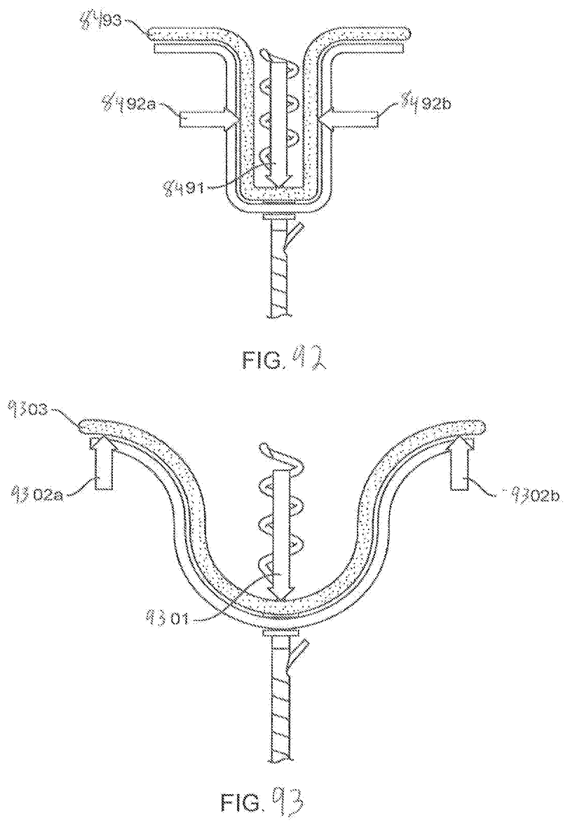

FIG. 94 shows an undulating template with tissue 9413 held in place by a tissue coupling mechanism, causing the template to exert tensile force 9411 normal to the original position of the tissue (via the tissue coupling mechanism) and compressive-inward forces 9412a and 9412b, between normal and tangential to the original position of the tissue.

FIG. 95 shows an undulating template with tissue 9423 held in place by a tissue coupling mechanism, causing the template to exert tensile force 9421 normal to the original position of the tissue (via the tissue coupling mechanism) and compressive-outward forces 9422a and 9422b, between normal and tangential to the original position of the tissue.

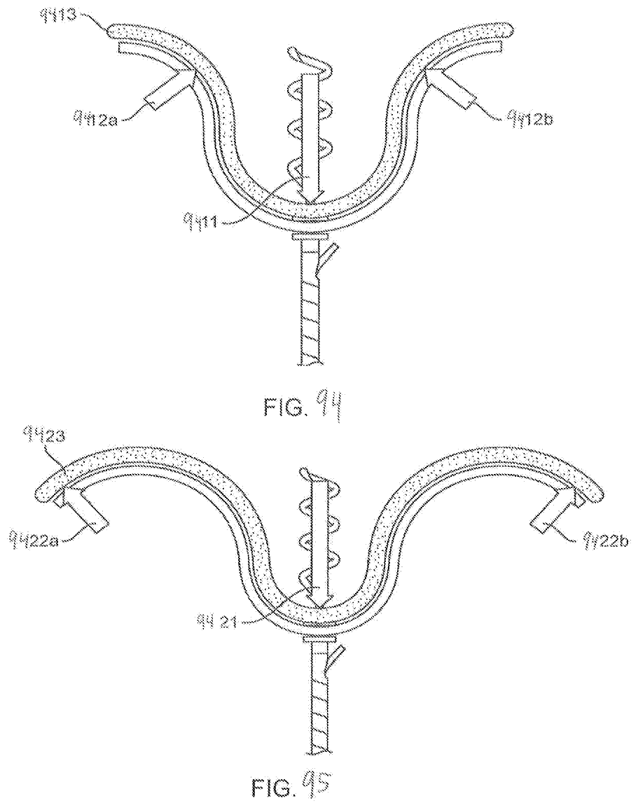

FIG. 96 shows an undulating template with stabilizing tissue coupling mechanisms 9431a and 9431b at each end of the body, in addition to the primary tissue coupling mechanism in the middle. The stabilizing tissue coupling mechanisms 9431a and 9431b each have a penetrating coil at their distal ends, and a coupling coil 9433 at the proximal end with the opposite handedness of the penetrating coil. The stabilizing tissue coupling mechanisms 9431a and 9431b are releasably coupled via coupling bushings 9434 attached to small torque members 9436. The coupling bushings 9434 guide and capture the coupling coil 9433 of the stabilizing tissue coupling mechanisms 9431a and 9431b against turning in one direction. They are prevented from turning relative to each other by a key wire 9435a and 9435b. Removing the key wire 9435a or 9435b allows the small torque members 9436 and attached coupling bushings 9434 to turn relative to the coupling coil 9433, releasing the stabilizing tissue coupling mechanisms 9431a and 9431b from the coupling bushing 9434. A slot in the undulating template is arranged so that the stabilizing tissue coupling mechanisms 9431a and 9431b will not slide freely through the undulating template when coupled to adjacent tissues by twisting.

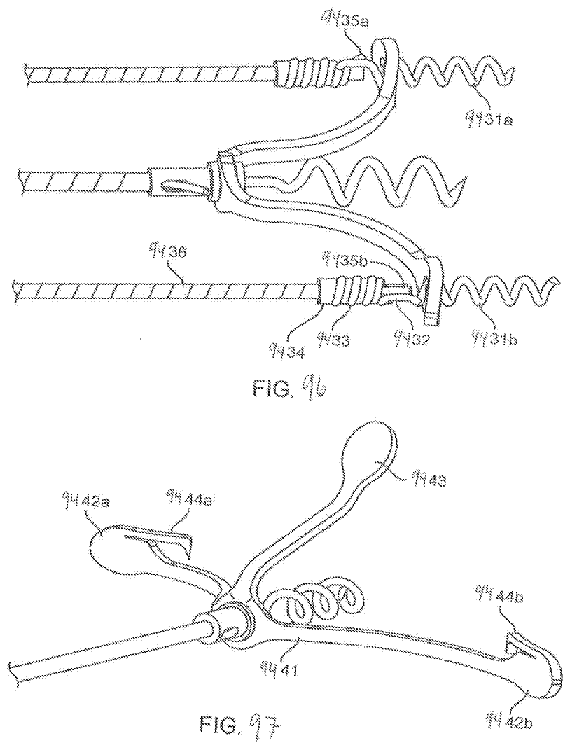

FIG. 97 shows and undulating template 9441 with two principal ends 9442a and 9442b, and an additional stabilizing arm 9443 extending from the body, as well as stabilizing penetrating points 9444a and 9444b, in this example disposed near the principal ends 9442a and 9442b. The body of the template 9441 may include a single stabilizing penetrating point, two stabilizing penetrating points 9444a and 9444b as shown, or more as required. The stabilizing penetrating points 9444a and 9444b may include curves, barbs, bends, or other such features to allow them to passively penetrate the tissue adjacent to the undulating template 9441, or may benefit from action on behalf of the user to actuate the stabilizing penetrating point 9444a and 9444b.

FIG. 98 shows an undulating template 9451 with the ends 9452a and 9452b folded away from the tissue coupling mechanism attachment 9453 to a delivery position as shown, where the tissue coupling mechanism attachment 9453 and or flexibility in the tissue coupling mechanism 9454 allow the template arms to fold together alongside the tissue coupling mechanism 9454. This configuration may allow for more compact delivery size of the implant compared to configurations where the delivery position has the tissue coupling mechanism 9454 disposed between the arms 9452a and 9452b of the undulating template 9451

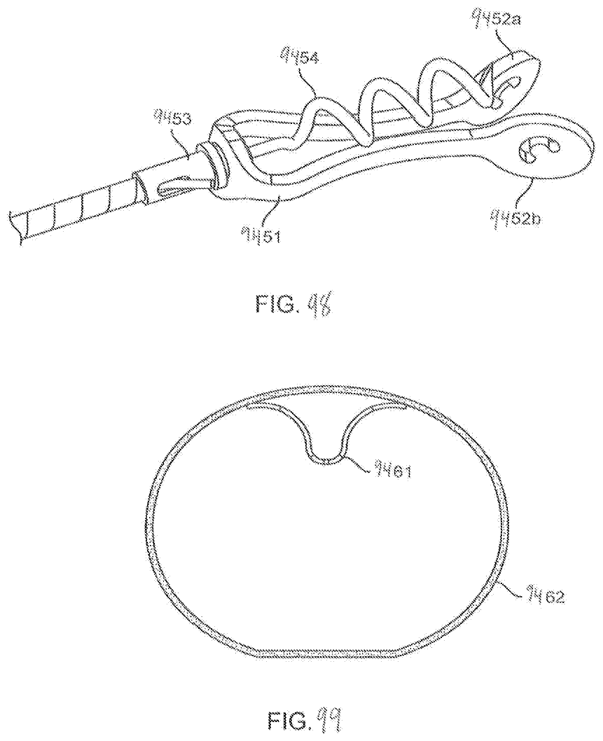

FIG. 99 shows an undulating template 9461 in position adjacent to a mitral annulus 9462 in the untreated state. As shown, the undulating template 9461 is not interacting with the tissue but is positioned approximately as it would be prior to coupling it to the tissue via a tissue coupling mechanism (not shown).

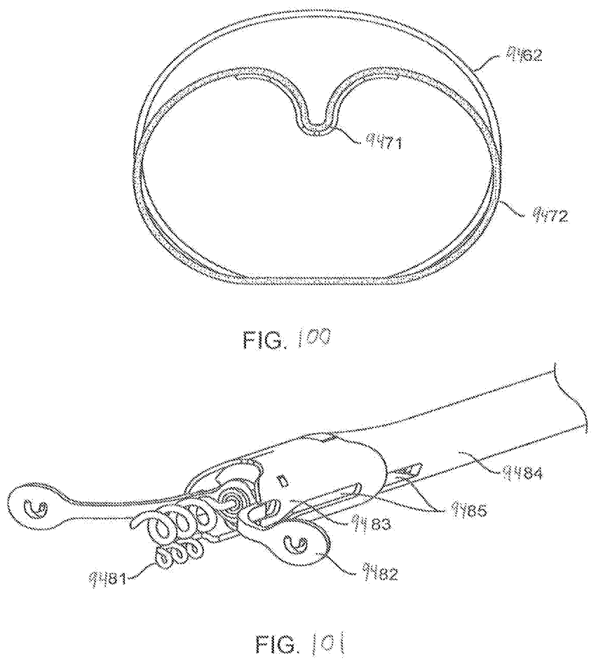

FIG. 100 shows an undulating template 9471 with a mitral annulus 9472, where the tissue coupling mechanism has drawn the annulus tightly against the template. As shown, the circumference of the annulus following the template is essentially unchanged, but the effective circumference of the annulus (bypassing the segment captured by the template) has decreased. The effect of decreasing the effective circumference of the annulus, in combination with deforming the central portion of the annulus toward the middle of the valve, reduces both the minor axis diameter of the valve and the area of the valve. The original position of the annulus 9462 from FIG. 99 is also shown for reference.

FIG. 101 illustrates a delivery device 9484 for placing an undulating template 9482 over a pre-anchor guide 9481. The pre-anchor guide 9481 runs through a receiving slot in the delivery device 9484. The pre-anchor guide 9481 consists of a tissue coupling feature (a penetrating coil as shown) and a long guide wire. It may be advantageous to place the pre-anchor guide 9481 with a separate delivery device prior to introducing the undulating template 9482. In that case, the delivery device 9484 for the undulating template 9482 may have reduced flexibility, steerability, diameter, torqueability, or other requirements since the target position has been pre-selected and verified during pre-anchor guide 9481 placement. The pre-anchor delivery device may include an outer steerable sheath, and an inner steerable sheath, the outer steerable sheath being steerable along a radius of from 1 cm to 3 cm, and capable of bending to and angle between 90 and 200 degrees. The inner steerable sheath is able to be rotated within the outer sheath, and extended or retracted relative to the outer sheath, allowing between 1 cm and 10 cm of the inner sheath to extend past the tip of the outer sheath. The inner steerable sheath may be steerable along a radius of from 0.5 cm to 3 cm, through an angle between 30 and 90 degrees. There may be features on the template 9482 that interact with the delivery device 9484 to stabilize the template 9482 for improved maneuverability during placement. Such features could also be used with a remotely actuated powered system for increased precision control.

The delivery device 9484 provides channels for the releasable torque member attached to the primary tissue coupling mechanism, for the small torque members attached to the stabilizing tissue coupling mechanisms (not shown), and for the pre-anchor guide 9481. These channels may be formed as an extrusion with four distinct inner lumens. The channels for the pre-anchor guide 9481 and the primary tissue coupling mechanism exit the distal end of the delivery device 9484, while the channels for the small torque members attached to the stabilizing tissue coupling mechanisms (not shown) have a side exit 9485 that communicates with the distal end of the delivery device 9484, allowing the small torque members (not shown) to be delivered within the outer diameter of the delivery device 9484 when the undulating template 9482 is folded forward in the delivery configuration and then to extend outside the diameter of the delivery device 9484 when the arms are in the placement position.

The delivery device 9484 also incorporates a rotational guide member 9483 which couples the undulating template 9482 to the delivery device 9484. Depending on the exact use configuration of the delivery device 9484, the body of the delivery device 9484 may be long and flexible to function as a catheter, or short and rigid for open surgical procedures. The delivery device 9484 may include an outer steerable sheath, and an inner steerable sheath, the outer steerable sheath being steerable along a radius of from 1 cm to 3 cm, and capable of bending to and angle between 90 and 200 degrees. The inner steerable sheath is able to be rotated within the outer sheath, and extended or retracted relative to the outer sheath, allowing between 1 cm and 10 cm of the inner sheath to extend past the tip of the outer sheath. The inner steerable sheath may be steerable along a radius of from 0.5 cm to 3 cm, through an angle between 30 and 90 degrees.

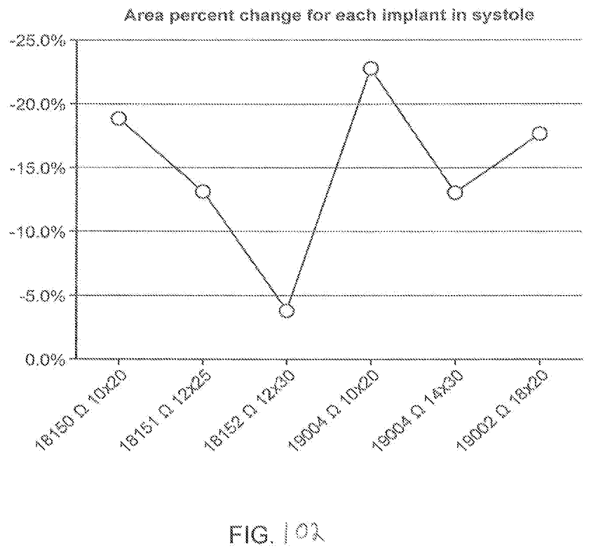

FIGS. 102-104 show percent change for annular dimensions in various templates implanted in-vivo. These data were collected during open heart implantations in the porcine model; the chest was opened, bypass readied, and pre-op measurements made. The animal was then put on bypass, the device implanted, the heart closed and taken off bypass. When the heart was pumping successfully on its own, post-operative measurements were taken and compared to the pre-op measurements. All measurements were taken in systole.

FIG. 102 shows percent area change for various templates implanted in-vivo.

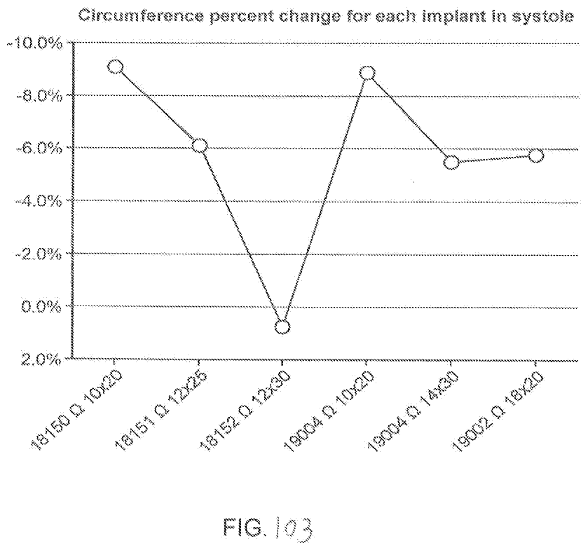

FIG. 103 shows percent circumference change for various templates implanted in-vivo.

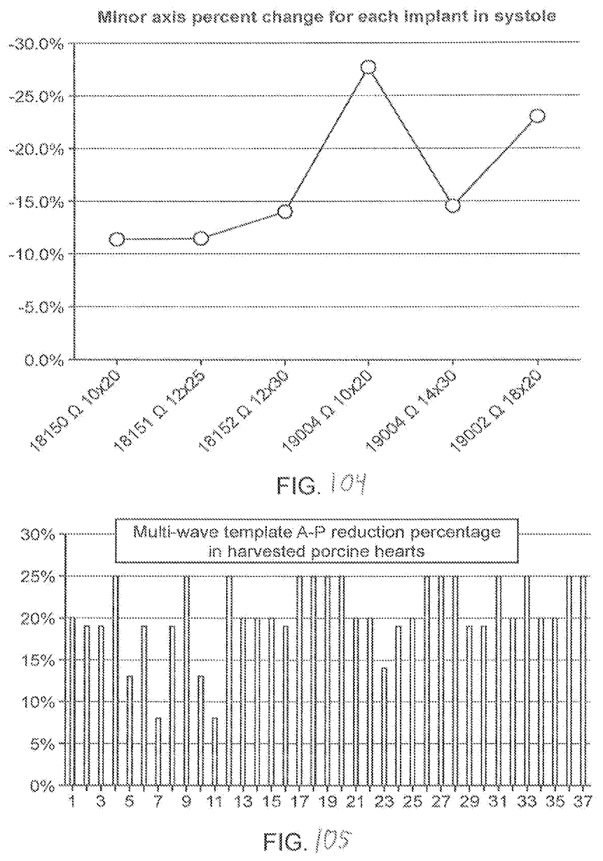

FIG. 104 shows percent minor axis change for various templates implanted in-vivo.

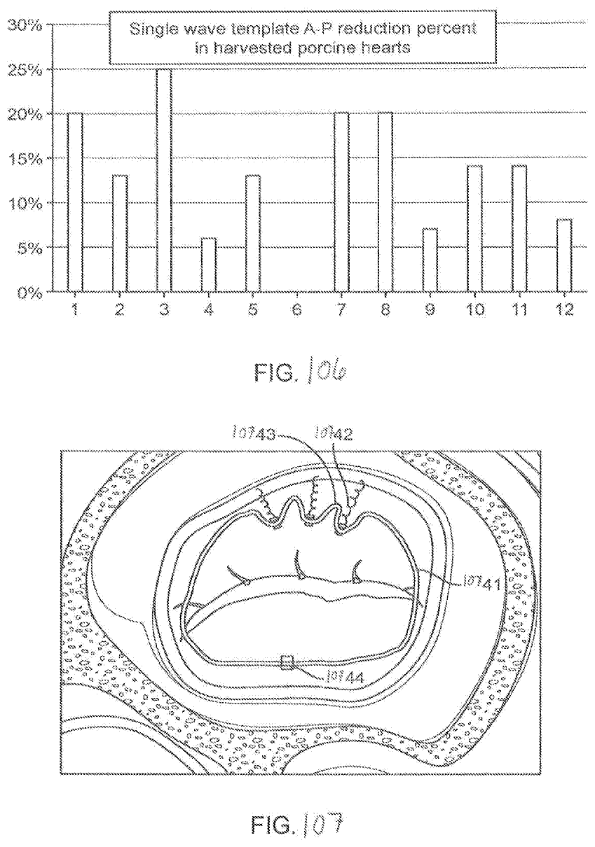

FIGS. 105-106 show percent change in the minor axis diameter for various templates in excised porcine hearts. Hearts were obtained fresh, mounted in a stand so that the mitral annulus was approximately horizontal, and held so that the pre-procedure ratio of major to minor axis was between 1.2:1 and 1.3:1 as verified by D-shaped valve sizers. The implants were placed, and the altered dimension of the mitral valve again measured by D-shaped valve sizers.

FIG. 105 shows percent A-P (minor axis) reduction for various multi-wave templates implanted in excised porcine mitral annuli.

FIG. 106 shows percent A-P (minor axis) reduction for various single-wave templates implanted in excised porcine mitral annuli.

FIG. 107 shows a continuous ring template 10741, having a single undulating region with one or more tissue anchors 10742 separated by one or more wave peaks 10743. This ring template 10741 may include a latching discontinuity 10744, allowing it to be inserted and deployed in a substantially straight configuration, and connected to form a semi-rigid structure. Such a structure may be used as a stabilizer for placement of a replacement valve as required.

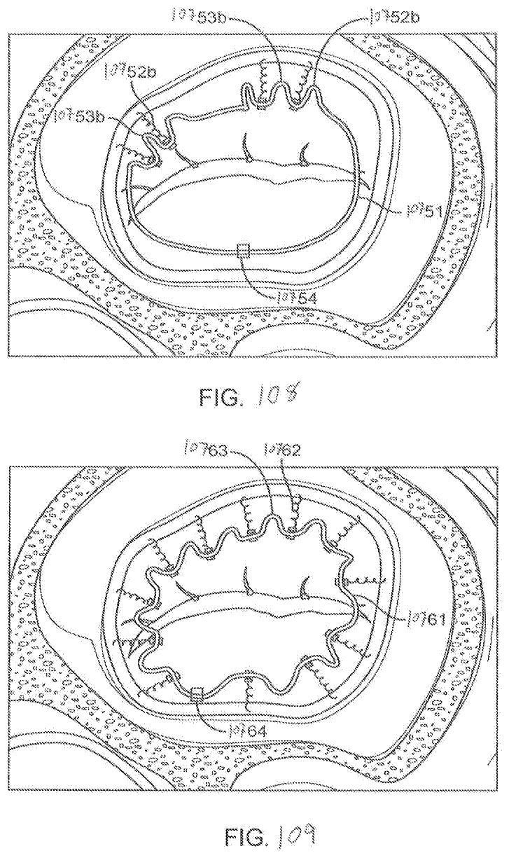

FIG. 108 shows a continuous ring template 10751, having multiple undulating region with one or more tissue anchors 10752 separated by one or more wave peaks 10753. This ring template 10751 may include a latching discontinuity 10754, allowing it to be inserted and deployed in a substantially straight configuration, and connected to form a semi-rigid structure. Such a structure may be used as a stabilizer for placement of a replacement valve as required.

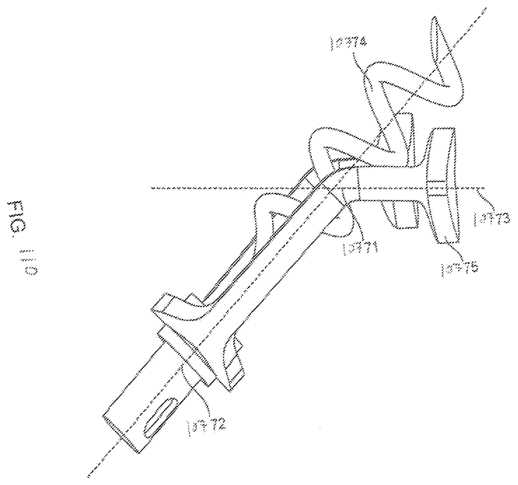

FIG. 109 shows a continuous ring template 10761, having one undulating region which covers essentially the entire circumference of the ring template 10761, with one or more tissue anchors 10762 separated by one or more wave peaks 10763. This ring template 10761 may include a latching discontinuity 10764, allowing it to be inserted and deployed in a substantially straight configuration, and connected to form a semi-rigid structure. Such a structure may be used as a stabilizer for placement of a replacement valve as required.

FIG. 110 shows an undulating template with an angle 10771 between the anchor 10774 and the compression pad feature 10773. This angle 10771 causes the line of tensile force 10772 and the line of compressive force 10773 to intersect, encouraging the anchor 10774 to form a desired angle with the target tissue. This angle 10771 can be built into the template, formed after the template is in position, or can be one stable state of a bi-stable system, which goes in straight and snaps to the angled configuration.

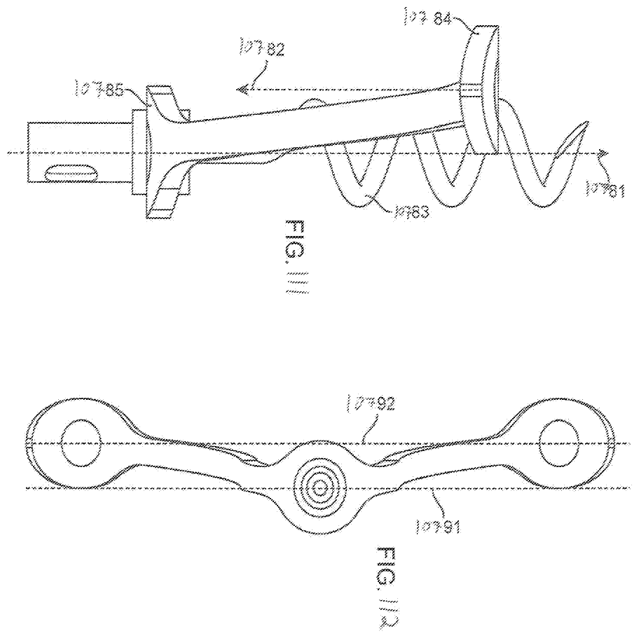

FIG. 111 shows an undulating template with a parallel offset between the tensile forces 10781 on the anchor 10783 and the compressive force 10782 on the compression pads 10784. The offset between these forces creates a moment that biases the anchor attachment point 10785 to move in a desired angular direction relative to the target tissue.

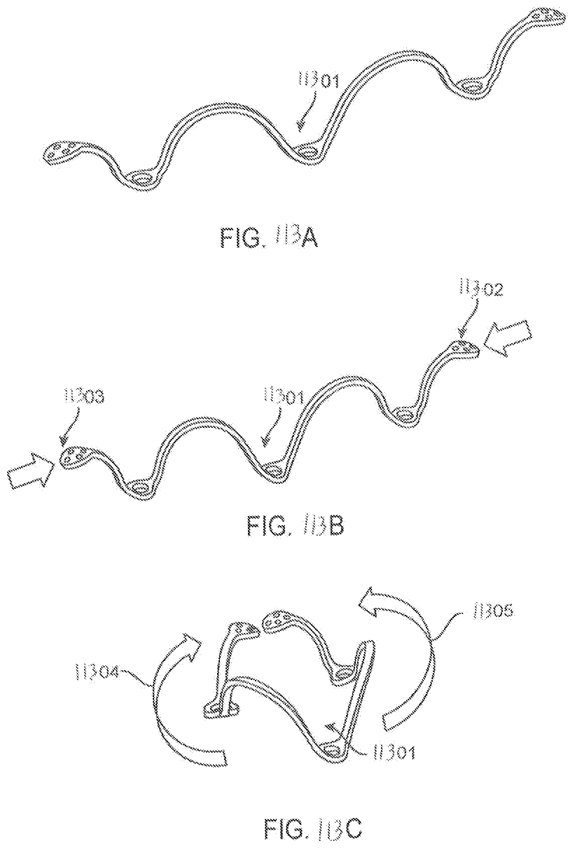

FIG. 112 shows an end-on view of the implant from FIG. 111, illustrating the separation between the plane of the center anchor 10791 and the plane of the side anchors 10792.

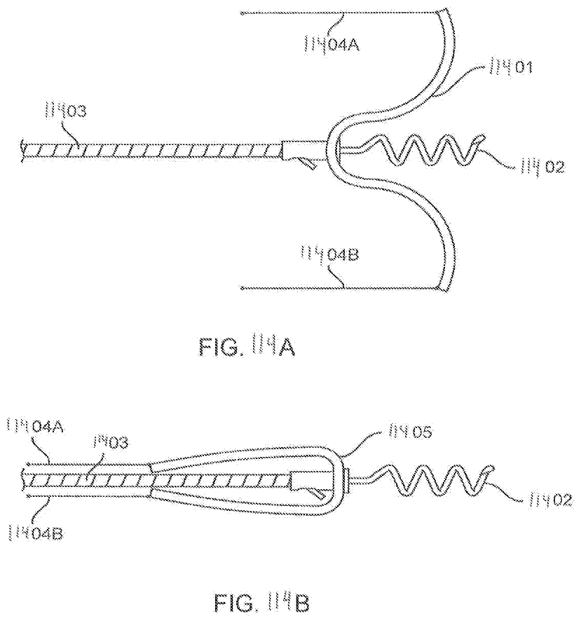

As shown in FIG. 113A, a tissue shaping template 11301 has a preformed shape it takes in the unconstrained configuration. The unconstrained configuration is optimized for tissue interaction, but not for delivery to the desired site on the tissue. FIG. 113 B shows the tissue shaping template 11301 in a first crimped position, having been constrained in a way that brings the two ends (11302 and 11303) closer together by forcing them toward the middle of the implant. In certain implant configurations, it may be advantageous to push the ends very closely together, arriving at a crimped configuration that is small enough for insertion through a delivery catheter or other device to the desired location in the tissue. FIG. 113 C shows the tissue shaping implant 11301 having been curved, bending one end in a clockwise direction 11304 and the opposite end in a counterclockwise direction 11305 to form a substantially circular crimped configuration. This crimped configuration may be easier to deliver through a small diameter tube than the unconstrained configuration.

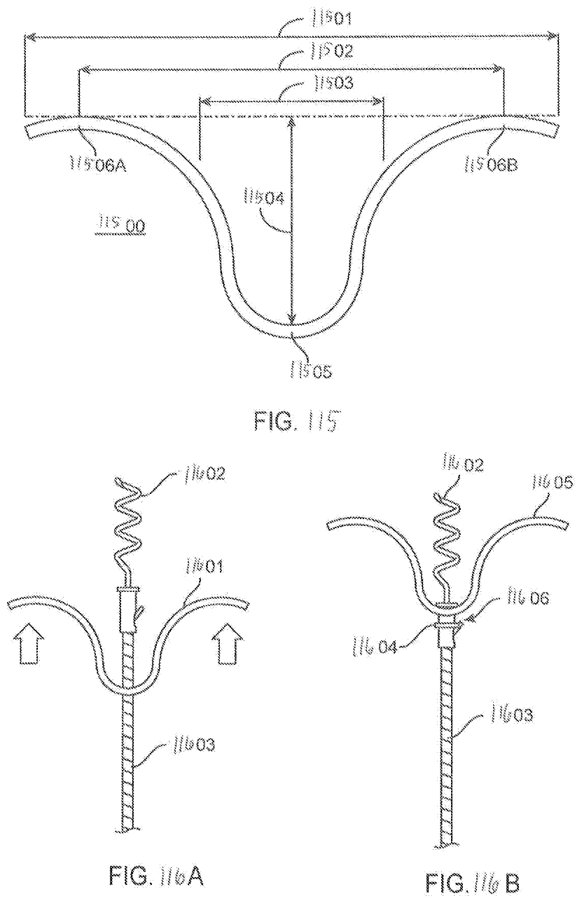

FIG. 114A shows an unconstrained preformed or pre-shaped template 11401. The unconstrained preformed or pre-shaped template 11401 is coupled to control wires 11404A and 11404B, as well as an anchor 11402 which is in turn coupled to an anchor control device 11403. FIG. 114 B shows the template of FIG. 114A in a crimped configuration 11405, which is constrained with the ends or wings of the template retracted proximally relative to the anchor 11402 by control wires 11404A and 11404B which apply a proximal tension to deform the wings proximally away from the anchor. This crimped configuration allows the anchor to be coupled to the desired tissue by initially or fully penetrating the anchor 11402 while the wings are in the constrained configuration 11405, which may simplify the placement of the template. After the anchor 11402 has been fully or partially penetrated into the target site, the retracted ends or wings of the template may be released from the control wires to return to the configuration of FIG. 114A. Depending upon how far the anchor 11402 has been penetrated, the tissue of the annulus will be fully or partially drawn into the convexity between the wings. The anchor 11402 may be further rotated to fully draw in the tissue as needed.

FIG. 115 shows various dimensions on a typical preformed or pre-shaped template 11500 having a concavity segment 11505 and two apex or convex segments 11506A and 11506B. The end-to-end length 11501, the peak-to-peak length 11502, the concavity width 11503 and concavity depth 11504 are illustrated on the diagram. The relationship between the concavity width 11503 and concavity depth 11504 may affect the magnitude of the tissue reshaping effect, as well as the suitability of the preformed or pre-shaped template 11500 for reshaping various different target tissues. Similarly, the relationship between the end-to-end length 11501 of the template 11500, and the overall length of the flattened template shape (not shown) may be indicative of the magnitude of the reshaping effect.

FIG. 116A shows a pre-delivery position of a preformed or pre-shaped template 11601 translatably or slidably engaged with a shaft of an anchor control device 11603. As shown, the template 11601 is spaced proximally from the anchor 11602, where the anchor 11602 may be releasably coupled to the shaft of the anchor control device 11603. FIG. 116 B shows the preformed or pre-shaped template 11601 in a final delivery position 11605, having slid distally to engage the anchor 11602. Of particular significance, by allowing the preformed or pre-shaped template 11601 to slide over the shaft anchor control device 11603, the anchor control device 11603 can act as a guide to properly position the template 11601 at a target tissue site in the annulus or other tissue.

The template 11601 in final delivery position 11605 may be coupled to the anchor control device 11603 by an anchor coupling device 11604. The anchor coupling device can take several forms, including elastic tabs that capture the template in final delivery position 11605, a nut that is screwed on to the anchor 11602, or other such mechanisms known to the art. When the anchor control device 11603 is released from the anchor 11602, for example by removing a key wire 11606, the anchor control device 11603 can be removed, while the template in final delivery position 11605 remains coupled to the anchor 11602 in the tissue.

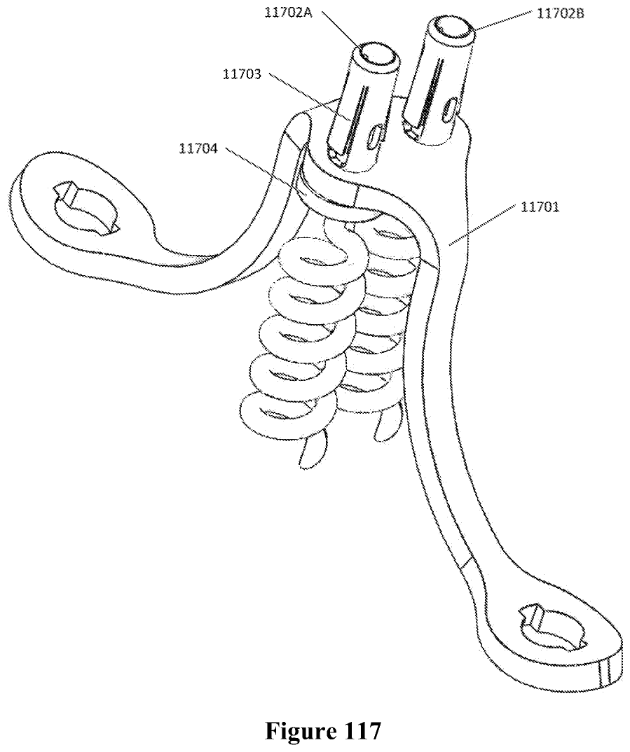

FIG. 117 shows a tissue shaping template 11701 having two central anchors 11702A and 11702B disposed through holes near the apex of the concavity. Tabs 11703 and washers 11704 couple the anchors to the tissue shaping template 11701, the tabs 11703 allowing the tissue shaping template 11701 to move distally relative to the anchors 11702A-B, but preventing proximal motion once the tissue shaping template 11701 moves distally past the distal aspect of the tabs 11703. The washers 11704 prevent further distal motion of the tissue shaping template 11701 relative to the anchors. Both anchors 11702A-B may be placed in tissue before the tissue shaping template 11701 is guided into place along one or more anchor control wires (not shown), or one anchor 11702A may be placed first, and the second anchor 11702B may be coupled to the tissue shaping template 11701 prior to coupling anchor 11702B to the target tissue.

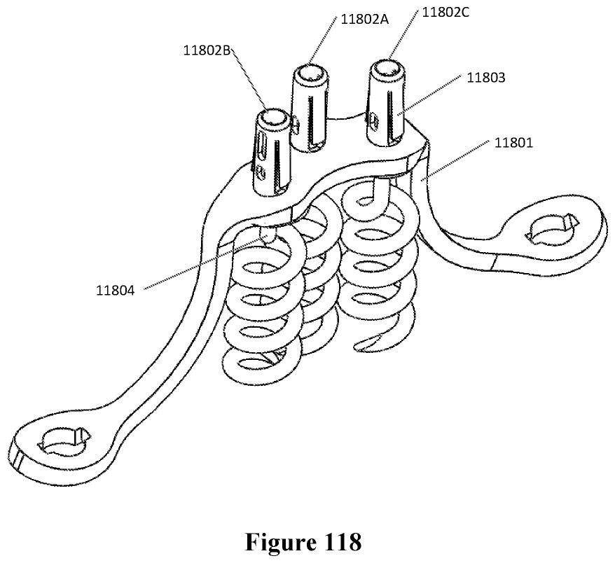

FIG. 118 shows a tissue shaping template 11801 having one central anchor 11802A disposed through a hole near the apex of the concavity, and two accessory anchors 11802B-C disposed through holes to either side of the center hole. Tabs 11803 and washers 11804 couple the anchors to the tissue shaping template 11801, the tabs 11803 allowing the tissue shaping template 11801 to move distally relative to the anchors 11802A-C, but preventing proximal motion once the tissue shaping template 11801 moves distally past the distal aspect of the tabs 11803. The washers 11804 prevent further distal motion of the tissue shaping template 11801 relative to the anchors. The center anchor 11802 is placed in tissue before the tissue shaping template 11801 is guided into place along the central anchor control wire (not shown), and the accessory anchors 11802B-C are coupled to the tissue as the tissue shaping template 11801 is advanced to couple to the center anchor 11802A.

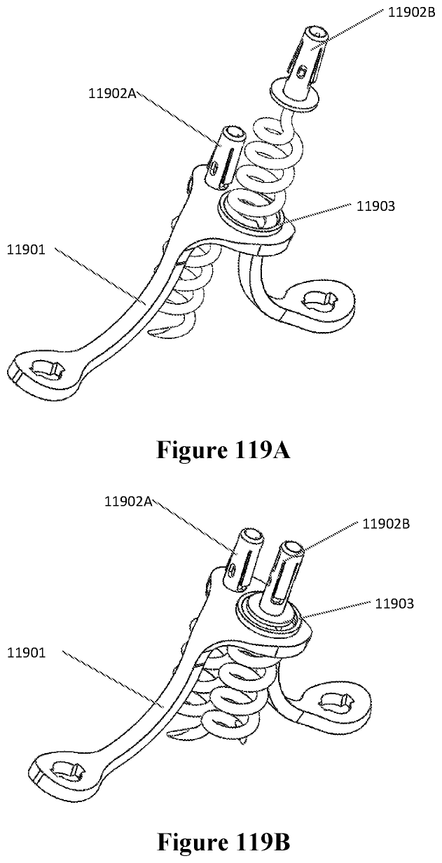

FIG. 119A shows a tissue shaping template 11901 having a first anchor 11902A which passes through a hole in the tissue shaping template 11901 and 11902B which is coupled to a threaded pod 11903 which is in turn coupled to the tissue shaping template and a second anchor 11902B coupled to the threaded pod 11903 is in the proximal position, and does not penetrate the target tissue (not shown).

FIG. 119 B shows the tissue shaping template 11901 of FIG. 119A. In FIG. 119B, the anchor 11902B coupled to the threaded pod 11903 is in the distal position and penetrates the target tissue (not shown).

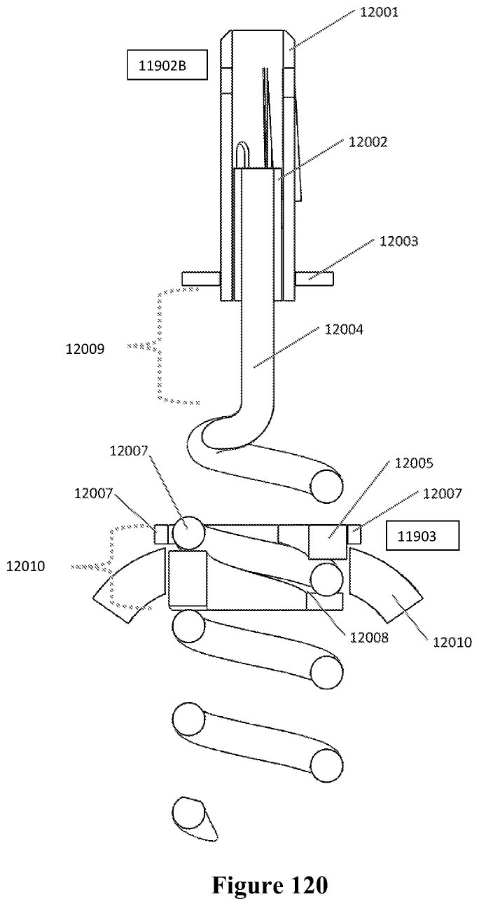

FIG. 120 shows a section view of the pod 11903 and anchor 11902B from FIG. 119. The anchor 11902B comprises a dock 12001 coupled to a spacer 12002, a washer 12003, and a wire form 12004. The wire form 12004 comprises a centered axial segment having a length 12009, and a number of helical coils 12007. The pod 11903 comprises a pod coil 12005, a pod ring 12007, and a pod boss (not shown). The anchor 11902B passes through the pod coil 12005, which has a helical thread 12008 that allows for passage of the helical coils 12007 of the anchor 11902B. The pod coil 12005 passes through a hole in the tissue shaping template 12010, and is prevented from moving distally through the hole by the pod ring 12007, and is prevented from moving proximally through the hole by a pod boss (not shown). The pod 11903 has a height 12010 which is less than or equal to the centered axial segment length 12009. When the anchor 11902B is positioned distally relative to the pod 11903, so that the washer 12003 prevents further distal relative motion, the helical coils 12007 of the anchor 11902B are disengaged from the pod coil 12005, allowing the anchor 11902B to rotate, with the centered axial segment 12004 passing through the inner diameter of the pod coil 12005. This allows additional rotation of the anchor 11902B as it engages tissue if required.

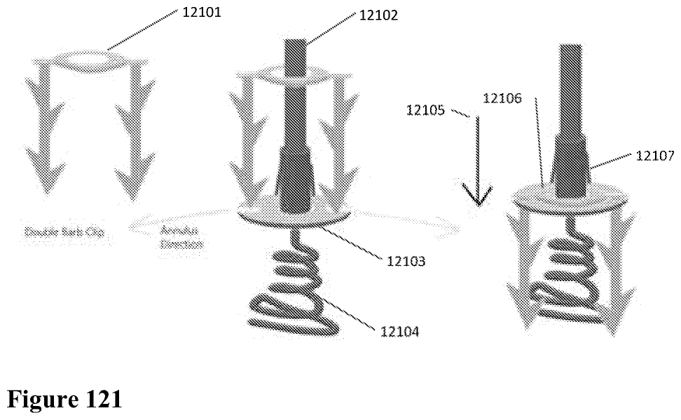

FIG. 121 shows accessory barbs 12101 for augmenting the tissue holding force of a tissue anchor 12104. The tissue anchor 12104 includes a washer 12103 and is coupled to tissue (not shown), and the accessory barbs 12101 are slidably or translatably coupled to the anchor control wire 12102 and are moved distally along the control wire 12102 to position adjacent the tissue anchor 12104. The tissue anchor 12104 stabilizes the tissue as the accessory barbs 12101 are pushed distally to penetrate the target tissue, until the connecting member 12106 of the accessory barbs 12101 couples with the dock tabs 12107 of the tissue anchor 12104.

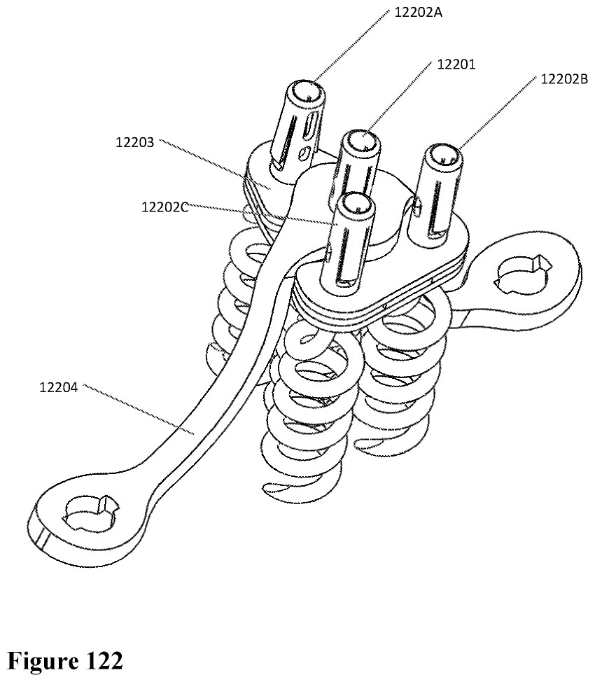

FIG. 122 shows an array of accessory anchors 12202 A-C coupled to a frame 12203 which is in turn coupled to a primary anchor 12201. The primary anchor 12201 is placed in tissue, then the frame 12203 and attached accessory anchors 12202 A-C are delivered adjacent the primary anchor 12201, and the accessory anchors 12202 A-C are coupled to the target tissue. The frame 12203 may be coupled directly to the primary anchor 12201 or may be captured between features (not shown) of the primary anchor 12201 distal to the frame 12203 and a therapeutic implant 12204 proximal to the frame 12203 coupled to the primary anchor 12201.

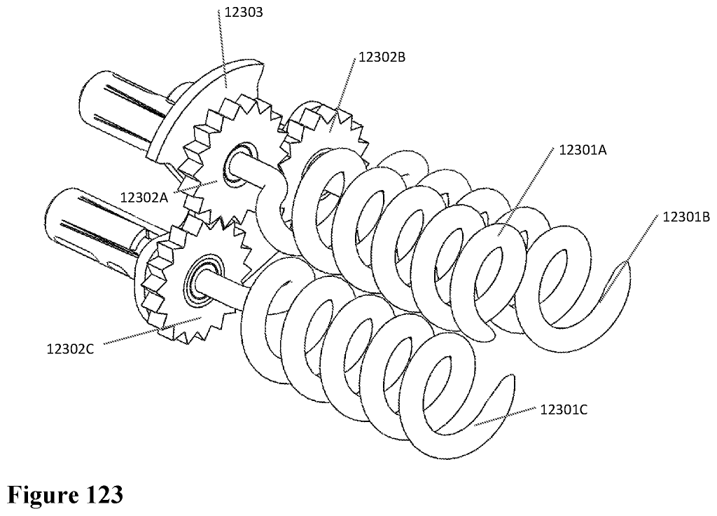

FIG. 123 shows an array of accessory anchors 12301 A-C having gears 12302 A-C coupled to a frame 12303 such that turning any one of the gears 12302 A-C causes the other gears and respective anchors to turn. As shown, turning gear 12302 A counter-clockwise would cause anchor 12301A to draw itself into tissue, while simultaneously turning gears 12302 B-C and the corresponding accessory anchors 12301 B-C to turn the opposite (clockwise) direction, pushing themselves out of the target tissue. It may be advantageous to construct the accessory anchors 12301 B-C with opposite handedness of accessory anchor A so that they draw or retract from tissue simultaneously when any one of the gears 12302 A-C is turned.

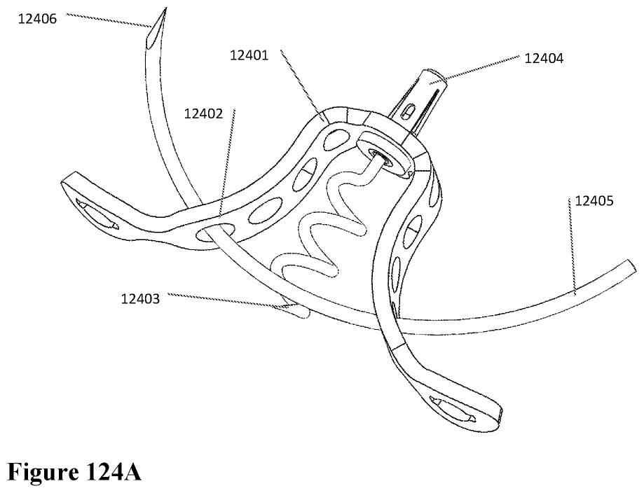

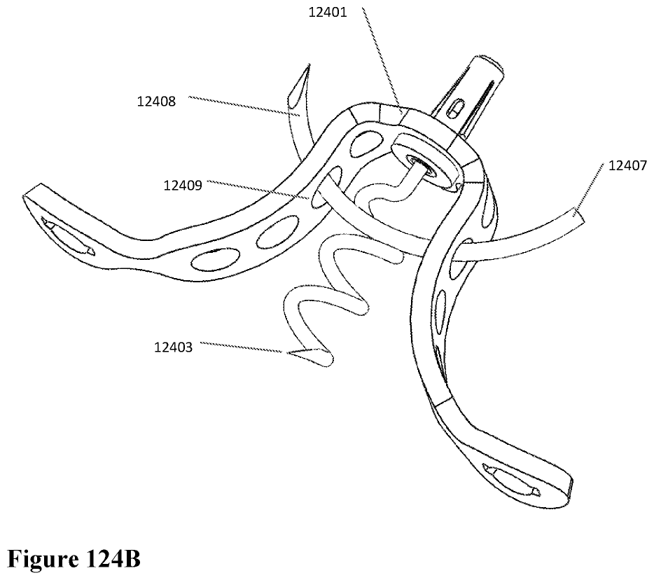

FIG. 124A shows a tissue shaping template 12401 with holes 12402 arranged along its length coupled to a tissue anchor 12403 via a dock 12404. A first accessory latch 12405 having a tissue penetrating tip 12406 is placed through opposed holes 12402 and through the tissue held against the tissue shaping template 12401, providing additional tissue holding capability.

FIG. 124 B shows the tissue shaping template 12401 of FIG. 124A, with a second accessory latch 12407 having a tissue penetrating tip 12408 placed through opposed holes 12409 close to the proximal end of the anchor, providing additional tissue holding capability. The first accessory latch 12405 of FIG. 124A and the second accessory latch 12407 of FIG. 124 B can be combined to provide further additional tissue holding capability.

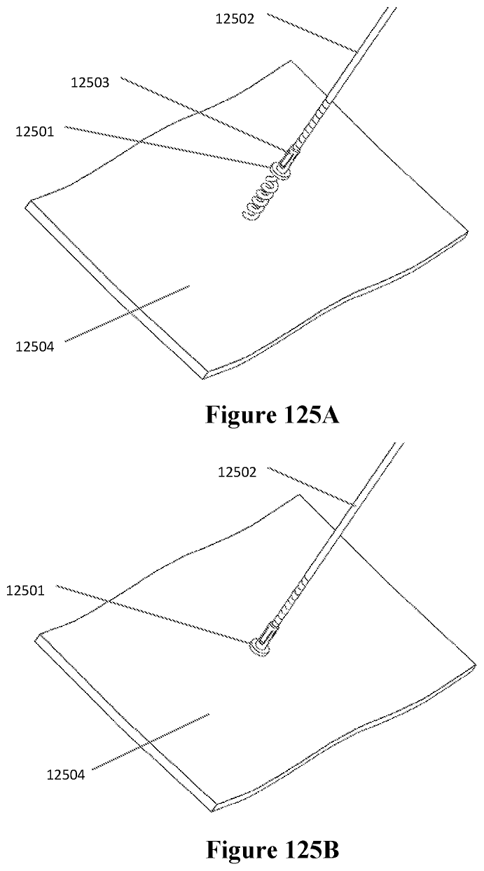

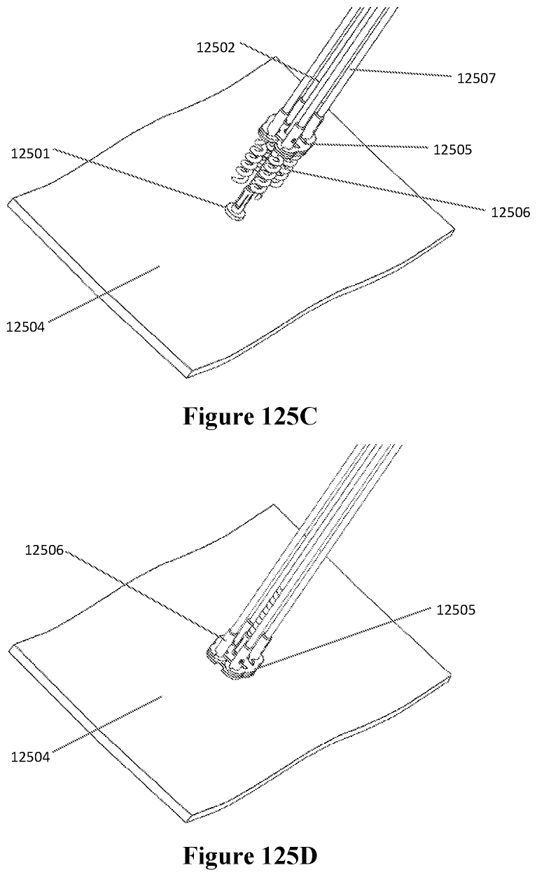

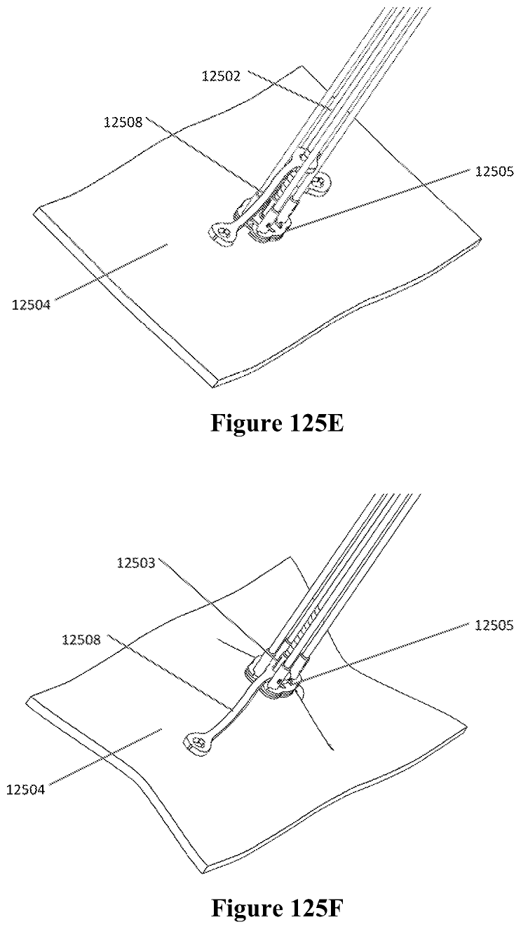

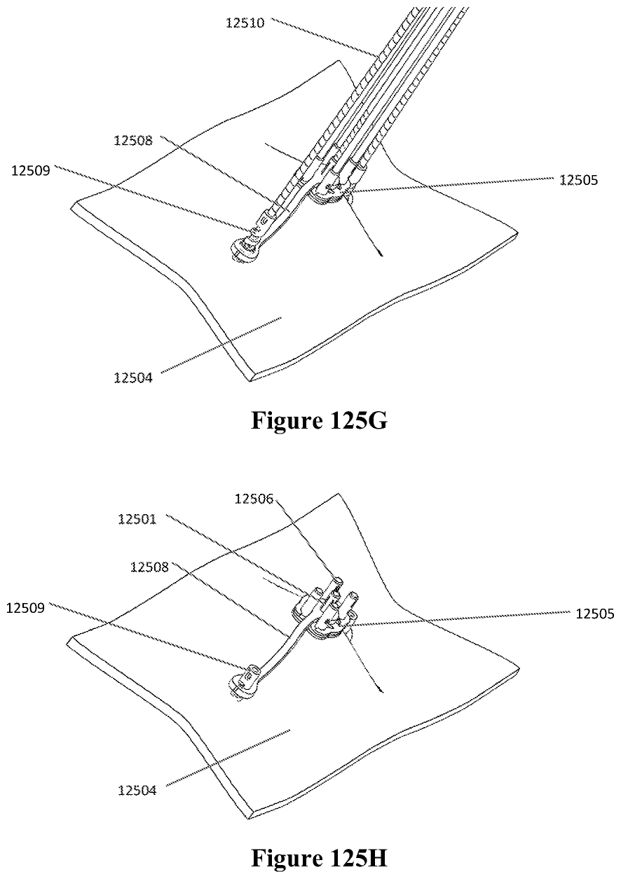

FIG. 125A shows a locating tissue anchor 12501 comprising a dock 12503 releasably coupled to a locating anchor control wire 12502 approaching target tissue 12504.

FIG. 125 B shows a locating tissue anchor 12501 coupled to target tissue 12504.

FIG. 125C shows a frame 12505 slidably coupled to an anchor control wire 12502 approaching target tissue 12504 adjacent a locating tissue anchor 12501. The frame is coupled to one or more (four as shown) accessory anchors 12506, which are releasably coupled to accessory control wires 12507.

FIG. 125D shows the frame 12505 coupled to the target tissue 12504 via the accessory anchors 12506.

FIG. 125E shows a tissue shaping template 12508 translatably or slidably coupled to a locating anchor control wire 12502 approaching target tissue 12504. A template handle (not shown) is releasably coupled to the tissue shaping template 12508 allowing the user to control the position of the tissue shaping template 12508.

FIG. 125F shows the tissue shaping template 12508 of FIG. 125E engaged with the target tissue 12504. Tab features on the dock 12503 deflect as the tissue shaping template 12508 moves distally over and past the tabs, then move outward to prevent the tissue shaping template 12508 from moving proximally relative to the dock 12503. The template handle (not shown) can be released from the tissue shaping template 12508 and removed at this point.

FIG. 125G shows the tissue shaping template 12508 of FIG. 125F with side anchors 12509 stabilizing the ends of the tissue shaping template 12508 by engaging the target tissue 12504, the side anchors 12509 being releasably coupled to side anchor control wires 12510.

FIG. 125H shows the locating tissue anchor 12501, the frame 12505, the accessory anchors tissue shaping template 12508, the tissue shaping template 12508, and the side anchors 12509 in place in the target tissue 12504 after removal of locating anchor control wire 12502, accessory control wires 12507, and side anchor control wires 12510.

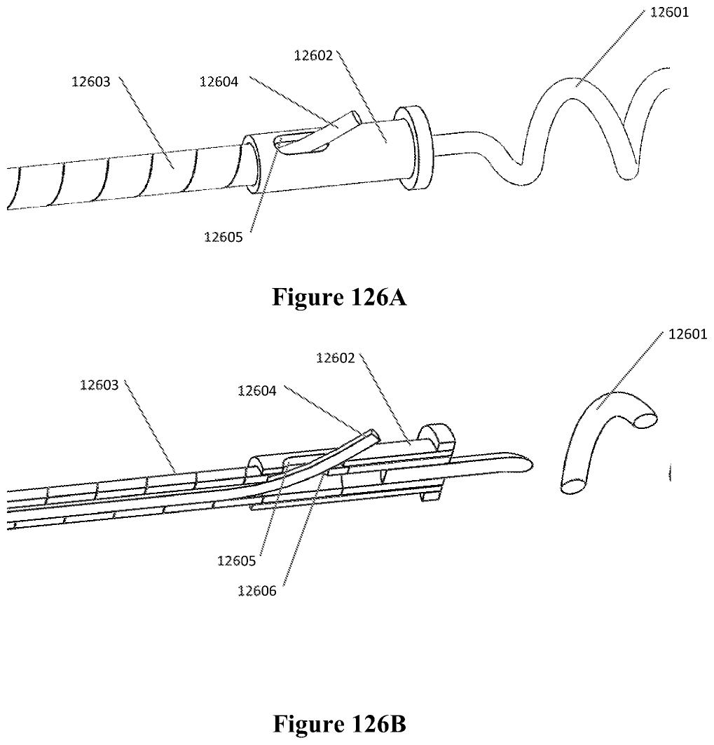

As shown in FIG. 126A and FIG. 126B, a tissue anchor 12601 with an anchor body 12602 is coupled to a torque tube 12603 by a key wire 12604. The key wire 12604 extends through a torque tube hole 12606 in the torque tube 12603 and a lock hole 12605 in the anchor body 12602. The rotational and longitudinal alignment of torque tube hole 12606 and lock hole 12605 allows passage of the key wire 12604, and the key wire 12604 through in place through torque tube hole 12606 and lock hole 12605 prevents or limits their misalignment, effectively coupling the anchor body 12602 to the torque tube 12603. Pulling the key wire 12604 proximally removes it from the torque tube hole 12606 and lock hole 12605, de-coupling the anchor body 12602 from the torque tube 12603.

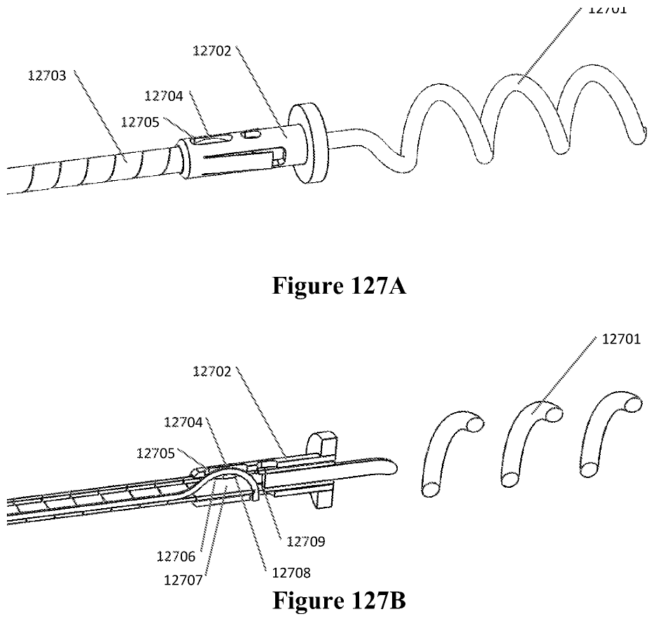

As shown in FIG. 127A and FIG. 127B, a tissue anchor 12701 with an anchor body 12702 is coupled to a torque tube 12703 by a key wire 12704. The key wire 12704 extends through a first torque tube hole 12706, over a bridging segment 12707, and back in through a second torque tube hole 12708. The key wire 12704 as it passes over the bridging segment 12707 extends into a lock slot 12705 in the anchor body 12702. The rotational and longitudinal alignment of first torque tube hole 12706, bridging segment 12707 and second torque tube hole 12708 with the lock slot 12705 allows passage of the key wire 12704, and the key wire 12704 through in place through the first torque tube hole 12706, over the bridging segment 12707 and back through the second torque tube hole 12708 while passing through the lock slot 12705 prevents or limits their misalignment, effectively coupling the anchor body 12702 to the torque tube 12703. Pulling the key wire 12704 proximally removes it from the torque tube holes 12706 and 12708 and lock slot 12705, de-coupling the anchor body 12702 from the torque tube 12703. The anchor body may include a key wire exit hole 12709 through which the key wire 12704 may pass, allowing for tension to be applied to the key wire during assembly. When the diameter of the key wire 12704 is approximately the same as the wall thickness of the anchor body 12702, the profile of the coupled assembly is comparable to the anchor body 12702 alone, allowing unrestricted passage of a tissue shaping template (not shown) over the anchor body 12702.

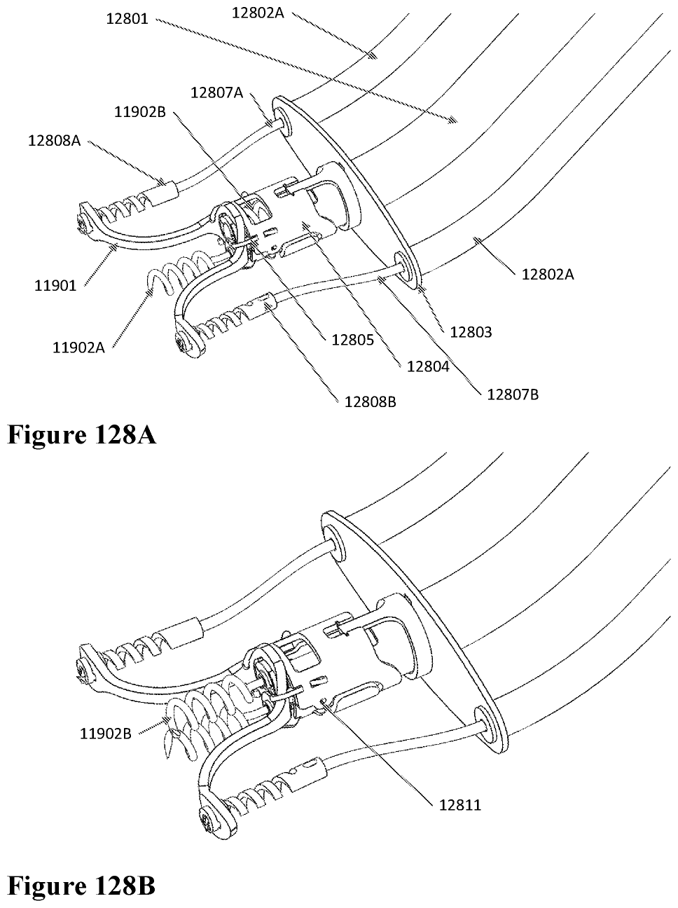

As shown in FIGS. 128A and 128B, a delivery device for delivering the template 11901 shown in FIG. 119 comprises a central shaft 12801 which forms an integral stationary jaw, two side anchor torque tube guides 12802A and 12802B, a torque tube guide holder 12803, a movable jaw 12804 having teeth 12805 and a window through which the accessory anchor 11902B can be observed. The template 11901 is shown with attached side anchors 12808A and 12808B, which are releasably coupled to side anchor torque tubes 12807A and 12807B which run through lumens in the torque tube guides 12802A and 12802B. The template 11901 is also coupled to a primary anchor 11902A, and a secondary anchor 11902B, each of which is releasably coupled to corresponding torque tubes, not shown. In FIG. 128A, the accessory anchor 11902B is in the retracted position (as delivered to the target tissue, not shown), and is visible through the window in the movable jaw 12804. In FIG. 128B, the accessory anchor 11902B is in the extended position having been coupled to the target tissue (not shown), and is no longer visible through the window in the movable jaw 12804. From some angles, it is possible to observe the proximal end of the accessory anchor 11902B and the corresponding anchor torque tube (not shown).

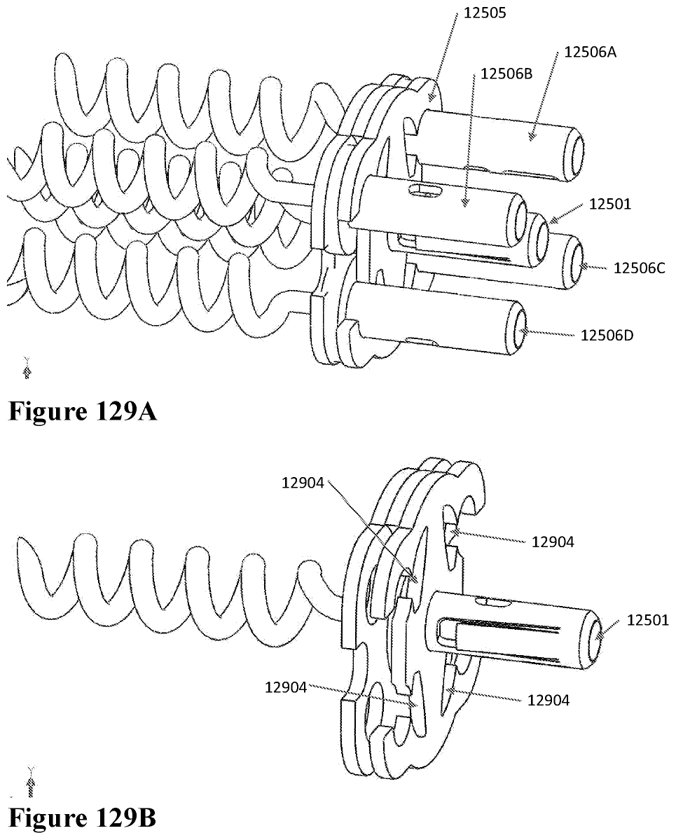

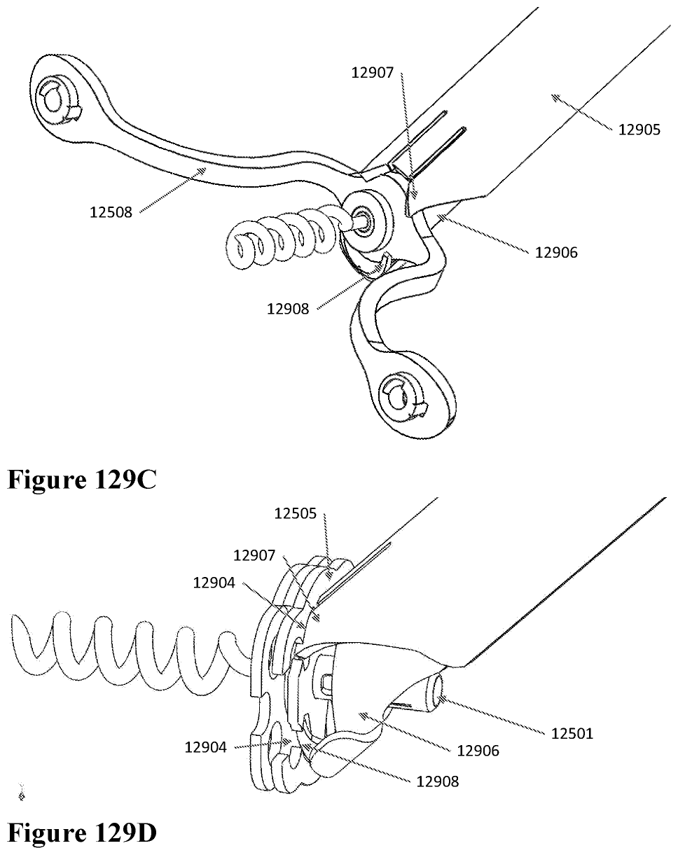

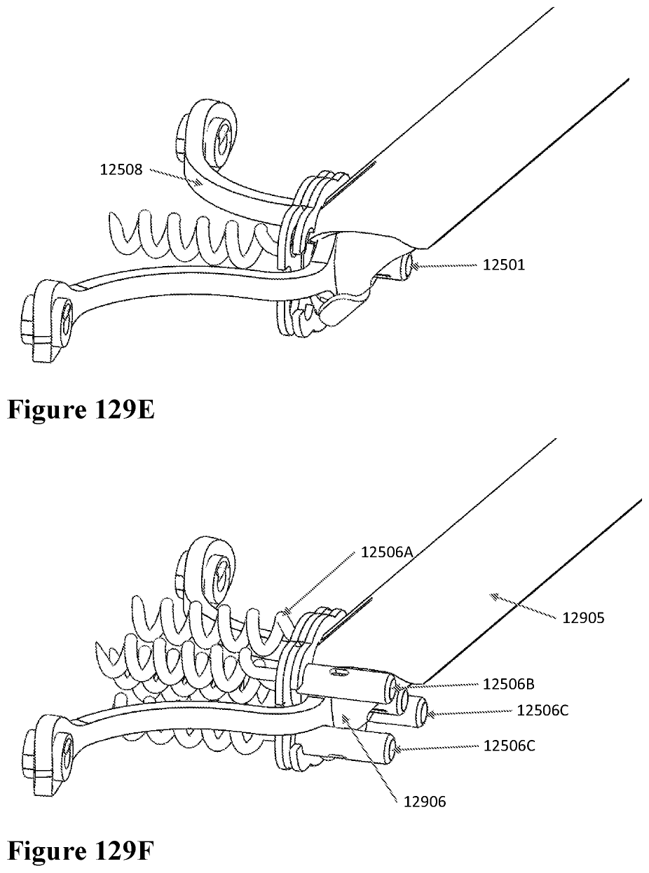

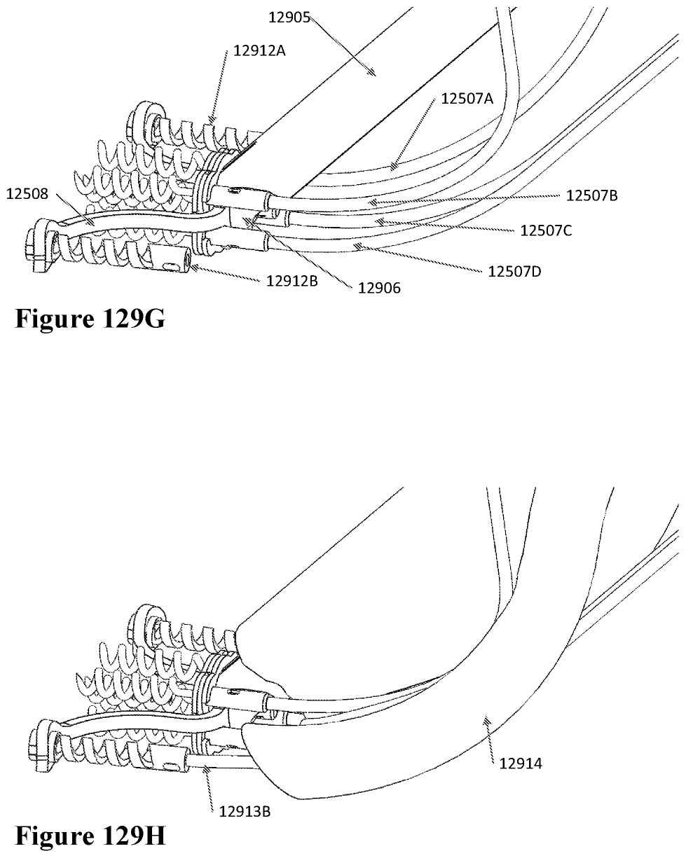

FIG. 129A shows the frame 12505 of FIG. 125, comprising a locating tissue anchor 12501 and four accessory anchors 12506A-12506D.

FIG. 129B shows the frame 12505 of FIG. 125, with the accessory anchors 12506A-12506D hidden to allow view of the tooth clearance areas 12904 in the frame 12505.

FIG. 129C shows a delivery device comprising two telescoping tubes, and outer tube 12905, and an inner tube 12906. The outer tube has one or more top teeth 12907 and the inner tube has one or more bottom teeth 12908 which wrap at least partially around the tissue shaping template 12508, releasably coupling the template 12508 to the delivery device. The template 12508 is coupled to the delivery device at an angle to the axis of the outer tube 12905 and the inner tube 12906.

FIG. 129D shows the delivery device of FIG. 129C in position to place a template 12508 (not shown) against a frame 12505. The top teeth 12907 and the bottom teeth 12908 engage the tooth clearance areas 12904 of the frame 12505, allowing the delivery device to move the template 12508 (not shown) closer to the frame 12505 than interference between the teeth 12907 and 12908 and the frame 12505 would allow without the clearance areas 12904. Also shown in FIG. 129D is the proximal dock end of the locating tissue anchor 12501 extending through a clearance hole in the inner tube 12906.

FIG. 129E shows the delivery device and frame with the template 12508, which was hidden in FIG. 129D.

FIG. 129F shows the delivery device, frame, and template of FIG. 129E with the accessory anchors 12506A-12506D, which were hidden in FIG. 129E.

FIG. 129G shows the delivery system of FIG. 129F, with accessory control wires 12507A-12507D in place on the accessory anchors, and with side anchors 12912A and 12912B in place on the template 12508. The design and configuration of outer tube 12905 and the inner tube 12906 allow them to fit within the accessory anchors 12506A-12506D and accessory anchor control wires 12507A-12507D without impairing the function of the accessory anchors 12506A-12506D or accessory anchor control wires 12507A-12507D.

FIG. 129H shows the delivery system of FIG. 129G with side anchor control wires 12913A (not shown) and 12913B, which pass into lumens in the side anchor control wire guide member 12914.

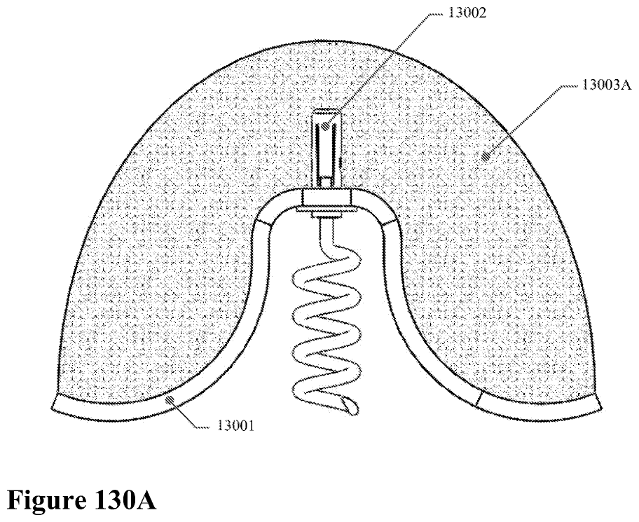

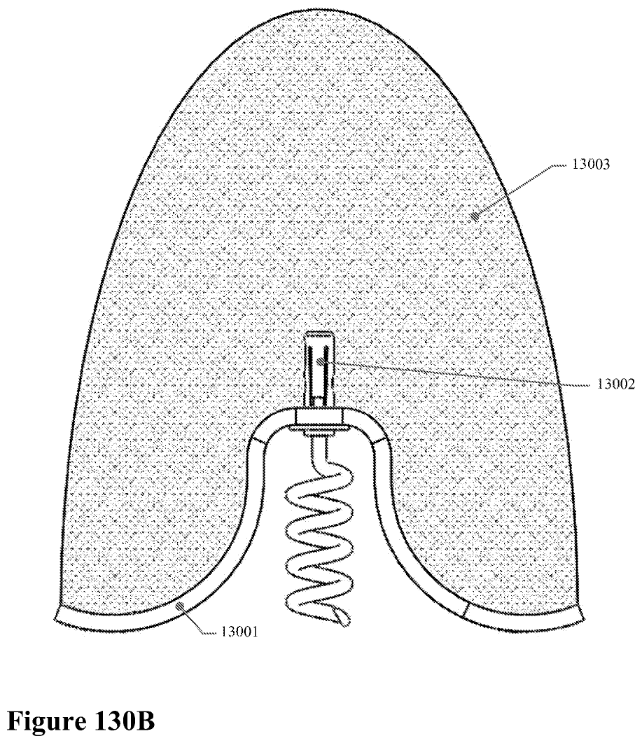

FIG. 130A shows a tissue shaping template 13001 having an anchor 13002 and an attached skirt 13003A to offer a valve sealing surface to one or more apposed valve leaflets. The length of the attached skirt is sufficient to coapt with one or more other valve leaflets in apposition to the skirt. The leaflet is constructed of a rigid, semi-rigid, or flexible material that is biocompatible and hemo-compatible. Examples of such materials include ePTFE, woven Dacron with metallic supports, PVA, Nitinol, Stainless-steel, cobalt chromium, and the like.

FIG. 130B shows a tissue shaping template 13001 having an anchor 13002 and an attached skirt 13003B of sufficient length and pliability to fold down act as a prosthetic valve leaflet. The leaflet is constructed of a semi-rigid or flexible material that is biocompatible and hemo-compatible. Examples of such materials include ePTFE, woven Dacron with metallic support wires, PVA, Nitinol, Stainless-steel, cobalt chromium, and the like.

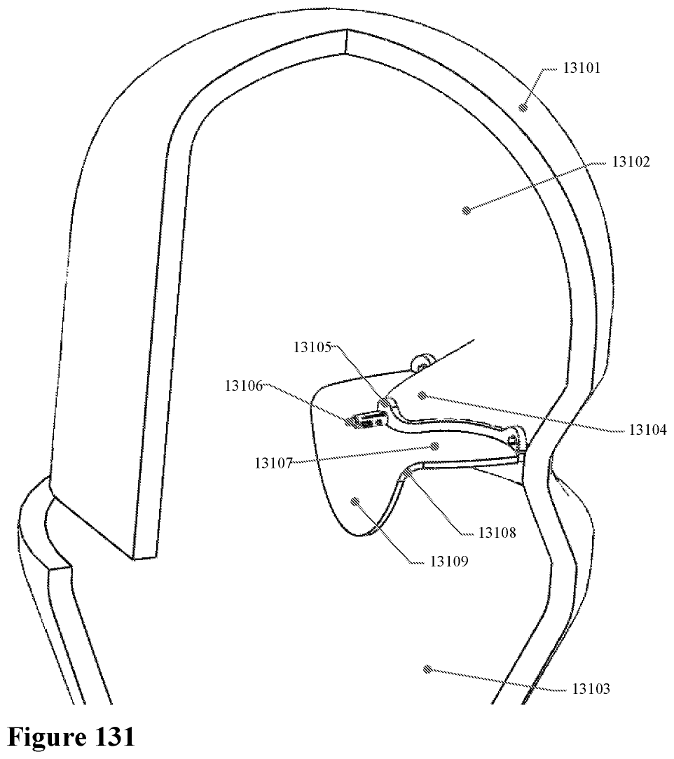

FIG. 131 shows a cutaway view of a heart 13101 having an atrium 13102, a ventricle 13103 and an annulus between them (not shown) with a tissue shaping template 13105 held in place at the valve annulus by an anchor 13106 and creating a tissue bump 13104. The template 13105 has an attached skirt 13107 to offer a valve sealing surface to one or more apposed valve leaflets, not shown. The attached skirt 13107 has a bend 13108 and a ventricular portion 13109. The ventricular portion 13109 may be rigid to provide a stationary sealing surface for one or more apposed valve leaflets, or may flex in response to blood flow or pressure to act as a functional prosthetic leaflet.

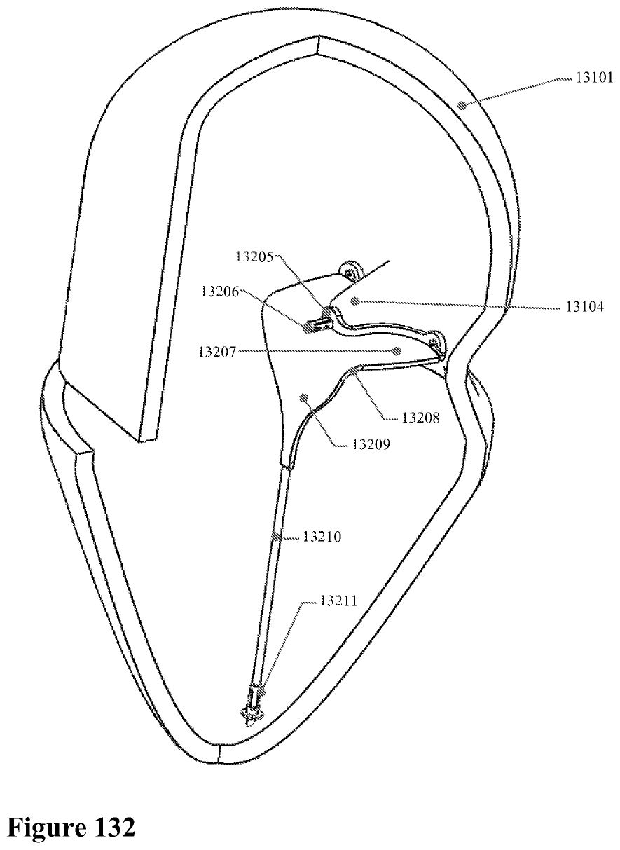

FIG. 132 shows a cutaway view of a heart 13101 with a tissue shaping template 13205 held in place at the valve annulus by an anchor 13206 and creating a tissue bump 13104. The template 13205 has an attached skirt 13207 to offer a valve sealing surface to one or more apposed valve leaflets, not shown, the skirt 13207 being coupled to a stabilizing chord 13210 which is anchored into the tissue of the ventricle by a chord anchor 13211. The tissue of the ventricle to which the chord anchor 13201 is coupled may be the apex of the ventricle, the wall of the ventricle, or one or more papillary muscle.

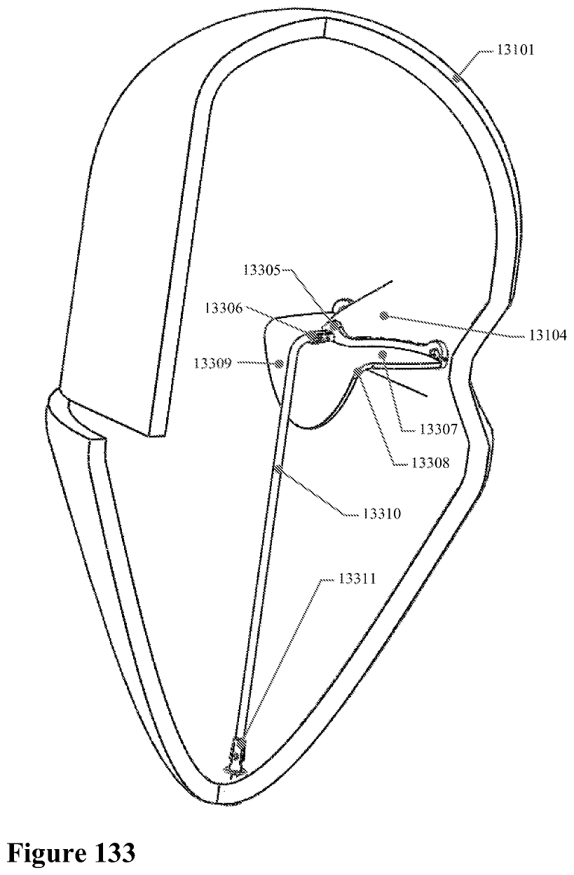

FIG. 133 shows a cutaway view of a heart 13101 with a tissue shaping template 13305 held in place at the valve annulus by an anchor 13306 and creating a tissue bump 13104. The template 13305 has an attached skirt 13307 to offer a valve sealing surface to one or more apposed valve leaflets, not shown, the template 13305 being coupled to a stabilizing chord 13310 which is anchored into the tissue of the ventricle by a chord anchor 13311. The tissue of the ventricle to which the chord anchor 13301 is coupled may be the apex of the ventricle, the wall of the ventricle, or one or more papillary muscle. This stabilizing chord also stabilizes the attached skirt in a bent configuration.

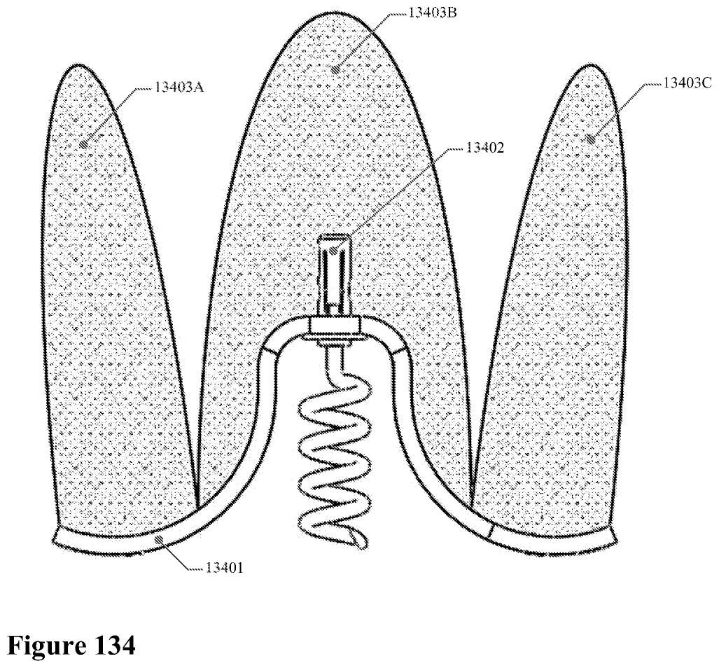

FIG. 134 shows a tissue shaping template 13401 having a tissue anchor 13402 and multiple attached skirts 13402A-C, to offer a valve sealing surface to one or more apposed valve leaflets (not shown). The leaflets are constructed of a rigid, semi-rigid, or flexible material that is biocompatible and hemo-compatible. Examples of such materials include ePTFE, woven Dacron with metallic supports, PVA, Nitinol, Stainless-steel, cobalt chromium, and the like.

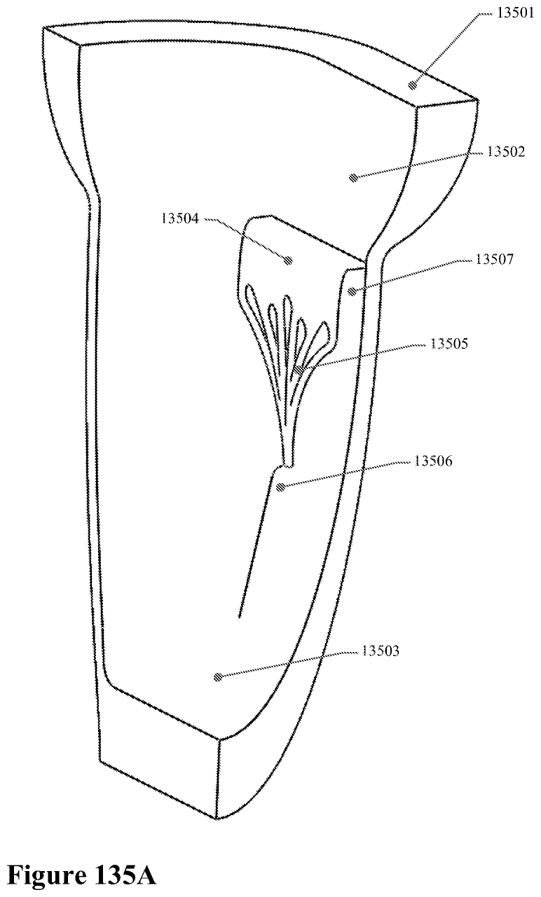

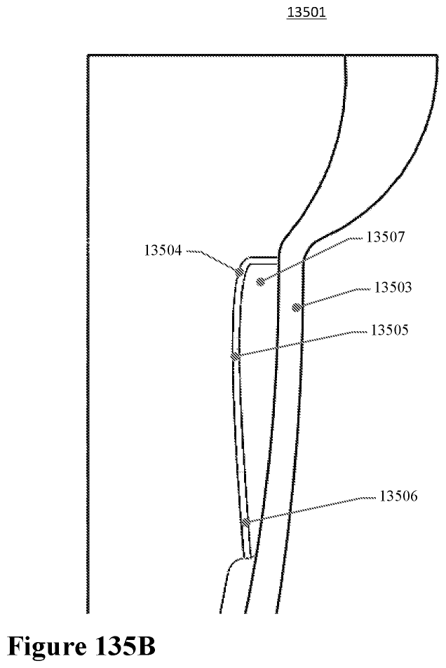

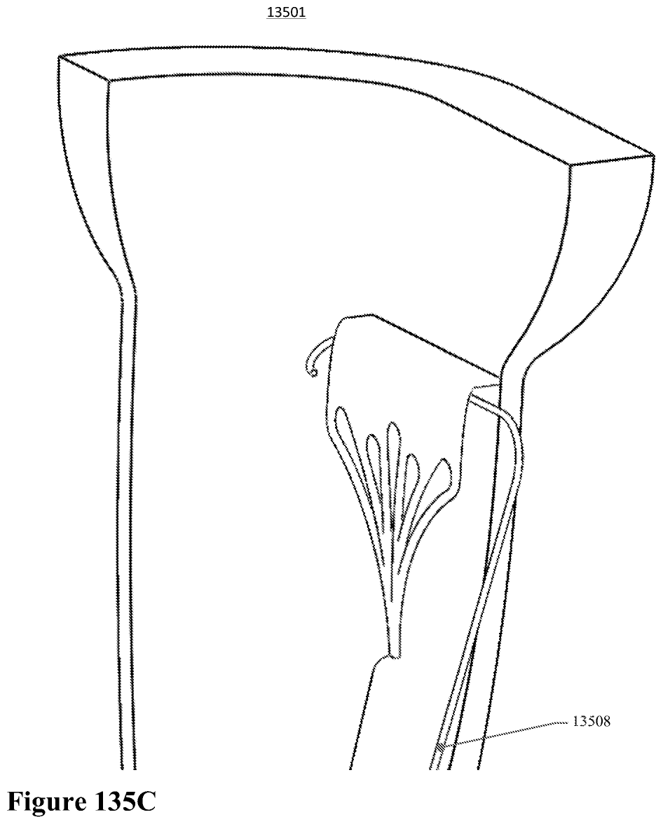

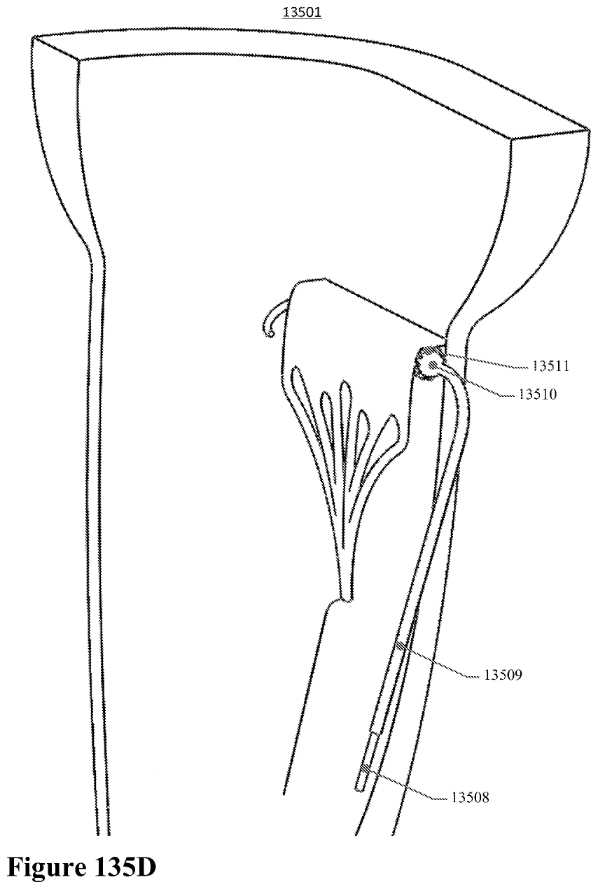

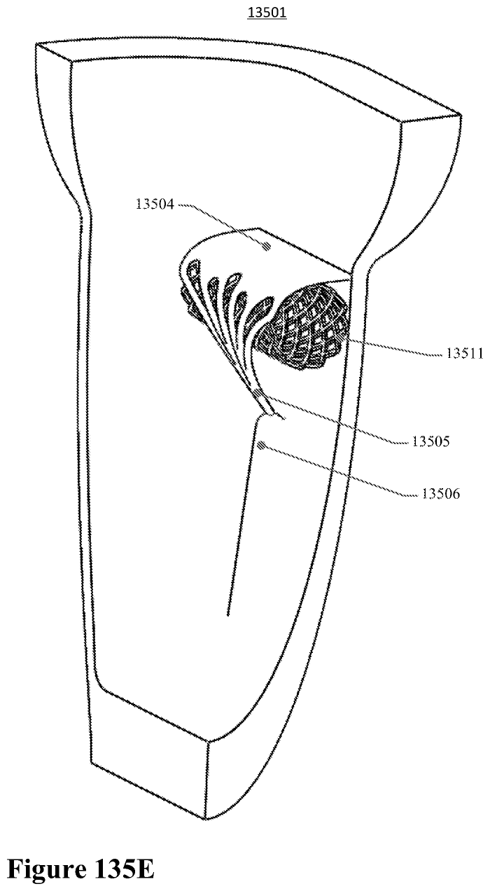

FIG. 135A shows a cutaway view of a heart 13501 having an atrium 13502, a ventricle 13503, and a valve leaflet 13504 that is tethered by chordae 13505 coupling the leaflet 13504 to a papillary muscle 13506 in the ventricle. The leaflet tethering as shown holds the leaflet 13504 in close apposition to the ventricular wall, limiting its motion. The space 13507 between the leaflet 13504 and the adjacent wall of the ventricle 13503 is small as a result of the leaflet tethering.

FIG. 135B shows a side view of the heart 13501 shown in FIG. 135A, illustrating the relative size of the space between the leaflet 13504 and the adjacent wall of the ventricle 13503.

FIG. 135C shows the heart 13501 of FIG. 135A, having a guidewire 13508 placed into the space 13507 between the tethered leaflet 13504 and the wall of the ventricle 13503.

FIG. 135D shows the heart 13501 and guidewire 13508 of FIG. 135C, with a stent catheter 13509 in place between the tethered leaflet 13504 and the wall of the ventricle 13503. The stent catheter 13509 comprises a catheter body, a stent expansion system 13510, and a stent 13511. The stent expansion system 13510 may be a balloon, a release tube, or other mechanisms known to the art. The stent 13511 is delivered in a crimped state, and may be plastically deformable to an expanded state, or may be held in the crimped state by the stent expansion system 13510 and released to self-expand into the expanded state.

FIG. 135E shows the heart 13501 of FIG. 135A with the stent 13511 of FIG. 135D in the fully expanded state, moving the tethered leaflet 13504 out away from the adjacent wall of the ventricle 13503 and closer to one or more other leaflets (not shown).

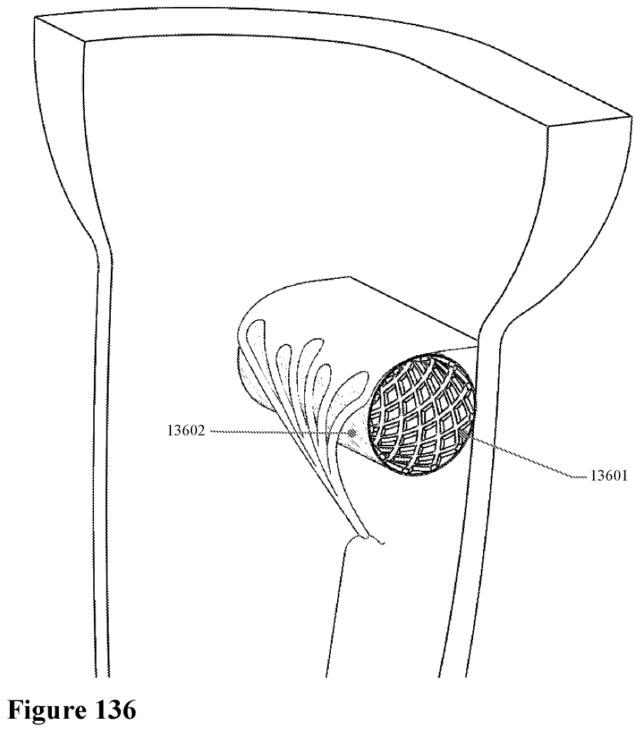

FIG. 136 shows a coated stent 13601 in place between a tethered leaflet 13504 and adjacent wall of the ventricle 13503 as shown in FIG. 135E, the coated stent 13601 having a hemostatic or semi-permeable coating 13602 on the outside surface of the stent 13601. This coating 13602 can offer an additional coaptation surface in situations where the tethered leaflet surface is inadequate for coaptation with one or more apposed leaflets.

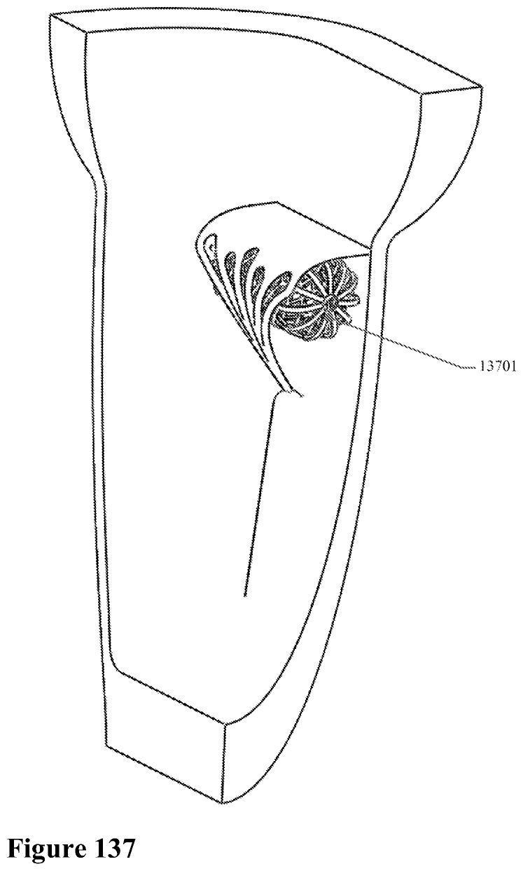

FIG. 137 shows a non-cylindrical stent 13708 in place between a tethered leaflet 13504 and the wall of the ventricle 13503, the non-cylindrical stent 13708 having a basket shape with substantially closed ends. The closed ends may reduce the possibility of embolized thrombus.

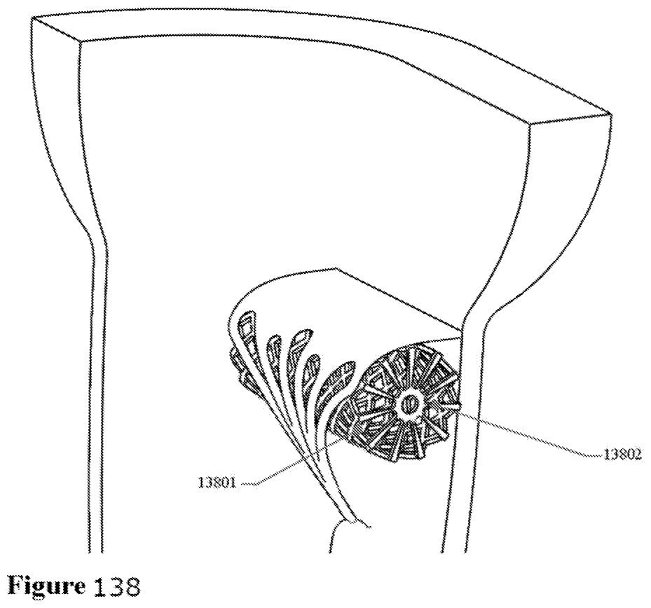

FIG. 138 shows a cylindrical stent 13801 with end caps 13802 to substantially close the ends of the stent 13801. The closed ends may reduce the possibility of embolized thrombus.

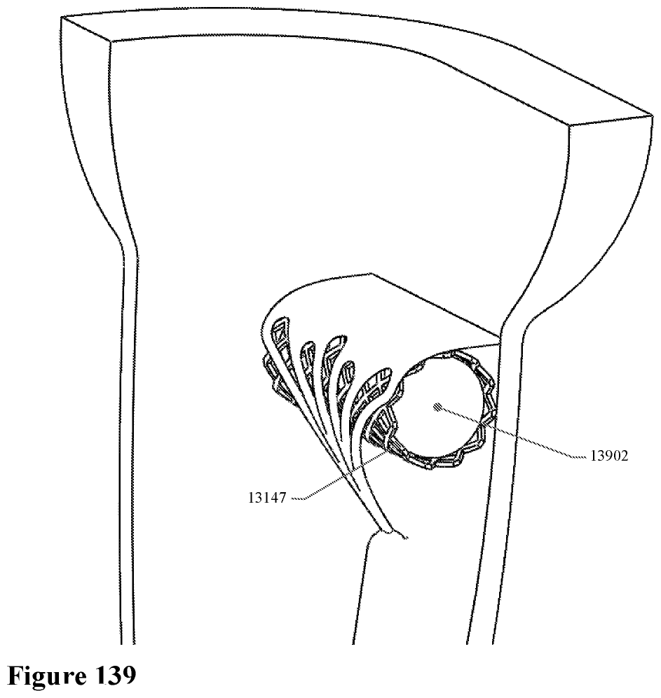

FIG. 139 shows the stent 13511 of FIG. 135E with a filler material 13902 in the stent. This material may be beneficial to control tissue healing and thrombus formation. The filler material may comprise one or more biocompatible materials, examples of which include hydrogel, silicone, fiber, tangled coils of polymer or metal, or a balloon filled with a saline solution, gel, silicone, or other such injectable biocompatible material. The filler material may be textured to encourage tissue ingrowth. The balloon, if present, may be permeable or hemostatic, and may encourage tissue ingrowth.

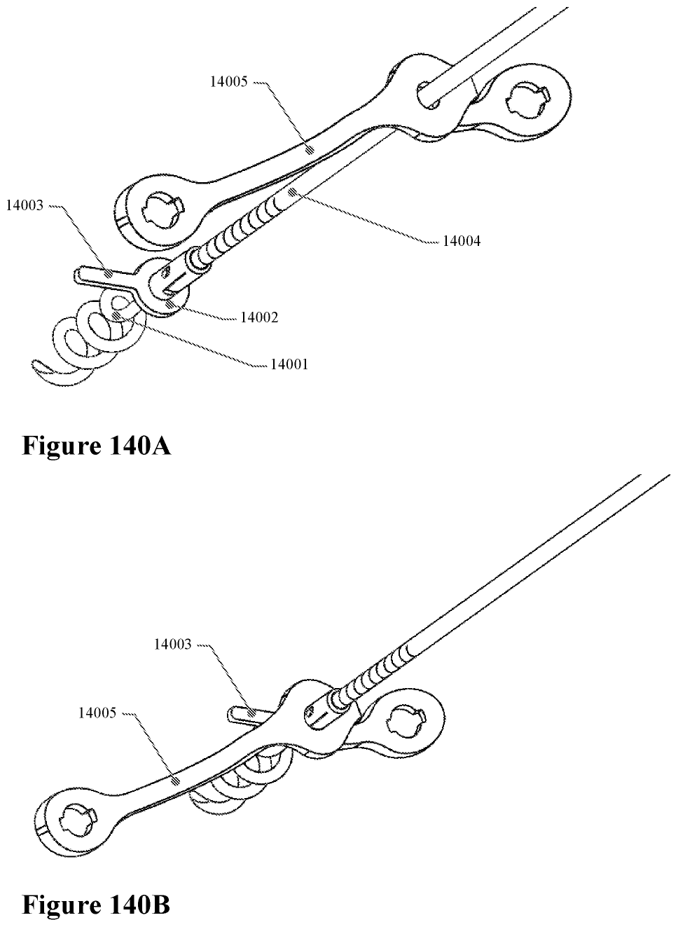

FIG. 140A shows a tissue anchor 14001 having a washer 14002 with a protrusion 14003, the tissue anchor 14007 being releasably coupled to a control wire 14004, and a tissue template 14005 slidably coupled to the control wire.

FIG. 140B shows the system of FIG. 140A after the tissue template 14005 has been moved distally against the washer 14002. The protrusion 14003 interacts with the tissue template 14005 to prevent rotation of the anchor 14001 relative to the tissue template 14005. As shown, the protrusion 14003 would prevent the anchor 14001 from rotating counter-clockwise, by apposing the left leg of the template 14005.

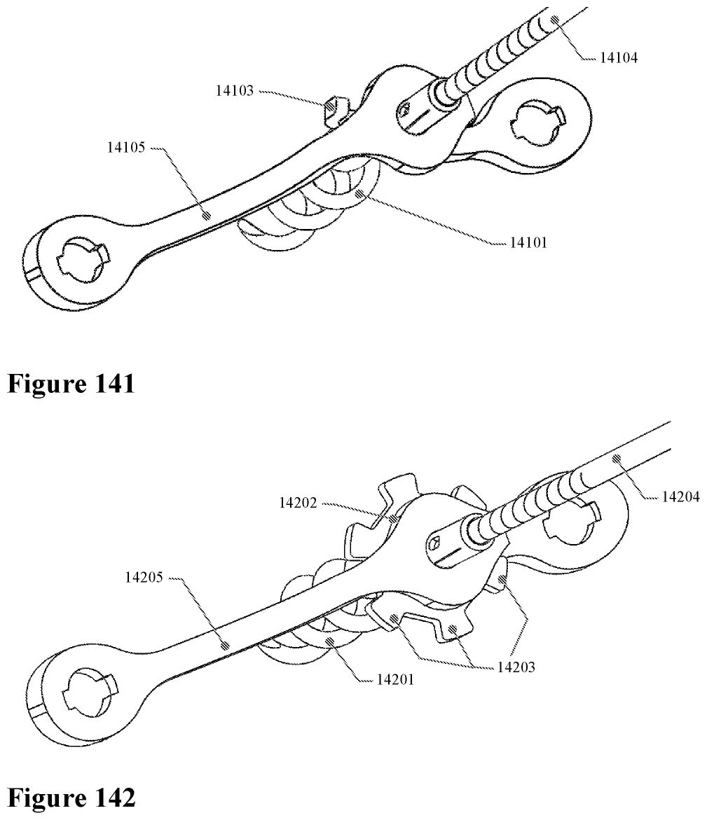

FIG. 141 shows a tissue anchor 14101 having a washer with a protrusion having a proximal extension 14103, the tissue anchor 14101 being releasably coupled to a control wire 14104, and a tissue template 14105 engaged with the anchor 14101 and in apposition with the washer. The proximally extension 14103 on the protrusion interacts with the tissue template 14105 to prevent rotation of the anchor 14101 relative to the tissue template 14105. As shown, the proximal extension 14103 would prevent the anchor 14101 from rotating counter-clockwise, by apposing the left leg of the template 14105.

FIG. 142 shows a tissue anchor 14201 having a washer 14202 with a multiple protrusions 14203, the tissue anchor 14201 being releasably coupled to a control wire 14204, and a tissue template 14205 engaged with the anchor 14201 and in apposition with the washer 14202. One or more of the protrusions 14203 interact with the tissue template 14205 to prevent rotation of the anchor 14201 relative to the tissue template 14205. As shown, the protrusions 14203 interact with both legs of the tissue template 14205 to prevent the anchor 14201 from rotating in either a clockwise or a counterclockwise direction.

FIG. 143 shows a leaflet 14301 of a heart valve secured to the ventricle by chordae 14302 connecting to papillary muscles 14303A and 14303B. Two or more cylindrical stents 14304 placed between the valve leaflet 14301 and the wall of the ventricle. The stents 14304 are held in place by their interaction with the chordae 14302 of the valve leaflet 14301.

FIG. 144 shows a stent 14401 having extension bosses 14402 which interact with valve chordae (not shown) to hold the stent in a position between a valve leaflet and the wall of the ventricle. The stent 14401 may be self-expanding or balloon expandable,

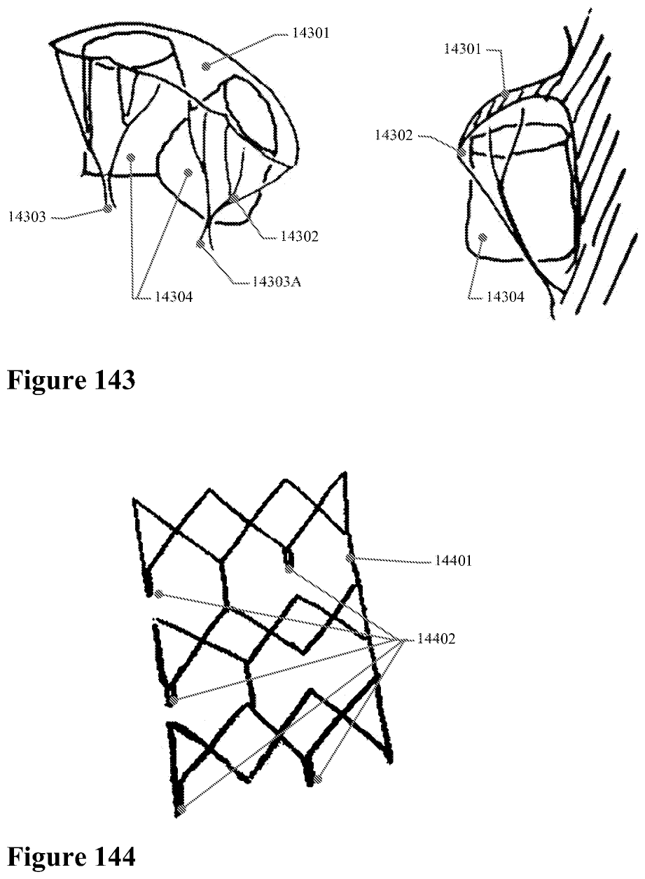

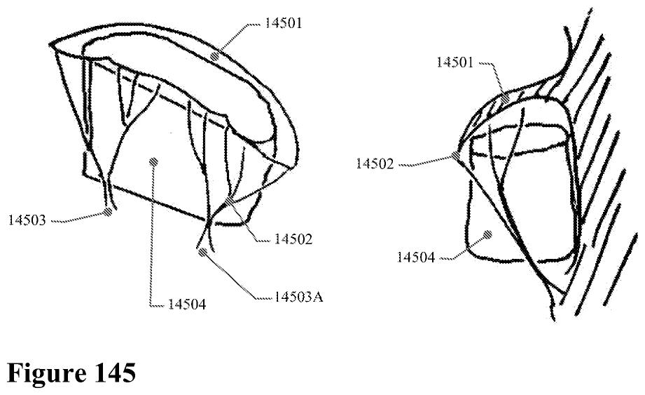

FIG. 145 shows a single stent 14504 having a flattened profile placed between a valve leaflet 14501 and the wall of the ventricle. The stent 14504 is held in place by interaction with the chordae 14502 which connect the leaflet 14501 with the papillary muscles 14503A-B. Other shapes having an elongate or flattened profile, including fan shapes, cone shapes, ellipsoids, ovoids, parabaloids, having an axis substantially perpendicular to the valve annulus and one diameter that is substantially less than the other diameter may have similar benefits.

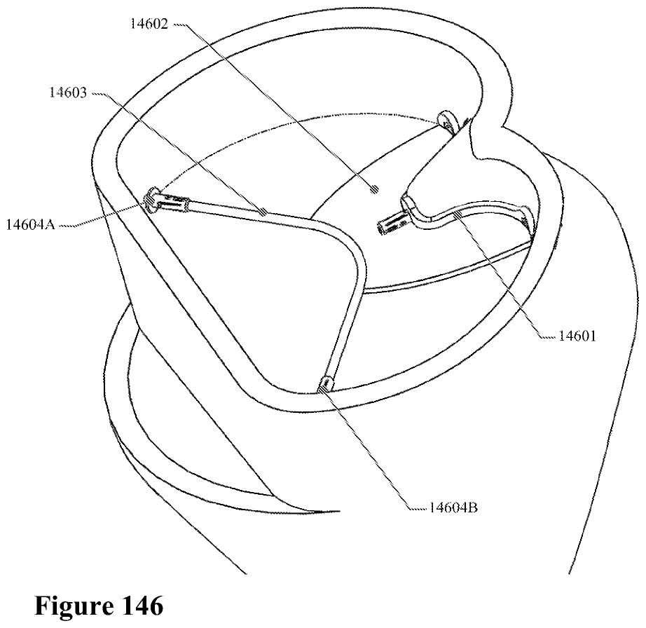

FIG. 146 shows a tissue shaping template 14601 in place on the posterior aspect of a mitral annulus, with a prosthetic leaflet 14602 coupled to the template 14601. The leaflet 14602 is supported in position in the annular plane by two tethers 14603 attached to anchors 14604A and 14604B in the fibrous trigones of the valve annulus. The tissue anchors 14604A and 14604B may comprise sutures, pledgets, staples, clips, helical coils, barbs or other anchoring mechanisms known to the art. The tethers 14603 may be continuous with each other, or separate, and the length of the tethers 14603 may be adjustable at one or both of the anchors 14604A and 14604B, or the adjustment may be made along the length of the tethers 14603, or at the junction of the tethers and the prosthetic leaflet 14602.

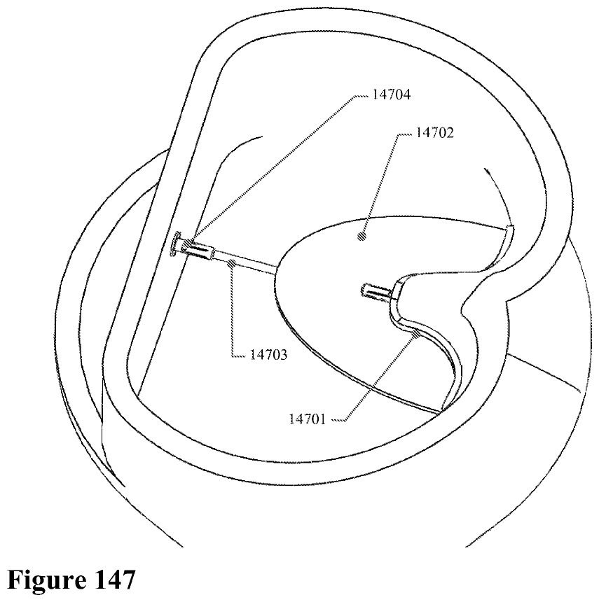

FIG. 147 shows a tissue shaping template 14701 in place on the posterior aspect of a mitral annulus, with a prosthetic leaflet 14702 coupled to the template. The leaflet 14702 is supported in position in the annular plane by one tether 14703 attached to an anchor 14704 in the anterior valve annulus. The anterior valve annulus is considered to run from one fibrous trigone to the other along the wall shared with the aorta. The tissue anchor 14704 may comprise sutures, pledgets, staples, clips, helical coils, barbs or other anchoring mechanisms known to the art. The tether 14603 may be adjustable at the anchor 14704, at the prosthetic leaflet 14702, or along the length of the tether 14603.

FIG. 148 shows a tissue shaping template 14801 in place on the posterior aspect of a mitral annulus, the template being stabilized in position in the annular plane by one or more tethers. Tethers 14804A and 14804B couple the template 14801 directly to the fibrous trigones. Tethers 14803A and 14803B couple the tissue anchor 14802 directly to the fibrous trigones. Tether 14805 couples either the tissue anchor 14802 or the template 14801 to the anterior valve annulus. The anterior valve annulus is considered to run from one fibrous trigone to the other along the wall shared with the aorta.

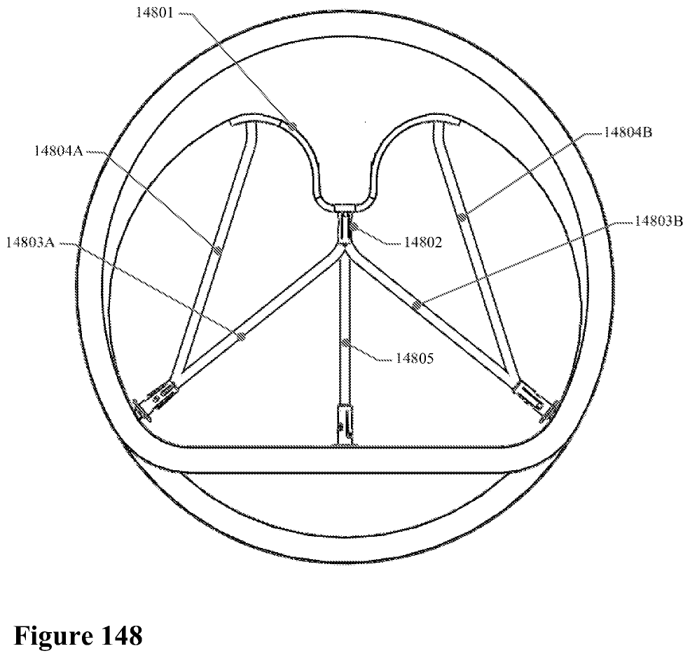

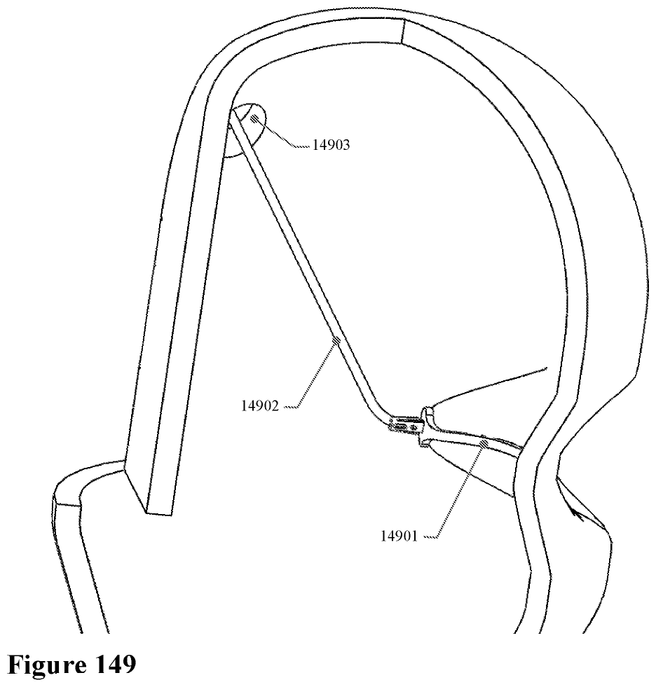

FIG. 149 shows a tissue shaping template 14901 in place on the posterior aspect of a mitral annulus, the template being stabilized against motion in the posterior direction by a tether 14902 coupled to the atrial septum 14903, by a basket, clip, suture, hook, anchor, clip, staple, or other method known to the art. This coupling technique may be beneficial when the template 14901 is placed into the annulus via a trans-septal catheter procedure.

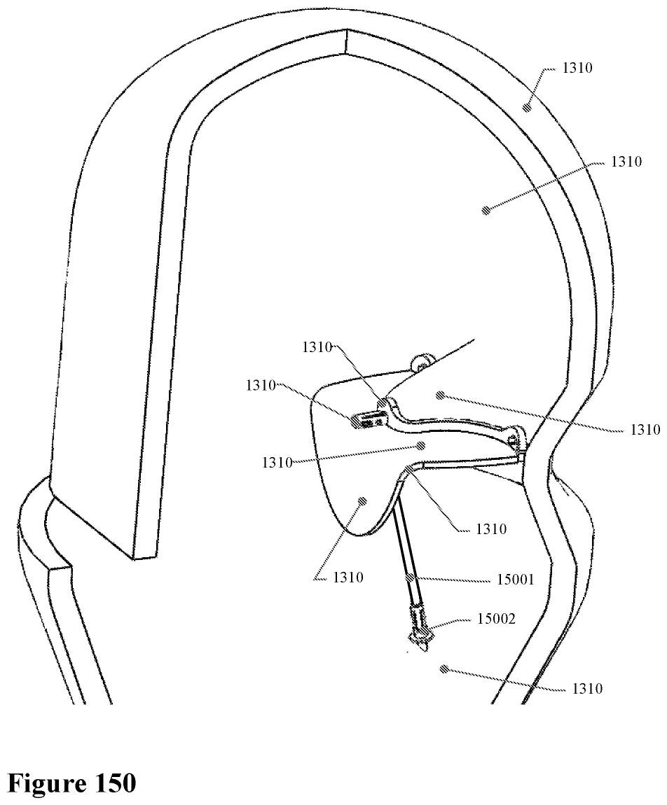

FIG. 150 shows the cutaway view of a heart 13101 from FIG. 131, having an atrium 13102, a ventricle 13103 and an annulus between them (not shown) with a tissue shaping template 13105 held in place at the valve annulus by an anchor 13106 and creating a tissue bump 13104. The template 13105 has an attached skirt 13107 to offer a valve sealing surface to one or more apposed valve leaflets, not shown. The attached skirt 13107 has a bend 13108 and a ventricular portion 13109. The ventricular portion 13109 may be rigid to provide a stationary sealing surface for one or more apposed valve leaflets or may flex in response to blood flow or pressure to act as a functional prosthetic leaflet. The underside of the prosthetic leaflet 13109 is coupled to a tether 15001 which is in turn coupled to an anchor 15002 attached to the tissue of the ventricle 13103. The tissue of the ventricle may include the apex of the ventricle, the muscular wall of the ventricle, and the papillary muscles.

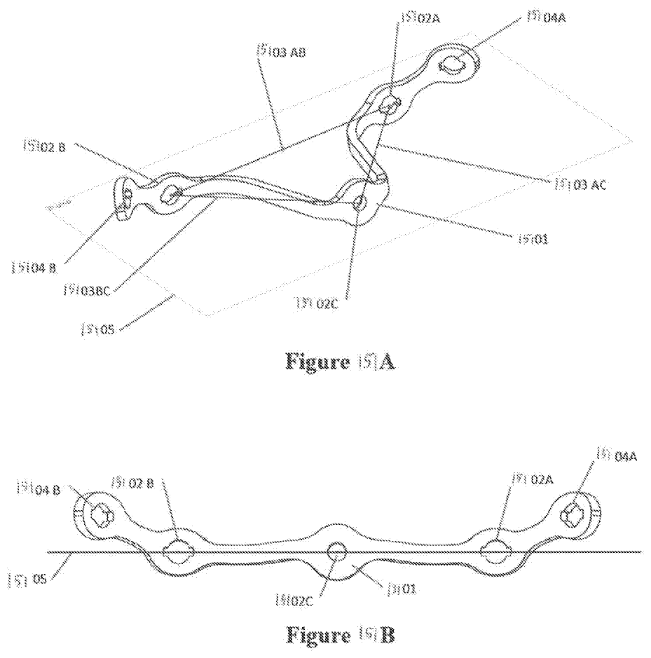

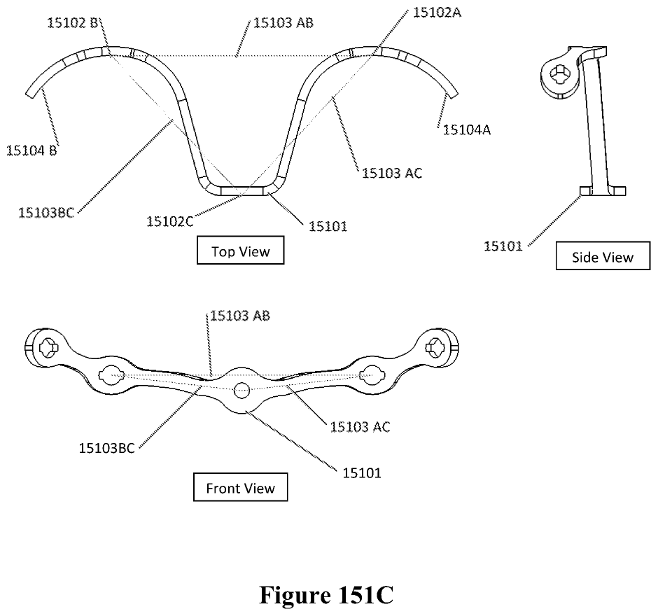

FIG. 151A shows a template 15101 having a center anchor coupling feature 15102C, two inner side anchor-coupling features 15102A and 15102B, and two outer side anchor-coupling features 15104A and 15104B. The three center points of coupling features 15102A, 15102A, and 15102A define a plane 15105, and the outer coupling features 15104A and 15104B extend to one side of plane 15105. A triangle with sides 15103AB, 15103BC, and 15103AC connects the coupling features 15102A, 15102A, and 15102A, and lies in plane 15105.

FIG. 151B shows the template 15101 of FIG. 151A from a view perpendicular to plane 15105, so that plane appears as a line. In this view, it is apparent that the ends of the template 15101 comprising the outer coupling features 15104A and 15104B lay outside plane 15105.

FIG. 151C shows the template 15101 of FIG. 151A in three orthogonal views, highlighting the shape of template 15101 and its relationship to the triangle having sides 15103AB, 15103BC, and 15103AC which defines plane 15105 (not shown).

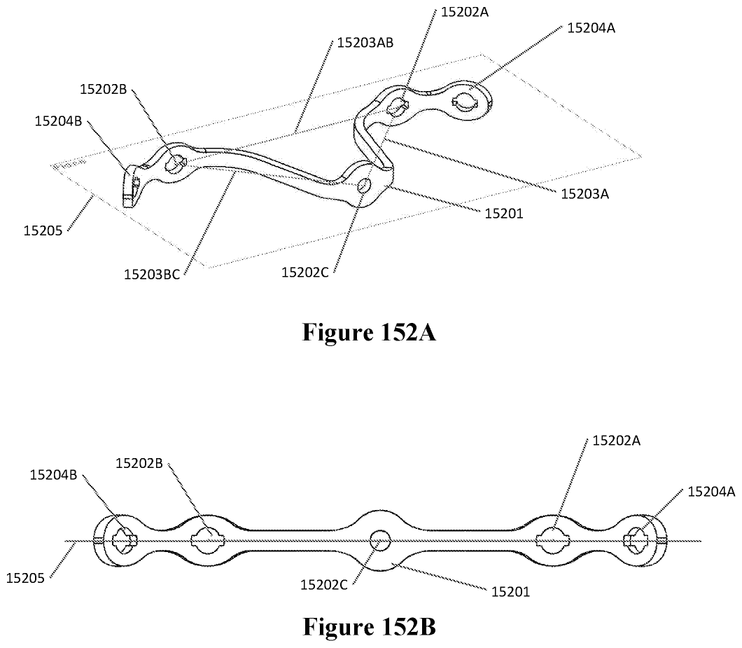

FIG. 152A shows a template 15201 having a center anchor-coupling feature 15202C, two inner side anchor-coupling features 15202A and 15202B, and two outer side anchor-coupling features 15204A and 15204B. The three center points of coupling features 15202A, 15202A, and 15202A define a plane 15205, and the outer coupling features 15204A and 15204B lie along plane 15205. A triangle with sides 15203AB, 15203BC, and 15203AC connects the coupling features 15202A, 15202A, and 15202A, and lies in plane 15205.

FIG. 152B shows the template 15201 of FIG. 152A from a view perpendicular to plane 15205, so that plane appears as a line. In this view, it is apparent that the ends of the template 15201 comprising the outer coupling features 15204A and 15204B lay along plane 15205.

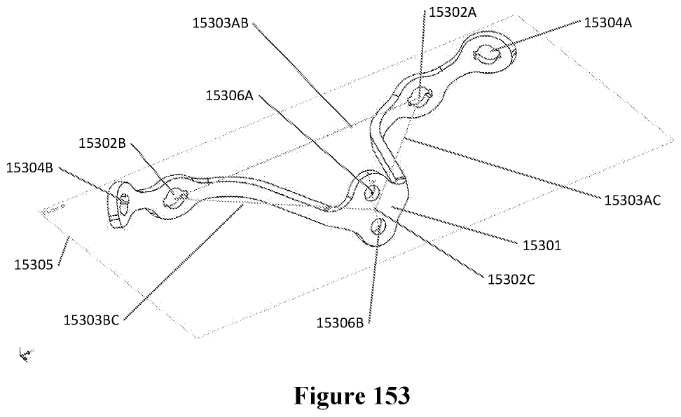

FIG. 153 shows a template 15301 having two center anchor-coupling features 15306A and 15306B, two inner side anchor-coupling features 15302A and 15302B, and two outer side anchor-coupling features 15304A and 15304B. The two center anchor-coupling features 15306A and 15306B lie at each end of a line with a midpoint at 15302C. The three points 15302A, 15302A, and 15302A define a plane 15305, and the outer coupling features 15304A and 15304B lie to one side of plane 15305. A triangle with sides 15303AB, 15303BC, and 15303AC connects the coupling features 15302A, 15302A, and 15302A, and lies in plane 15305.

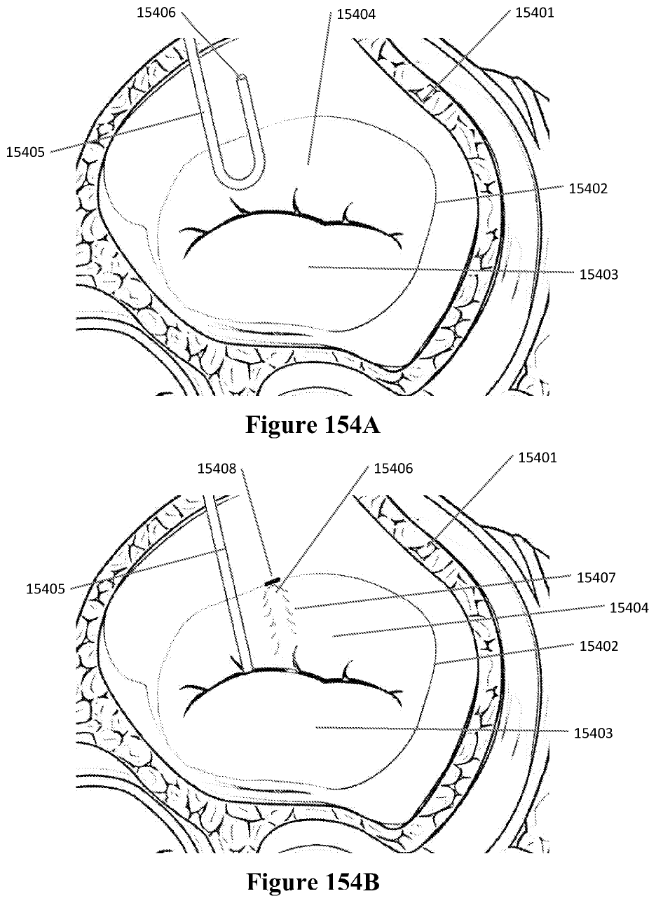

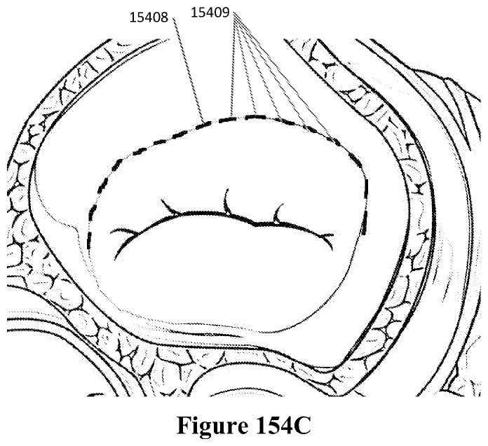

FIG. 154A shows a left atrial view of a heart 15401 having a mitral valve comprising an annulus 15402, an anterior leaflet 15403, and a posterior leaflet 15404. A probe 15405 is shaped to pass through the left atrium, between the valve leaflets 15403 and 15404, and into the left ventricle. The probe 15405 has an atraumatic tip 15406.

FIG. 154B shows the heart 15401 and the probe 15405 of FIG. 154A, the probe having been passed through the leaflets 15403 and 15404, between the ventricle and the posterior valve leaflet 15404, and raised to lift the leaflet 15404, allowing accurate visualization of the attachment point between the leaflet 15404 and ventricle. The outline 15407 of a portion of the probe 15405 is visible as a raised portion of the posterior leaflet 15404. A mark 15408 is placed at the attachment point of the posterior leaflet 15404 and the ventricle, guided by the outline 15407.

FIG. 154C shows the heart 15401 of FIGS. 154A and 154B, with a series of additional marks 15409 adjacent the mark 15408 shown in FIG. 154B. As many additional marks 15409 as required to define the target section of the annulus may be made in an analogous manner to the mark 15408 shown in FIG. 154B.

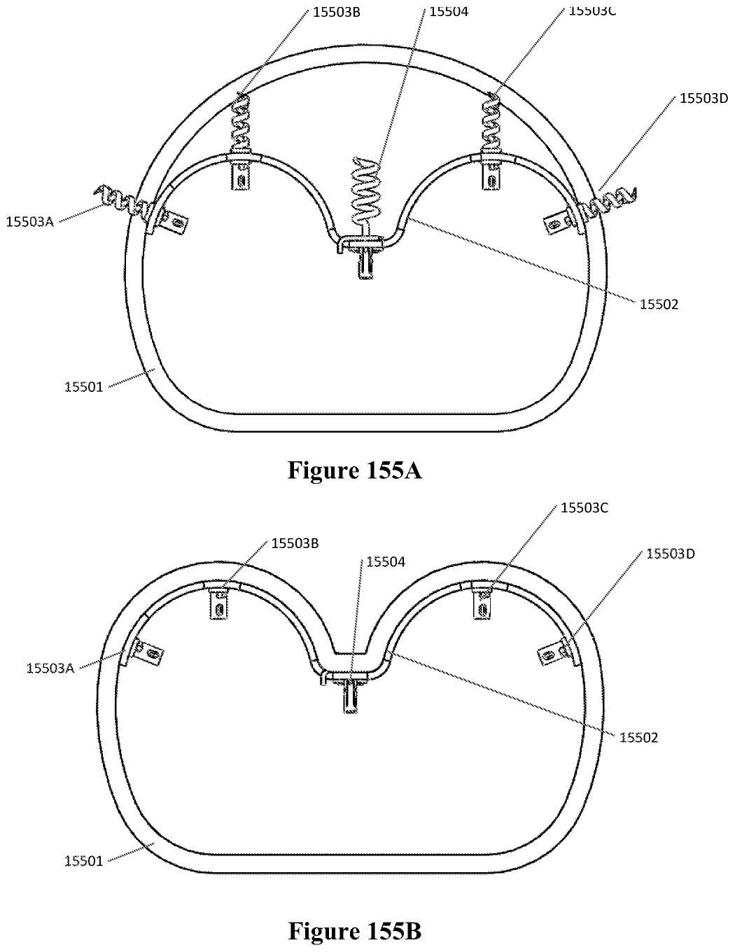

FIG. 155A shows a tissue shaping template 15502 in place above a valve annulus 15501, the template having center anchor 15504, inner side anchors 15503B and 15503C, and outer anchors 15503A and 15503D along a broadly curved shape. This template 15502 has a width that is comparable to the major axis of the valve annulus 15501, and may be constructed in a range of sizes to match the major axis of a range of valve annuli suitable for treatment. Further, the overall height of the template 15502 as measured from the center anchor 15504 coupling point to the inner side anchors 15503B and 15503C may be offered in various sizes even within a single major axis size to adjust the desired level of treatment of the target annulus. The outer anchors 15503A and 15503B and the center anchor 15504 are releasably coupled to torque tubes (not shown) and are placed prior to placement of the tissue shaping template 15502. The outer anchors 15503A and 15503B and the center anchor 15504 may be positioned relative to relevant dimensions of the tissue shaping template 15502 or the annular anatomy as needed to achieve the desired effect. Tissue shaping template 15502 is slidably coupled to the three torque tubes (not shown) which help guide the tissue shaping template 15502 into position adjacent the valve annulus 15501. The tissue shaping template 15502 is coupled to the two outer anchors 15503A and 15503B and the center anchor 15504, and the torque tubes (not shown) are released.

FIG. 155 B shows the tissue shaping template 15502 of FIG. 155A, having been coupled via the anchors 15503 A-D and 15564 to the valve annulus 15501. The re-shaping caused by this coupling brings the template 15502 closer to the opposite wall of the annulus 15501.

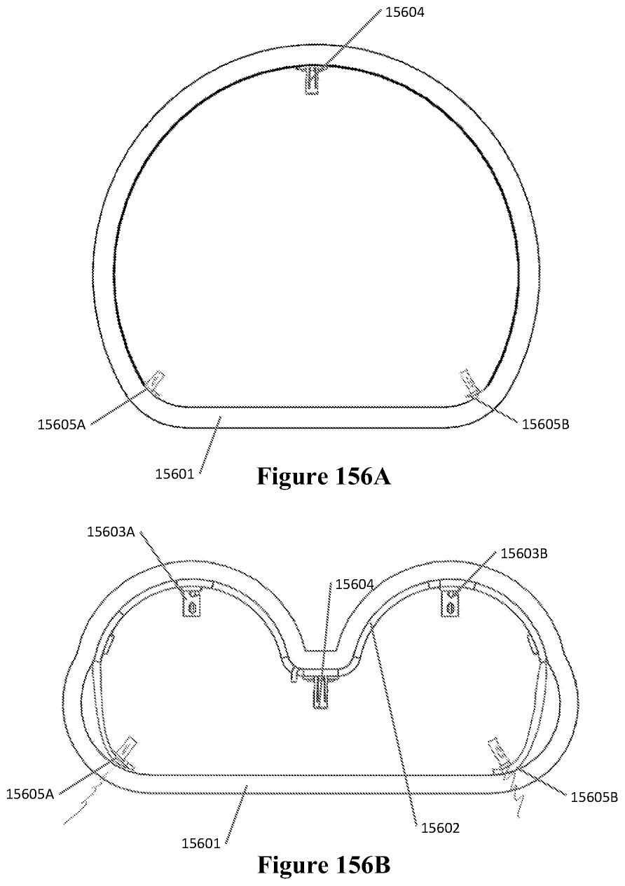

FIG. 156A shows a valve annulus 15601 with two trigonal anchors 15605A and 15605B and a center anchor 15604 in place along the annulus. The arc of the valve annulus from 15605A, through 15604, and along to 15605B define a semi-perimeter of the valve annulus between the trigonal anchors 15605A and 15605B.

FIG. 156B shows tissue shaping template 15602 coupled to a valve annulus 15601, the template having center anchor 15604, inner anchors 15603A and 15603B, and trigonal anchors 15605A and 15605B along a curved shape. The trigonal anchors 15605A and 15605B are coupled to tissue in the fibrous region of the fibrous trigones of the valve annulus 15601. The trigonal anchors 15605A and 15605B and the center anchor 15604 are releasably coupled to torque tubes (not shown) and are placed prior to placement of the tissue shaping template 15502. The trigonal anchors 15605A and 15605B and the center anchor 15604 may be positioned relative to relevant dimensions of the tissue shaping template 15602 or the annular anatomy as needed to achieve the desired effect. Tissue shaping template 15602 is slidably coupled to the three torque tubes (not shown) which help guide the tissue shaping template 15602 into position adjacent the valve annulus 15601. The tissue shaping template 15602 is coupled to the two trigonal anchors 15605A and 15605B and the center anchor 15604, and the torque tubes (not shown) are released. The height of the template 15602 from the coupling point of the center anchor 15604 to the coupling points of the trigonal anchors 15605A and 15605B is configured to give an appropriate therapeutic effect, and may range from 25% of the minor axis of the valve to 75% of the minor axis of the valve. The curves of the template 15602 from the attachment point for anchor 15605A through the attachment points for anchors 15603A, 15604, 15604B, and up to the attachment point for anchor 15605B define a length along the implant, which may be compared to the semi-perimeter of the annulus between the trigonal anchors 15605A and 15605B.

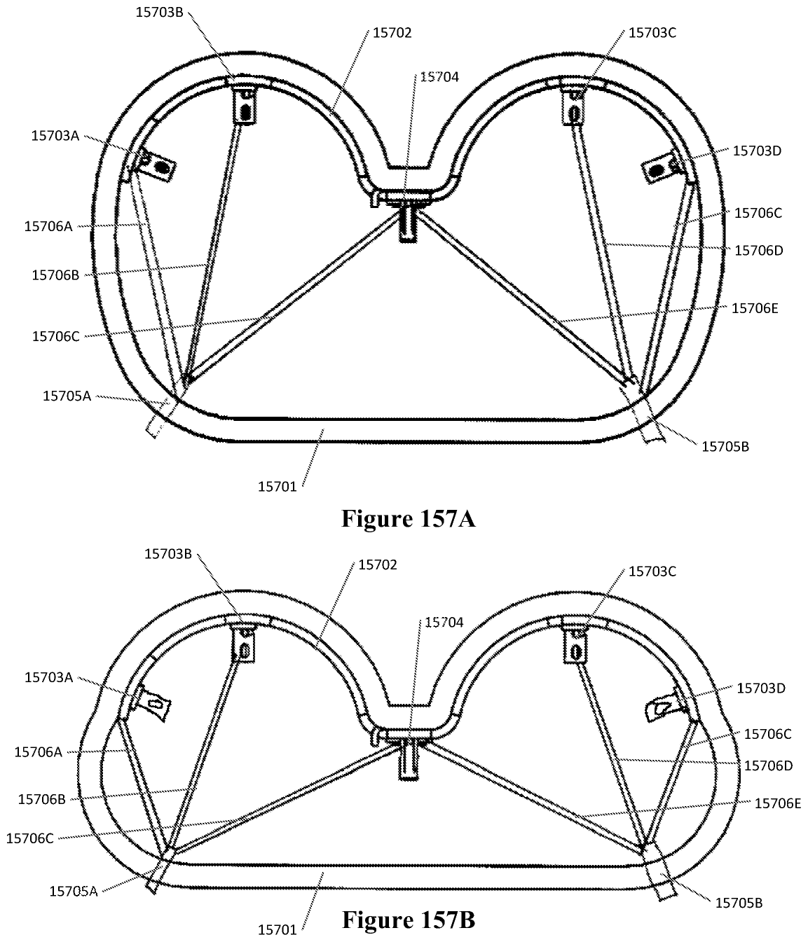

FIG. 157A shows tissue shaping template 15702 coupled to a valve annulus 15701, the template having center anchor 15704, inner anchors 15703A-D along a curved shape. The implant additionally comprises trigonal anchors 15705A and 15705B coupled to tissue in the fibrous region of the fibrous trigones of the valve annulus 15701. The trigonal anchors 15705A and 15705B are coupled to the template 15702 via one or more tension members 15706 A-E. These tension members 15706 A-E are adjustable to move the template 15702 closer to the trigonal anchors 15705A and 15705B.

FIG. 157 B shows the implant of FIG. 157A, the tension members 15706 A-E having been adjusted to move the template 15702 closer to the trigonal anchors 15705 A-B to achieve the desired therapeutic effect. This implant system could be configured with all, some, or one of the tension members 15706 A-E. For example, the desired therapeutic affect may be realized in an implant system where tension members 15706 A and 15706 B couple the trigonal anchors 15705 A-B to the outer anchors 15703A and 15703D alone. Similarly, the tension members (not shown) could couple the trigonal anchors 15705 A-B directly to the template 15702.

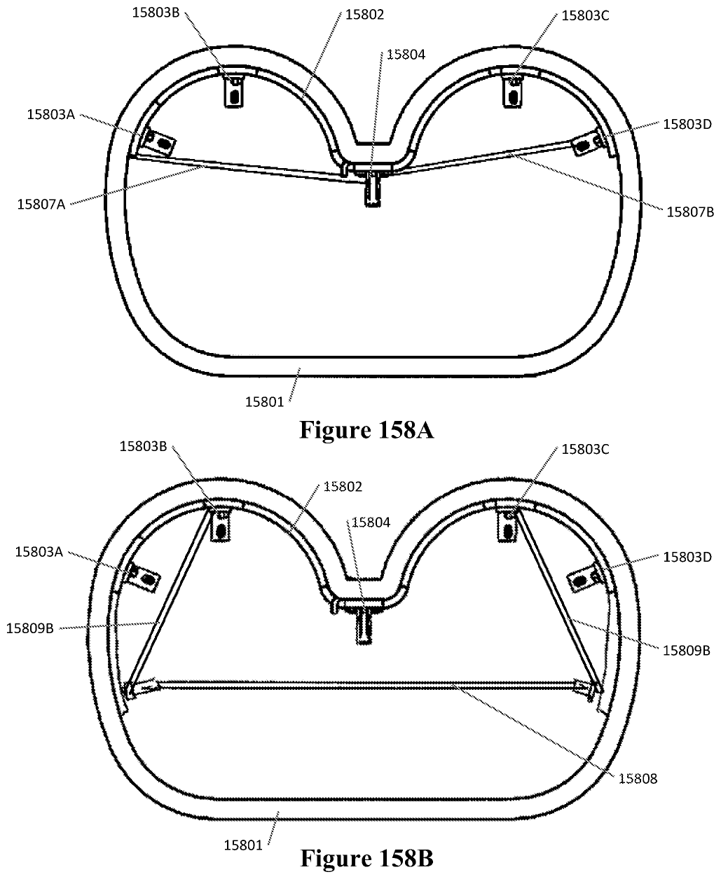

FIG. 158A shows a tissue shaping template 15802 coupled to a valve annulus 15801, the template having center anchor 15804, inner anchors 15803A-D along a curved shape. In addition, one or more tension members 15807 A-B couple different areas of the template 15802 to each other. As shown, the tension members 15807 A-B couple the middle of the template 15802 to either end of the template 15802. Adjusting the tension members 15807 A-B changes the shape of the template 15802 to achieve the desired therapeutic effect.

FIG. 158 B shows an alternate tissue shaping template 15802 coupled to a valve annulus 15801, the template having center anchor 15804, side anchors 15803A-D along a curved shape which extends past the outermost side anchors 15803A and 15803D. In addition, one or more tension members 15808A, 15808B, and 15809 couple different areas of the template 15802 to each other. As shown, the tension members 15808A and 15808B couple a mid-lateral portion of the template 15802 to either end of the template 15802, while tension member 15809 couples each end of the template 15802 together. Adjusting the tension members 15808A, 15808B, and 15809 changes the shape of the template 15802 to achieve the desired therapeutic effect.

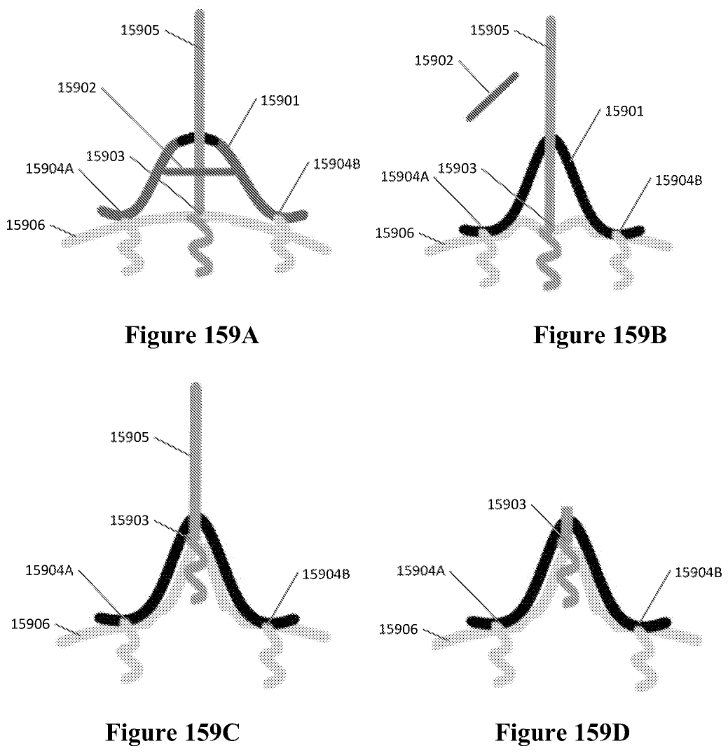

FIG. 159A shows a tissue shaping template 15901 having a stretching member 15902, a center anchor 15903 having a detachable control wire 15905, and two side anchors 15904A and 15904B which couple the template 15901 in the stretched configuration to the annulus 15906.

FIG. 159 B shows the tissue shaping template 15901 of FIG. 159A after the stretching member 15902 has been removed, allowing the two side anchors 15904A and 15904B to move closer together and creating a lower tension segment of the valve annulus 15906.

FIG. 159 C shows the tissue shaping template 15901 of FIG. 159 B, the center anchor 15903 having been coupled to the template 15901 by retracting anchor 15903 by applying tension to the control wire 15905. The tension required to retract anchor 15903 having been lowered by the creation of a low tension segment of the valve annulus 15906 as shown in FIG. 159 B.

FIG. 159 D shows the tissue shaping template 15901 of FIG. 159 C, the center anchor 15903 having been de-coupled from the control wire 15905.

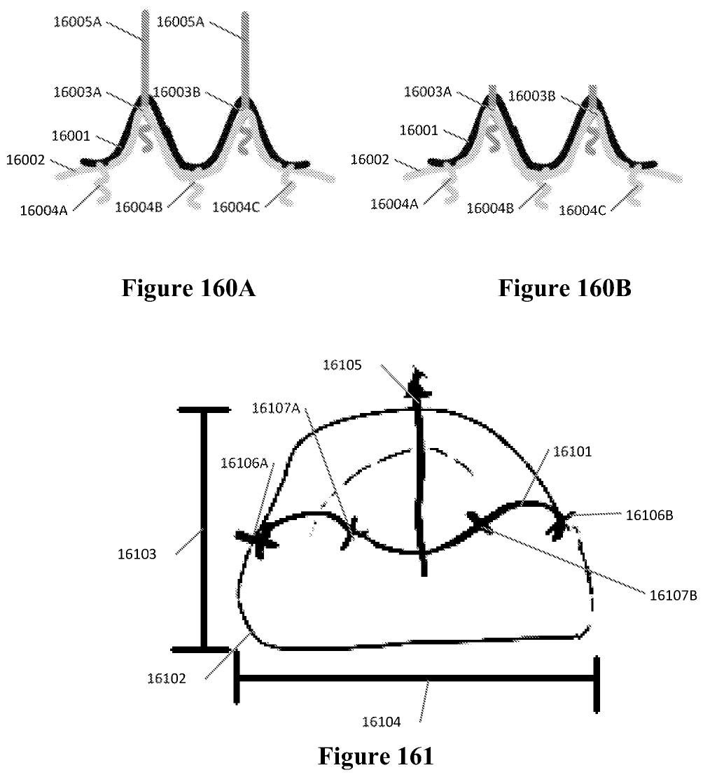

FIG. 160A shows a tissue shaping template 16001 having three convexity anchors 16004 A-C and two concavity anchors 16003 A-B, the concavity anchors each having releasable anchor control wires 16005A and 16005B. Once the concavity 16003 A-B anchors are coupled to the template, said concavity anchors 16003 A-B are decoupled from the anchor control wires 16005A and 16005B, and the anchor control wires 16005A and 16005B are removed. This tissue shaping template 16001 may also include one or more stretching members as shown in FIGS. 159 A-D, not shown in this figure.

FIG. 161 shows a tissue shaping template 16101 having one or more central anchors 16105, outer side anchors 16106A and 16106B coupled to a valve annulus 16102 at approximately the midpoint along the minor diameter dimension 16103 of the annulus 16102. The center anchor 16105 couples tissue to a concavity on the template 16101. The side anchors 16106A and 16106B may be placed either before or after the center anchor 16105. The template may have additional inner side anchors 16107A and 16107B to further stabilize the template 16101 on the annulus 16102.

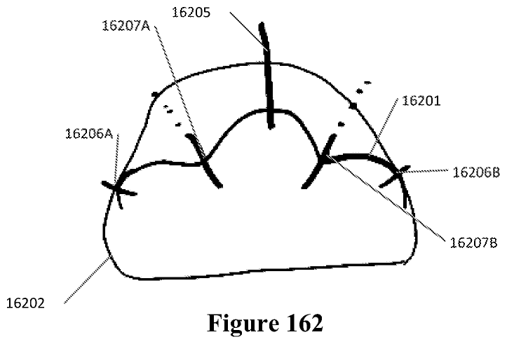

FIG. 162 shows a tissue shaping template 16201 having outer side anchors 16206A and 16206B coupled to a valve annulus 16202 at approximately the midpoint along the minor diameter dimension of the annulus 16202 and two or more inner side anchors 16207A and 16207B which couple tissue to corresponding concavities on the template 16201. The template may have one or more center anchors 16205 to further stabilize the template 16201 on the annulus 16202. The side anchors 16206A and 16206B may be placed either before or after the center anchor 16205 (if present), as well as before or after the inner side anchors 16207A and 16207B. It may be advantageous to place the side anchors 16206A and 16206B and center anchor 16205 before placing the inner side anchors 16207A and 16207B.

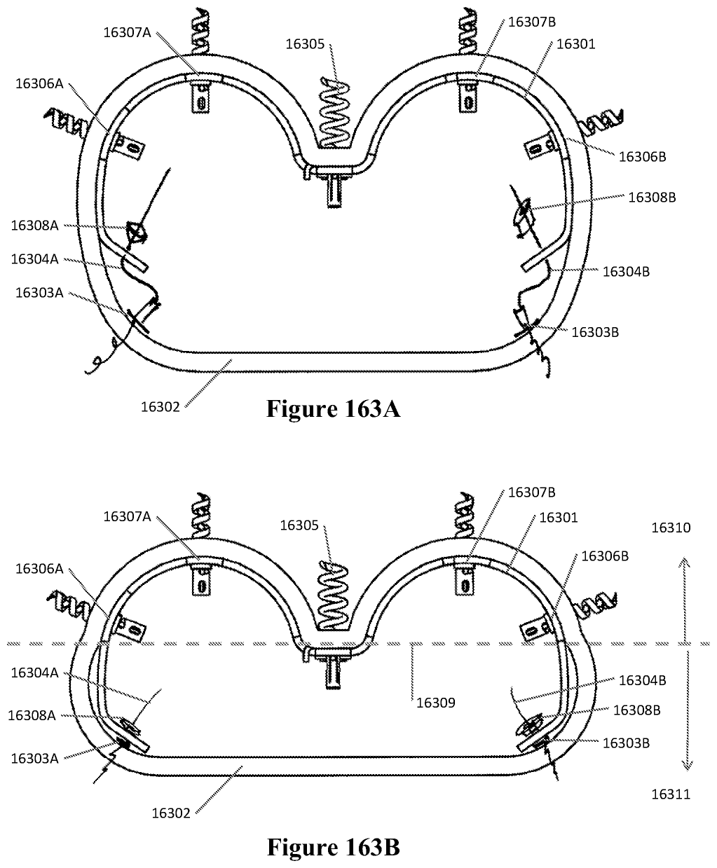

FIG. 163A shows a tissue shaping template 16301 coupled to one or more center anchors 16305, outside anchors 16306A-B, inside anchors 16307A-B, trigonal anchors 16303A-B, said trigonal anchors 16303A-B being coupled to the fibrous tissue in the trigon region of a valve annulus 16302. The trigonal anchors 16303A-B are releasably coupled to control wires 16304A-B which are in turn movably coupled to the tissue shaping template 16301 in a manner that allows the template 16301 to move closer to or farther from the trigonal anchors 16303A-B. Stop mechanisms 16308A-B are placed along the control wires 16304A-B and coupled to the respective trigonal anchor 16303A-B to limit the template 16301 motion in the direction away from the trigonal anchors 16303A-B. As shown in FIG. 163A, the trigonal anchors 16303A-B are not directly coupled to the template 16301, and a length of control wires 16304A-B runs between the trigonal anchors 16303A-B and the template 16301.

FIG. 163 B shows the tissue shaping template 16301, the trigonal anchors 16303A-B having been coupled to the template 16301 by applying tension to the control wires 16304A-B, and moving the stop mechanisms 16308A-B distally along the control wires 16304A-B into engagement with the trigonal anchors 16303A-B. At this point, the control wires 16304A-B may be decoupled from the trigonal anchors 16303A-B. Examples of suitable stop mechanism 16308A-B include threaded fasteners such as nuts, one way spring clips, ratchet and pawl devices, and other mechanisms known to the art. A dashed line 16309 through the point where the center anchor 16305 couples to the tissue shaping template 16301 and approximately perpendicular to the axis of the center anchor 16305 defines a direction 16310 which is distal relative to the implant and a second direction 16311 which is proximal relative to the implant. When referencing the valve anatomy, direction 16310 is posterior and 16311 is anterior relative to the valve anatomy.

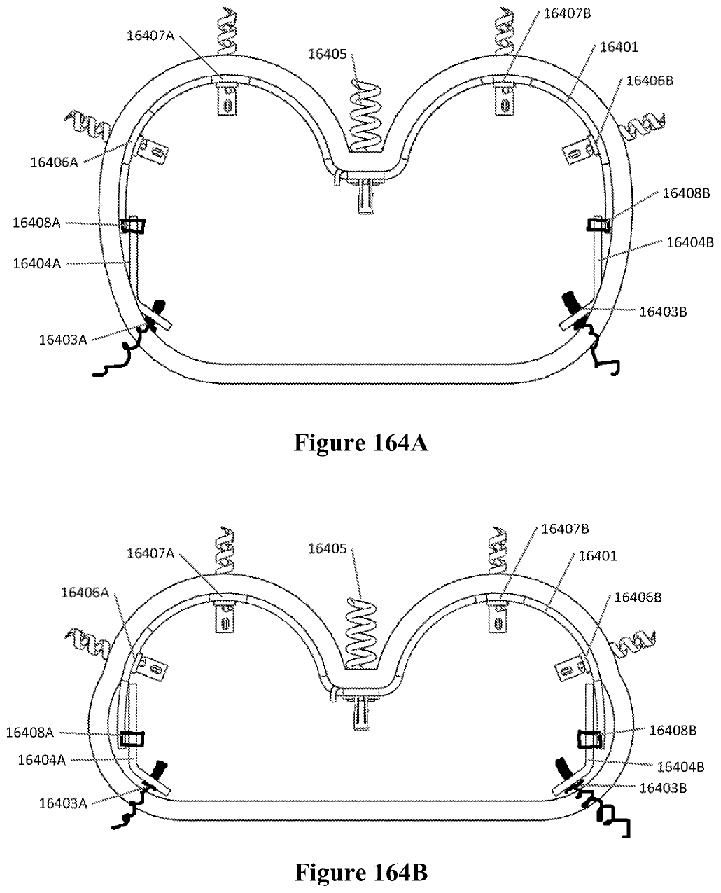

FIG. 164 A shows a tissue shaping template 16401 coupled to one or more center anchors 16405, outside anchors 16406A-B, and inside anchors 16407A-B, the template 16401 being coupled to extensions 16404A-B, and the extensions 16404A-B being coupled to trigonal anchors 16403A-B, said trigonal anchors 16403A-B being coupled to the fibrous tissue in the trigon region of a valve annulus 16402. The extensions 16404A-B are slidably coupled to the template 16401 via coupling mechanisms 16408A-B. As shown in FIG. 164A, the extensions 16404A-B are in the fully extended state, maximizing the distance between the template 16401 and the trigonal anchors 16403A and 16403B.

FIG. 164 B shows the tissue shaping template 16401, the extensions 16404A-B having been retracted, bringing the trigonal anchors 16403A and 16403B closer to the template 16401 to achieve the desired therapeutic effect.

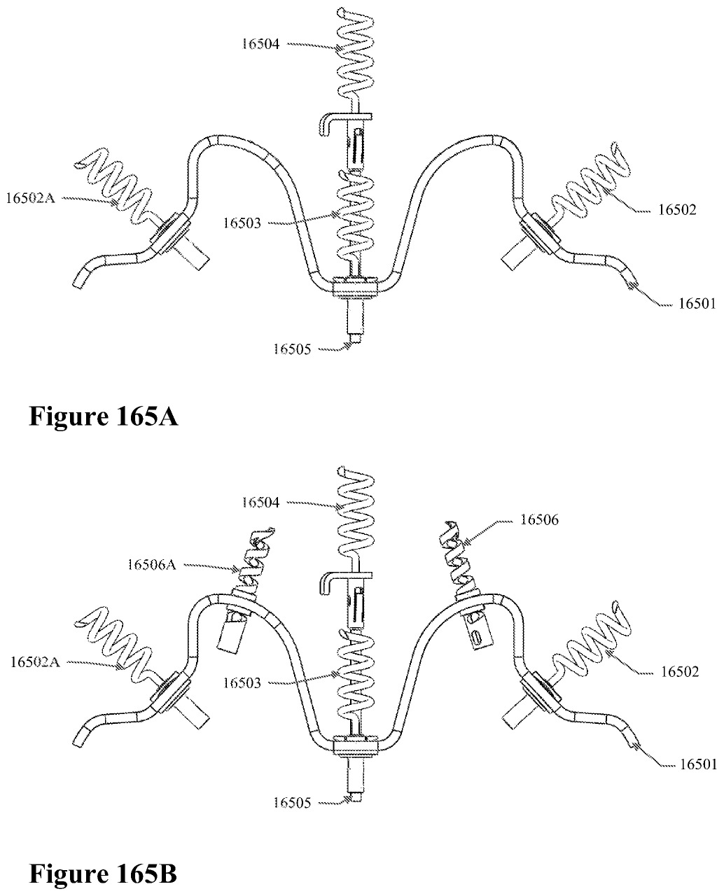

FIG. 165A shows a tissue shaping template 16501 having a central concavity with a center anchor 16504 and an accessory anchor 16503. The center anchor 16504 is releasably coupled to a torque tube 16505 that passes at least partially through a coupling feature on the template 16501. In addition, the template 16501 has two lateral concavities, each comprising a lateral accessory anchor 16502A and 16502B.

FIG. 165B shows the implant of FIG. 165A with the addition of mid-lateral stabilizing anchors 16506A and 16506B. The mid-lateral stabilizing anchors 16506 A-B are coupled to the template 16501 in the regions of the convexities which separate the central concavity from the lateral concavities.

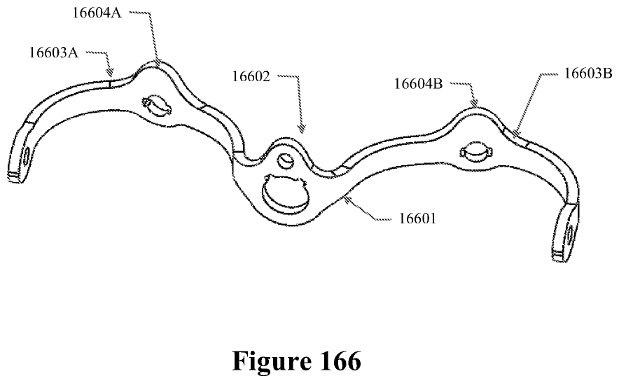

FIG. 166 shows a tissue shaping template 16601 having a concavity 16602 and two convexities 16603A and 16603B. Along each convexity is an atrial stabilizing protrusion 16604A and 16604B, which may help prevent pivoting of the implant relative to the valve annulus by interacting with the atrial wall of the heart.

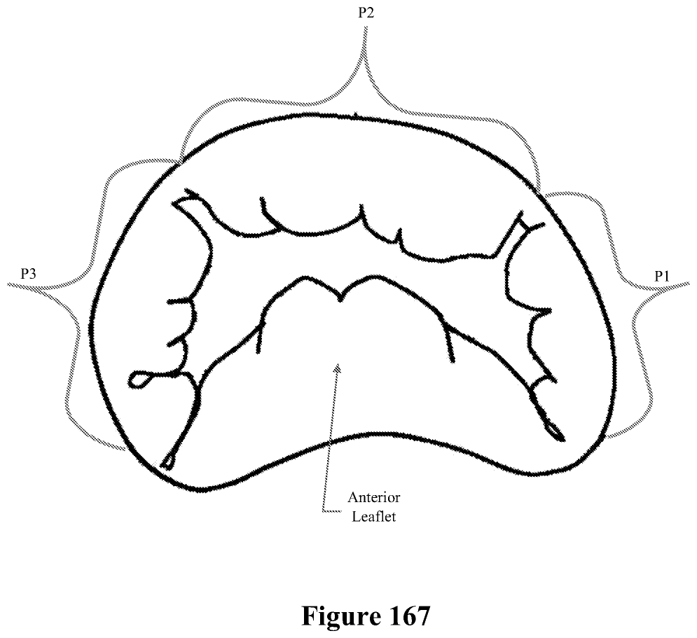

FIG. 167 shows a mitral annulus with three posterior leaflets (P3 on the left, P2 in the middle, and P1 on the right.) An anterior leaflet is labeled on the bottom. Each of the leaflets defines a proximate region of the valve annulus, and placing anchors in a designated proximate region of the valve annulus may confer certain benefits.

### Examples

In a preferred example, the template outline was laser cut from a 0.020″ thick sheet of superelastic nickel titanium to the desired flat shape, which was cleaned and polished by ultrasonic cleaning, manual polishing (including rounding sharp edges with a hand tool and chamfering the center hole on the distal side), and electro-polishing, then the flat was clamped into a shaping fixture made of heat resistant aluminum that held the flat shape in a configuration with a single concavity and two convexity or apex or convex segment regions, and the heat set assembly was heated to 485° C. for 4 minutes by submerging in a fluidized bed of aluminum oxide, then was rapidly quenched in a room temperature water bath to set the shape. The now preformed shape was removed from the shaping fixture, inspected, and guide coils for wire anchors were attached to each of the two convexities. The template then inspected, and partially covered with an ePTFE sleeve.

In a preferred example, the template outline was laser cut from a 0.66 mm thick 12 mm outer diameter tubing made from high fatigue cycle superelastic nickel titanium (Euroflex GmbH) to a slightly curvature shape, which was cleaned and polished by ultrasonic cleaning with Microclean BS (RBP Chemicals), and electro-polishing with Electropolish Ti2 (RBP Chemicals), then the laser cut component was clamped into a shaping fixture made of heat resistant steel that held the flat shape in a configuration with a single concavity and two convexity or apex or convex segment regions, and the heat set assembly was heated to 500° C. for 10 minutes by submerging in a molten salt bath, then was rapidly quenched in a chilled water bath to set the shape. The now preformed shape was removed from the shaping fixture, inspected, and guide coils for wire anchors were attached to each of the two convexities. The template then inspected, and partially covered with a knitted Dacron sleeve.

In a preferred example, a docking anchor was formed of 0.4 mm diameter stainless-steel wire into a helical coil 2.3 mm in diameter, having a pitch of 0.75 turns per millimeter of length. The proximal end of the coil was deflected toward the axis of the coil and was bent to lie along the axis of the coil, forming an axle section of the wire. A stainless-steel tube with an outer diameter of 0.6 mm and an inner diameter of 0.4 mm was crimped to the axle section of the wire 1 mm from the coil, and trimmed to 1.7 mm length. The crimped tube was then laser welded to a stainless-steel dock tube (outer diameter 1 mm, inner diameter 0.7 mm) having laser cut features including two extensible tabs, a key wire slot, a wire exit hole, and two holes for welding. A stainless-steel washer with 1 mm inner diameter and 2 mm outer diameter was welded to the distal end of the dock tube, and the completed docking anchor assembly was electro-polished. The distal end of the coil was then sharpened with two facets to a tissue-penetrating point.

In one example, a docking torque tube was made by laser cutting a pattern into a stainless-steel tube with an outer diameter of 0.6 mm and an inner diameter of 0.3 mm. The laser cut features included a spiral cut with a helix angle of 70 degrees and a height of 10 cm, starting 2.5 mm from the distal tip of the docking torque tube, a pair of holes with a diameter of approximately 0.2 mm separated from each other axially by a bridge 0.2 mm in length, and a notch in the distal end of the docking torque tube, on the opposite side of the tube from the pair of holes. The docking torque tube was then electro-polished and cleaned.

In another example, the docking anchor was placed on the distal tip of the docking torque tube so that the key wire slot on the anchor aligns with the two holes on the docking torque tube. A 0.15 mm diameter nickel titanium key wire was passed through the key wire slot in the anchor and the proximal most of the two holes and fed proximally through the docking torque tube until it exited the proximal end of the docking torque tube. The distal end of the key wire was then fed through the key wire slot in the anchor, through the distal most of the two holes, through the notch in the distal end of the docking torque tube, and out the wire exit hole of the anchor. The slack in the key wire was pulled out, and the distal end of the key wire was trimmed flush with the docking anchor.

In another example, a wire anchor was formed of 0.25 mm diameter super-elastic nickel titanium wire to a helical coil with an outer diameter of 1.2 mm and a pitch of 0.9 turns per millimeter in the distal section. The distal section had a length of 6 mm before transitioning to two closed coils, a single open coil (pitch 1 turn per millimeter), a single closed coil with the opposite handedness of the other coils, and a straight segment 2 mm long that runs parallel to the axis of the coil. The distal tip of the wire anchor was then sharpened with two facets to a tissue penetrating point.

In another example, a wire anchor was laser cut from a 2 mm outer diameter as drawn stainless-steel tubing to a helical coil with an outer diameter of 1.5 mm and a pitch of 0.8 turns per millimeter in the distal section. The distal section had a length of 8 mm. The distal tip of the anchor was then sharpened with three facets to a tissue penetrating point.

In one example, a wire torque tube was made by laser cutting a pattern into a stainless-steel tube with an outer diameter of 0.6 mm and an inner diameter of 0.3 mm. The laser cut features included a spiral cut with a helix angle of 70 degrees and a height of 10 cm, starting 2.5 mm from the distal tip of the docking torque tube, and a key wire slot. A stainless-steel lock tube with an outer diameter of 0.5 mm, an inner diameter of 0.3 mm, and a length of 2 mm was then welded to the outside of the wire torque tube, aligned with the axis of the wire torque tube, and such that the distal end of the lock tube was approximately 0.3 mm proximal to the proximal end of the key wire slot. The docking torque tube was then electro-polished and cleaned.

In another example, the wire anchor was placed on the distal end of the wire torque tube so that the key wire slot on the wire torque tube aligns with the two closed coils on the wire anchor, and the straight segment runs through the lock tube welded to the side of the wire torque tube. A 0.15 mm diameter nickel titanium key wire was passed proximal to the two closed coils on the wire anchor, through the key wire slot in the wire torque tube and fed proximally through the wire torque tube until it exited the proximal end of the wire torque tube. The distal end of the key wire was then fed distal to the two closed coils, through the key wire slot in the wire torque tube and out of the distal end of the wire torque tube. The slack in the key wire was pulled out, and the distal end of the key wire was trimmed flush with the distal end of the wire torque tube.

In a preferred example, the docking anchor coupled to a docking torque tube was placed through a steerable sheath and secured adjacent a mitral annulus. The steerable sheath was removed, and a template (gripped in the jaws of a delivery catheter and having wire anchors placed partially through guide coils at each end of the template) was placed over the docking torque tube. The wire torque tubes were tensioned to partially flatten the concavity of the template, and the template pushed distally relative to the anchor to engage the elongate tabs. The tabs compressed as the template pushed distally past them and extended again when the template had fully passed the tabs, effectively coupling the template to the anchor. The arms of the template were aligned by rotating the delivery catheter, and the wire torque tubes were advanced to fully engage the convexities of the template with the annular tissue. The wire torque tubes were rotated to couple the wire anchors to the annulus. The jaws of the delivery catheter were released, de-coupling the template from the delivery catheter. The key wires of the two wire anchors and the docking anchor were pulled, de-coupling the anchors from the torque tubes. The torque tubes and delivery catheter were withdrawn, leaving the template anchored in place adjacent the mitral annulus.

In one example, a wire-form template was manufactured from two lengths of 0.020″ drawn nickel titanium wire which was clamped into a shaping fixture made of heat resistant aluminum that held the wires in a configuration with a single concavity and two convexity or apex or convex segment regions, and the heat set assembly was heated to 485° C. for 4 minutes by submerging in a fluidized bed of aluminum oxide, then was rapidly quenched in a room temperature water bath to set the shape. The two shaped wires were then crimped to three stainless-steel bosses, the center one having a hole configured to mate with a docking anchor, and the side ones having threaded holes configured to mate with wire anchors. The assembly was inspected, then covered with an ePTFE sleeve.

In one example, a wire-form template was manufactured from two lengths of 0.040″ outer diameter and 0.031″ inner diameter drawn nickel titanium tubing which was clamped into a shaping fixture made of heat resistant aluminum that held the wires in a configuration with a single concavity and two convexity or apex or convex segment regions, and the heat set assembly was heated to 520° C. for 6 minutes in a vacuum furnace, then was rapidly quenched in a container filled with dry ice and water to set the shape. The two shaped tubes were then crimped to three stainless-steel bosses, the center one having a hole configured to mate with a docking anchor, and the side ones having threaded holes configured to mate with wire anchors. The assembly was inspected, then covered with an ePTFE sleeve.

In one example, three docking tissue anchors were releasably coupled to docking torque tubes, placed in the annular region of a mitral valve, and coupled to a partial annular ring cut from nickel titanium sheet, which had a receiving hole for each docking anchor. One of the receiving holes was an elongate slot to allow for some tolerance in the initial positioning of the anchors, and to secure the anchor in the slot, a washer with an inner diameter of 1 mm and an outer diameter of 2 mm was placed over the anchor dock to engage the extensible tabs so that they could not fall into the elongate slot. The stability of this partial ring, both in diametral dimensions and connection to the anchors and annulus was verified and judged appropriate for docking with the cage of an implanted replacement valve.

In one example, a first tissue shaping template was fastened to a mitral valve annulus, and the size of the annulus measured and evaluated. A second template was then placed adjacent to the first tissue shaping template, and the size of the annulus measured, and shown to have been further reduced.

In one example, two docking tissue anchors (each releasably coupled to a docking torque tube) were placed adjacent a valve annulus at a separation distance of approximately 15 mm. A flat template with mating holes 10 mm apart was placed over the docking torque tubes and docked to the anchors so that it ran substantially along the valve annulus. When the anchors were maintained in a substantially parallel alignment, the annulus was drawn together by approximately 5 mm. Additional flat templates placed adjacent the first had similar effect.

In an additional example, two docking anchors (each releasably coupled to a docking torque tube) were placed across a valve annulus, one at a fibrous trigone and the second near the middle of the posterior aspect of the annulus, having a separation distance of approximately 22 mm. A flat template with mating holes 15 mm apart was placed over the docking torque tubes and docked to the anchors so that it traversed the valve. When the anchors were maintained in a substantially parallel alignment, the annulus was drawn together by approximately 7 mm

In one example, three tissue anchors were coupled to tissue adjacent a valve annulus, one centered above the P2 leaflet of the valve, and each of the others adjacent the valve commissures. The three anchors were then connected by a braided suture. The suture was tensioned until the valve annulus shape was visibly changed.

In one example, a docking anchor was placed adjacent a mitral valve annulus in the region of the P2 leaflet of the valve, and approximately in plane with the valve annulus. A tissue shaping template was coupled to the anchor, reshaping the annulus substantially in the annular plane. When explanted after 60 days, tissue had healed around the tissue shaping template, coupling it to the leaflet, and effectively reinforcing the leaflet.

In another example, a docking anchor was placed adjacent a mitral valve annulus in the region of the P2 leaflet of the valve and an angle of approximately 60 degrees to the plane of the valve annulus. A tissue shaping template was coupled to the anchor, reshaping the annulus and raising the P2 region of the annulus substantially above the annular plane.

In one example, a tissue anchor was placed adjacent a valve annulus from the ventricular side of the valve through an arterial catheter placed through the aorta, into the left ventricle, and brought into approximation with the posterior leaflets of the mitral annulus. The tissue anchor comprised a stainless-steel helical coil and an anchor body laser cut from a tube, and was coupled to a flexible torque tube by a nickel titanium key wire. Rotating the torque tube from outside the body rotated the anchor, coupling it to the target tissue. A tissue shaping template was advanced over the torque tube and over extendible tabs in the anchor body until they compressed to allow passage of the template, and recovered to their extended position to prevent proximal motion of the template relative to the anchor. The key wire was pulled, decoupling the anchor from the torque tube, and the tube removed. In another example, the anchor was placed into the muscular wall of the ventricle, and the template coupled to the anchor as in the previous example.

In one example, a tissue shaping template was laser cut from sheet nickel titanium, heat set to shape, and covered with a woven fabric stretched over the arms and sutured in place. When placed in a valve annulus, this woven fabric will create a smoother surface for a potential future valve implant to seal against.

In one example, a tissue shaping template was laser cut from a nickel titanium tube, heat set to shape, and covered with a woven fabric stretched over the arms and sutured in place. When placed in a valve annulus, this woven fabric will create a smoother surface for a potential future valve implant to seal against.

In one example, a tissue shaping template was laser cut from sheet nickel titanium, heat set to shape, and covered with an electrospun PVDF-HF copolymer non-woven fabric on both sides, so that the two layers of electrospun material bonded to each other and to the template. This covering will encourage tissue ingrowth and hemostatic sealing against the valve.

In one example, a delivery catheter was constructed with a reinforced jacket, an internal Pebax extrusion, and stainless-steel tip with movable laser cut jaws holding a tissue template. Key wires held the jaws closed on the template during delivery. An anchor, releasably coupled to a torque tube, was delivered to tissue adjacent a heart annulus, the delivery catheter was placed over the proximal end of the torque tube, so that the torque tube exited a side port 2 inches from the distal end of the delivery catheter. In this way, control over the proximal end of the torque tube was maintained without the need for a torque tube long enough to exit the full length of the delivery catheter.

In one example, an anchor releasably coupled to a torque tube was delivered adjacent a mitral valve annulus through a steerable sheath traversing the inferior vena cava, entering the right atrium, crossing the septum and entering the left atrium. Tension was applied to the torque tube while the tissue was observed on 3D ultrasound. The tissue moved slightly, and the sheath advanced farther into the left atrium to meet the anchor in response to the tension. A stiffening member was placed in the sheath to stabilize it, and it was observed that the user could apply more tension to the torque tube and observe greater tissue motion than without the stiffening member.

In one example, a nickel titanium basket was laser cut from a tube and heat set to a diameter of approximately 2 cm. The basket was attached to a steerable sheath so that the distal end of the basket was 1.5 cm proximal to the distal end of the steerable sheath. A wire was attached to the proximal end of the basket, so that tensioning the wire would reduce the size of the basket for insertion and removal. The basket, when extended, would brace the sheath against the septum to reduce sheath motion into the left atrium when tension was applied to the torque tube.

In another example, an inflatable balloon 20 mm diameter was formed of a resilient polymer and 1.5 cm proximal to the distal end of the steerable sheath. A tube for inflating the balloon was run along the length of the catheter to a port on the catheter handle. The balloon, when inflated, would brace the sheath against the septum to reduce sheath motion into the left atrium when tension was applied to the torque tube.

In another example, an anchor releasably coupled to a torque tube was placed adjacent a valve annulus. An elongate tissue template analog was attached to the distal end of a support catheter and fed through a sheath and over the torque tube to contact the tissue on either side of the anchor. Tension on the anchor, balanced by forward pressure on the support catheter, deformed the valve annulus in a way analogous to placing a tissue shaping template coupled to the valve anchor. In this way, the system performance could be assessed prior to template delivery.

In one example, a tissue shaping template has 3 annulus coupling areas which define a plane in space. In a further example, the tissue shaping template lies along said plane for its entire length. In an alternate example, portions of the tissue shaping template lie to one side of this plane. In a further example, the non-planar aspects of the tissue shaping template allow it to lie along a non-planar valve annulus. In a further example, the non-planar shape is a partial saddle shape.

In one example, a sub-leaflet probe has a tip which can be visualized under a leaflet and is used to identify the line along which a valve leaflet couples to the wall of a heart chamber. In a further example, said wall is a ventricular wall. In a further example, a marking device is used to temporarily mark the line along which the valve leaflet couples to the ventricular wall to guide implant placement. In a further example, the marking device deposits ink where the tip of the sub-leaflet probe indicates the leaflet attachment is located. In a further example, the marking device is a pen containing ink. In another example, the marking device is a needle covered in ink. In another example, the marking device is a porous material soaked with ink. In an alternate example, the marking device applies suction to create a tissue mark without ink. In a further example, the marking device creates an abrasion to mark tissue without ink.

In one example, a tissue shaping template is coupled along its length to one or more tension members. In a preferred example, the tension members extend to one or more anchors coupled to tissue, and act to move the template closer to the anchors coupled in said tissue. In a further example, the one or more anchors are coupled in the fibrous skeleton of the heart. In a further example, the one or more anchors are coupled to the fibrous skeleton of the heart adjacent the fibrous trigones.

In another example, a tissue shaping template is coupled to a tension member at two or more points along the length of the template. In a further example the tension member alters the shape of the template. In a further example, the tension member is adjustable to alter the shape of the template to achieve a desired therapeutic effect. In a further example, the tension member is coupled adjacent opposing ends of the template. In another example, one or more tension members couple one or more ends of the template to a point near the middle of the template. In another example, one or more tension members couple one or more ends of the template to one or more mid-lateral points on the template. In another example, one or more tension members couple one or points near the middle of the template to one or more mid-lateral points on the template.

In one example, a tissue shaping template has a first and second end coupled to the fibrous skeleton of the heart adjacent the fibrous trigones. In a further example, said tissue shaping template comprises one or more concavities disposed between the first and second ends and coupled to the valve annulus by tissue anchors within the concavities. In a further example, said tissue shaping template comprises one or more stabilizing anchors disposed between the first and second ends outside of the one or more concavities.

In a further example, a central tissue anchor is releasably coupled to a torque tube and anchored in a target annulus. A tissue shaping template is slidably coupled to the torque tube and guided into place adjacent the target tissue, and further anchored to the target tissue via an accessory anchor disposed in close proximity to the central tissue anchor. The accessory anchor comprises a helical coil which is coupled to the template via an internal thread, and the accessory anchor further comprises a central axis coupled to the helical coil by a transition zone, so that the transition zone can pass through the internal threaded coupler, allowing the central axis to rotate within the internal thread coupling once the helical coil and transition zone have passed completely through the threaded coupling. Further rotation of the accessory anchor will draw the target tissue up against the template. Similar side accessory anchors are disposed at least partially within lateral concavities along the template. Stabilizing anchors at one or more convexities may also be used to further stabilize the template system.

While preferred examples of the present invention have been shown and described herein, it will be obvious to those skilled in the art that such examples are provided by way of example only. Numerous variations, changes, and substitutions will now occur to those skilled in the art without departing from the invention. It should be understood that various alternatives to the examples of the invention described herein may be employed in practicing the invention. It is intended that the following claims define the scope of the invention and that methods and structures within the scope of these claims and their equivalents be covered thereby.

1. An implant comprising:
an implant body configured for engagement against a tissue surface;
an anchor array support configured to be attached to the implant body; and
one or more tissue anchors operatively coupled to the anchor array support.

2.-183. (canceled)
